(19) United States
(12) Patent Application Publication
(10) Pub. No.: US 2006/0008815 A1
(43) Pub. Date: Jan. 12, 2006

(54) Compositions, methods, and systems for inferring canine breeds for genetic traits and verifying parentage of canine animals

(75) Inventors: David Rosenfeld, Sacramento, CA (US); Richard Kerr, Davis, CA (US); Michelle Hutton, Davis, CA (US); Sue DeNise, Davis, CA (US); Stephen Bates, Davis, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Metamorphix, Inc., Savage, MD (US)

(21) Appl. No.: 10/972,767

(22) Filed: Oct. 25, 2004

Related U.S. Application Data
(60) Provisional application No. 60/617,383, filed on Oct. 8, 2004.
(60) Provisional application No. 60/514,180, filed on Oct. 24, 2003.

(51) Int. Cl. C12Q 1/68

(52) U.S. Cl. 435/6

(57) ABSTRACT

Methods and systems are provided for managing companion animal subjects in order to maximize their individual health and potential performance and to maximize profits obtained in breeding and marketing the companion animal subjects. The methods and systems draw an inference of a phenotype for a genetic trait of a companion animal subject by determining the nucleotide occurrence of at least one companion animal SNP that is determined to be associated with the phenotype. The methods and systems described can be utilized to identify individual animals, determine or verify parentage of a single dog from any breed if the putative parent(s) are also available for testing, and are associated with, and predictive of, canine breeds. The inference is used in some aspects to diagnose a health condition or predisposition of a companion animal subject.

### CROSS REFERENCE TO RELATED APPLICATIONS

This application claims the benefit of priority under 35 U.S.C. § 119(e) of U.S. Ser. No. 60/524,180, filed Oct. 24, 2003, and U.S. Ser. No. 60/617,383, filed Oct. 8, 2004, the entire content of which are incorporated herein by reference.

### FIELD OF THE INVENTION

The invention relates generally to gene association analyses and more specifically to the use of single nucleotide polymorphisms as a determinant of trait identification, parentage identity and breed determination in companion animals.

### BACKGROUND INFORMATION

The generation of deep coverage, high quality, genomic information, and its associated application to gene discovery and polymorphic analysis, will create an unparalleled ability to manage animal health and nutrition through an entire lifetime. The use of better management of companion animal health throughout the life cycle will impact a number of criteria for such animals. These include but are not limited to: treatment selection; monitor effectiveness of therapy; focus more on preventative therapeutics rather than acute treatment; prediction of disease predisposition; earlier disease detection; disease characterization; create value at different points in animal health industry (vets, pharma, pet nutrition, registries ID plus) and creates lower costs for pet owners. Breed specific markers have been identified in bovine subjects (DeNise et al., 2003. U.S. patent application Ser. No. 10/750,622) and can be similarly applied to canine subjects.

Parentage and identity panels are the first applied technology of using genomic analysis to begin managing canine animals. For example, panels have been developed utilizing microsatellite marker panels (DeNise et al., 2004. Anim. Genetics. 35(1): 14-17; Halverson et al., 1995. U.S. Pat. No. 05,874,217; Ostrander et al., 1993. Genomics, 16: 207-213, Ostrander et al., 1995. Mammalian Genome, 6: 192-195; Franscisco et al., 1996. Mammalian Genome 7:359-362). In particular, parentage and identity panels can be used to:
1) assign or verify parentage in disputed cases or as a quality control check for breed certification. These panels are currently utilized by the American Kennel Club, Professional Kennel Club and the United Kennel Club for verifying parentage of a defined set of parents and progeny;
2) match and verify the identity of a lost or stolen animal. When combined with a database of genotypes and animals, the panel can be used to match unknown animals to itself, if a genotype has been previously recorded, or to parents and siblings;
3) verify the identity of a cloned animal or frozen or split and/or cloned embryo;
4) characterize tissues that may undergo intra- or inter-transplantation or propagation to other mammals;
5) verify the identity of banked and/or frozen semen, or verify cultured cell lines; and
6) link an animal, animal hair or animal biological samples to a crime scene for forensic applications.

Classification of individual dogs in a population has often relied on a priori groupings of individual animals on the basis of parentage and registration with a Breed Association for example, the American Kennel Club. If these criteria are not known or not available, animals can be classified as a member of a breed or combination of breeds based on phenotype or geographic location. For example, a dog with a long hair coat, pointed nose, white with black saddle hair color, with an ability to herd animals may be assumed to be of the Border Collie breed. These phenotypes such as size, coat color, coat length, ear length, head shape, body shape, sound of bark, etc. are readily observable by owners and breeders and are frequently used for the basis of breed classification with various degrees of success.

There are two possible options for classifying an individual canine animal into a population are. The first includes assigning an animal to a population based on known or assumed parentage, physical appearance, disposition or special ability. The second includes obtaining from a set of predefined populations (such as breeds) sample DNA from a number of members of each population to estimate allele frequencies in each population. Using the allele frequencies, it is possible to compute the likelihood a given genotype originated in each population and individuals can be assigned to population on the basis of these likelihoods (Parker et al., 2004. Science. 304:1160-1164; Pritchard, J. K., et al., Genetics 155: 945-959 (2000)).

Both strategies (above) rely on defining a set of populations. A classification based on phenotype or geographic locality may not accurately describe the genetic structure of a population if similar phenotypes can arise despite differences in genotype (Rosenberg, N. A., et al., Genetics. 159: 699-713 (2001); Parker, H. G. et al. Science. 1160-1164, (2004)).

To date, the only methods available to qualify animals for these systems are known or assumed parentage or phenotypic appearance. There is an opportunity to improve accuracy of individual animal qualification using the allele frequencies to compute the likelihood that a given genotype originated in specific breed population.

It is important to canine owners to know the breed from which a particular animal may arise because animals of the same breed have similar behavioral and predispositions to disease characteristics. For example, knowledge of breed composition is important to verify claims for breed of a canine animal when parentage is in dispute. Verification of claims for breed or breed composition has not been possible because no available technology could classify a canine animal to a particular population or infer the breed composition of an individual animal. Currently, the only canines accepted by breed are those where the records of individual animals are maintained by Breed Associations.

In addition, breed information is important for understanding the disposition and safety of canines prior to purchase. Canines within a breed often have common personality traits that can be utilized for matching animals to proper homes and understanding and providing the proper environment for breed types. In an extreme example of breed type behaviors, Pit Bulls and Wolf crosses are often banned from communities and may affect homeowners and liability insurance.

Further, lost and found searches for canine animals become more accurate when the exact breed is known. It allows animal shelters to screen animals and announce the results of the search to potential owners and to specific breed rescue groups.

Moreover, mixed breed groups are developing registries that will rely on technology to group animals by the percentage of their breeding, which may lead to new breed development. For example, Doodles (Poodle crosses) have become a popular breed type and this technology could lead to certification programs.

Finally, canines of specific breed types may have characteristics important in toxicology and research model studies. Breed verification and identification could ensure that the animals utilized in these studies will fit the experimental protocol.

Accordingly, there remains a need for methods and compositions that provide information regarding canine breed. For animals not registered with a breed association, information concerning breed can be useful to manage the health and nutrition of an animal. For example, Laborador Retrievers are prone to hip dysplasia; the symptoms can be reduced by adjusting the nutritional regiment of the young canine; owners could use a breed identity tool to determine appropriate preventative measures for ensuring the health of their dog.

### SUMMARY OF THE INVENTION

The present invention provides methods and systems for managing, selecting and breeding companion animals. These methods for identification and monitoring of key characteristics of individual animals and management of individual animals maximize their individual potential performance and health. The invention methods allow predictive (predisposition) diagnostics, nutritional therapies and veterinary pharmaceutical therapeutics as applied to companion animals. The methods of the invention provide systems to collect, record and store such data by individual animal identification so that it is usable to improve future animals bred by a breeder, for example, and managed by animal owners and breeders. The methods and systems of the present invention utilize information regarding genetic diversity among companion animals, particularly single nucleotide polymorphisms (SNPs), and the effect of nucleotide occurrences of SNPs on important genetic traits and determining the parentage, identity and breed of companion animals.

The present invention is based, in part, on the discovery of canine single nucleotide polymorphism (SNP) markers that can be utilized to identify individual animals; determine or verify parentage of a single dog from any breed if the putative parent(s) are also available for testing; and are associated with, and predictive of, canine breeds including, but not limited to: Afghan Hound, Basenji, Basset Hound, Beagle, Belgian Tervuren, Bernese Mountain Dog, Borzoi, Chihuahua, Chinese Shar-Pei, Cocker Spaniel, Dachshund, Doberman Pinscher, German Shepherd Dog, German Shorthaired Pointer, Golden Retriever, Labrador Retriever, Mastiff, Miniature Schnauzer, Poodle, Pug, Rottweiler, Saluki, Samoyed, Shetland Sheepdog, Siberian Husky, St. Bernard, Whippet, Yorkshire Terrier breeds. Accordingly, the present invention provides methods to discover and use single nucleotide polymorphisms (SNP) for identifying breed, or line and breed, or line composition of a canine subject. The present invention further provides specific nucleic acid sequences, SNPs, and SNP patterns that can be used for parentage, identity, breed identity and identifying breed or breed combinations to manage the health and well being of individual animals based on their breed composition.

Accordingly, in one embodiment the present invention provides a method to match an individual canine from a nucleic acid sample of the canine subject, that includes identifying in the nucleic acid sample, at least one nucleotide occurrence of at least one single nucleotide polymorphism (SNP) in any one of the nucleic acid sequences (SEQ ID NOs:1-101) encompassed by the GenBank Accession numbers provided in Table 1 or sequences listed in Table 8, under parentage and identity marker. The SNP is the last (most 3′) nucleotide listed in any one of SEQ ID NOs:1-101. The sequences containing the canine SNPs provided in Table 1 and Table 8 can be found on the world wide web at ftp://ftp.ncbi.nih.gov/snp/dog/XML. The contents of these files are encoded in XML, and contain the following information: SNP Id, Contig Name denoting the location of the SNP, and 60 bases of sequence flanking 5′ end of SNP, and the alleles comprising the SNP. The position of the SNP in the contig is determined by blasting the 5′ flanking sequence to the contig sequence. The location of the SNP is the base following the last matching base of the 60 bases. Contigs can be found on the world wide web at http://www.ncbi.nlm.nih.gov/entrez/query.fcgidb=Nucleotide&cmd=Search&term=AACN01000 0001:AACN011089636[PACC].

For example, the SNP can be identical to a genotype that is stored within a database of previously identified animals, or an archived nucleic acid or tissue sample of the subject can be identified with at least one SNP in any one of the markers listed in Table 1 or Table 8, thereby matching the canine subject to the archived sample. A SNP is matched to a canine subject when all nucleotide occurrences of the SNP occur in the archived sample and the canine subject. Therefore, in certain aspects, the methods include matching the identity of a subject using the nucleotide occurrence. The probability of matching can be statistically calculated based on the frequencies of nucleotides of each SNP.

Accordingly, in another embodiment the present invention provides a method to assign or verify the parentage of an individual canine from a nucleic acid sample of the canine subject, that includes identifying in the nucleic acid sample, at least one nucleotide occurrence of at least one single nucleotide polymorphism (SNP) in any one of markers listed in Table 1 or Table 8, under parentage and identity markers, wherein the SNP is consistent with the inheritance of the parental nucleotide. Potential parents are excluded from the parent list when the occurrence of the nucleotides in the nucleic acid sample of the canine subject is different than the potential parent in both nucleotides. These nucleotides can be compared through a database of previously identified animals, or an archived nucleic acid or tissue sample of the subject can be identified with at least one SNP in any one of the markers listed in Table 1 or Table 8, thereby matching the potential parents to the canine subject. Parents are verified or identified when all possible parents have been excluded from parentage except a single individual. Therefore, in certain aspects, the methods include matching the canine subject to potential parents using the nucleotide occurrence. The probability of exclusion can be statistically calculated based on the frequencies of nucleotides of each SNP.

In another embodiment, the present invention provides an isolated polynucleotide that includes a fragment of at least 20 contiguous nucleotides of any one of sequences associated with the accession numbers set forth in Table 1 or Table 8 (SEQ ID NOs:1-101), a polynucleotide at least 90% identical to the 20 contiguous nucleotide fragment, or a complement thereof. In certain aspects, the isolated polynucleotide, for example, includes a fragment of at least 5, 10, 15, 20, 25, 30, 35, 40, 45, 50, 100, 200, 250, 500, or 600 contiguous nucleotides of any one of Table 1 Table 8 sequences. In another aspect, the isolated polynucleotide is at least 65, 70, 75, 80, 85, 90, 95, 96, 97, 98, 99, or 99.5% identical to the sequences that correlate with the accession numbers set forth in Table 1 Table 8 (SEQ ID NOs:1-101), for example.

In another embodiment the present invention provides a method to infer breed of a canine subject from a nucleic acid sample of the canine subject, that includes identifying in the nucleic acid sample, at least one nucleotide occurrence of at least one single nucleotide polymorphism (SNP) in any one of markers listed in Table 1 or Table 8, breed specific markers, wherein the SNP is associated with a breed, thereby inferring the breed of the canine subject. A SNP is associated with a breed when at least one nucleotide occurrence of the SNP occurs more frequently in subjects of a particular breed than other breeds in a statistically significant manner, for example with greater than 50%, 55%, 60%, 65%, 70%, 75%, 80%, 85%, 90%, 95%, or 99% confidence. Therefore, in certain aspects, the methods include identifying whether the nucleotide occurrence is a canine SNP allele identified herein as associated with canine breed. In certain aspects, the identified breed includes, but is not limited to, Afghan Hound, Basenji, Basset Hound, Beagle, Belgian Tervuren, Bernese Mountain Dog, Borzoi, Chihuahua, Chinese Shar-Pei, Cocker Spaniel, Dachshund, Doberman Pinscher, German Shepherd Dog, German Shorthaired Pointer, Golden Retriever, Labrador Retriever, Mastiff, Miniature Schnauzer, Poodle, Pug, Rottweiler, Saluki, Samoyed, Shetland Sheepdog, Siberian Husky, St. Bernard, Whippet, Yorkshire Terrier.

In another embodiment, the present invention provides a method for determining a nucleotide occurrence of a single nucleotide polymorphism (SNP) in a canine sample, that includes contacting a nucleic acid obtained from the sample with an oligonucleotide that binds to a target region comprising any one of the sequences set forth in the GenBank Accession numbers provided in Table 1. The determination typically includes analyzing binding of the oligonucleotide, or detecting an amplification product generated using the oligonucleotide, thereby determining the nucleotide occurrence of the SNP.

In another embodiment, the present invention provides an isolated polynucleotide that includes a fragment of at least 20 contiguous nucleotides, a polynucleotide at least 90% identical to the fragment of 20 contiguous nucleotides, or a complement thereof, wherein the isolated polynucleotide includes a nucleotide occurrence of a single nucleotide polymorphism (SNP) associated with breed, wherein the SNP corresponds to the last nucleotide provided in any one of SEQ ID NOs:1-101.

As used herein, the term ‘companion animal’ refers to animals commonly domesticated by people and used as companionship pets. This could include, for example, dogs and cats, but otherwise may also include more exotic pets such as various fish, reptiles, birds, horses, rabbits, hamsters, gerbils, mice, rats and the like.

For example, the invention identifies animals that have superior genetic traits, predicted very accurately, that can be used to identify parents of the next generation through selection. These methods can be used to sort companion animals to determine performance for dog shows and breed club shows or for working dogs such as guide dogs, sheep dogs and police dogs. This invention provides a method for determining the optimum male and female parent to maximize the genetic components of dominance and epistasis thus maximizing heterosis and hybrid vigor in the animals.

In one aspect, the invention provides methods to draw an inference of a trait based on genotype of a companion animal subject by determining the nucleotide occurrence of at least one companion animal SNP that is determined using methods disclosed herein, to be associated with the trait. For example, the inference can be drawn regarding a health characteristic, for example, hip dysplasia (bone and joint health); diabetes; hypertension; atherosclerosis; autoimmune disorders; kidney disease and neurological disease. The invention is also useful for assessing complex traits such as energy metabolism; aging and breed-specific traits.

Methods of the present invention that relate to companion animal management, for example management in breeding, typically include managing at least one of food intake, diet composition, administration of feed additives or pharmacological treatments such as vaccines, antibiotics, age and weight at which diet changes or pharmacological treatments are imposed, days fed specific diets, castration, feeding methods and management, imposition of internal or external measurements and environment of the companion animal subject based on the inferred trait.

The inference is used in methods of the present invention for the following aspects of the invention: to improve profits related to selling a companion animal subject; to manage companion animal subjects; to sort companion animal subjects; to improve the genetics of a companion animal population by selecting and breeding of companion animal subjects; to clone a companion animal subject with a specific genetic trait, a combination of genetic traits, or a combination of SNP markers that predict a genetic trait; to track a companion animal subject or offspring; and to diagnose a health condition of a companion animal subject.

In another aspect, the present invention provides a method for identifying a companion animal genetic marker that influences a phenotype of a genetic trait. The method includes analyzing companion animal genetic markers for association with the genetic trait. Preferably, the method involves determining nucleotide occurrences of single nucleotide polymorphisms (SNPs). Preferably, nucleotide occurrences of at least two SNPs are identified that influence the genetic trait or a group of traits.

In another aspect, the present invention provides a high-throughput system for determining the nucleotide occurrences at a series of companion animal single nucleotide polymorphisms (SNPs). The system includes one of the following: solid support to which a series of oligonucleotides can be directly or indirectly attached, homogeneous assays and microfluidic devices. Each of these methods is used to determine the nucleotide occurrence of companion animal SNPs that are associated with a genetic trait.

In another aspect, the present invention provides a computer system that includes a database having records containing information regarding a series of companion animal single nucleotide polymorphisms (SNPs), and a user interface allowing a user to input nucleotide occurrences of the series of companion animal SNPs for a companion animal subject. The user interface can be used to query the database and display results of the query. The database can include records representing some or preferably all of the SNPs of a companion animal SNP map, preferably a high-density companion animal SNP map. The database can also include information regarding haplotypes and haplotype alleles from the SNPs. Furthermore, the database can include information regarding phenotypes and/or genetic traits that are associated with some or all of the SNPs and/or haplotypes. In these embodiments the computer system can be used, for example, for any of the aspects of the invention that infer a trait of a companion animal subject.

In one embodiment, a method for inferring a phenotype or genetic trait of a canine subject from a target nucleic acid sample of the subject is provided. The method includes identifying, in the nucleic acid sample, at least one nucleotide occurrence of a single nucleotide polymorphism (SNP) corresponding to the first nucleotide, or complement thereof, in the most 3′ position of any one of SEQ ID NOs:1-101. In some embodiments the nucleotide occurrence of at least 2 SNPs is determined. In other embodiments, the at least 2 SNPs provide a haplotype, thereby identifying a haplotype allele that is associated with the trait. In additional embodiments, a diploid pair of haplotype alleles are identified.

In another embodiment, a method for identifying a phenotype or genetic trait of a canine test subject is provided. The method includes obtaining a target nucleic acid sample from the test subject by a method that includes identifying in the nucleic acid sample at least one single nucleotide polymorphism (SNP) corresponding to the first nucleotide, or complement thereof, in the most 3′ position of any one of SEQ ID NOs:1-101. The identification can optionally be repeated for additional subjects. The method further includes determining the allele frequency corresponding to each SNP identified and comparing the allele frequency of the test subject with each additional subject.

In yet another embodiment, a kit for determining nucleotide occurrences of canine SNPs is provided. The kit includes an oligonucleotide probe, primer, or primer pair, or combinations thereof, for identifying the nucleotide occurrence of at least one canine single nucleotide polymorphism (SNP) corresponding to the first nucleotide, or complement thereof, in the most 3′ position of any one of SEQ ID NOs:1-101. The kit can include one or more detectable labels. The detectable label can be a non-extendible nucleotide. The non-extendible nucleotide can be a ddNTP that is fluorescently or chemically labeled, or labeled by biotinylation.

In yet another embodiment, a database including each single nucleotide polymorphism (SNP) corresponding to the first nucleotide, or complement thereof, in the most 3′ position of any one of SEQ ID NOs:1-101, is provided. Also provided is a database including allele frequencies generated by analyzing the aforementioned database of SNPs.

In one embodiment, a method for inferring a phenotype or genetic trait of a canine subject from a target nucleic acid sample of the subject is provided. The method includes identifying, in the nucleic acid sample, at least one nucleotide occurrence of a single nucleotide polymorphism (SNP) corresponding to the first nucleotide, or complement thereof, in the most 3′ position of any one of the sequences set forth in SEQ ID NOs:1-101 and associated with the GenBank Accession numbers of Table 1 and Table 8.

In yet another embodiment, a computer-based method for identifying or inferring a trait of a canine test subject is provided. The method includes obtaining a nucleic acid sample from the subject and identifying in the nucleic acid sample at least one nucleotide occurrence of at least one single nucleotide polymorphism (SNP) corresponding to the first nucleotide, or complement thereof, in the most 3′ position of any one of SEQ ID NOs:1-101. The method further includes searching a database comprising canine allele frequencies and retrieving the information from database. The method further includes optionally storing the information in a memory location associated with a user such that the information may be subsequently accessed and viewed by the user.

In one embodiment, a method for identifying or inferring a trait of a canine test subject from a nucleic acid sample obtained from the subject is provided. The method includes contacting the nucleic acid sample with a pair of oligonucleotides that comprise a primer pair, wherein amplified target nucleic acid molecules are produced. The further includes hybridizing at least one oligonucleotide primer selected from the group consisting of SEQ ID NOS:306-407 to one or more amplified target nucleic acid molecules, wherein each oligonucleotide primer is complementary to a specific and unique region of each target nucleic acid molecule such that the 3′ end of each primer is proximal to a specific and unique target nucleotide of interest. The method also includes extending each oligonucleotide with a template-dependent polymerase and determining the identity of each nucleotide of interest by determining, for each extension primer employed, the identity of the nucleotide proximal to the 3′ end of each primer. A primer pair includes any of the forward and reverse primer pairs listed in Table 7. For example, a first primer of the primer pair can be selected from SEQ ID NOS:102-203 and the second primer of the primer pair can be selected from SEQ ID NOS:204-305.

In another embodiment, an isolated oligonucleotide comprising any one of SEQ ID NOS:306-407, wherein each oligonucleotide further includes one additional nucleotide positioned proximal to the 3′ end of each oligonucleotide, and wherein the oligonucleotide specifically hybridizes to a nucleic acid sequence derived from a canine subject, is provided. Also provided are the complement of the aforementioned oligonucleotide.

In another embodiment, an isolated single nucleotide polymorphism (SNP) corresponding to the first nucleotide, or complement thereof, in the most 3′ position of any one of SEQ ID NOs:1-101, is provided. Oligonucleotides including the SNP corresponding to the first nucleotide, or complement thereof, in the most 3′ position of any one of SEQ ID NOs:1-101, are provided. The complement of these oligonucleotides are also provided.

In another embodiment, a panel comprising at least one single nucleotide polymorphism (SNP) corresponding to the first nucleotide, or complement thereof, in the most 3′ position of any one of SEQ ID NOs:1-101, is provided.

In another embodiment, a computer-based method for identifying or inferring a trait of a canine test subject is provided. The method includes obtaining a nucleic acid sample from the canine subject and identifying in the nucleic acid sample at least one nucleotide occurrence of at least one single nucleotide polymorphism (SNP) corresponding to the first nucleotide, or complement thereof, in the most 3′ position of any one of SEQ ID NOs:1-101. The method further includes searching a database comprising a plurality of single nucleotide polymorphism (SNP) markers selected from at least two of the SNP markers at the 3′ position to any one of SEQ ID NOs:1-101, wherein the database is generated from a nucleic acid sample obtained from a canine non-test subject. The method also includes retrieving the information from the database and optionally storing the information in a memory location associated with a user such that the information may be subsequently accessed and viewed by the user.

In another embodiment, a method for identifying the parentage of a canine test subject is provided. The method includes obtaining a nucleic acid sample from the test subject and identifying in the nucleic acid sample at least one single nucleotide polymorphism (SNP) corresponding to the first nucleotide, or complement thereof, in the most 3′ position of any one of SEQ ID NOs:1-101. The method further includes determining the alleles corresponding to each SNP identified and comparing the alleles to putative parents of the test subjects. The parents not possessing at least one allele in common with the test subject is excluded.

In another embodiment, a method to infer breed or line of a canine test subject from a nucleic acid sample obtained from the subject is provided. The method includes identifying in the nucleic acid sample, at least one nucleotide occurrence of at least one single nucleotide polymorphism (SNP) corresponding to the first nucleotide, or complement thereof, in the most 3′ position of any one of SEQ ID NOs:1-101.

In yet another embodiment, a method of generating a genome discovery map is provided. The method includes selecting a plurality of single nucleotide polymorphism (SNP) markers selected from at least two of the SNP markers corresponding to the first nucleotide, or complement thereof, in the most 3′ position of any one of SEQ ID NOs:1-101, wherein each marker in the series will be separated by approximately 150,000 bp and generating the genome discovery map based upon the selected markers. The discovery map can be a whole genome discovery map. The plurality of single nucleotide polymorphism (SNP) markers can include about 10, 100, 1000, 5000 or about 10000 markers. The plurality of single nucleotide polymorphism (SNP) markers, or the number of markers indicated by the amount of linkage disequilibrium in a canine species, can further be selected based upon their dispersion across the entire genome.

### DETAILED DESCRIPTION OF THE INVENTION

The methods of the invention are particularly well suited for predictive diagnostics, novel therapeutics, nutritional therapies and breeding genetic information of companion animal subjects. The methods allow for the ability to identify and monitor key characteristics of individual animals and manage those individual animals to maximize their individual potential health and breeding characteristics. Furthermore, the methods of the inventions provide systems to collect, record and store such data by individual animal identification so that it is usable to improve future animals bred. Specific embodiments of the invention are exemplified in Exhibit A, as provided in U.S. Provisional Ser. No. 60/524,180, filed Oct. 24, 2003 and incorporated herein by reference.

Accordingly, a method according to this aspect of the invention includes inferring a trait of the companion animal subject, such as a canine subject, from a nucleic acid sample of the subject. The inference is drawn by a method that includes identifying in the sample, a nucleotide occurrence for at least one single nucleotide polymorphism (SNP), wherein the nucleotide occurrence is associated with the genetic trait; and wherein the genetic trait affects the physical characteristic. Furthermore, the method includes managing at least one of food intake, diet composition, administration of feed additives or pharmacological treatments such as vaccines, antibiotics, age and weight at which diet changes or pharmacological treatments are imposed, days fed specific diets, castration, feeding methods and management, imposition of internal or external measurements and environment of the companion animal subject based on the inferred trait. This management results in a maximization of physical characteristics of a companion animal subject.

The method includes identification of the causative mutation influencing the trait directly or the determination of one or more SNPs that are in linkage disequilibrium with the associated genetic trait.

Preferably, the method includes a determination of the nucleotide occurrence of at least two SNPs. More preferably that at least two SNPs form all or a portion of a haplotype, wherein the method identifies a haplotype allele that is in linkage disequilibrium and thus associated with the genetic trait. Furthermore, the method can include identifying a diploid pair of haplotype alleles.

A method according to this aspect of the invention can further include using traditional factors affecting the economic value of the companion animal subject in combination with the inference based on nucleotide occurrence data to determine the economic value of the companion animal subject.

As used herein, the term ‘at least one’, when used in reference to a gene, SNP, haplotype, or the like, means 1, 2, 3, 4, 5, 6, 7, 8, 9, 10, etc., up to and including all of the haplotype alleles, genes, and/or SNPs of the companion animal genome. Reference to ‘at least a second’ gene, SNP, or the like, means two or more, i.e., 2, 3, 4, 5, 6, 7, 8, 9, 10, etc., companion animal genes, SNPs, or the like.

Polymorphisms are allelic variants that occur in a population that can be a single nucleotide difference present at a locus, or can be an insertion or deletion of one, a few or many consecutive nucleotides. As such, a single nucleotide polymorphism (SNP) is characterized by the presence in a population of one or two, three or four nucleotides (i.e., adenosine, cytosine, guanosine or thymidine), typically less than all four nucleotides, at a particular locus in a genome such as the human genome. It will be recognized that, while the methods of the invention are exemplified primarily by the detection of SNPs, the disclosed methods or others known in the art similarly can be used to identify other types of canine polymorphisms, which typically involve more than one nucleotide. A SNP is associated with a breed when at least one nucleotide occurrence of the SNP occurs more frequently in subjects of a particular breed in a statistically significant manner, for example with greater than 80%, 85%, 90%, 95%, or 99% confidence. A canine “SNP allele” is a nucleotide occurrence of a SNP within a population of canine animals.

The term ‘haplotypes’ as used herein refers to groupings of two or more SNPs that are physically present on the same chromosome which tend to be inherited together except when recombination occurs. The haplotype provides information regarding an allele of the gene, regulatory regions or other genetic sequences affecting a genetic trait. The linkage disequilibrium and, thus, association of a SNP or a haplotype allele(s) and a companion animal genetic trait can be strong enough to be detected using simple genetic approaches, or can require more sophisticated statistical approaches to be identified.

Numerous methods for identifying haplotype alleles in nucleic acid samples are known in the art. In general, nucleic acid occurrences for the individual SNPs are determined, and then combined to identify haplotype alleles. The Stephens and Donnelly algorithm (Am. J. Hum. Genet. 68:978-989, 2001, which is incorporated herein by reference) can be applied to the data generated regarding individual nucleotide occurrences in SNP markers of the subject, in order to determine alleles for each haplotype in a subject's genotype. Other methods can be used to determine alleles for each haplotype in the subject's genotype, for example Clarks algorithm, and an EM algorithm described by Raymond and Rousset (Raymond et al. 1994. GenePop. Ver 3.0. Institut des Siences de l'Evolution. Universite de Montpellier, France. 1994).

As used herein, the term ‘infer’ or ‘inferring’, when used in reference to a phenotype of a genetic trait, means drawing a conclusion about a trait or phenotype using a process of analyzing individually or in combination, nucleotide occurrence(s) of one or more SNP(s), which can be part of one or more haplotypes, in a nucleic acid sample of the subject, and comparing the individual or combination of nucleotide occurrence(s) of the SNP(s) to known relationships of nucleotide occurrence(s) of the SNP(s) and the phenotype. As disclosed herein, the nucleotide occurrence(s) can be identified directly by examining nucleic acid molecules, or indirectly by examining a polypeptide encoded by a particular gene where the polymorphism is associated with an amino acid change in the encoded polypeptide.

A ‘trait’ is a characteristic of an organism that manifests itself in a phenotype. Many traits are the result of the expression of a single gene, but some are polygenic (i.e., result from simultaneous expression of more than one gene). A ‘phenotype’ is an outward appearance or other visible characteristic of an organism. As used herein, a phenotype and a trait may be used interchangeably in some instances.

Methods of the present invention can be used to infer more than one trait. For example a method of the present invention can be used to infer a series of traits. Accordingly, a method of the present invention can infer, for example, coat quality/texture/color; bone/joint health, or predisposition to obesity. This inference can be made using one SNP or a series of SNPs. Thus, a single SNP can be used to infer multiple traits; multiple SNPs can be used to infer multiple traits; or a single SNP can be used to infer a single trait.

Relationships between nucleotide occurrences of one or more SNPs or haplotypes and a breed can be identified using known statistical methods. A statistical analysis result which shows an association of one or more SNPs or haplotypes with a breed with at least 80%, 85%, 90%, 95%, or 99% confidence, or alternatively a probability of insignificance less than 0.05, can be used to identify SNPs and haplotypes. These statistical tools may test for significance related to a null hypothesis that an on-test SNP allele or haplotype allele is not significantly different between groups with different traits. If the significance of this difference is low, it suggests the allele is not related to a breed. Statistical significance can be determined in both Bayesian and Frequentist ways.

As another example, associations between nucleotide occurrences of one or more SNPs or haplotypes and a phenotype (i.e. selection of significant markers) can be identified using a two part analysis in the first part, DNA from animals at the extremes of a genetic trait are pooled, and the allele frequency of one or more SNPs or haplotypes for each tail of the distribution is estimated. Alleles of SNPs and/or haplotypes that are apparently associated with extremes of a genetic trait are identified and are used to construct a candidate SNP and/or haplotype set. Statistical cut-offs are set relatively low to assure that significant SNPs and/or haplotypes are not overlooked during the first part of the method.

During the second stage, individual animals are genotyped for the candidate SNP and/or haplotype set. The second stage is set up to account for as much of the genetic variation as possible in a specific trait without introducing substantial error. This is a balancing act of the prediction process. Some animals are predicted with high accuracy and others with low accuracy.

In diploid organisms such as canines, somatic cells, which are diploid, include two alleles for each single-locus haplotype. As such, in some cases, the two alleles of a haplotype are referred to herein as a genotype or as a diploid pair, and the analysis of somatic cells, typically identifies the alleles for each copy of the haplotype. Methods of the present invention can include identifying a diploid pair of haplotype alleles. These alleles can be identical (homozygous) or can be different (heterozygous). Haplotypes that extend over multiple loci on the same chromosome include up to 2 to the Nth power alleles where N is the number of loci. It is beneficial to express polymorphisms in terms of multi-locus (i.e. multi SNP) haplotypes because haplotypes offer enhanced statistical power for genetic association studies. Multi-locus haplotypes can be precisely determined from diploid pairs when the diploid pairs include 0 or 1 heterozygous pairs, and N or N-1 homozygous pairs. When multi-locus haplotypes cannot be precisely determined, they can sometimes be inferred by statistical methods. Methods of the invention can include identifying multi-locus haplotypes, either precisely determined, or inferred.

A sample useful for practicing a method of the invention can be any biological sample of a subject, for example a canine subject, that contains nucleic acid molecules, including portions of the gene sequences to be examined, or corresponding encoded polypeptides, depending on the particular method. As such, the sample can be a cell, tissue or organ sample, or can be a sample of a biological material such as blood, milk, semen, saliva, hair, tissue, and the like. A nucleic acid sample useful for practicing a method of the invention can be deoxyribonucleic (DNA) acid or ribonucleic acids (RNA). The nucleic acid sample generally is a deoxyribonucleic acid sample, particularly genomic DNA or an amplification product thereof. However, where heteronuclear ribonucleic acid which includes unspliced mRNA precursor RNA molecules and non-coding regulatory molecules such as RNA is available, a cDNA or amplification product thereof can be used.

Where each of the SNPs of the haplotype is present in a coding region of a gene(s), the nucleic acid sample can be DNA or RNA, or products derived therefrom, for example, amplification products. Furthermore, while the methods of the invention generally are exemplified with respect to a nucleic acid sample, it will be recognized that particular haplotype alleles can be in coding regions of a gene and can result in polypeptides containing different amino acids at the positions corresponding to the SNPs due to non-degenerate codon changes. As such, in another aspect, the methods of the invention can be practiced using a sample containing polypeptides of the subject.

In one embodiment, DNA samples are collected and stored in a retrievable barcode system, either automated or manual, that ties to a database. Collection practices include systems for collecting tissue, hair, mouth cells or blood samples from individual animals at the same time that ear tags, electronic identification or other devices are attached or implanted into the animal. Tissue collection devices can be integrated into the tool used for placing the ear tag. Body fluid samples are collected and can be stored on a membrane bound system. All methods could be automatically uploaded into a primary database.

The sample can then be sent to a laboratory where a high-throughput genotyping system is used to analyze the sample. Genetic traits are predicted in the laboratory and forwarded electronically to a breeder, for example. The breeder then uses this information to sort and manage animals to maximize profitability and marketing potential. The information is also useful to a veterinarian, for example, to diagnose or treat a condition associated with a particular breed of companion animal. An exemplary subject of the present invention can be any canine subject, for example a sire, dam, pup, or any canine embryo or tissue. Nevertheless, the methods described herein are applicable to identify traits or breed of any companion animal subject, such as a dog, cat, horse, rabbit, fish, bird, reptile and the like. Thus, the present invention can also be used to provide information to breeders to make breeding, mating, and or cloning decisions. This invention can also be combined with traditional genetic evaluation methods to improve selection, mating, or cloning strategies associated with companion animals.

In another aspect, the present invention provides a method for improving profits related to breeding a companion animal subject. The method includes drawing an inference regarding a trait of the companion animal subject from a nucleic acid sample of the companion animal subject. The method is typically performed by a method that includes identifying a nucleotide occurrence for at least one single nucleotide polymorphism (SNP), wherein the nucleotide occurrence is associated with the genetic trait, and wherein the genetic trait affects the value of the animal or its products.

In one example, the present invention provides a system for determining the nucleotide occurrences in a population of canine single nucleotide polymorphisms (SNPs). The system typically includes a hybridization medium and/or substrate that includes at least two oligonucleotides of the present invention, or oligonucleotides used in the methods of the present invention. The hybridization medium and/or substrate are used to determine the nucleotide occurrence of canine SNPs that are associated with breed. Accordingly, the oligonucleotides are used to determine the nucleotide occurrence of canine SNPs that are associated with a breed. The determination can be made by selecting oligonucleotides that bind at or near a genomic location of each SNP of the series of canine SNPs. The system of the present invention typically includes a reagent handling mechanism that can be used to apply a reagent, typically a liquid, to the solid support. The binding of an oligonucleotide of the series of oligonucleotides to a polynucleotide isolated from a genome can be affected by the nucleotide occurrence of the SNP. The system can include a mechanism effective for moving a solid support and a detection mechanism. The detection method detects binding or tagging of the oligonucleotides.

Methods according to this aspect of the present invention can utilize a bioeconomic model, such as a model that estimates the net value of one or more companion animal subjects based on one or more phenotypes. By this method, phenotypes of one, or preferably a series of genetic traits are inferred. The model is typically a computer model. Values for the companion animal subjects can be used to segregate the animals. Furthermore, various parameters that can be controlled during maintenance and growth of the companion animal subjects can be input into the model in order to affect the way the animals are raised in order to obtain maximum health for the companion animal subject.

In another aspect, the present invention provides methods that allow effective measurement and sorting of animals individually, accurate and complete record keeping of genotypes and phenotypes or characteristics for each animal, and production of an economic end point determination for each animal using growth performance data. Accordingly, the present invention provides a method for sorting companion animal subjects. The method includes inferring a phenotype of a genetic trait for both a first companion animal subject and a second companion animal subject from a nucleic acid sample of the first companion animal subject and the second companion animal subject. The inference is made by a method that includes identifying the nucleotide occurrence of at least one single nucleotide polymorphism (SNP), wherein the nucleotide occurrence is associated with the genetic trait. The method further includes sorting the first companion animal subject and the second companion animal subject based on the inferred phenotype.

The method can further include measuring a physical characteristic of the first companion animal subject and the second companion animal subject, and sorting the first companion animal subject and the second companion animal subject based on both the inferred phenotype and the measured physical characteristic. The physical characteristic can be, for example, weight, breed; type or frame size, and can be measured using many methods known in the art, such as by using ultrasound. Sorting companion animals based on predicted phenotype allows selected companion animals to be chosen for programs such as guide dogs, police dogs and for dog and breed club shows.

In another aspect, the present invention provides methods that use analysis of companion animal genetic variation to improve the genetics of the population to produce animals with consistent desirable characteristics. Accordingly, in one aspect the present invention provides a method for selection and breeding of companion animal subjects for a genetic trait. The method includes inferring a phenotype of the genetic trait of a group of companion animal candidates for use in breeding programs from a nucleic acid sample of the companion animal candidates. The inference is made by a method that includes identifying the nucleotide occurrence of at least one single nucleotide polymorphism (SNP), wherein the nucleotide occurrence is associated with the phenotype. Individuals are then selected from the group of candidates with a desired phenotype for the genetic trait for use in breeding programs.

In another aspect the present invention provides a method for cloning a companion animal subject with a specific genetic trait or series of traits. The method includes identifying nucleotide occurrences of at least two SNPs for the companion animal subject, isolating a progenitor cell from the companion animal subject, and generating a cloned companion animal from the progenitor cell. The method can further include before identifying the nucleotide occurrences, identifying the phenotype of the companion animal subject, wherein the companion animal subject has a desired phenotype for a genetic trait and wherein at least two SNPs affect the phenotype. Methods of breeding and cloning companion animals are known in the art and can be used for the present invention.

This invention identifies animals that may have superior genetic traits, predicted very accurately, that can be used to identify parents of the next generation through selection.

In another aspect, the present invention provides a method of tracking a companion animal subject. The method includes identifying nucleotide occurrences for a series of genetic markers of the companion animal subject, identifying the nucleotide occurrences for the series of genetic markers for a sample, and determining whether the nucleotide occurrences of the companion animal subject are the same as the nucleotide occurrences of the sample. In this method identical nucleotide occurrences indicate that the sample is from the companion animal subject. For example, parentage can be confirmed by this method.

In certain preferred embodiments the series of genetic markers is a series of single nucleotide polymorphisms (SNPs). The method can further include comparing the results of the above determination with a determination of whether the sample is from the companion animal subject made using another tracking method. In this embodiment, the present invention provides quality control information that improves the accuracy of tracking the source of the sample.

The nucleotide occurrence data for the companion animal subject can be stored in a computer readable form, such as a database. Therefore, in one example, an initial nucleotide occurrence determination can be made for the series of genetic markers for a young companion animal subject and stored in a database along with information identifying the companion animal subject.

A series of markers or a series of SNPs as used herein, can include a series of at least 2, 3, 4, 5, 6, 7, 8, 9, 10, 15, 20, 25, 30, 35, 40, 45, 50, 75, 100, 150, 200, 250, 500, 1000, 2000, 2500, 5000 or 6000 markers, for example.

In another aspect, the present invention provides a method for diagnosing a health condition of a companion animal subject. The method includes drawing an inference regarding a phenotype of the companion animal subject for the health condition, from a nucleic acid sample of the subject. The inference is drawn by identifying, in the nucleic acid sample, at least one nucleotide occurrence of a single nucleotide polymorphism (SNP), wherein the nucleotide occurrence is associated with the phenotype.

The nucleotide occurrence of at least 2 SNPs can be determined. In some methods, at least 2 SNPs form a haplotype, wherein the method identifies a haplotype allele that is associated with the genetic trait. Preferably, the method includes identifying a diploid pair of haplotype alleles for one or more haplotypes.

The health condition for this aspect of the invention, is resistance to disease or infection, susceptibility to infection, regulation of immune status and response to antigens, previous exposure to infection or parasites, or bone/joint health, coat color/health, body mass, and health of respiratory and digestive tissues, for example.

The present invention in another aspect provides a method for inferring a phenotype of a genetic trait of a companion animal subject from a nucleic acid sample of the subject, that includes identifying, in the nucleic acid sample, at least one nucleotide occurrence of a single nucleotide polymorphism (SNP). The nucleotide occurrence is associated with the phenotype, thereby allowing an inference of the phenotype.

These embodiments of the invention are based, in part, on a determination that single nucleotide polymorphisms (SNPs), including haploid or diploid SNPs, and haplotype alleles, including haploid or diploid haplotype alleles, allow an inference to be drawn as to the phenotype of a subject, particularly a companion animal subject. Accordingly, methods of the invention can involve determining the nucleotide occurrence of at least 2, 3, 4, 5, 10, 20, 30, 40, 50, etc. SNPs. The SNPs can form all or part of a haplotype, wherein the method can identify a haplotype allele that is associated with the genetic trait. Furthermore, the method can include identifying a diploid pair of haplotype alleles.

In another aspect, the present invention provides a method for identifying a companion animal genetic marker that influences a phenotype of a genetic trait. The method includes analyzing companion animal genetic markers for association with the genetic trait. Preferably, as discussed above for other aspects of the invention, the genetic marker is a single nucleotide polymorphism (SNPs). Preferably, at least two SNPs are identified that influence the genetic trait. Because the method can identify at least two SNPs, and in some embodiments, many SNPs, the method can identify not only additive genetic components, but non-additive genetic components such as dominance (i.e. dominating phenotype of an allele of one gene over an allele of a another gene) and epistasis (i.e. interaction between genes at different loci). Furthermore, the method can uncover pleiotropic effects of SNP alleles (i.e. effects on many different genetic traits), because many genetic traits can be analyzed for their association with many SNPs using methods disclosed herein.

Nucleotide occurrences are determined for essentially all, and most preferably all of the SNPs of a high-density, whole genome SNP map. This approach has the advantage over traditional approaches in that since it encompasses the whole genome, it identifies potential interactions of gene products expressed from genes located anywhere on the genome without requiring preexisting knowledge regarding a possible interaction between the gene products. An example of a high-density, whole genome SNP map is a map of at least about 1 SNP per 10,000 kb, preferably at least 1 SNP per 500 kb or about 10 SNPs per 500 kb, most preferably at least about 25 SNPs or more per 500 kb. Definitions of densities of markers may change across the genome and are determined by the degree of linkage disequilibrium from marker to marker.

The invention includes methods for creating a high density map. The SNP markers and their surrounding sequence are compared to model organisms, for example human and mouse genomes, where the complete genomic sequence is known and syntenic regions identified. The model organism map may serve as a template for ensuring complete coverage of the animal genome. The finished map has markers spaced in such a way to maximize the amount of linkage disequilibrium in a specific genetic region.

This map is used to mark all regions of the chromosomes in a single experiment utilizing thousands of experimental animals in an association study, to correlate genomic regions with complex and simple genetic traits. These associations can be further analyzed to unravel complex interactions among genomic regions that contribute to the targeted genetic trait or other traits, epistatic genetic interactions and pleiotropy. The invention of regional high density maps can also be used to identify targeted regions of chromosomes that influence genetic traits.

Accordingly, in embodiments where SNPs that affect the same phenotype are identified that are located in different genes, the method can further include analyzing expression products of genes near the identified SNPs, to determine whether the expression products interact. As such, the present invention provides methods to detect epistatic genetic interactions. Laboratory methods are well known in the art for determining whether gene products interact.

In another aspect, the present invention provides a method for identifying a companion animal gene associated with a genetic trait. The method includes identifying a companion animal single nucleotide polymorphism (SNP) that influences a phenotype of a genetic trait by analyzing a genome-wide companion animal SNP map for association with the genetic trait, wherein the SNP is found on a target region of a companion animal chromosome. Genes present on the target region are then identified. The presence of a gene on the target region of the companion animal chromosome indicates that the gene is a candidate gene for association with the genetic trait. The candidate gene can then be analyzed using methods known in the art to determine whether it is associated with the genetic trait.

In another aspect, the present invention provides a high-throughput system for determining the nucleotide occurrences at a series of companion animal single nucleotide polymorphisms (SNPs). The system typically includes a hybridization medium comprising a series of oligonucleotides, which is typically one of the following: a solid support to which a series of oligonucleotides can be directly or indirectly attached, a homogeneous assay or a microfluidic device. Each of these hybridization mediums is used to determine the nucleotide occurrence of companion animal SNPs that are associated with a genetic trait.

Accordingly, the oligonucleotides are used to determine the nucleotide occurrence of companion animal SNPs that are associated with a genetic trait. The determination can be made by selecting oligonucleotides that bind at or near a genomic location of each SNP of the series of companion animal SNPs. The high-throughput system of the present invention typically includes a reagent handling mechanism that can be used to apply a reagent, typically a liquid, to the solid support. The binding of an oligonucleotide of the series of oligonucleotides to a polynucleotide isolated from a genome can be affected by the nucleotide occurrence of the SNP. The high-throughput system can include a mechanism effective for moving a solid support and a detection mechanism. The detection method detects binding or tagging of the oligonucleotides.

Medium to high-throughput systems for analyzing SNPs, known in the art such as the SNPStreamÒ UHT Genotyping System (Beckman/Coulter, Fullerton, Calif.) (Boyce-Jacino and Goelet Patents), the Mass Array™ system (Sequenom, San Diego, Calif.) (Storm, N. et al. (2002) Methods in Molecular Biology. 212: 241-262.), the BeadArrayÔ SNP genotyping system available from Illumina (San Diego, Calif.) (Oliphant, A., et al. (June 2002) (supplement to Biotechniques), and TaqMan™ (Applied Biosystems, Foster City, Calif.) can be used with the present invention. However, the present invention provides a medium to high-throughput system that is designed to detect nucleotide occurrences of canine SNPs, or a series of canine SNPs that can make up a series of haplotypes. Therefore, as indicated above the system includes a solid support or other method to which a series of oligonucleotides can be associated that are used to determine a nucleotide occurrence of a SNP for a series of canine SNPs that are associated with a trait. The system can further include a detection mechanism for detecting binding of the series of oligonucleotides to the series of SNPs. Such detection mechanisms are known in the art.

In certain preferred embodiments, the high-throughput system is a microfluidics device. Numerous microfluidic devices are known that include solid supports with microchannels (See e.g., U.S. Pat. Nos. 5,304,487, 5,110745, 5,681,484, and 5,593,838, incorporated herein by reference in their entirety). The high-throughput systems of the present invention are designed to determine nucleotide occurrences of one SNP and preferably a series of SNPs. In certain preferred embodiments, the systems can determine nucleotide occurrences of an entire genome-wide high-density SNP map.

Numerous methods are known in the art for determining the nucleotide occurrence for a particular SNP in a sample. Such methods can utilize one or more oligonucleotide probes or primers, including, for example, an amplification primer pair, that selectively hybridize to a target polynucleotide, which corresponds to one or more companion animal SNP positions. Oligonucleotide probes useful in practicing a method of the invention can include, for example, an oligonucleotide that is complementary to and spans a portion of the target polynucleotide, including the position of the SNP, wherein the presence of a specific nucleotide at the position (i.e., the SNP) is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe.

An oligonucleotide ligation assay (Grossman, P. D. et al. (1994) Nucleic Acids Research 22:4527-4534) also can be used to identify a nucleotide occurrence at a polymorphic position, wherein a pair of probes that selectively hybridize upstream and adjacent to and downstream and adjacent to the site of the SNP, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site. An example of this type of assay is the SNPlex System (Applied Biosystems, Foster City, Calif.).

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP loci can be sequenced using traditional sequence methodologies (e.g., the ‘dideoxy-mediated chain termination method,’ also known as the ‘Sanger Method’ (Sanger, F., et al., J. Molec. Biol. 94:441 (1975); Prober et al. Science 238:336-340 (1987)) and the ‘chemical degradation method,° ‘also known as the ‘Maxam-Gilbert method’ (Maxam, A. M., et al., Proc. Natl. Acad. Sci. (U.S.A.) 74:560 (1977)), both references herein incorporated by reference) to determine the nucleotide occurrence at the SNP locus.

Methods of the invention can identify nucleotide occurrences at SNPs using genome-wide sequencing or “microsequencing” methods. Whole-genome sequencing of individuals identifies all SNP genotypes in a single analysis. Microsequencing methods determine the identity of only a single nucleotide at a “predetermined” site. Such methods have particular utility in determining the presence and identity of polymorphisms in a target polynucleotide. Such microsequencing methods, as well as other methods for determining the nucleotide occurrence at a SNP locus are discussed in Boyce-Jacino, et al., U.S. Pat. No. 6,294,336, incorporated herein by reference, and summarized herein.

Microsequencing methods include the Genetic Bit Analysis method disclosed by Goelet, P. et al. (WO 92/15712, herein incorporated by reference). Additional, primer-guided, nucleotide incorporation procedures for assaying polymorphic sites in DNA have also been described (Komher, J. S. et al, Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al, Hum. Mutat. 1:159-164 (1992); Ugozzoli, L. et al., GATA 9:107-112 (1992); Nyren, P. et al., Anal. Biochem. 208:171-175 (1993); and Wallace, WO89/10414). These methods differ from Genetic Bit™. Analysis in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al. Amer. J. Hum. Genet. 52:46-59 (1993)).

Alternative microsequencing methods have been provided by Mundy, C. R. (U.S. Pat. No. 4,656,127) and Cohen, D. et al (French Patent 2,650,840; PCT Appln. No. WO91/02087) which discusses a solution-based method for determining the identity of the nucleotide of a polymorphic site. As in the Mundy method of U.S. Pat. No. 4,656,127, a primer is employed that is complementary to allelic sequences 3′-to a polymorphic site.

In response to the difficulties encountered in employing gel electrophoresis to analyze sequences, alternative methods for microsequencing have been developed. Macevicz (U.S. Pat. No. 5,002,867), for example, describes a method for determining nucleic acid sequence via hybridization with multiple mixtures of oligonucleotide probes. In accordance with such method, the sequence of a target polynucleotide is determined by permitting the target to sequentially hybridize with sets of probes having an invariant nucleotide at one position, and variant nucleotides at other positions. The Macevicz method determines the nucleotide sequence of the target by hybridizing the target with a set of probes, and then determining the number of sites that at least one member of the set is capable of hybridizing to the target (i.e., the number of ‘matches°). This procedure is repeated until each member of a sets of probes has been tested.

Boyce-Jacino, et al., U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

The occurrence of a SNP can be determined using denaturing HPLC such as described in Nairz K et al (2002) Proc. Natl. Acad. Sci. (U.S.A.) 99:10575-80, and the Transgenomic WAVE® System (Transgenomic, Inc. Omaha, Nebr.). Oliphant et al. report a method that utilizes BeadArray™ Technology that can be used in the methods of the present invention to determine the nucleotide occurrence of a SNP. (supplement to Biotechniques, June 2002). Additionally, nucleotide occurrences for SNPs can be determined using a DNAMassARRAY system (SEQUENOM, San Diego, Calif.). This system combines proprietary SpectroChips™, microfluidics, nanodispensing, biochemistry, and MALDI-TOF MS (matrix-assisted laser desorption ionization time of flight mass spectrometry).

As another example, the nucleotide occurrences of canine SNPs in a sample can be determined using the SNP-IT™ method (Beckman Coulter, Fullerton, Calif.). In general, SNP-IT™ is a 3-step primer extension reaction. In the first step a target polynucleotide is isolated from a sample by hybridization to a capture primer, which provides a first level of specificity. In a second step the capture primer is extended from a terminating nucleotide triphosphate at the target SNP site, which provides a second level of specificity. In a third step, the extended nucleotide trisphosphate can be detected using a variety of known formats, including: direct fluorescence, indirect fluorescence, an indirect colorimetric assay, mass spectrometry, fluorescence polarization, etc. Reactions can be processed in 384 well format in an automated format using a SNPstream™ instrument (Beckman Coulter, Fullerton, Calif.). Reactions can also be analyzed by binding to Luminex biospheres (Luminex Corporation, Austin, Tex., Cai. H. (2000) Genomics 66(2): 135-43.). Other formats for SNP detection include TaqMan™ (Applied Biosystems, Foster City, Calif.), Rolling circle (Hatch et al (1999) Genet. Anal. 15: 35-40, Qi et al (2001) Nucleic Acids Research Vol. 29 e116), fluorescence polarization (Chen, X., et al. (1999) Genome Research 9:492-498), SNaPShot (Applied Biosystems, Foster City, Calif.) (Makridakis, N. M. et al. (2001) Biotechniques 31:1374-80.), oligo-ligation assay (Grossman, P. D., et al. (1994) Nucleic Acids Research 22:4527-4534), locked nucleic acids (LNA™, Link, Technologies LTD, Lanarkshire, Scotland, EP patent 1013661, U.S. Pat. No. 6,268,490), Invader Assay (Aclara Biosciences, Wilkinson, D. (1999) The Scientist 13:16), padlock probes (Nilsson et al. Science (1994), 265: 2085), Sequence-tagged molecular inversion probes (similar to padlock probes) from ParAllele Bioscience (South San Francisco, Calif.; Hardenbol, P. et al. (2003) Nature Biotechnology 21:673-678), Molecular Beacons (Marras, S. A. et al. (1999 Genet Anal. 14:151-156), the READIT™ SNP Genotyping System from Promega (Madison, Wis.) (Rhodes R. B. et al. (2001) Mol Diagn. 6:55-61), Dynamic Allele-Specific Hybridization (DASH) (Prince, J. A. et al. (2001) Genome Research 11:152-162), the Qbead™ system (quantum dot encoded microspheres conjugated to allele-specific oligonucleotides) (Xu H. et al. (2003) Nucleic Acids Research 31 :e43), Scorpion primers (similar to molecular beacons except unimolecular) (Thelwell, N. et al. (2000) Nucleic Acids Research 28:3752-3761), and Magiprobe (a novel fluorescence quenching-based oligonucleotide probe carrying a fluorophore and an intercalator) (Yamane A. (2002) Nucleic Acids Research 30:e97). In addition, Rao, K. V. N. et al. ((2003) Nucleic Acids Research. 31:e66), recently reported a microsphere-based genotyping assay that detects SNPs directly from human genomic DNA. The assay involves a structure-specific cleavage reaction, which generates fluorescent signal on the surface of microspheres, followed by flow cytometry of the microspheres. With a slightly different twist on the Sequenom technology (MALDI), Sauer et al. ((2003) Nucleic Acids Research 31:e63) generate charge-tagged DNA (post PCR and primer extension), using a photocleavable linker.

Accordingly, using the methods described above, the companion animal, such as a canine companion animal, haplotype allele or the nucleotide occurrence of a companion animal SNP can be identified using an amplification reaction, a primer extension reaction, or an immunoassay. The companion animal haplotype allele or companion animal SNP can also be identified by contacting polynucleotides in the sample or polynucleotides derived from the sample, with a specific binding pair member that selectively hybridizes to a polynucleotide region comprising the companion animal SNP, under conditions wherein the binding pair member specifically binds at or near the companion animal SNP. The specific binding pair member can be an antibody or a polynucleotide.

The nucleotide occurrence of a SNP can be identified by other methodologies as well as those discussed above. For example, the identification can use microarray technology, which can be performed with PCR, for example using Affymetrix technologies and GenFlex Tag arrays (See e.g., Fan et al (2000) Genome Res. 10:853-860), or using a canine gene chip containing proprietary SNP oligonucleotides (See e.g., Chee et al (1996), Science 274:610-614; and Kennedy et al. (2003) Nature Biotech 2,1:1233-1237) or without PCR, or sequencing methods such as mass spectrometry, scanning electron microscopy, or methods in which a polynucleotide flows past a sorting device that can detect the sequence of the polynucleotide. The occurrence of a SNP can be identified using electrochemical detection devices such as the eSensor™ DNA detection system (Motorola, Inc., Yu, C. J. (2001) J. Am Chem. Soc. 123:11155-11161). Other formats include melting curve analysis using fluorescently labeled hybridization probes, or intercalating dyes (Lohmann, S. (2000) Biochemica 4, 23-28, Herrmann, M. (2000) Clinical Chemistry 46: 425).

The SNP detection systems of the present invention typically utilize selective hybridization. As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying a nucleotide occurrence of a SNP. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

The term ‘polynucleotide’ is used broadly herein to mean a sequence of deoxyribonucleotides or ribonucleotides that are linked together by a phosphodiester bond. For convenience, the term ‘oligonucleotide’ is used herein to refer to a polynucleotide that is used as a primer or a probe. Generally, an oligonucleotide useful as a probe or primer that selectively hybridizes to a selected nucleotide sequence is at least about 15 nucleotides in length, usually at least about 18 nucleotides, and particularly about 21 nucleotides or more in length.

A polynucleotide can be RNA or can be DNA, which can be a gene or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′ deoxyribose, or ribonucleotides such as adenine, cytosine, guanine or uracil linked to ribose. However, a polynucleotide or oligonucleotide also can contain nucleotide analogs, including non naturally occurring synthetic nucleotides or modified naturally occurring nucleotides. Such nucleotide analogs are well known in the art and commercially available, as are polynucleotides containing such nucleotide analogs (Lin et al., Nucleic Acids Research (1994) 22:5220-5234 Jellinek et al., Biochemistry (1995) 34:11363-11372; Pagratis et al., Nature Biotechnol. (1997) 15:68-73, each of which is incorporated herein by reference). Primers and probes can also be comprised of peptide nucleic acids (PNA) (Nielsen P E and Egholm M. (1999) Curr. Issues Mol. Biol. 1:89-104).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide or oligonucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995). Thus, the term polynucleotide as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR).

In various embodiments for identifying nucleotide occurrences of SNPs, it can be useful to detectably label a polynucleotide or oligonucleotide. Detectable labeling of a polynucleotide or oligonucleotide is well known in the art. Particular non-limiting examples of detectable labels include chemiluminescent labels, fluorescent labels, radiolabels, enzymes, haptens, or even unique oligonucleotide sequences. Thus, a polynucleotide or an oligonucleotide of the invention can further include a detectable label. For example, the detectable label can be associated with the polynucleotide at a position corresponding to the SNP in Table 8 sequences. As discussed in more detail herein, the labeled polynucleotide can be generated, for example, during a microsequencing reaction, such as SNP-IT™ reaction.

A method of the identifying a SNP also can be performed using a specific binding pair member. As used herein, the term ‘specific binding pair member’ refers to a molecule that specifically binds or selectively hybridizes to another member of a specific binding pair. Specific binding pair member include, for example, probes, primers, polynucleotides, antibodies, etc. For example, a specific binding pair member includes a primer or a probe that selectively hybridizes to a target polynucleotide that includes a SNP loci, or that hybridizes to an amplification product generated using the target polynucleotide as a template.

As used herein, the term ‘specific interaction,’ or ‘specifically binds’ or the like means that two molecules form a complex that is relatively stable under physiologic conditions. The term is used herein in reference to various interactions, including, for example, the interaction of an antibody that binds a polynucleotide that includes a SNP site; or the interaction of an antibody that binds a polypeptide that includes an amino acid that is encoded by a codon that includes a SNP site. According to methods of the invention, an antibody can selectively bind to a polypeptide that includes a particular amino acid encoded by a codon that includes a SNP site. Alternatively, an antibody may preferentially bind a particular modified nucleotide that is incorporated into a SNP site for only certain nucleotide occurrences at the SNP site, for example using a primer extension assay.

A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known and include, for example, equilibrium dialysis, surface plasmon resonance, and the like.

The invention also relates to kits, which can be used, for example, to perform a method of the invention such as parentage, identity, breed determination and the determination of trait identification. Thus, in one embodiment, the invention provides a kit for identifying nucleotide occurrences or haplotype alleles of canine SNPs. Such a kit can contain, for example, an oligonucleotide probe, primer, or primer pair (see e.g., Table 7, SEQ ID NOs:102-407), or combinations thereof, for identifying the nucleotide occurrence of at least one canine single nucleotide polymorphism (SNP) associated with breed, such as a SNP corresponding to the first nucleotide, or complement thereof, in the most 3′ position of any one of SEQ ID NOs:1-101 (see Table 1 or Table 8). Such oligonucleotides being useful, for example, to identify a SNP or haplotype allele as disclosed herein; or can contain one or more polynucleotides corresponding to a portion of a canine gene containing one or more nucleotide occurrences associated with a canine trait, such polynucleotide being useful, for example, as a standard (control) that can be examined in parallel with a test sample. In addition, a kit of the invention can contain, for example, reagents for performing a method of the invention, including, for example, one or more detectable labels, which can be used to label a probe or primer or can be incorporated into a product generated using the probe or primer (e.g., an amplification product); one or more polymerases, which can be useful for a method that includes a primer extension or amplification procedure, or other enzyme or enzymes (e.g., a ligase or an endonuclease), which can be useful for performing an oligonucleotide ligation assay or a mismatch cleavage assay; and/or one or more buffers or other reagents that are necessary to or can facilitate performing a method of the invention. The primers or probes can be included in a kit in a labeled form, for example with a label such as biotin or an antibody. In one embodiment, a kit of the invention provides a plurality of oligonucleotides of the invention, including one or more oligonucleotide probes or one or more primers, including forward and/or reverse primers, or a combination of such probes and primers or primer pairs. Such a kit also can contain probes and/or primers that conveniently allow a method of the invention to be performed in a multiplex format.

The kit can also include instructions for using the probes or primers to determine a nucleotide occurrence of at least one canine SNPs. In one embodiment, a kit of the invention provides a plurality of oligonucleotides of the invention, including one or more oligonucleotide probes or one or more primers, including forward and/or reverse primers, or a combination of such probes and primers or primer pairs. Such a kit also can contain probes and/or primers that conveniently allow a method of the invention to be performed in a multiplex format. The kit can also include instructions for using the probes or primers to determine a nucleotide occurrence of at least one companion animal SNP, such as an SNP from a canine subject.

In another embodiment, the present invention provides a primer pair that binds to a first target region and a second target region, thereby supporting amplification of a nucleic acid sequence that includes the sequence of an SNP corresponding to any one of the SNPs set forth in SEQ ID NOs:1-101. For example, SEQ ID NO:1 encompasses the nucleic acid sequence

TCTATACCTCTAAAGAATCGCTGCTACTTTGTGCAAGACTTTTAAAGTTT
AAATGAATTAA/G.

Thus, nucleotides A or G correspond to the single nucleotide polymorphism (SNP) of SEQ ID NO:1 because the SNP corresponds to the first nucleotide, or complement thereof, in the most 3′ position of SEQ ID NO:1. Table 8 lists the SNP accession number and the 5′ sequence associated with each SNP (i.e., SEQ ID NOs:1-101). The single nucleotide polymorphism (SNP) corresponds to the first nucleotide, or complement thereof, in the most 3′ position of any one of SEQ ID NOs:1-101. Primer pairs include the forward (SEQ ID NOs:102-203) and reverse (SEQ ID NOs:204-305) primers provided in Table 7. For example, a primer for the SNP having the accession number ss9048431 can include

| | |
|---|---|
| (TATTGACTCTATACCTCTAA AGAATCGC) | SEQ ID NO:102 |
| and | |
| (AGAGTTTCATACTGGGGTAACTTTG). | SEQ ID NO:204 |
| (AGACTTTTAAAGTTTAAA TGAATTA). | SEQ ID NO:306 |

The extension primer for this SNP can include In general, the first primer of the primer pair and a second primer of the primer pair are at least 10 nucleotides in length and bind opposite strands of the target region located within about 3000 nucleotides of a position corresponding to the position of the SNP set forth in any one of the sequences set forth in SEQ ID NOs:1-101. In certain aspects, the terminal nucleotide of an oligonucleotide binds tb the SNP. In these aspects, the method can include detecting an extension product generated using the oligonucleotide as a primer.

In another embodiment, provided herein is a primer pair that binds to a first target region and a second target region within about 3000 base pairs of SEQ ID NOs:1-101, wherein a first primer of the primer pair and a second primer of the primer pair are at least 10 nucleotides in length, bind opposite strands of the target region, and prime polynucleotide synthesis from the target region in opposite directions across the SNP identified in any one of SEQ ID NOs:1-101.

In another embodiment, the present invention provides an isolated oligonucleotide that selectively binds to a target polynucleotide that comprises at least 5, 10, 15, 20, 25, 30, 35, 40, 45, 50, 100, 150, 300, 500, or 600 nucleotides of any one of SEQ ID NOs:1-101, or a complement thereof. In another embodiment, the present invention provides an isolated oligonucleotide that includes 10 nucleotides, which selectively binds to a target polynucleotide of any one of the sequences provided in Table 8. The oligonucleotide can be, for example, 10, 15, 20, 25, 50, or 100 nucleotides in length.

In another embodiment, the present invention provides an isolated oligonucleotide pair effective for determining a nucleotide occurrence at a single nucleotide polymorphism (SNP) corresponding to the first nucleotide, or complement thereof, in the most 3′ position of any one of SEQ ID NOs:1-101 (Table 1 and Table 8). In certain aspects, the specific binding pair member is a substrate for a primer extension reaction.

In another embodiment, the present invention provides an isolated vector that includes a polynucleotide disclosed hereinabove. The term “vector” refers to a plasmid, virus or other vehicle known in the art that has been manipulated by insertion or incorporation of a nucleic acid sequence. Methods that are well known in the art can be used to construct vectors, including in vitro recombinant DNA techniques, synthetic techniques, and in vivo recombination/genetic techniques (See, for example, the techniques described in Maniatis et al. 1989 Molecular Cloning A Laboratory Manual, Cold Spring Harbor Laboratory, N.Y., incorporated herein in its entirety by reference). Further, the present invention provides an isolated cell that includes the vector. The cell can be prokaryotic or eukaryotic. Techniques for incorporated vectors into prokaryotic and eukaryotic cells are well known in the art. In certain aspects, the cells are canine cells. In other aspects, the cells are bacterial cells. In still other aspects, the cells are human cells.

Methods and compositions provided herein are also useful to infer a trait of a canine subject from a nucleic acid sample of the canine subject is provided. An exemplary method includes contacting the nucleic acid sample with a pair of oligonucleotides that comprise a primer pair, wherein amplified target nucleic acid molecules are produced; hybridizing at least one oligonucleotide primer selected from the group consisting of SEQ ID NOS:306-407 (see Table 7) to one or more amplified target nucleic acid molecules, wherein each oligonucleotide primer is complementary to a specific and unique region of each target nucleic acid molecule such that the 3′ end of each primer is proximal to a specific and unique target nucleotide of interest; extending each oligonucleotide with a template-dependent polymerase; and determining the identity of each nucleotide of interest by determining, for each extension primer employed, the identity of the nucleotide proximal to the 3′ end of each primer. The primer pair can be any of the forward and reverse oligonucleotide primer pairs listed in Table 7. For example, a first primer of the primer pair can be selected from SEQ ID NOS:1-101 and the second primer of the primer pair can be selected from SEQ ID NOS:102-203.

Population-specific alleles can be used to assign, for example, a canine animal to a particular breed. These population specific alleles are fixed in the population of interest and absent in comparison populations. The absence of an allele in a sample of individuals from any one population may be because those alleles are truly population-specific or because the frequency of those alleles is low and the sample obtained from any given population was small (Taylor, J. F., Patent: PCT/US01/47521). For admixed populations, population-specific alleles rarely occur, however the difference in allele frequency between populations may still enable their use to infer assignment of individual canines based to a breed, these are known as population associated alleles (Kumar, P., Heredity 91: 43-50 (2003)). Both population specific alleles and population-associated alleles are herein referred to as Breed-Specific Markers.

In the present invention, a marker is breed specific if it has a different allele frequency in one breed relative to one or more other breeds. A similar logic was employed by Kumar, P. (Heredity 91: 43-50 (2003)) to genetically distinguish cattle from European Bos taurus breeds and Indian Bos indicus breeds of cattle (see e.g., DeNise et al., 2003. U.S. patent application Ser. No. 10/750,622; Parker et al., Science 304, 1161-1164 (2004)).

In the present invention there are about 60 parentage and identity markers and about 101 breed-specific SNP markers, not mutually exclusive. One or more of these markers could be used to determine parentage or identity or breed specificity and/or to assign an individual to one or more breeds with an associated probability. These markers could be used alone or in any combination.

In general, there are two broad classes of clustering methods that are used to assign individuals to populations (Pritchard, J. K., et al., Genetics 155: 945-959 (2000)). These are: 1) Distance-based methods: These calculate a pairwise distance matrix, whose entries give the distance between every pair of individuals. 2) Model-based methods: These proceed by assuming that observations from each cluster are random draws from some parametric model. Inference for the parameters corresponding to each cluster is then done jointly with inference for the cluster membership of each individual, using standard statistical methods. The preset disclosure includes the use of all standard statistical methods including maximum likelihood, bootstrapping methodologies, Bayesian methods and any other statistical methodology that can be employed to analyze such genome data. These statistical techniques are well known to those in the art.

Many software programs for molecular population genetics studies have been developed, their advantage lies in their pre-programmed complex mathematical techniques and ability to handle large volumes of data. Popular programs used by those in the field include, but are not limited to: TFPGA, Arlequin, GDA, GENEPOP, GeneStrut, POPGENE (Labate, J. A., Crop Sci. 40: 1521-1528. (2000)) and Structure. The present disclosure incorporates the use of all of the software disclosed above used to classify canines into populations based on DNA polymorphisms as well as other software known in the art. “Structure” has been used to determine population structure and infer assignment of individual animals to populations for livestock species including poultry (Rosenberg, N. A., et al., Genetics. 159: 699-713 (2001)) and canines from South Asia (Kumar, P., Heredity 91: 43-50 (2003)).

In another aspect, the present invention provides a computer system that includes a database having records containing information regarding a series of companion animal single nucleotide polymorphisms (SNPs), and a user interface allowing a user to input nucleotide occurrences of the series of companion animal SNPs for a companion animal subject. The user interface can be used to query the database and display results of the query. The database can include records representing some or preferably all of the SNP of a companion animal SNP map, preferably a high-density companion animal SNP map. The database can also include information regarding haplotypes and haplotype alleles from the SNPs. Furthermore, the database can include information regarding phenotypes and/or genetic traits that are associated with some or all of the SNPs and/or haplotypes. In these embodiments the computer system can be used, for example, for any of the aspects of the invention that infer a phenotype of a genetic trait of a companion animal subject.

The computer system of the present invention can be a stand-alone computer, a conventional network system including a client/server environment and one or more database servers, and/or a handheld device. A number of conventional network systems, including a local area network (LAN) or a wide area network (WAN), are known in the art. Additionally, client/server environments, database servers, and networks are well documented in the technical, trade, and patent literature. For example, the database server can run on an operating system such as UNIX, running a relational database management system, a World Wide Web application, and a World Wide Web Server. When the computer system is a handheld device it can be a personal digital assistant (PDA) or another type of handheld device, of which many are known.

Typically, the database of the computer system of the present invention includes information regarding the location and nucleotide occurrences of companion animal SNPs. Information regarding genomic location of the SNP can be provided for example by including sequence information of consecutive sequences surrounding the SNP, that only 1 part of the genome provides 100% match, or by providing a position number of the SNP with respect to an available sequence entry, such as a Genbank sequence entry, or a sequence entry for a private database, or a commercially licensed database of DNA sequences. The database can also include information regarding nucleotide occurrences of SNPs, since as discussed herein typically nucleotide occurrences of less than all four nucleotides occur for a SNP.

The database can include other information regarding SNPs or haplotypes such as information regarding frequency of occurrence in a companion animal population. Furthermore, the database can be divided into multiple parts, one for storing sequences and the others for storing information regarding the sequences. The database may contain records representing additional information about a SNP, for example information identifying the gene in which a SNP is found, or nucleotide occurrence frequency information, or characteristics of the library or clone which generated the DNA sequence, or the relationship of the sequence surrounding the SNP to similar DNA sequences in other species.

The parts of the database of the present invention can be flat file databases or relational databases or object-oriented databases. The parts of the database can be internal databases, or external databases that are accessible to users. An internal database is a database maintained as a private database, typically maintained behind a firewall, by an enterprise. An external database is located outside an internal database, and is typically maintained by a different entity than an internal database. A number of external public biological sequence databases, particularly SNP databases, are available and can be used with the current invention. For example, the dbSNP database available from the National Center for Biological Information (NCBI), part of the National Library of Medicine, can be used with the current invention to provide comparative genomic information to assist in identifying companion animal SNPs.

In another aspect, the current invention provides a population of information regarding companion animal SNPs and haplotypes. The population of information can include an identification of genetic traits associated with the SNPs and haplotyopes. The population of information is typically included within a database, and is preferably identified using the methods of the current invention. The population of sequences can be a subpopulation of a larger database, that contains only SNPs and haplotypes related to a particular genetic trait. For example, the subpopulation can be identified in a table of a relational database. A population of information can include all of the SNPs and/or haplotypes of a genome-wide SNP map.

In addition to the database discussed above, the computer system of the present invention includes a user interface capable of receiving entry of nucleotide occurrence information regarding at least one, preferably two companion animal SNPs. The interface can be a graphic user interface where entries and selections are made using a series of menus, dialog boxes, and/or selectable buttons, for example. The interface typically takes a user through a series of screens beginning with a main screen. The user interface can include links that a user may select to access additional information relating a companion animal SNP map.

The function of the computer system of the present invention that carries out the phenotype inference methods typically includes a processing unit that executes a computer program product, itself representing another aspect of the invention, that includes a computer-readable program code embodied on a computer-usable medium and present in a memory function connected to the processing unit. The memory function can be ROM or RAM.

The computer program product, itself another aspect of the invention, is read and executed by the processing unit of the computer system of the present invention, and includes a computer-readable program code embodied on a computer-usable medium. The computer-readable program code relates to a plurality of sequence records stored in a database. The sequence records can contain information regarding the relationship between nucleotide occurrences of a series of companion animal single nucleotide polymorphisms (SNPs) and a phenotype of one or more genetic traits. The computer program product can include computer-readable program code for providing a user interface capable of allowing a user to input nucleotide occurrences of the series of companion animal SNPs for a companion animal subject, locating data corresponding to the entered query information, and displaying the data corresponding to the entered query. Data corresponding to the entered query information is typically located by querying a database as described above.

In another embodiment of the present invention, the computer system and computer program products are used to perform bioeconomic valuations used to perform methods described herein, such as methods for estimating the value of a companion animal subject that will be obtained therefrom.

An exemplary canine panel of SNPs for determining, for example, parentage or breed, is provided herein. DNA analysis provides a powerful tool for verifying the parentage and identification of individual animals. Microsatellite marker panels have been developed for canine that are highly polymorphic and amenable to standardization among laboratories performing these tests (DeNise et al., 2004, Anim Genet. 35(1): 14-17). However, microsatellite scoring requires considerable human oversight and microsatellite markers have high mutation rates. Single nucleotide polymorphisms (SNP) are likely to become the standard marker for parentage verification and identity because of the ease of scoring, low cost assay development and high-throughput capability.

The present invention is based in part on the discovery of single nucleotide polymorphisms (SNPs) that can be used to verify parentage or identity of canine subjects or infer breed of a canine subject. For example, SNPs have been used to verify parentage and breed in bovine subjects (see, e.g., U.S. patent application Ser. No. 10/750,622 or U.S. patent application Ser. No. 10/750,623, both of which are incorporated herein in their entirety). Accordingly, provided herein is a method for excluding putative parents of a canine breed and/or verifying identity of a canine; or inferring the breed of a canine subject from a nucleic acid sample of the canine subject, by identifying in the sample, a nucleotide occurrence for at least one single nucleotide polymorphism (SNP), wherein the nucleotide occurrence is associated with the breed.

Teachings for genetic identity and parentage exclusion are well known in the art. (DeNise et al., 2004. Anim. Genetics. 35(1): 14-17; Halverson et al., 1995. U.S. Pat. No. 05,874,217; Ostrander et al., 1993. Genomics, 16: 207-213, Ostrander et al., 1995. Mammalian Genome, 6: 192-195; Franscisco et al., 1996. Mammalian Genome 7:359-362). Statistical probability of identity is calculated as the probability of having a canine animal with the specific genotype of a canine subject. Parentage verification and identification is statistically characterized by the exclusion probability. Both of these statistical estimates are calculated from nucleotide frequencies within the population.

The methods of the present invention for inferring breed of a canine subject can be used to infer the breed of any canine subject. For example, the methods can be used to infer a breed including, but not limited to, Afghan Hound, Basenji, Basset Hound, Beagle, Belgian Tervuren, Bernese Mountain Dog, Borzoi, Chihuahua, Chinese Shar-Pei, Cocker Spaniel, Dachshund, Doberman Pinscher, German Shepherd Dog, German Shorthaired Pointer, Golden Retriever, Labrador Retriever, Mastiff, Miniature Schnauzer, Poodle, Pug, Rottweiler, Saluki, Samoyed, Shetland Sheepdog, Siberian Husky, St. Bernard, Whippet and Yorkshire Terrier.

Furthermore, the methods of the present invention can be used to assign a breed or breeds to an individual animal with a specific probability. Typically, an identified nucleotide occurrence is compared to multiple known SNP alleles associated with multiple breeds, for example the breed associated alleles identified herein in Table 4, to infer a breed for a subject from multiple possible breeds.

SNP markers were identified from whole-genome shotgun sequencing of the canine genome (Kirkness, et al., 2003, Science 5641:1898-903). Over 650,000 putative bi-alleleic SNP markers, excluding insertion/deletions, were identified from the 974,000 putative SNPs assigned Genbank accession numbers between ss8830321 and ss9805720. The contigs containing these SNPs were syntenically mapped to the sequence of the human genome. The present study evaluated 384 SNP markers for their robust assay development, allele frequency among 30 canine breeds, exclusion probabilities and probability match rate. Out of these markers, about 60 SNPs were selected for a parentage panel that can be used across a number of breeds and systems for parentage verification and animal identity and 101 breed specific markers were identified. Briefly, markers were assayed on Beckman Coulter GenomeLab™ SNPstream® Genotyping System. Markers were amplified in a 5 μl reaction volume of a 12-marker multiplex in a 384-well format. The PCR is performed as follows: 95° C. for 10 min, followed by 34 cycles of 94° C. for 30 s, 55° C. for 30 s, and 72° C. for 1 min. The DNA products are cleaned using 3 μl of diluted SNP-IT™ Clean-Up (USB), incubated at 37° C. for 30 min with a final inactivation step of 96° C. for 10 min. The extension reaction is performed as described by the manufacturer, with 0.2 μl of the G/A extension mix 3.762 μl extension mix diluent, 0.021 ml DNA polymerase, 3 μl of extension primer working stock, and 0.018 ml water added to the 8 ml volume in the well after clean-up. This 15 μl extension reaction is then thermal cycled as follows: 96° C. for 3 min, followed by 45 cycles of 94° C. for 20 s and 40° C. for 11 s. Following extension, 8 μl of hybridization cocktail is added and mixed. Ten microliters of this mixture is then transferred to the 384-well SNPStream® Tag Array plate. The plate is then incubated at 42° C. for 2 hr. Each of the 384 wells in a Tag Array plate contains 16 unique oligonucleotides of a known sequence, or tag. After hybridization, the Tag Array plate is then washed, dried (1 hr), and read on the SNPstream® SNPScope Array Imager. The raw image data is then analyzed and genotype calls generated using the software provided, then reviewed by scientists before data is uploaded into the database.

Three hundred eighty four SNP markers were selected for study based on their dispersion pattern throughout the canine genome as determined by their human location, and all markers contained a guanine/adenine purine transition for ease of assay development. Trios of 23 parent, offspring combinations were used to verify mendelian inheritance. Canine animals, representing 30 breeds, 38 animals per breed, were used to validate and select markers. Allele frequencies within breed were determined using simple counting methods. Sixty markers were identified that can be utilized for parentage and identity and 101 breed specific markers were identified. These markers are not mutually exclusive. Accession numbers for parentage and identity markers are listed in Table 1 and Table 8. The sequences of the parentage and identity markers can be found on the world wide web at ftp://ftp.ncbi.nlm.nih.gov/snp/dog/ss_fasta. The contents of these files are encoded in XML, and contain the following information: SNP Id, Contig Name denoting the location of the SNP, and 60 bases of sequence flanking 5′ end of SNP, and the alleles comprising the SNP. The position of the SNP in the contig is determined by blasting the 5′ flanking sequence to the contig sequence. The location of the SNP is the base following the last matching base of the 60 bases. Contigs can be found on the world wide web at http://www.ncbi.nlm.nih.gov/entrez/query.fcgidb=Nucleotide&cmd=Search&term=AACN010000001:AACN011089636[PACC]. An example of the information provided for Accession number ss9048431 includes the following information related to reference information and contig analysis:
<NSE-rs_refsnp-id>8499601</NSE-rs_refsnp-id>
<NSE-rs_taxid>9615</NSE-rs_taxid>
<NSE-rs_organism>dg</NSE-rs_organism>
<NSE-rs_snp-class value=“snp”/>
<NSE-rs_snp-type value=“notwithdrawn”/>
<NSE-rs_moltype value=” genomic”/>
<NSE-rs_create-date>
<NSE-Date>
<NSE-Date_str>10/27/2003 15:57:00</NSE-Date_str>
</NSE-Date>
</NSE-rs_create-date>
<NSE-rs_update-date>
<NSE-Date>
<NSE-Date_str>10/27/2003 15:57:00</NSE-Date_str>
</NSE-Date>
</NSE-rs_update-date>
<NSE-rs_create-build>118</NSE-rs_create-build>
<NSE-rs_update-build>118</NSE-rs_update-build>
<NSE-rs_observed>A/G</NSE-rs_observed>

<NSE-rs_seq-5>

<NSErs_seq5_E>TCTATACCTCTAAAGAATCGCTGCTACTTTGTGCAA
GACTTTTAAAGTTTAAATGAATTA</NSE-rs_seq-5_E>

</NSE-rs_seq-5>

<NSE-rs_seq-3>

<NSErs_seq3_E>GTGAATTCCAGGTAGTAAAACAATCTCTGAGCCTCA
AGTAGAAGTCATTTTTTTTTCCT</NSE-rs_seq-3_E>

</NSE-rs_seq-3>
<NSE-rs_seq-ss-exemplar>9048431</NSE-rs_seq-ss-exemplar>
<NSE-rs_valid-prob-min>0</NSE-rs_valid-prob-min>
<NSE-rs_genotype value=“false”/>
<NSE-rs_linkout value=“false”/>
<NSE-rs_last-action>
<NSE-Date>
<NSE-Date_str>10/27/2003 15:57:00</NSE-Date_str>
</NSE-Date>
</NSE-rs_last-action>
<NSE-rs_ss-list>
<NSE-ss>
<NSE-ss_handle>TIGR</NSE-ss_handle>
<NSE-ss_batch-id>7987</NSE-ss_batch-id>
<NSE-ss_subsnp-id>9048431</NSE-ss_subsnp-id>
<NSE-ss_loc-snp-id>19866850052491_{—}821</NSE-ss_loc-snp-id>
<NSE-ss_subsnp-class value=“snp”/>
<NSE-ss_orient value=“forward”/>
<NSE-ss_moltype value=“genomic”/>
<NSE-ss_build-id>118</NSE-ss_build-id>
<NSE-ss_method-class value=“sequence”/>
<NSE-ss_validated value=“by-submitter”/>
<NSE-ss_accession>
<NSE-ss_accession_E>AACN010362728</NSE-ss_accession_E>
</NSE-ss_accession>
<NSE-ss_comment>
<NSEss_comment_E>confidence=1.000coverage_depth=4Chr4:3UTR:ENSG000001 56140:NM_{—}014243</NSE-ss_comment_E>
</NSE-ss_comment>
<NSE-ss_genename>NM_{—}014243</NSE-ss_genename>
<NSE-ss_locus-id>9508</NSE-ss_locus-id>
<NSE-ss_flank-5>

<NSEss_flank5

E>TCTATACCTCTAAAGAATCGCTGCTACTTTGTGCAAGACTTTTAAAGT
TTAAATGAATTA

</NSE-ss_flank-5_E>(e.g., the region immediately 5′ to the SNP)
</NSE-ss_flank-5>
<NSE-ss_observed>A/G</NSE-ss_observed>(e.g., the position of the SNP)

<NSE-ss_flank-3>

<NSEss_flank3_E>GTGAATTCCAGGTAGTAAAACAATCTCTGAGCCT
CAAGTAGAAGTCATTTTTTTTTCCT</NSE-ss_flank-3_E>

</NSE-ss_flank-3>
</NSE-ss>
</NSE-rs_ss-list>
</NSE-rs>

With regard to the information associated with each accession number, the sequence associated with a particular sequence identifier can be found at the lined labeled “<NSEss_flank5_E>” and the SNP can be found at the line labeled “<NSE-ss_observed>.” For example, for SEQ ID NO:1 the line labeled “<NSEss_flank5_E>” has the sequence

“TCTATACCTCTAAAGAATCGCTGCTACTTTGTGCAAGACTTTTAAAGTT
TAAATGAATTA”

associated with it. In addition, the line labeled “<NSE-ss_observed>” has the SNP “A/G” associated with it. Thus, SEQ ID NO:1 encompasses the nucleic acid sequence

TCTATACCTCTAAAGAATCGCTGCTACTTTGTGCAAGACTTTTAAAGTTT
AAATGAATTAA/G.

Thus, nucleotides A or G correspond to the single nucleotide polymorphism (SNP) of SEQ ID NO:1 because the SNP corresponds to the first nucleotide, or complement thereof, in the most 3′ position of SEQ ID NO:1. Similar information for the remaining accession numbers in provided in the aforementioned database. Table 8 lists the SNP accession number and the 5′ sequence associated with each SNP (i.e., SEQ ID NOs:1-101). The single nucleotide polymorphism (SNP) corresponds to the first nucleotide, or complement thereof, in the most 3′ position of any one of SEQ ID NOs:1-101.

Table 2 provides the identified parentage and identity markers and allele frequencies within breed. Table 3 summarizes the data as to exclusion probability rate and probability match rate within breed and across all breeds. Exclusion probability at any locus l, (Qₗ), is the probability of excluding a random individual from the population as a potential parent of an animal based on the genotype of one parent and offspring. Following Weir (Weir, 1996, Genetic Data Analysis II. Sinauer, Sunderland, Mass.).
Qₗ=pₗ−2pₗ²+2pₗ³−pₗ⁴
where pₗ is the frequency of the guanine allele at locus l. The overall probability of exclusion is one minus the probability that none of the loci allows exclusion and is calculated as

$$Q = 1 - Π ( 1 - {Q}_{l} )$$

Match probability ratio (MPR) was calculated, using the ceiling principle, as the square of the most frequent allele frequency to provide the most conservative estimate of match rate within a breed. Overall match probability ratio was estimated as the product of MPR at each SNP marker.

Sixty markers with the highest exclusion probability computed across all breeds were selected for the parentage panel. For example, with the 60 marker panel, most or all breeds can reach an exclusion probability of about 0.994 and identity match rate of about 6.42×10⁻⁵. This panel provides a powerful tool that can be used efficiently in parentage and identity programs.

In one example, a nucleic acid sample from a canine subject from the Doberman Pincher breed can be accurately matched to a previously identified sample 99.9% of the time. Using these same markers for parentage verification and identity, the probability of an individual selected at random from the Doberman Pincher breed with nucleotide occurrences at the SNP parentage and identity markers consistent with the canine subject is greater than 1 in 1,000,000.

The potential parents of a canine subject can be excluded thereby assuring the direct ancestral line and insuring the integrity of the registration database. Nucleic acid hypermutable sequences are currently utilized by the American Kennel Club, Professional Kennel Club and the United Kennel Club. As used herein, the term “hypermutable” refers to a nucleic acid sequence that is susceptible to instability, thus resulting in nucleic acid alterations. Such alterations include the deletion and addition of nucleotides. The hypernutable sequences of the invention are most often microsatellite DNA sequences which, by definition, are small tandem repeat DNA sequences. Thus, a combination of SNP analysis and microsatellite analysis may be used to infer a breed of a canine subject. Nucleic acid or tissue samples from an unknown canine subject can be matched to verify the ownership or identity of an individual canine. Because of the reproducibility and standardization of the SNP panel markers, these nucleic acid differences can be stored in a database linking animal id and owner, parents and siblings, with genotype allowing for ease of comparison and reducing the need for additional testing.

A panel generated from the canine SNPs provided herein can be utilized to verify the identity of a cloned animal or frozen or split and/or cloned embryo, or characterize tissues that may undergo intra- or inter-transplantation or propagation to other mammals, or verify the identity of banked and/or frozen semen, or verify cultured cell lines. In addition, an SNP identity and parentage panel can be used to link an animal, animal hair or animal biological samples to a crime scene for forensic analysis.

Examples of the probability of correct breed assignment is presented in Table 4 for 28 breeds evaluated. The probability of assignment ranged from 0.676 for the Chihuahua breed to 0.946 for the Basenji breed. In addition, Table 5 depicts each individual canine tested and the probability of assignment to a specific cluster. As shown in Table 6, all 38 canine subjects in eleven of the 28 breeds presented reached at least 0.7 probability of falling into the correct cluster. Canine subjects in 18 of the breeds evaluated had at least 90% of the canine subjects within breed falling into the correct cluster. The SNP breed identity panel can be used to verify claims for breed of a canine animal when parentage is unknown. Currently, the only canines accepted by breed are those where the records of individual animals are maintained by Breed Associations, this could open up new avenues for dog owners. Further, information regarding canine breed could allow canine owners to identify health characteristics associated with specific breed designations. Preventative measures could reduce the trauma to the animal and owner, and provide the owner with insight into the behaviors of the canine subject. The disposition and safety of the canine subject can be broadly determined by breed characteristics. At one extreme, communities have a vested role in safeguarding their citizens against vicious behaviors; and at the other extreme, canine owners may be able to reduce negative impacts from normal behaviors found within specific breeds.

A panel provided herein also aids in the placement, lost and found searches, and animal shelter reporting for canine animals become more accurate when the exact breed is known. Such means of identification allows animal shelters to screen animals and announce the results of the search to potential owners and to specific breed rescue groups. Further, mixed breed groups could determine the percentages of specific breeds of composition and breed development using such panels. These programs could lead to certification programs that can broadly group characteristics of specific crosses of canines.

Methods of the present invention further encompass identifying a nucleotide sequence of a hypermutable sequence in the sample, and inferring breed based on at least one SNP nucleotide occurrence and the nucleotide sequence of the hypermutable sequence. Hypermutable sequences include, for example, microsatellite nucleic acid sequences. The method can include a determination of the nucleotide occurrence of at least 2 SNPs. At least 2 SNPs can form all or a portion of a haplotype, wherein the method identifies a haplotype allele that is associated with a specific breed. Furthermore, the method can include identifying a diploid pair of haplotype alleles.

Also provided provided are methods for identifying a canine single nucleotide polymorphism (SNP) informative of breed, that includes performing whole genome shotgun sequencing of a canine genome, and genotyping at least two canine subjects from at least two breeds, thereby identifying the canine single nucleotide polymorphisms informative of breed. The Example provided herein, illustrates the use of this method to identify breed SNPs.

The following tables provide exemplary data generated by the compositions and methods provided herein.

TABLE 1
GenBank Accession numbers of markers utilized to verify and
assign parentage and identity and determine breed specificity.

| Parentage and | |
|---|---|
| Identity Markers | Breed Identity Markers |
| | (SEQ ID NO: 1) ss9048431 |
| ss9053109 | (SEQ ID NO: 2) ss9053109 |
| ss9067589 | (SEQ ID NO: 3) ss9067589 |
| ss9069201 | (SEQ ID NO: 4) ss9069201 |
| ss9084075 | (SEQ ID NO: 5) ss9084075 |
| | (SEQ ID NO: 6) ss9090942 |
| | (SEQ ID NO: 7) ss9101730 |
| ss9108332 | (SEQ ID NO: 8) ss9108332 |
| ss9132982 | (SEQ ID NO: 9) ss9132982 |
| ss9139126 | (SEQ ID NO: 10) ss9139126 |
| ss9142796 | (SEQ ID NO: 11) ss9142796 |
| ss9152677 | (SEQ ID NO: 12) ss9152677 |
| ss9156891 | (SEQ ID NO: 13) ss9156891 |
| | (SEQ ID NO: 14) ss9171081 |
| | (SEQ ID NO: 15) ss9177956 |
| | (SEQ ID NO: 16) ss9186525 |
| ss9191087 | (SEQ ID NO: 17) ss9191087 |
| ss9200241 | (SEQ ID NO: 18) ss9200241 |
| ss9230071 | (SEQ ID NO: 19) ss9230071 |
| | (SEQ ID NO: 20) ss9233837 |
| | (SEQ ID NO: 21) ss9235114 |
| ss9244345 | (SEQ ID NO: 22) ss9244345 |
| | (SEQ ID NO: 23) ss9245977 |
| ss9251154 | (SEQ ID NO: 24) ss9251154 |
| ss9259716 | (SEQ ID NO: 25) ss9259716 |
| | (SEQ ID NO: 26) ss9270557 |
| ss9278814 | (SEQ ID NO: 27) ss9278814 |
| ss9281595 | (SEQ ID NO: 28) ss9281595 |
| | (SEQ ID NO: 29) ss9282411 |
| | (SEQ ID NO: 30) ss9285114 |
| ss9290112 | (SEQ ID NO: 31) ss9290112 |
| ss9290361 | (SEQ ID NO: 32) ss9290361 |
| | (SEQ ID NO: 33) ss9292376 |
| | (SEQ ID NO: 34) ss9294456 |
| ss9296487 | (SEQ ID NO: 35) ss9296487 |
| | (SEQ ID NO: 36) ss9300915 |
| | (SEQ ID NO: 37) ss9301348 |
| ss9307596 | (SEQ ID NO: 38) ss9307596 |
| | (SEQ ID NO: 39) ss9308314 |
| ss9313462 | (SEQ ID NO: 40) ss9313462 |
| ss9313564 | (SEQ ID NO: 41) ss9313564 |
| | (SEQ ID NO: 42) ss9313781 |
| | (SEQ ID NO: 43) ss9328275 |
| ss9335917 | (SEQ ID NO: 44) ss9335917 |
| ss9339680 | (SEQ ID NO: 45) ss9339680 |
| ss9362797 | (SEQ ID NO: 46) ss9362797 |
| ss9366135 | (SEQ ID NO: 47) ss9366135 |
| ss9366251 | (SEQ ID NO: 48) ss9366251 |
| ss9378306 | (SEQ ID NO: 49) ss9378306 |
| ss9380511 | (SEQ ID NO: 50) ss9380511 |
| ss9382377 | (SEQ ID NO: 51) ss9382377 |
| | (SEQ ID NO: 52) ss9389583 |
| | (SEQ ID NO: 53) ss9398291 |
| ss9403022 | (SEQ ID NO: 54) ss9403022 |
| | (SEQ ID NO: 55) ss9406226 |
| ss9409752 | (SEQ ID NO: 56) ss9409752 |
| | (SEQ ID NO: 57) ss9419451 |
| ss9419768 | (SEQ ID NO: 58) ss9419768 |
| ss9423342 | (SEQ ID NO: 59) ss9423342 |
| ss9427809 | (SEQ ID NO: 60) ss9427809 |
| | (SEQ ID NO: 61) ss9432314 |
| | (SEQ ID NO: 62) ss9438029 |
| | (SEQ ID NO: 63) ss9441594 |
| | (SEQ ID NO: 64) ss9442450 |
| | (SEQ ID NO: 65) ss9451328 |
| | (SEQ ID NO: 66) ss9454084 |
| ss9432314 | (SEQ ID NO: 67) ss9475014 |
| ss9480981 | (SEQ ID NO: 68) ss9480981 |
| ss9490183 | (SEQ ID NO: 69) ss9490183 |
| ss9496479 | (SEQ ID NO: 70) ss9496479 |
| ss9502221 | (SEQ ID NO: 71) ss9502221 |
| ss9519462 | (SEQ ID NO: 72) ss9519462 |
| ss9527721 | (SEQ ID NO: 73) ss9527721 |
| | (SEQ ID NO: 74) ss9550651 |
| ss9565630 | (SEQ ID NO: 75) ss9565630 |
| | (SEQ ID NO: 76) ss9574955 |
| ss9586065 | (SEQ ID NO: 77) ss9586065 |
| | (SEQ ID NO: 78) ss9595292 |
| | (SEQ ID NO: 79) ss9602306 |
| | (SEQ ID NO: 80) ss9609977 |
| ss9627150 | (SEQ ID NO: 81) ss9627150 |
| ss9628837 | (SEQ ID NO: 82) ss9628837 |
| | (SEQ ID NO: 83) ss9641213 |
| ss9645529 | (SEQ ID NO: 84) ss9645529 |
| ss9646032 | (SEQ ID NO: 85) ss9646032 |
| | (SEQ ID NO: 86) ss9652166 |
| ss9671733 | (SEQ ID NO: 87) ss9671733 |
| | (SEQ ID NO: 88) ss9672435 |
| ss9678528 | (SEQ ID NO: 89) ss9678528 |
| | (SEQ ID NO: 90) ss9684533 |
| ss9695373 | (SEQ ID NO: 91) ss9695373 |
| ss9705100 | (SEQ ID NO: 92) ss9705100 |
| ss9714487 | (SEQ ID NO: 93) ss9714487 |
| ss9719095 | (SEQ ID NO: 94) ss9719095 |
| | (SEQ ID NO: 95) ss9733605 |
| ss9734846 | (SEQ ID NO: 96) ss9734846 |
| | (SEQ ID NO: 97) ss9735989 |
| ss9759816 | (SEQ ID NO: 98) ss9759816 |
| ss9780984 | (SEQ ID NO: 99) ss9780984 |
| | (SEQ ID NO: 100) ss9788546 |
| ss9800286 | (SEQ ID NO: 101) ss9800286 |

TABLE 2
SNP parentage markers allele frequency by breed allele.

| | German | | | | | | |
|---|---|---|---|---|---|---|---|
| GenBank | Shorthaired | Golden | Labrador | Cocker | Miniature | Chinese | Afghan |
| Accession | Pointer | Retriever | Retriever | Spaniel | Schnauzer | Shar-Pei | Hound |
| ss9053109 | 0.776 | 0.149 | 0.395 | 0.553 | 0.487 | 0.171 | 0.421 |
| ss9067589 | 0.316 | 0.365 | 0.342 | 0.486 | 0.421 | 0.908 | 0.645 |
| ss9069201 | 0.243 | 0.676 | 0.25 | 0.447 | 0.013 | 0.676 | 0.667 |
| ss9084075 | 0.447 | 0.921 | 0.194 | 0.689 | 0.079 | 0.959 | 0.847 |
| ss9108332 | 0.526 | 0.342 | 0.513 | 0.25 | 0.919 | 0.73 | 1 |
| ss9132982 | 0.276 | 0.487 | 0.263 | 0.956 | 0.026 | 0.847 | 0.013 |
| ss9139126 | 0.342 | 0.595 | 0.342 | 0.645 | 1 | 0.797 | 0.145 |
| ss9142796 | 0.487 | 0.974 | 1 | 0.974 | 0.961 | 0.974 | 0.816 |
| ss9152677 | 0.458 | 0.014 | 0.263 | 0.042 | 0.027 | 0.517 | 0.392 |
| ss9156891 | 0.635 | 0.947 | 0.987 | 0.946 | 1 | 0.328 | 0.829 |
| ss9191087 | 0.605 | 0.855 | 0.882 | 1 | 1 | 0.579 | 0.921 |
| ss9200241 | 0.529 | 0.842 | 0.276 | 0.716 | 0.473 | 0.513 | 0.724 |
| ss9230071 | 0.882 | 1 | 0.5 | 0.513 | 0.684 | 0.639 | 0.922 |
| ss9244345 | 0.592 | 0.553 | 0.622 | 0.527 | 0.081 | 0.316 | 0.811 |
| ss9251154 | 0.921 | 0.632 | 1 | 0.947 | 1 | 0.711 | 0.684 |
| ss9259716 | 0.583 | 0.671 | 0.342 | 0.784 | 0.892 | 0.875 | 0.311 |
| ss9278814 | 0.25 | 0.662 | 0.855 | 0.079 | 0.757 | 0.403 | 0.882 |
| ss9281595 | 0.355 | 0.757 | 0.382 | 0.23 | 0.583 | 0.892 | 0.216 |
| ss9290112 | 0.541 | 0.618 | 0.368 | 0.947 | 0.566 | 0.579 | 0.684 |
| ss9290361 | 0.289 | 0.158 | 0.263 | 0.316 | 0.132 | 0.737 | 0.553 |
| ss9296487 | 0.197 | 0 | 0 | 0 | 0.013 | 0.276 | 0.145 |
| ss9307596 | 0.039 | 0.197 | 0.013 | 0.054 | 0.592 | 0.776 | 0.5 |
| ss9313462 | 0.319 | 0.329 | 0.368 | 0.568 | 1 | 1 | 0.526 |
| ss9313564 | 0.211 | 0.421 | 0.461 | 0 | 0 | 0.342 | 0 |
| ss9335917 | 0.894 | 0.658 | 0.959 | 0.985 | 0.986 | 0.833 | 0.983 |
| ss9339680 | 0.392 | 0.316 | 0.25 | 0.026 | 0 | 0 | 0.145 |
| ss9362797 | 0.842 | 0.905 | 0.461 | 0.135 | 0.847 | 0.959 | 0.211 |
| ss9366135 | 0.809 | 0.562 | 0.763 | 0.757 | 0.554 | 0.981 | 0.211 |
| ss9366251 | 0.697 | 0.263 | 0.539 | 0.271 | 0.324 | 0.75 | 0.658 |
| ss9378306 | 0.5 | 0.487 | 0.5 | 0.658 | 0.432 | 0.527 | 0.697 |
| ss9380511 | 0.541 | 0.405 | 0.974 | 0.608 | 0.921 | 0.603 | 0.838 |
| ss9382377 | 0.816 | 0.276 | 0.895 | 0.878 | 0.676 | 0.649 | 0.434 |
| ss9403022 | 0.408 | 0.649 | 0.882 | 0.014 | 0.611 | 0.861 | 0.324 |
| ss9409752 | 0.181 | 0.145 | 0.474 | 0.816 | 0.861 | 0.789 | 0.946 |
| ss9419768 | 0.066 | 0.171 | 0.276 | 0.042 | 0.068 | 0.908 | 0.684 |
| ss9423342 | 0.784 | 0.987 | 0.579 | 0.649 | 0.081 | 0.842 | 0.566 |
| ss9427809 | 0.586 | 0.395 | 0.513 | 0.737 | 0.026 | 0.81 | 0.284 |
| ss9432314 | 0.303 | 0.23 | 0.149 | 0.092 | 0.066 | 0.568 | 0.829 |
| ss9480981 | 0.392 | 0.421 | 0.132 | 0.184 | 0.847 | 0.684 | 0.145 |
| ss9490183 | 0.184 | 0.382 | 0.605 | 0.271 | 0 | 0.316 | 0.014 |
| ss9496479 | 0.763 | 1 | 0.905 | 0.838 | 0.122 | 0.934 | 0.776 |
| ss9502221 | 0.724 | 0.895 | 0.579 | 0.447 | 0.934 | 0.819 | 0.079 |
| ss9519462 | 0.658 | 0.378 | 0.263 | 0.513 | 0.382 | 0.421 | 0.342 |
| ss9527721 | 0.724 | 0.039 | 0.882 | 0.77 | 0.5 | 0.5 | 0.9 |
| ss9565630 | 0.908 | 0.986 | 0.697 | 0.486 | 1 | 1 | 1 |
| ss9586065 | 0.987 | 0.959 | 0.632 | 1 | 0.905 | 0.892 | 0.263 |
| ss9627150 | 0.263 | 0 | 0.013 | 0.014 | 0 | 0.237 | 0.066 |
| ss9628837 | 0.632 | 0.934 | 0.581 | 1 | 0.622 | 0.211 | 0.171 |
| ss9645529 | 0.541 | 0.203 | 0.039 | 0.355 | 0.554 | 0.778 | 1 |
| ss9646032 | 0.513 | 0.921 | 0.75 | 0.108 | 0.855 | 1 | 1 |
| ss9671733 | 0.486 | 0.289 | 0.763 | 0.803 | 1 | 0.434 | 0.597 |
| ss9678528 | 0.473 | 0.527 | 0.595 | 0.216 | 0.056 | 0.784 | 0.645 |
| ss9695373 | 0.75 | 0.934 | 0.289 | 0.838 | 0.554 | 0 | 0.276 |
| ss9705100 | 0.763 | 0.842 | 0.763 | 0.789 | 0.081 | 0.452 | 0.908 |
| ss9714487 | 0.697 | 0.553 | 0.697 | 0.026 | 0.189 | 0.303 | 0.554 |
| ss9719095 | 0.645 | 0.889 | 0.861 | 0.986 | 0.486 | 0.514 | 0.608 |
| ss9734846 | 0.351 | 0.539 | 0.197 | 0.041 | 0.382 | 0.186 | 0.703 |
| ss9759816 | 0.013 | 0.284 | 0.487 | 0.014 | 0 | 0 | 0.595 |
| ss9780984 | 0.434 | 0.671 | 0.605 | 0.041 | 0.392 | 1 | 1 |
| ss9800286 | 1 | 0.789 | 0.514 | 0.947 | 0.987 | 0.158 | 0.961 |

| GenBank | | Basset | | | | |
|---|---|---|---|---|---|---|
| Accession | Basenji | Hound | Beagle | Borzoi | Dachshund | Saluki |
| ss9053109 | 0.053 | 0.513 | 0.026 | 0.697 | 0.605 | 0.29 |
| ss9067589 | 1 | 0.597 | 0.737 | 0.5 | 0.203 | 0.683 |
| ss9069201 | 0.961 | 0.649 | 0.316 | 0.214 | 0.211 | 0.968 |
| ss9084075 | 1 | 0.917 | 0.778 | 0.865 | 0.829 | 0.819 |
| ss9108332 | 1 | 0.527 | 0.662 | 0.443 | 0.568 | 0.531 |
| ss9132982 | 0.987 | 0.014 | 0.026 | 0.222 | 0.167 | 0.125 |
| ss9139126 | 0.066 | 0.987 | 0.829 | 0.329 | 0.724 | 0.861 |
| ss9142796 | 1 | 0.474 | 1 | 0.973 | 0.689 | 1 |
| ss9152677 | 0.092 | 0 | 0.257 | 0 | 0.676 | 0.113 |
| ss9156891 | 0.338 | 0.351 | 0.473 | 0.528 | 0.757 | 0.931 |
| ss9191087 | 1 | 0.987 | 0.986 | 0.662 | 0.829 | 0.946 |
| ss9200241 | 0.987 | 0.743 | 0.816 | 1 | 0.681 | 0.972 |
| ss9230071 | 1 | 0.986 | 0.694 | 0.857 | 0.789 | 0.839 |
| ss9244345 | 1 | 0.395 | 0.378 | 0.392 | 0.676 | 0.857 |
| ss9251154 | 0.961 | 0.25 | 0.711 | 0.908 | 0.829 | 0.316 |
| ss9259716 | 0.974 | 0.811 | 0.697 | 0.357 | 0.554 | 0.735 |
| ss9278814 | 0.987 | 0.895 | 0.908 | 0.703 | 0.667 | 0.75 |
| ss9281595 | 0.868 | 0.526 | 0.338 | 0.444 | 0.324 | 0.422 |
| ss9290112 | 0.711 | 0.653 | 0.486 | 0.743 | 0.868 | 0.542 |
| ss9290361 | 0.566 | 0.816 | 0.684 | 0.237 | 0.592 | 0.378 |
| ss9296487 | 0.671 | 0.513 | 0.284 | 0.421 | 0.355 | 0.188 |
| ss9307596 | 1 | 0 | 0.474 | 0.25 | 0.132 | 0.176 |
| ss9313462 | 0.842 | 0.569 | 0.882 | 0.716 | 0.662 | 0.852 |
| ss9313564 | 1 | 0.486 | 0.184 | 0 | 0.039 | 0.149 |
| ss9335917 | 0.571 | 0.972 | 0.241 | 0.933 | 1 | 0.781 |
| ss9339680 | 0.895 | 0 | 0.527 | 0.271 | 0.176 | 0.383 |
| ss9362797 | 0.092 | 0.351 | 0.554 | 0.324 | 0.639 | 0.516 |
| ss9366135 | 0.703 | 0.974 | 0.513 | 0.539 | 0.5 | 0.786 |
| ss9366251 | 1 | 0.184 | 0.842 | 0.878 | 0.446 | 0.717 |
| ss9378306 | 0.974 | 0.947 | 0.737 | 0.446 | 0.737 | 0.613 |
| ss9380511 | 0.053 | 0.403 | 0.737 | 0.716 | 0.743 | 0.819 |
| ss9382377 | 0.237 | 0.855 | 0.803 | 0.5 | 0.919 | 0.562 |
| ss9403022 | 0.066 | 0.419 | 0.276 | 0.868 | 0.446 | 0.55 |
| ss9409752 | 0.763 | 0.405 | 0.697 | 0.608 | 0.229 | 0.887 |
| ss9419768 | 0.987 | 0.118 | 0.408 | 0 | 0.216 | 0.111 |
| ss9423342 | 0.737 | 0.986 | 0.421 | 1 | 0.959 | 0.861 |
| ss9427809 | 0.118 | 0.513 | 0.419 | 0.292 | 0.711 | 0.662 |
| ss9432314 | 0.671 | 0.75 | 0.276 | 0.566 | 0.539 | 0.441 |
| ss9480981 | 1 | 0.378 | 0.361 | 0.444 | 0.474 | 0.71 |
| ss9490183 | 0.357 | 0.672 | 0.824 | 0.283 | 0.459 | 0.617 |
| ss9496479 | 0.276 | 0.987 | 0.579 | 0.171 | 0.921 | 0.797 |
| ss9502221 | 0.697 | 0.526 | 0.703 | 0.25 | 0.716 | 0.23 |
| ss9519462 | 0.961 | 0.645 | 0.697 | 0.526 | 0.697 | 0.487 |
| ss9527721 | 0.75 | 0.486 | 0.684 | 0.814 | 0.622 | 0.986 |
| ss9565630 | 0.865 | 0.842 | 0.851 | 0.355 | 0.487 | 0.467 |
| ss9586065 | 0.803 | 1 | 1 | 0.414 | 0.649 | 0.443 |
| ss9627150 | 0.382 | 0 | 0.343 | 0.591 | 0.081 | 0.117 |
| ss9628837 | 1 | 0.868 | 0.237 | 0.892 | 0.568 | 0.4 |
| ss9645529 | 0.971 | 0.632 | 0.189 | 0.647 | 0.5 | 1 |
| ss9646032 | 1 | 1 | 0.921 | 0.986 | 0.289 | 0.842 |
| ss9671733 | 0.066 | 0.579 | 0.579 | 0.757 | 0.736 | 0.895 |
| ss9678528 | 0.943 | 0.908 | 0.105 | 0.932 | 0.347 | 0.788 |
| ss9695373 | 0 | 0.539 | 0.987 | 0.571 | 0.865 | 0.345 |
| ss9705100 | 0.974 | 0.803 | 0.921 | 1 | 0.568 | 0.677 |
| ss9714487 | 0.526 | 0.868 | 0.658 | 0.276 | 0.579 | 0.724 |
| ss9719095 | 0.778 | 0.932 | 1 | 0.378 | 0.392 | 0.645 |
| ss9734846 | 0.694 | 0.042 | 0.722 | 0.181 | 0.447 | 0.444 |
| ss9759816 | 0.566 | 0.382 | 0.303 | 0.191 | 0.153 | 0.167 |
| ss9780984 | 0.974 | 0.882 | 0.25 | 0.447 | 0.513 | 0.947 |
| ss9800286 | 0.189 | 0.526 | 0.645 | 0.568 | 0.903 | 0.608 |

| GenBank | | | | | Yorkshire | Bernese |
|---|---|---|---|---|---|---|
| Accession | Whippet | Chihuahua | Pug | Shih Tzu | Terrier | Mountain |
| ss9053109 | 0.662 | 0.306 | 0.105 | 0 | 0.789 | 0.184 |
| ss9067589 | 0.539 | 0.603 | 0.671 | 0.851 | 0.514 | 0.224 |
| ss9069201 | 0.276 | 0.545 | 0.118 | 0.816 | 0.5 | 0.122 |
| ss9084075 | 0.211 | 0.578 | 0.284 | 0.736 | 0.158 | 0.264 |
| ss9108332 | 0.289 | 0.833 | 0 | 0.645 | 0.632 | 0.931 |
| ss9132982 | 0 | 0.344 | 0.487 | 1 | 0.257 | 0.806 |
| ss9139126 | 1 | 0.529 | 0.329 | 0.724 | 0.816 | 1 |
| ss9142796 | 0.474 | 0.406 | 1 | 0.855 | 0.921 | 1 |
| ss9152677 | 0.014 | 0.29 | 0 | 0.092 | 0.434 | 0 |
| ss9156891 | 0.789 | 0.6 | 1 | 0.855 | 0.382 | 0.891 |
| ss9191087 | 0.868 | 0.47 | 0.908 | 0.25 | 0.697 | 0.737 |
| ss9200241 | 0.842 | 0.891 | 0.645 | 0.895 | 0.974 | 0.676 |
| ss9230071 | 0.224 | 0.561 | 1 | 1 | 0.554 | 0.971 |
| ss9244345 | 0.25 | 0.5 | 0.447 | 0.539 | 0.329 | 0.842 |
| ss9251154 | 0.513 | 0.515 | 0.408 | 0.382 | 0.724 | 0.987 |
| ss9259716 | 0.763 | 0.5 | 0.132 | 0.5 | 0.408 | 0.386 |
| ss9278814 | 0.447 | 0.439 | 1 | 0.895 | 0.605 | 0.297 |
| ss9281595 | 0.026 | 0.403 | 0.118 | 0.921 | 0.842 | 0.514 |
| ss9290112 | 0.316 | 0.594 | 0.421 | 0.342 | 0.579 | 1 |
| ss9290361 | 0.329 | 0.191 | 0.224 | 0.763 | 0.724 | 0.697 |
| ss9296487 | 0 | 0.088 | 0 | 0.263 | 0.145 | 0 |
| ss9307596 | 0.053 | 0.242 | 0.5 | 0.079 | 0.158 | 0 |
| ss9313462 | 0.605 | 0.672 | 0.974 | 0.513 | 0.816 | 0.571 |
| ss9313564 | 0.303 | 0.176 | 0 | 0.013 | 0.066 | 0.068 |
| ss9335917 | 0.959 | 0.983 | 0.118 | 0.75 | 0.724 | 1 |
| ss9339680 | 0.118 | 0.532 | 0.108 | 0 | 0.461 | 0 |
| ss9362797 | 0.324 | 0.456 | 0.711 | 1 | 0.819 | 0.316 |
| ss9366135 | 0.622 | 0.383 | 0.568 | 0.635 | 0.368 | 0.908 |
| ss9366251 | 0.829 | 0.682 | 0.526 | 0.921 | 0.342 | 0.919 |
| ss9378306 | 0.763 | 0.212 | 0.211 | 0.763 | 0.461 | 0.526 |
| ss9380511 | 0.378 | 0.515 | 0.487 | 0.258 | 0.527 | 0.592 |
| ss9382377 | 0.211 | 0.409 | 1 | 0.974 | 0.737 | 0.865 |
| ss9403022 | 0.421 | 0.303 | 0.553 | 0.316 | 0.838 | 0.273 |
| ss9409752 | 0.203 | 0.516 | 0.092 | 0.092 | 0.684 | 0.857 |
| ss9419768 | 0.118 | 0.25 | 0.421 | 0.132 | 0.079 | 0.237 |
| ss9423342 | 0.605 | 0.952 | 0.908 | 0.724 | 0.908 | 0.527 |
| ss9427809 | 0.895 | 0.5 | 0 | 0.855 | 0.868 | 0.447 |
| ss9432314 | 0.447 | 0.559 | 0.013 | 0.829 | 0.276 | 0.158 |
| ss9480981 | 0.75 | 0.226 | 0.145 | 0.724 | 0.27 | 0.743 |
| ss9490183 | 0.446 | 0.578 | 0 | 0.5 | 0.789 | 0.895 |
| ss9496479 | 0.855 | 0.676 | 0.921 | 0.487 | 0.908 | 1 |
| ss9502221 | 0.224 | 0.613 | 0.684 | 0.671 | 0.75 | 0.947 |
| ss9519462 | 0.237 | 0.647 | 0.118 | 0.724 | 0.355 | 0.039 |
| ss9527721 | 0.461 | 0.833 | 0.743 | 0.408 | 0.211 | 0.526 |
| ss9565630 | 0.316 | 0.924 | 0.895 | 0.816 | 0.8 | 0.811 |
| ss9586065 | 0.947 | 0.941 | 1 | 0.671 | 1 | 0.75 |
| ss9627150 | 0 | 0.125 | 0.171 | 0.338 | 0.118 | 0.819 |
| ss9628837 | 0.066 | 0.594 | 0.211 | 0.447 | 0.658 | 1 |
| ss9645529 | 0.237 | 0.515 | 0.895 | 0.513 | 0.973 | 0.662 |
| ss9646032 | 0.658 | 0.922 | 0.947 | 1 | 0.842 | 0.829 |
| ss9671733 | 0.632 | 0.971 | 0.816 | 0.645 | 0.461 | 0.347 |
| ss9678528 | 0.158 | 0.435 | 0.743 | 0.566 | 0.474 | 0.622 |
| ss9695373 | 0.908 | 0.552 | 0.5 | 0.054 | 0.553 | 0 |
| ss9705100 | 0.921 | 0.812 | 0.776 | 1 | 0.865 | 0.365 |
| ss9714487 | 0.039 | 0.152 | 0.171 | 0.513 | 0.724 | 0.316 |
| ss9719095 | 0.487 | 0.758 | 0.368 | 0.908 | 0.474 | 0.158 |
| ss9734846 | 0.105 | 0.203 | 0.139 | 0.092 | 0.342 | 0.527 |
| ss9759816 | 0.013 | 0.152 | 0 | 0.592 | 0.066 | 0 |
| ss9780984 | 0.447 | 0.625 | 1 | 1 | 0.763 | 0.947 |
| ss9800286 | 1 | 0.879 | 1 | 0.842 | 0.842 | 0.579 |

| GenBank | Doberman | | | St | | Siberian |
|---|---|---|---|---|---|---|
| Accession | Pinscher | Mastiff | Rottweiler | Bernard | Samoyed | Husky |
| ss9053109 | 0.592 | 0.789 | 1 | 0.919 | 0.297 | 0.041 |
| ss9067589 | 0.579 | 0.054 | 0.622 | 0.066 | 0.697 | 0.944 |
| ss9069201 | 0.961 | 0.421 | 0.554 | 0.122 | 0.405 | 0.974 |
| ss9084075 | 0.111 | 0.329 | 0.842 | 0.595 | 0.516 | 0.961 |
| ss9108332 | 0 | 0.554 | 0.77 | 0.5 | 0.892 | 0.618 |
| ss9132982 | 0.711 | 0.378 | 0.444 | 0.25 | 0.736 | 0.684 |
| ss9139126 | 0.303 | 1 | 0.947 | 0.803 | 0.882 | 0.566 |
| ss9142796 | 0.408 | 0.829 | 0.671 | 1 | 1 | 0.895 |
| ss9152677 | 0.987 | 0 | 0.041 | 0 | 0.557 | 0.671 |
| ss9156891 | 1 | 0.946 | 0.553 | 0.974 | 0.878 | 0.312 |
| ss9191087 | 0.868 | 0.776 | 0.316 | 0.092 | 0.474 | 0.895 |
| ss9200241 | 1 | 0.149 | 0.676 | 0.908 | 0.5 | 0.105 |
| ss9230071 | 0.865 | 0.539 | 0.592 | 0.971 | 0.297 | 0.711 |
| ss9244345 | 0.214 | 0.434 | 0.122 | 0.447 | 0.408 | 0.724 |
| ss9251154 | 0.684 | 0.806 | 0.868 | 1 | 0.342 | 0.118 |
| ss9259716 | 0.189 | 0.905 | 0.583 | 0.653 | 0.432 | 0.243 |
| ss9278814 | 0.974 | 0.712 | 0.972 | 1 | 0.973 | 0.741 |
| ss9281595 | 0.224 | 0.868 | 0.132 | 0.608 | 0.581 | 0.895 |
| ss9290112 | 0.934 | 0.764 | 0.947 | 0.842 | 1 | 0.868 |
| ss9290361 | 0.789 | 0 | 0.486 | 0.711 | 0.303 | 0.689 |
| ss9296487 | 0.579 | 0.276 | 0.487 | 0.122 | 0.395 | 0.632 |
| ss9307596 | 0.039 | 0.026 | 0.039 | 0.434 | 0.329 | 0.553 |
| ss9313462 | 0.224 | 0.236 | 0.75 | 0.684 | 0.961 | 0.763 |
| ss9313564 | 0.382 | 0.724 | 0 | 0.158 | 0.25 | 0.474 |
| ss9335917 | 0.958 | 0.935 | 1 | 1 | 0.959 | 0.967 |
| ss9339680 | 0 | 0.054 | 0.081 | 0.541 | 0.139 | 0.224 |
| ss9362797 | 0.843 | 0.421 | 0.329 | 0.776 | 0.868 | 0.972 |
| ss9366135 | 0.934 | 0.922 | 1 | 0.736 | 0.935 | 1 |
| ss9366251 | 1 | 0.419 | 0.662 | 0.878 | 0.784 | 0.868 |
| ss9378306 | 0.434 | 0.947 | 0.73 | 0.25 | 0.263 | 0.632 |
| ss9380511 | 0.947 | 1 | 0.961 | 1 | 0.855 | 1 |
| ss9382377 | 0.408 | 1 | 0.351 | 0.974 | 1 | 0.797 |
| ss9403022 | 0.368 | 0.541 | 0.486 | 0.743 | 0.903 | 0.243 |
| ss9409752 | 0.908 | 0.014 | 0.429 | 0.097 | 0.263 | 0.25 |
| ss9419768 | 0.026 | 0.118 | 0.541 | 0 | 0.224 | 0.405 |
| ss9423342 | 0.829 | 1 | 1 | 1 | 0.513 | 1 |
| ss9427809 | 0.066 | 0.833 | 0 | 0.934 | 0.224 | 0.737 |
| ss9432314 | 0.303 | 0.297 | 0.882 | 0.513 | 0.092 | 0.689 |
| ss9480981 | 1 | 0.811 | 0.528 | 0.158 | 0.514 | 0.368 |
| ss9490183 | 0.043 | 0.514 | 0.543 | 0.095 | 0.789 | 0.737 |
| ss9496479 | 0.408 | 0.855 | 0.961 | 0.658 | 0.368 | 0.789 |
| ss9502221 | 0.197 | 0.645 | 0.645 | 0.919 | 0.842 | 0.932 |
| ss9519462 | 0.316 | 0.329 | 0.684 | 0.145 | 0.816 | 0.434 |
| ss9527721 | 0.25 | 0.908 | 0.987 | 0.986 | 0.314 | 0.342 |
| ss9565630 | 0.079 | 0.243 | 0.013 | 0.257 | 0.397 | 0.789 |
| ss9586065 | 0.868 | 1 | 0.855 | 0.724 | 0.794 | 0.257 |
| ss9627150 | 0 | 0.158 | 0.278 | 0.014 | 0.038 | 0.211 |
| ss9628837 | 0.816 | 0.378 | 0.568 | 0.303 | 0.408 | 0.895 |
| ss9645529 | 0.763 | 0.292 | 0.905 | 0.446 | 1 | 0.368 |
| ss9646032 | 0.25 | 0.351 | 0.934 | 1 | 0.947 | 0.974 |
| ss9671733 | 0.974 | 0.118 | 0.895 | 0.987 | 0.703 | 0.645 |
| ss9678528 | 0.553 | 0.118 | 0.129 | 0.026 | 0.013 | 0.784 |
| ss9695373 | 0.974 | 0.943 | 0.329 | 0.868 | 0.73 | 0 |
| ss9705100 | 0.434 | 0.811 | 0.946 | 1 | 1 | 0.961 |
| ss9714487 | 0.763 | 0.158 | 0.171 | 0.434 | 0.342 | 0.461 |
| ss9719095 | 0.378 | 0.303 | 0.776 | 0.566 | 0.903 | 0.75 |
| ss9734846 | 0 | 0.079 | 0.392 | 0.662 | 0.539 | 0.25 |
| ss9759816 | 0.132 | 0.236 | 0.514 | 0.276 | 0 | 0 |
| ss9780984 | 1 | 0.784 | 1 | 0.75 | 0.921 | 1 |
| ss9800286 | 0.895 | 0.947 | 0.905 | 0.868 | 0.934 | 0.5 |

| GenBank | | | Belgian | German | Shetland | |
|---|---|---|---|---|---|---|
| Accession | Akita | Poodle | Tervuren | Shepherd | Sheepdog | All |
| ss9053109 | 0.211 | 0.592 | 0.724 | 0.934 | 0.316 | 0.459 |
| ss9067589 | 1 | 0.5 | 0.486 | 0.014 | 0.053 | 0.52 |
| ss9069201 | 0.868 | 0.605 | 0.184 | 0.513 | 0.824 | 0.5 |
| ss9084075 | 1 | 0.645 | 0.649 | 0.737 | 0.974 | 0.638 |
| ss9108332 | 0.708 | 0.786 | 0.632 | 0.351 | 0.789 | 0.593 |
| ss9132982 | 0.412 | 0.395 | 0.513 | 0.081 | 0.618 | 0.419 |
| ss9139126 | 0.908 | 0.632 | 0.776 | 0.355 | 0.105 | 0.647 |
| ss9142796 | 0.908 | 0.176 | 0.697 | 0.804 | 0.961 | 0.805 |
| ss9152677 | 0.458 | 0.037 | 0.216 | 0.038 | 0.443 | 0.238 |
| ss9156891 | 0.776 | 0.028 | 0.895 | 1 | 0.447 | 0.72 |
| ss9191087 | 0.971 | 0.882 | 0.171 | 1 | 0.474 | 0.739 |
| ss9200241 | 0.157 | 0.711 | 1 | 0.2 | 0.316 | 0.667 |
| ss9230071 | 0.779 | 0.871 | 0.892 | 0.789 | 0.947 | 0.753 |
| ss9244345 | 0.513 | 0.5 | 0.153 | 0.571 | 0.474 | 0.49 |
| ss9251154 | 0.961 | 0.697 | 0.632 | 0.368 | 0.973 | 0.695 |
| ss9259716 | 0.875 | 0.838 | 0.351 | 0.682 | 0.447 | 0.584 |
| ss9278814 | 0.653 | 0.5 | 0.711 | 0.968 | 0.27 | 0.694 |
| ss9281595 | 0.592 | 0.487 | 0 | 0.271 | 0.541 | 0.483 |
| ss9290112 | 0.53 | 0.763 | 0.608 | 0.618 | 0.297 | 0.656 |
| ss9290361 | 0.447 | 0.658 | 1 | 0.838 | 0.342 | 0.499 |
| ss9296487 | 0.25 | 0.118 | 0.158 | 0.013 | 0 | 0.216 |
| ss9307596 | 0.878 | 0.237 | 0.434 | 0.882 | 0 | 0.301 |
| ss9313462 | 0.711 | 0.594 | 0.592 | 0.435 | 0.903 | 0.649 |
| ss9313564 | 0.403 | 0.676 | 0.171 | 0.382 | 0 | 0.252 |
| ss9335917 | 0.608 | 0.983 | 0.896 | 0.208 | 0.973 | 0.837 |
| ss9339680 | 0.014 | 0.632 | 0.515 | 0.722 | 0 | 0.249 |
| ss9362797 | 0.597 | 0.197 | 0.716 | 0.865 | 0.289 | 0.575 |
| ss9366135 | 0.797 | 0.879 | 0.724 | 0.765 | 0.758 | 0.715 |
| ss9366251 | 0.389 | 0.338 | 0.25 | 1 | 0.737 | 0.638 |
| ss9378306 | 0.703 | 0.895 | 0.513 | 0.987 | 0.068 | 0.59 |
| ss9380511 | 0.176 | 0.697 | 0.708 | 0.974 | 0.697 | 0.675 |
| ss9382377 | 1 | 0.868 | 0.895 | 0.77 | 0.882 | 0.722 |
| ss9403022 | 0.943 | 0.527 | 0.892 | 0.319 | 1 | 0.53 |
| ss9409752 | 0.649 | 0.408 | 0.25 | 0.456 | 0.068 | 0.47 |
| ss9419768 | 0.914 | 0.189 | 0.224 | 0.041 | 0.276 | 0.279 |
| ss9423342 | 1 | 0.789 | 0.789 | 0.608 | 1 | 0.785 |
| ss9427809 | 0.263 | 0.63 | 0.513 | 0.522 | 0 | 0.472 |
| ss9432314 | 0.974 | 0.671 | 0.474 | 0.921 | 0.568 | 0.472 |
| ss9480981 | 0.473 | 0.132 | 0.892 | 0.878 | 0.737 | 0.514 |
| ss9490183 | 0.361 | 0.632 | 0.5 | 0 | 0.013 | 0.4 |
| ss9496479 | 0.868 | 0.632 | 0.934 | 0.855 | 0.934 | 0.74 |
| ss9502221 | 1 | 0.5 | 0.421 | 0.865 | 0.224 | 0.623 |
| ss9519462 | 0.25 | 0.763 | 0.303 | 0.513 | 0.816 | 0.485 |
| ss9527721 | 0.987 | 0.408 | 0.382 | 0.73 | 0.653 | 0.62 |
| ss9565630 | 0.917 | 0.703 | 0.539 | 0.851 | 0.819 | 0.668 |
| ss9586065 | 0.875 | 0.934 | 0.592 | 0.959 | 0.868 | 0.805 |
| ss9627150 | 0.838 | 0.158 | 0 | 0.284 | 0.184 | 0.188 |
| ss9628837 | 0.419 | 0.211 | 0.855 | 0.851 | 0.861 | 0.581 |
| ss9645529 | 0.986 | 0.355 | 0.632 | 0.013 | 0 | 0.561 |
| ss9646032 | 1 | 0.316 | 0.974 | 0.961 | 1 | 0.802 |
| ss9671733 | 0.818 | 0.711 | 0.816 | 0.961 | 0.645 | 0.675 |
| ss9678528 | 0.471 | 0.329 | 0.289 | 0.814 | 0.473 | 0.473 |
| ss9695373 | 0.014 | 0.408 | 0.487 | 0.273 | 1 | 0.525 |
| ss9705100 | 0.716 | 0.944 | 0.987 | 0.868 | 0.895 | 0.802 |
| ss9714487 | 0.184 | 0.395 | 0.303 | 0.039 | 0.684 | 0.416 |
| ss9719095 | 0.819 | 0.487 | 0.592 | 0.579 | 0.595 | 0.63 |
| ss9734846 | 0.811 | 0.703 | 0.329 | 0 | 0.114 | 0.34 |
| ss9759816 | 0 | 0.697 | 0.263 | 0.041 | 0.25 | 0.214 |
| ss9780984 | 0.382 | 0.697 | 0.618 | 0.553 | 0.882 | 0.717 |
| ss9800286 | 0.355 | 0.635 | 1 | 1 | 0.579 | 0.756 |

TABLE 3
Summary of exclusion probability and probability match rate by breed.

| Breed | EPR | MPR |
|---|---|---|
| Afghan Hound | 1.40E−14 | 0.99960 |
| Akita | 3.69E−14 | 0.99939 |
| Basenji | 4.61E−09 | 0.99375 |
| Basset Hound | 2.27E−16 | 0.99953 |
| Beagle | 3.46E−17 | 0.99988 |
| Belgian Tervuren | 6.89E−18 | 0.99986 |
| Bernese Mountain Dog | 3.68E−13 | 0.99901 |
| Borzoi | 5.21E−18 | 0.99986 |
| Chihuahua | 2.76E−21 | 0.99995 |
| Chinese Shar-Pei | 1.71E−15 | 0.99968 |
| Cocker Spaniel | 8.47E−13 | 0.99876 |
| Dachshund | 1.27E−19 | 0.99994 |
| Doberman Pinscher | 8.07E−12 | 0.99854 |
| German Shepherd Dog | 7.55E−13 | 0.99894 |
| German Shorthaired Pointer | 4.62E−20 | 0.99994 |
| Golden Retriever | 5.98E−16 | 0.99972 |
| Labrador Retriever | 2.93E−18 | 0.99987 |
| Mastiff | 4.99E−13 | 0.99927 |
| Miniature Schnauzer | 2.66E−12 | 0.99750 |
| Poodle | 2.84E−19 | 0.99994 |
| Pug | 1.89E−12 | 0.99832 |
| Rottweiler | 4.04E−15 | 0.99940 |
| Saluki | 1.32E−16 | 0.99983 |
| Samoyed | 2.92E−15 | 0.99961 |
| Shetland.Sheepdog | 2.86E−13 | 0.99906 |
| Shih-Tzu | 4.54E−15 | 0.99957 |
| Siberian Husky | 2.82E−14 | 0.99956 |
| St. Bernard | 2.80E−12 | 0.99857 |
| Whippet | 2.34E−15 | 0.99967 |
| Yorkshire Terrier | 1.20E−17 | 0.99989 |

EPR = Exclusion probability rate
MPR = Match probability rate

TABLE 4
Proportion of predefined populations in computed clusters

| | Probability |
|---|---|
| | of |
| Breed | Assignment |
| Afghan Hound | 0.9 |
| Basenji | 0.946 |
| Basset Hound | 0.881 |
| Beagle | 0.86 |
| Belgian Tervuren | 0.865 |
| Bernese Mountain Dog | 0.913 |
| Borzoi | 0.83 |
| Chihuahua | 0.676 |
| Chinese Shar-Pei | 0.802 |
| Cocker Spaniel | 0.884 |
| Dachshund | 0.697 |
| Doberman Pinscher | 0.914 |
| German Shepherd Dog | 0.906 |
| German Shorthaired Pointer | 0.746 |
| Golden Retriever | 0.852 |
| Labrador Retriever | 0.809 |
| Mastiff | 0.881 |
| Miniature Schnauzer | 0.914 |
| Poodle | 0.805 |
| Pug | 0.928 |
| Rottweiler | 0.897 |
| Saluki | 0.568 |
| Samoyed | 0.847 |
| Shetland Sheepdog | 0.918 |
| Siberian Husky | 0.861 |
| St Bernard | 0.896 |
| Whippet | 0.863 |
| Yorkshire Terrier | 0.776 |

TABLE 5
Probability of assignment to specific cluster groups.

| | % | Cluster | | | | | | | | | | | | | | |
|---|---|---|---|---|---|---|---|---|---|---|---|---|---|---|---|---|
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Afghan Hound | 0 | 0.004 | 0.005 | 0.003 | 0.005 | 0.002 | 0.006 | 0.001 | 0.003 | 0.001 | 0.001 | 0.003 | 0.002 | 0.003 | 0.006 | 0.002 |
| Afghan Hound | 0 | 0.002 | 0.001 | 0.008 | 0.005 | 0.007 | 0.002 | 0.001 | 0.001 | 0.001 | 0.001 | 0.002 | 0.001 | 0.001 | 0.001 | 0.011 |
| Afghan Hound | 0 | 0.002 | 0.006 | 0.002 | 0.002 | 0.001 | 0.004 | 0.005 | 0.004 | 0.003 | 0.003 | 0.001 | 0.002 | 0.002 | 0.001 | 0.002 |
| Afghan Hound | 0 | 0.003 | 0.001 | 0.004 | 0.003 | 0.001 | 0.002 | 0.002 | 0.002 | 0.001 | 0.001 | 0.002 | 0.002 | 0.002 | 0.003 | 0.002 |
| Afghan Hound | 0 | 0.003 | 0.01 | 0.004 | 0.008 | 0.002 | 0.003 | 0.001 | 0.002 | 0.001 | 0.003 | 0.001 | 0.002 | 0.006 | 0.005 | 0.007 |
| Afghan Hound | 0 | 0.003 | 0.004 | 0.003 | 0.001 | 0.002 | 0.008 | 0.003 | 0.003 | 0.003 | 0.002 | 0.005 | 0.002 | 0.003 | 0.007 | 0.004 |
| Afghan Hound | 0 | 0.005 | 0.023 | 0.001 | 0.003 | 0.001 | 0.007 | 0.004 | 0.002 | 0.001 | 0.004 | 0.002 | 0.005 | 0.015 | 0.002 | 0.001 |
| Afghan Hound | 0 | 0.002 | 0.026 | 0.008 | 0.005 | 0.003 | 0.003 | 0.001 | 0.002 | 0.001 | 0.004 | 0.002 | 0.002 | 0.002 | 0.006 | 0.004 |
| Afghan Hound | 0 | 0.001 | 0.009 | 0.001 | 0.001 | 0.001 | 0.009 | 0.015 | 0.002 | 0.001 | 0.002 | 0.008 | 0.001 | 0.006 | 0.009 | 0.003 |
| Afghan Hound | (1) | 0.004 | 0.016 | 0.026 | 0.01 | 0.002 | 0.007 | 0.003 | 0.003 | 0.001 | 0.004 | 0.003 | 0.004 | 0.003 | 0.003 | 0.004 |
| Afghan Hound | (2) | 0.002 | 0.004 | 0.002 | 0.003 | 0.003 | 0.003 | 0.002 | 0.001 | 0.001 | 0.001 | 0.001 | 0.003 | 0.001 | 0.002 | 0.003 |
| Afghan Hound | (3) | 0.003 | 0.008 | 0.046 | 0.024 | 0.004 | 0.006 | 0.004 | 0.007 | 0.003 | 0.003 | 0.005 | 0.002 | 0.003 | 0.003 | 0.005 |
| Afghan Hound | 0 | 0.002 | 0.002 | 0.006 | 0.004 | 0.003 | 0.002 | 0.003 | 0.002 | 0.003 | 0.002 | 0.004 | 0.005 | 0.002 | 0.002 | 0.004 |
| Afghan Hound | 0 | 0.002 | 0.003 | 0.005 | 0.003 | 0.001 | 0.002 | 0.002 | 0.002 | 0.002 | 0.001 | 0.001 | 0.003 | 0.002 | 0.002 | 0.001 |
| Afghan Hound | (1) | 0.003 | 0.004 | 0.013 | 0.009 | 0.002 | 0.003 | 0.002 | 0.007 | 0.002 | 0.012 | 0.006 | 0.001 | 0.003 | 0.004 | 0.006 |
| Afghan Hound | 0 | 0.001 | 0.011 | 0.003 | 0.007 | 0.006 | 0.005 | 0.002 | 0.002 | 0.001 | 0.002 | 0.002 | 0.003 | 0.002 | 0.003 | 0.006 |
| Afghan Hound | 0 | 0.001 | 0.002 | 0.006 | 0.004 | 0.001 | 0.002 | 0.002 | 0.002 | 0.002 | 0.003 | 0.005 | 0.003 | 0.002 | 0.003 | 0.002 |
| Afghan Hound | 0 | 0.006 | 0.003 | 0.002 | 0.004 | 0.002 | 0.004 | 0.003 | 0.003 | 0.003 | 0.01 | 0.004 | 0.013 | 0.004 | 0.002 | 0.002 |
| Afghan Hound | 0 | 0.009 | 0.002 | 0.003 | 0.003 | 0.003 | 0.007 | 0.003 | 0.003 | 0.001 | 0.003 | 0.002 | 0.002 | 0.001 | 0.001 | 0.003 |
| Afghan Hound | (1) | 0.002 | 0.002 | 0.003 | 0.002 | 0.002 | 0.003 | 0.003 | 0.005 | 0.009 | 0.001 | 0.007 | 0.002 | 0.002 | 0.002 | 0.003 |
| Afghan Hound | (1) | 0.003 | 0.001 | 0.003 | 0.012 | 0.008 | 0.002 | 0.003 | 0.002 | 0.002 | 0.003 | 0.002 | 0.001 | 0.003 | 0.002 | 0.004 |
| Afghan Hound | (1) | 0.004 | 0.006 | 0.011 | 0.006 | 0.004 | 0.006 | 0.006 | 0.007 | 0.006 | 0.003 | 0.023 | 0.002 | 0.004 | 0.006 | 0.005 |
| Afghan Hound | 0 | 0.005 | 0.003 | 0.001 | 0.001 | 0.002 | 0.002 | 0.001 | 0.002 | 0.001 | 0.002 | 0.001 | 0.002 | 0.002 | 0.001 | 0.003 |
| Afghan Hound | 0 | 0.004 | 0.002 | 0.001 | 0.021 | 0.001 | 0.003 | 0.001 | 0.002 | 0.001 | 0.003 | 0.004 | 0.001 | 0.002 | 0.001 | 0.001 |
| Afghan Hound | 0 | 0.005 | 0.003 | 0.002 | 0.024 | 0.007 | 0.004 | 0.003 | 0.002 | 0.001 | 0.001 | 0.001 | 0.002 | 0.01 | 0.001 | 0.002 |
| Afghan Hound | (1) | 0.003 | 0.004 | 0.004 | 0.002 | 0.003 | 0.002 | 0.003 | 0.002 | 0.001 | 0.002 | 0.004 | 0.001 | 0.001 | 0.001 | 0.027 |
| Afghan Hound | 0 | 0.003 | 0.003 | 0.001 | 0.001 | 0.003 | 0.003 | 0.002 | 0.006 | 0.002 | 0.002 | 0.011 | 0.001 | 0.002 | 0.002 | 0.004 |
| Afghan Hound | 0 | 0.003 | 0.002 | 0.002 | 0.003 | 0.002 | 0.002 | 0.001 | 0.003 | 0.003 | 0.002 | 0.005 | 0.002 | 0.002 | 0.001 | 0.002 |
| Afghan Hound | (1) | 0.001 | 0.002 | 0.003 | 0.005 | 0.005 | 0.002 | 0.001 | 0.002 | 0.002 | 0.004 | 0.004 | 0.003 | 0.002 | 0.004 | 0.007 |
| Afghan Hound | (5) | 0.002 | 0.001 | 0.002 | 0.002 | 0.002 | 0.001 | 0.001 | 0.002 | 0.002 | 0.002 | 0.004 | 0.001 | 0.001 | 0.001 | 0.002 |
| Afghan Hound | 0 | 0.002 | 0.002 | 0.002 | 0.005 | 0.002 | 0.006 | 0.001 | 0.002 | 0.002 | 0.003 | 0.002 | 0.001 | 0.002 | 0.001 | 0.001 |
| Afghan Hound | (1) | 0.003 | 0.003 | 0.002 | 0.004 | 0.002 | 0.006 | 0.001 | 0.002 | 0.004 | 0.007 | 0.004 | 0.001 | 0.002 | 0.001 | 0.002 |
| Afghan Hound | (2) | 0.001 | 0.002 | 0.002 | 0.002 | 0.003 | 0.002 | 0.001 | 0.002 | 0.001 | 0.001 | 0.004 | 0.001 | 0.001 | 0.001 | 0.004 |
| Afghan Hound | (6) | 0.003 | 0.003 | 0.004 | 0.004 | 0.002 | 0.001 | 0.001 | 0.002 | 0.001 | 0.007 | 0.004 | 0.002 | 0.002 | 0.002 | 0.003 |
| Afghan Hound | (2) | 0.002 | 0.002 | 0.008 | 0.004 | 0.004 | 0.003 | 0.002 | 0.004 | 0.002 | 0.001 | 0.002 | 0.001 | 0.001 | 0.001 | 0.002 |
| Afghan Hound | (1) | 0.001 | 0.004 | 0.018 | 0.006 | 0.008 | 0.018 | 0.003 | 0.003 | 0.003 | 0.003 | 0.008 | 0.002 | 0.002 | 0.002 | 0.049 |
| Afghan Hound | 0 | 0.003 | 0.004 | 0.001 | 0.002 | 0.001 | 0.001 | 0.003 | 0.004 | 0.002 | 0.004 | 0.002 | 0.002 | 0.004 | 0.03 | 0.003 |
| Afghan Hound | 0 | 0.002 | 0.003 | 0.002 | 0.003 | 0.004 | 0.003 | 0.002 | 0.002 | 0.001 | 0.004 | 0.001 | 0.001 | 0.001 | 0.002 | 0.002 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Afghan Hound | | 0.002 | 0.001 | 0.002 | 0.907 | 0.001 | 0.008 | 0.002 | 0.004 | 0.01 | 0.002 | 0.002 | 0.004 | 0.002 | 0.002 | 0.002 |
| Afghan Hound | | 0.001 | 0.001 | 0.001 | 0.917 | 0.002 | 0.002 | 0.001 | 0.003 | 0.01 | 0.001 | 0.001 | 0.001 | 0.007 | 0.007 | 0.002 |
| Afghan Hound | | 0.002 | 0.003 | 0.003 | 0.902 | 0.002 | 0.003 | 0.012 | 0.005 | 0.002 | 0.002 | 0.003 | 0.007 | 0.003 | 0.004 | 0.009 |
| Afghan Hound | | 0.001 | 0.002 | 0.002 | 0.947 | 0.002 | 0.002 | 0.001 | 0.002 | 0.002 | 0.001 | 0.001 | 0.002 | 0.002 | 0.001 | 0.003 |
| Afghan Hound | | 0.001 | 0.001 | 0.002 | 0.906 | 0.001 | 0.002 | 0.001 | 0.003 | 0.008 | 0.005 | 0.002 | 0.001 | 0.002 | 0.003 | 0.003 |
| Afghan Hound | | 0.002 | 0.002 | 0.007 | 0.892 | 0.001 | 0.002 | 0.003 | 0.001 | 0.013 | 0.009 | 0.001 | 0.002 | 0.004 | 0.002 | 0.007 |
| Afghan Hound | | 0.002 | 0.002 | 0.004 | 0.868 | 0.001 | 0.002 | 0.021 | 0.003 | 0.001 | 0.003 | 0.005 | 0.002 | 0.001 | 0.002 | 0.007 |
| Afghan Hound | | 0.002 | 0.006 | 0.007 | 0.872 | 0.002 | 0.01 | 0.005 | 0.002 | 0.003 | 0.002 | 0.001 | 0.001 | 0.002 | 0.001 | 0.011 |
| Afghan Hound | | 0.003 | 0.004 | 0.002 | 0.892 | 0.002 | 0.002 | 0.008 | 0.002 | 0.002 | 0.001 | 0.004 | 0.001 | 0.004 | 0.002 | 0.001 |
| Afghan Hound | | 0.001 | 0.015 | 0.013 | 0.833 | 0.005 | 0.003 | 0.004 | 0.006 | 0.008 | 0.001 | 0.003 | 0.003 | 0.006 | 0.002 | 0.003 |
| Afghan Hound | | 0.002 | 0.002 | 0.003 | 0.945 | 0.002 | 0.002 | 0.001 | 0.002 | 0.002 | 0.001 | 0.001 | 0.001 | 0.001 | 0.001 | 0.001 |
| Afghan Hound | | 0.002 | 0.024 | 0.009 | 0.803 | 0.005 | 0.004 | 0.002 | 0.003 | 0.003 | 0.002 | 0.004 | 0.004 | 0.004 | 0.001 | 0.005 |
| Afghan Hound | | 0.001 | 0.001 | 0.001 | 0.917 | 0.004 | 0.003 | 0.004 | 0.002 | 0.003 | 0.002 | 0.001 | 0.003 | 0.004 | 0.006 | 0.001 |
| Afghan Hound | | 0.001 | 0.005 | 0.002 | 0.931 | 0.002 | 0.002 | 0.003 | 0.003 | 0.002 | 0.001 | 0.002 | 0.002 | 0.005 | 0.002 | 0.003 |
| Afghan Hound | | 0.002 | 0.021 | 0.005 | 0.751 | 0.007 | 0.004 | 0.07 | 0.006 | 0.004 | 0.028 | 0.002 | 0.01 | 0.006 | 0.005 | 0.003 |
| Afghan Hound | | 0.001 | 0.002 | 0.002 | 0.885 | 0.007 | 0.014 | 0.016 | 0.002 | 0.004 | 0.003 | 0.001 | 0.001 | 0.001 | 0.001 | 0.002 |
| Afghan Hound | | 0.001 | 0.004 | 0.003 | 0.928 | 0.001 | 0.001 | 0.004 | 0.002 | 0.002 | 0.002 | 0.001 | 0.002 | 0.002 | 0.005 | 0.002 |
| Afghan Hound | | 0.001 | 0.002 | 0.002 | 0.903 | 0.002 | 0.004 | 0.003 | 0.003 | 0.002 | 0.004 | 0.002 | 0.002 | 0.002 | 0.003 | 0.002 |
| Afghan Hound | | 0.001 | 0.001 | 0.002 | 0.927 | 0.002 | 0.002 | 0.004 | 0.001 | 0.002 | 0.003 | 0.002 | 0.001 | 0.002 | 0.004 | 0.002 |
| Afghan Hound | | 0.003 | 0.002 | 0.004 | 0.906 | 0.002 | 0.002 | 0.007 | 0.002 | 0.002 | 0.008 | 0.003 | 0.003 | 0.002 | 0.003 | 0.006 |
| Afghan Hound | | 0.003 | 0.002 | 0.001 | 0.909 | 0.002 | 0.002 | 0.003 | 0.003 | 0.001 | 0.004 | 0.001 | 0.003 | 0.009 | 0.003 | 0.004 |
| Afghan Hound | | 0.001 | 0.015 | 0.007 | 0.801 | 0.002 | 0.002 | 0.033 | 0.006 | 0.004 | 0.003 | 0.002 | 0.002 | 0.02 | 0.004 | 0.003 |
| Afghan Hound | | 0.001 | 0.002 | 0.001 | 0.952 | 0.001 | 0.001 | 0.002 | 0.001 | 0.002 | 0.001 | 0.001 | 0.001 | 0.002 | 0.001 | 0.002 |
| Afghan Hound | | 0.011 | 0.001 | 0.003 | 0.918 | 0.002 | 0.001 | 0.002 | 0.001 | 0.004 | 0.002 | 0.001 | 0.001 | 0.001 | 0.001 | 0.001 |
| Afghan Hound | | 0.002 | 0.001 | 0.001 | 0.907 | 0.001 | 0.002 | 0.001 | 0.002 | 0.002 | 0.003 | 0.001 | 0.003 | 0.002 | 0.001 | 0.003 |
| Afghan Hound | | 0.001 | 0.002 | 0.002 | 0.906 | 0.002 | 0.007 | 0.002 | 0.001 | 0.002 | 0.001 | 0.001 | 0.001 | 0.003 | 0.007 | 0.002 |
| Afghan Hound | | 0.001 | 0.001 | 0.003 | 0.918 | 0.003 | 0.002 | 0.003 | 0.002 | 0.003 | 0.002 | 0.002 | 0.001 | 0.005 | 0.005 | 0.002 |
| Afghan Hound | | 0.001 | 0.002 | 0.003 | 0.937 | 0.001 | 0.001 | 0.003 | 0.002 | 0.002 | 0.001 | 0.001 | 0.001 | 0.002 | 0.005 | 0.001 |
| Afghan Hound | | 0.001 | 0.002 | 0.008 | 0.901 | 0.002 | 0.002 | 0.002 | 0.006 | 0.003 | 0.001 | 0.005 | 0.002 | 0.003 | 0.014 | 0.002 |
| Afghan Hound | | 0.001 | 0.001 | 0.002 | 0.953 | 0.003 | 0.002 | 0.001 | 0.001 | 0.001 | 0.001 | 0.001 | 0.001 | 0.002 | 0.002 | 0.001 |
| Afghan Hound | | 0.001 | 0.003 | 0.001 | 0.922 | 0.004 | 0.001 | 0.001 | 0.001 | 0.001 | 0.002 | 0.002 | 0.002 | 0.021 | 0.001 | 0.001 |
| Afghan Hound | | 0.001 | 0.005 | 0.001 | 0.899 | 0.011 | 0.002 | 0.002 | 0.002 | 0.004 | 0.005 | 0.004 | 0.005 | 0.011 | 0.002 | 0.002 |
| Afghan Hound | | 0.001 | 0.003 | 0.002 | 0.95 | 0.001 | 0.001 | 0.001 | 0.001 | 0.002 | 0.001 | 0.001 | 0.002 | 0.002 | 0.002 | 0.002 |
| Afghan Hound | | 0.001 | 0.004 | 0.002 | 0.931 | 0.002 | 0.002 | 0.002 | 0.003 | 0.002 | 0.002 | 0.001 | 0.002 | 0.004 | 0.002 | 0.002 |
| Afghan Hound | | 0.002 | 0.002 | 0.002 | 0.935 | 0.003 | 0.002 | 0.002 | 0.002 | 0.002 | 0.001 | 0.002 | 0.001 | 0.002 | 0.002 | 0.002 |
| Afghan Hound | | 0.002 | 0.002 | 0.005 | 0.812 | 0.003 | 0.002 | 0.005 | 0.001 | 0.005 | 0.002 | 0.002 | 0.001 | 0.002 | 0.024 | 0.003 |
| Afghan Hound | | 0.001 | 0.001 | 0.003 | 0.862 | 0.001 | 0.004 | 0.004 | 0.01 | 0.008 | 0.02 | 0.001 | 0.004 | 0.004 | 0.003 | 0.005 |
| Afghan Hound | | 0.001 | 0.002 | 0.002 | 0.941 | 0.002 | 0.002 | 0.002 | 0.001 | 0.002 | 0.002 | 0.001 | 0.001 | 0.001 | 0.003 | 0.003 |
| | % | Cluster | | | | | | | | | | | | | | |
| | missing | Assignment | | | | | | | | | | | | | | |
| Breed | data | 1 | 2 | 3 | 4 | 5 | 6 | 7 | 8 | 9 | 10 | 11 | 12 | 13 | 14 | 15 |
| Basenji | (1) | 0.002 | 0.016 | 0.003 | 0.002 | 0.881 | 0.001 | 0.002 | 0.008 | 0.002 | 0.002 | 0.003 | 0.002 | 0.003 | 0.005 | 0.005 |
| Basenji | (3) | 0.001 | 0.001 | 0.001 | 0.001 | 0.949 | 0.002 | 0.001 | 0.002 | 0.002 | 0.002 | 0.003 | 0.001 | 0.003 | 0.003 | 0.002 |
| Basenji | 0 | 0.001 | 0.004 | 0.001 | 0.004 | 0.925 | 0.004 | 0.002 | 0.003 | 0.005 | 0.003 | 0.001 | 0.01 | 0.001 | 0.003 | 0.003 |
| Basenji | 0 | 0.002 | 0.005 | 0.001 | 0.002 | 0.848 | 0.002 | 0.002 | 0.005 | 0.002 | 0.008 | 0.012 | 0.001 | 0.004 | 0.006 | 0.009 |
| Basenji | 0 | 0.003 | 0.009 | 0.003 | 0.005 | 0.874 | 0.002 | 0.003 | 0.01 | 0.002 | 0.002 | 0.003 | 0.003 | 0.007 | 0.002 | 0.016 |
| Basenji | 0 | 0.001 | 0.001 | 0.001 | 0.002 | 0.973 | 0.001 | 0.001 | 0.001 | 0.001 | 0 | 0.001 | 0.001 | 0.001 | 0.001 | 0.001 |
| Basenji | 0 | 0.004 | 0.002 | 0.017 | 0.005 | 0.771 | 0.008 | 0.005 | 0.003 | 0.005 | 0.006 | 0.003 | 0.002 | 0.003 | 0.037 | 0.02 |
| Basenji | 0 | 0.004 | 0.003 | 0.002 | 0.003 | 0.93 | 0.002 | 0.003 | 0.004 | 0.002 | 0.002 | 0.001 | 0.003 | 0.001 | 0.004 | 0.002 |
| Basenji | 0 | 0.001 | 0.001 | 0.003 | 0.001 | 0.963 | 0.001 | 0.001 | 0.001 | 0.001 | 0.001 | 0.002 | 0.002 | 0.001 | 0.001 | 0.001 |
| Basenji | 0 | 0.001 | 0.002 | 0.002 | 0.001 | 0.972 | 0.001 | 0.001 | 0.001 | 0.001 | 0.001 | 0.001 | 0.001 | 0.001 | 0.001 | 0.001 |
| Basenji | 0 | 0.001 | 0.001 | 0.001 | 0.001 | 0.983 | 0.001 | 0 | 0.001 | 0.001 | 0 | 0.001 | 0.001 | 0 | 0.001 | 0.001 |
| Basenji | 0 | 0.001 | 0.001 | 0.001 | 0.001 | 0.977 | 0.001 | 0.001 | 0.001 | 0.001 | 0.001 | 0.001 | 0.001 | 0.001 | 0.001 | 0.001 |
| Basenji | 0 | 0.001 | 0.001 | 0.001 | 0.001 | 0.978 | 0.001 | 0.001 | 0.001 | 0.001 | 0.001 | 0.001 | 0.001 | 0.001 | 0.001 | 0.001 |
| Basenji | (2) | 0.001 | 0.001 | 0.001 | 0.001 | 0.98 | 0.001 | 0.001 | 0.001 | 0.001 | 0.001 | 0.001 | 0 | 0.001 | 0.001 | 0.001 |
| Basenji | (1) | 0.007 | 0.004 | 0.006 | 0.004 | 0.887 | 0.002 | 0.002 | 0.003 | 0.002 | 0.002 | 0.005 | 0.002 | 0.003 | 0.002 | 0.004 |
| Basenji | (2) | 0.001 | 0.001 | 0.001 | 0.001 | 0.974 | 0.001 | 0.002 | 0.001 | 0.001 | 0.001 | 0.001 | 0.001 | 0.001 | 0.001 | 0.001 |
| Basenji | 0 | 0.002 | 0.002 | 0.003 | 0.005 | 0.933 | 0.003 | 0.004 | 0.001 | 0.001 | 0.004 | 0.002 | 0.003 | 0.002 | 0.002 | 0.002 |
| Basenji | (1) | 0.001 | 0.002 | 0.002 | 0.001 | 0.954 | 0.002 | 0.002 | 0.001 | 0.001 | 0.001 | 0.003 | 0.001 | 0.002 | 0.002 | 0.001 |
| Basenji | 0 | 0.002 | 0.002 | 0.007 | 0.001 | 0.957 | 0.001 | 0.001 | 0.002 | 0 | 0.001 | 0.002 | 0.001 | 0.001 | 0.001 | 0.002 |
| Basenji | 0 | 0.001 | 0.001 | 0.001 | 0.001 | 0.982 | 0.001 | 0.001 | 0.001 | 0.001 | 0.001 | 0.001 | 0.001 | 0.001 | 0.001 | 0.001 |
| Basenji | 0 | 0.001 | 0.001 | 0.001 | 0.001 | 0.982 | 0.001 | 0.001 | 0.001 | 0.001 | 0.001 | 0.001 | 0.001 | 0 | 0.001 | 0.001 |
| Basenji | 0 | 0.001 | 0.001 | 0.001 | 0.001 | 0.981 | 0.001 | 0.001 | 0.001 | 0.001 | 0 | 0.001 | 0.001 | 0 | 0.001 | 0.001 |
| Basenji | 0 | 0.001 | 0.001 | 0.001 | 0.001 | 0.97 | 0.001 | 0.001 | 0.001 | 0.001 | 0.001 | 0.001 | 0.001 | 0.001 | 0.001 | 0.001 |
| Basenji | (1) | 0.003 | 0.001 | 0.001 | 0.005 | 0.958 | 0.002 | 0.001 | 0.001 | 0.001 | 0.001 | 0.001 | 0.002 | 0.001 | 0.002 | 0.001 |
| Basenji | (1) | 0.001 | 0.001 | 0.001 | 0.001 | 0.979 | 0.001 | 0.001 | 0.001 | 0.001 | 0.001 | 0.001 | 0.001 | 0.001 | 0.001 | 0.001 |
| Basenji | 0 | 0.001 | 0.001 | 0.003 | 0.001 | 0.954 | 0.001 | 0.001 | 0.001 | 0.002 | 0.001 | 0.004 | 0.002 | 0.002 | 0.002 | 0.004 |
| Basenji | 0 | 0.001 | 0.001 | 0.001 | 0.001 | 0.978 | 0.001 | 0.001 | 0.001 | 0.001 | 0 | 0.001 | 0.001 | 0.001 | 0.001 | 0.001 |
| Basenji | 0 | 0.001 | 0.001 | 0.001 | 0.001 | 0.973 | 0.001 | 0.001 | 0.001 | 0.001 | 0.001 | 0.001 | 0.001 | 0.001 | 0.001 | 0.001 |
| Basenji | 0 | 0.001 | 0.001 | 0.001 | 0.001 | 0.978 | 0.001 | 0.001 | 0.001 | 0.001 | 0.001 | 0.001 | 0.001 | 0.001 | 0.001 | 0.001 |
| Basenji | 0 | 0.001 | 0.001 | 0.001 | 0.001 | 0.98 | 0.001 | 0.001 | 0.001 | 0.001 | 0 | 0.001 | 0 | 0.001 | 0.001 | 0.001 |
| Basenji | 0 | 0.009 | 0.002 | 0.003 | 0.003 | 0.851 | 0.004 | 0.002 | 0.004 | 0.006 | 0.003 | 0.005 | 0.004 | 0.004 | 0.006 | 0.004 |
| Basenji | (4) | 0.001 | 0.001 | 0.001 | 0.001 | 0.975 | 0.001 | 0.001 | 0.001 | 0.001 | 0.001 | 0.001 | 0.001 | 0.001 | 0.001 | 0.001 |
| Basenji | 0 | 0.001 | 0.001 | 0.001 | 0.001 | 0.972 | 0.001 | 0.001 | 0.001 | 0.001 | 0.001 | 0.001 | 0.002 | 0.001 | 0.001 | 0.001 |
| Basenji | 0 | 0.002 | 0.001 | 0.003 | 0.001 | 0.958 | 0.001 | 0.001 | 0.001 | 0.001 | 0.003 | 0.001 | 0.001 | 0.001 | 0.001 | 0.002 |
| Basenji | 0 | 0.002 | 0.001 | 0.002 | 0.002 | 0.907 | 0.002 | 0.002 | 0.002 | 0.007 | 0.009 | 0.002 | 0.002 | 0.003 | 0.003 | 0.002 |
| Basenji | (1) | 0.001 | 0.002 | 0.002 | 0.001 | 0.907 | 0.006 | 0.002 | 0.002 | 0.003 | 0.018 | 0.001 | 0.002 | 0.003 | 0.001 | 0.004 |
| Basenji | 0 | 0.001 | 0.001 | 0.001 | 0.001 | 0.977 | 0.001 | 0.001 | 0.001 | 0.001 | 0.001 | 0.001 | 0.001 | 0.001 | 0.001 | 0.001 |
| Basenji | 0 | 0.001 | 0.001 | 0.001 | 0.001 | 0.982 | 0.001 | 0.001 | 0 | 0.001 | 0 | 0.001 | 0.001 | 0.001 | 0.001 | 0.001 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Basenji | | 0.001 | 0.007 | 0.016 | 0.003 | 0.003 | 0.002 | 0.003 | 0.004 | 0.003 | 0.003 | 0.001 | 0.003 | 0.008 | 0.003 | 0.002 |
| Basenji | | 0.002 | 0.002 | 0.001 | 0.001 | 0.001 | 0.003 | 0.001 | 0.001 | 0.002 | 0.001 | 0.001 | 0.004 | 0.001 | 0.001 | 0.003 |
| Basenji | | 0.001 | 0.001 | 0.002 | 0.005 | 0.001 | 0.001 | 0.005 | 0.002 | 0.002 | 0.002 | 0.001 | 0 | 0.002 | 0.004 | 0.002 |
| Basenji | | 0.002 | 0.015 | 0.015 | 0.006 | 0.004 | 0.003 | 0.003 | 0.006 | 0.002 | 0.001 | 0.002 | 0.011 | 0.003 | 0.013 | 0.004 |
| Basenji | | 0.002 | 0.005 | 0.009 | 0.004 | 0.004 | 0.002 | 0.002 | 0.004 | 0.001 | 0.003 | 0.003 | 0.002 | 0.011 | 0.002 | 0.002 |
| Basenji | | 0.001 | 0.001 | 0.001 | 0.001 | 0.001 | 0.001 | 0.001 | 0.001 | 0.002 | 0.001 | 0.001 | 0.001 | 0.001 | 0.001 | 0.001 |
| Basenji | | 0.004 | 0.001 | 0.01 | 0.016 | 0.006 | 0.015 | 0.003 | 0.006 | 0.009 | 0.001 | 0.003 | 0.002 | 0.004 | 0.003 | 0.023 |
| Basenji | | 0.001 | 0.002 | 0.001 | 0.004 | 0.003 | 0.002 | 0.003 | 0.002 | 0.004 | 0.003 | 0.001 | 0.002 | 0.002 | 0.002 | 0.003 |
| Basenji | | 0.001 | 0.001 | 0.002 | 0.002 | 0.002 | 0.001 | 0.001 | 0.001 | 0.002 | 0.001 | 0.001 | 0.001 | 0.001 | 0.001 | 0.001 |
| Basenji | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Basenji | | 0 | 0 | 0.001 | 0.001 | 0.001 | 0.001 | 0.001 | 0.001 | 0.001 | 0 | 0.001 | 0.001 | 0.001 | 0.001 | 0.001 |
| Basenji | | 0.001 | 0.001 | 0.001 | 0.001 | 0.001 | 0.001 | 0.001 | 0.001 | 0.001 | 0.001 | 0.001 | 0 | 0.001 | 0.001 | 0.001 |
| Basenji | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Basenji | | 0.001 | 0 | 0.001 | 0.001 | 0.001 | 0.001 | 0.001 | 0.001 | 0.001 | 0 | 0.001 | 0.001 | 0.001 | 0.001 | 0.001 |
| Basenji | | 0.001 | 0.004 | 0.006 | 0.005 | 0.011 | 0.001 | 0.003 | 0.003 | 0.003 | 0.002 | 0.001 | 0.008 | 0.007 | 0.002 | 0.005 |
| Basenji | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Basenji | | 0.001 | 0.001 | 0.001 | 0.004 | 0.004 | 0.002 | 0.002 | 0.002 | 0.001 | 0.003 | 0.002 | 0.002 | 0.002 | 0.003 | 0.002 |
| Basenji | | 0.001 | 0.002 | 0.001 | 0.006 | 0.002 | 0.001 | 0.001 | 0.001 | 0.001 | 0.001 | 0.002 | 0.001 | 0.002 | 0.001 | 0.001 |
| Basenji | | 0.001 | 0.001 | 0.001 | 0.001 | 0.001 | 0.001 | 0.001 | 0.001 | 0.001 | 0.001 | 0.001 | 0.001 | 0.001 | 0.002 | 0.001 |
| Basenji | | 0 | 0.001 | 0.001 | 0.001 | 0.001 | 0.001 | 0 | 0 | 0.001 | 0.001 | 0 | 0.001 | 0.001 | 0.001 | 0.001 |
| Basenji | | 0.001 | 0 | 0 | 0.001 | 0 | 0.001 | 0.001 | 0.001 | 0.001 | 0 | 0.001 | 0.001 | 0.001 | 0.001 | 0.001 |
| Basenji | | 0 | 0.001 | 0.001 | 0.001 | 0.001 | 0.001 | 0.001 | 0 | 0.001 | 0 | 0.001 | 0 | 0.001 | 0.001 | 0.001 |
| Basenji | | 0.001 | 0.001 | 0.001 | 0.001 | 0.002 | 0.001 | 0.001 | 0.001 | 0.001 | 0.001 | 0.001 | 0.001 | 0.001 | 0.002 | 0.001 |
| Basenji | | 0.001 | 0.001 | 0.001 | 0.002 | 0.002 | 0.002 | 0.002 | 0.001 | 0.004 | 0.001 | 0.001 | 0.001 | 0.001 | 0.001 | 0.002 |
| Basenji | | 0.001 | 0.001 | 0.001 | 0.001 | 0.001 | 0.001 | 0.001 | 0.001 | 0.001 | 0.001 | 0 | 0.001 | 0.001 | 0.001 | 0.001 |
| Basenji | | 0.001 | 0.001 | 0.001 | 0.002 | 0.001 | 0.001 | 0.002 | 0.001 | 0.002 | 0.002 | 0.001 | 0.001 | 0.001 | 0.001 | 0.001 |
| Basenji | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Basenji | | 0.001 | 0.001 | 0.001 | 0.001 | 0.001 | 0.001 | 0.001 | 0.001 | 0.002 | 0.001 | 0.001 | 0.001 | 0.001 | 0.002 | 0.001 |
| Basenji | | 0.001 | 0.001 | 0.001 | 0.001 | 0.001 | 0.001 | 0.001 | 0.001 | 0.001 | 0 | 0.001 | 0 | 0.001 | 0.001 | 0.001 |
| Basenji | | 0.001 | 0.001 | 0.001 | 0.001 | 0.001 | 0.001 | 0.001 | 0.001 | 0.001 | 0 | 0 | 0.001 | 0.001 | 0.001 | 0.001 |
| Basenji | | 0.001 | 0.003 | 0.004 | 0.008 | 0.006 | 0.008 | 0.007 | 0.004 | 0.02 | 0.003 | 0.006 | 0.005 | 0.007 | 0.004 | 0.003 |
| Basenji | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Basenji | | 0.001 | 0.001 | 0.001 | 0.002 | 0.001 | 0.001 | 0.001 | 0.001 | 0.001 | 0.001 | 0.001 | 0.001 | 0.001 | 0.001 | 0.001 |
| Basenji | | 0.001 | 0.001 | 0.001 | 0.002 | 0.001 | 0.002 | 0.001 | 0.001 | 0.002 | 0.001 | 0.001 | 0.001 | 0.001 | 0.003 | 0.002 |
| Basenji | | 0.001 | 0.002 | 0.004 | 0.005 | 0.008 | 0.002 | 0.001 | 0.007 | 0.003 | 0.002 | 0.008 | 0.002 | 0.001 | 0.002 | 0.002 |
| Basenji | | 0.001 | 0.001 | 0.003 | 0.002 | 0.006 | 0.004 | 0.001 | 0.005 | 0.002 | 0.002 | 0.011 | 0.001 | 0.001 | 0.001 | 0.001 |
| Basenji | | 0.001 | 0.001 | 0.001 | 0.001 | 0.001 | 0.001 | 0.001 | 0.001 | 0.001 | 0.001 | 0.001 | 0 | 0.001 | 0.001 | 0.001 |
| Basenji | | 0 | 0 | 0.001 | 0.001 | 0.001 | 0.001 | 0.001 | 0.001 | 0.001 | 0 | 0.001 | 0 | 0.001 | 0.001 | 0.001 |
| | % | Cluster | | | | | | | | | | | | | | |
| | missing | Assignment | | | | | | | | | | | | | | |
| Breed | data | 1 | 2 | 3 | 4 | 5 | 6 | 7 | 8 | 9 | 10 | 11 | 12 | 13 | 14 | 15 |
| Basset Hound | 0 | 0.966 | 0.001 | 0.002 | 0.001 | 0.002 | 0.001 | 0.001 | 0.001 | 0.001 | 0.001 | 0.001 | 0.001 | 0.001 | 0.001 | 0.001 |
| Basset Hound | 0 | 0.965 | 0.001 | 0.002 | 0.001 | 0.001 | 0.001 | 0.001 | 0.002 | 0.001 | 0.001 | 0.002 | 0.001 | 0.001 | 0.001 | 0.001 |
| Basset Hound | 0 | 0.939 | 0.007 | 0.002 | 0.001 | 0.001 | 0.002 | 0.002 | 0.002 | 0.001 | 0.002 | 0.002 | 0.002 | 0.003 | 0.003 | 0.002 |
| Basset Hound | 0 | 0.958 | 0.001 | 0.004 | 0.001 | 0.001 | 0.001 | 0.001 | 0.002 | 0.001 | 0.001 | 0.002 | 0.002 | 0.001 | 0.001 | 0.002 |
| Basset Hound | 0 | 0.885 | 0.002 | 0.006 | 0.004 | 0.006 | 0.002 | 0.004 | 0.004 | 0.002 | 0.005 | 0.004 | 0.002 | 0.001 | 0.002 | 0.007 |
| Basset Hound | 0 | 0.914 | 0.002 | 0.003 | 0.01 | 0.003 | 0.004 | 0.004 | 0.002 | 0.002 | 0.003 | 0.002 | 0.001 | 0.002 | 0.002 | 0.009 |
| Basset Hound | 0 | 0.941 | 0.001 | 0.001 | 0.001 | 0.001 | 0.001 | 0.002 | 0.002 | 0.004 | 0.002 | 0.003 | 0.001 | 0.003 | 0.001 | 0.003 |
| Basset Hound | (2) | 0.78 | 0.002 | 0.006 | 0.003 | 0.004 | 0.004 | 0.005 | 0.013 | 0.009 | 0.003 | 0.021 | 0.002 | 0.002 | 0.007 | 0.019 |
| Basset Hound | 0 | 0.937 | 0.003 | 0.002 | 0.004 | 0.002 | 0.002 | 0.002 | 0.002 | 0.001 | 0.002 | 0.002 | 0.001 | 0.005 | 0.002 | 0.008 |
| Basset Hound | 0 | 0.886 | 0.003 | 0.003 | 0.005 | 0.001 | 0.002 | 0.003 | 0.006 | 0.003 | 0.004 | 0.003 | 0.001 | 0.006 | 0.002 | 0.001 |
| Basset Hound | (3) | 0.944 | 0.001 | 0.002 | 0.002 | 0.002 | 0.001 | 0.002 | 0.002 | 0.001 | 0.001 | 0.001 | 0.001 | 0.001 | 0.002 | 0.003 |
| Basset Hound | (1) | 0.938 | 0.001 | 0.007 | 0.005 | 0.001 | 0.001 | 0.002 | 0.001 | 0.002 | 0.002 | 0.001 | 0.001 | 0.003 | 0.001 | 0.003 |
| Basset Hound | 0 | 0.945 | 0.001 | 0.002 | 0.007 | 0.002 | 0.001 | 0.002 | 0.002 | 0.002 | 0.001 | 0.002 | 0.001 | 0.001 | 0.001 | 0.002 |
| Basset Hound | 0 | 0.946 | 0.002 | 0.002 | 0.003 | 0.001 | 0.003 | 0.001 | 0.001 | 0.002 | 0.001 | 0.002 | 0.001 | 0.003 | 0.001 | 0.001 |
| Basset Hound | 0 | 0.907 | 0.002 | 0.001 | 0.002 | 0.001 | 0.004 | 0.002 | 0.003 | 0.002 | 0.001 | 0.004 | 0.003 | 0.004 | 0.002 | 0.003 |
| Basset Hound | 0 | 0.895 | 0.002 | 0.002 | 0.005 | 0.002 | 0.013 | 0.001 | 0.001 | 0.002 | 0.001 | 0.005 | 0.002 | 0.005 | 0.002 | 0.004 |
| Basset Hound | (4) | 0.883 | 0.002 | 0.001 | 0.002 | 0.002 | 0.003 | 0.003 | 0.008 | 0.002 | 0.003 | 0.003 | 0.003 | 0.003 | 0.004 | 0.002 |
| Basset Hound | 0 | 0.88 | 0.005 | 0.001 | 0.003 | 0.001 | 0.002 | 0.004 | 0.016 | 0.002 | 0.005 | 0.009 | 0.004 | 0.007 | 0.003 | 0.002 |
| Basset Hound | 0 | 0.758 | 0.004 | 0.002 | 0.005 | 0.001 | 0.003 | 0.004 | 0.027 | 0.004 | 0.008 | 0.048 | 0.003 | 0.008 | 0.001 | 0.005 |
| Basset Hound | 0 | 0.774 | 0.055 | 0.006 | 0.003 | 0.002 | 0.006 | 0.011 | 0.01 | 0.001 | 0.025 | 0.001 | 0.003 | 0.004 | 0.004 | 0.004 |
| Basset Hound | 0 | 0.899 | 0.009 | 0.003 | 0.002 | 0.002 | 0.001 | 0.004 | 0.003 | 0.001 | 0.003 | 0.002 | 0.003 | 0.002 | 0.001 | 0.007 |
| Basset Hound | (2) | 0.824 | 0.008 | 0.002 | 0.003 | 0.007 | 0.002 | 0.003 | 0.003 | 0.002 | 0.002 | 0.007 | 0.002 | 0.01 | 0.007 | 0.003 |
| Basset Hound | 0 | 0.823 | 0.007 | 0.002 | 0.003 | 0.001 | 0.004 | 0.014 | 0.008 | 0.002 | 0.003 | 0.003 | 0.008 | 0.015 | 0.006 | 0.002 |
| Basset Hound | (4) | 0.937 | 0.003 | 0.003 | 0.003 | 0.001 | 0.001 | 0.002 | 0.002 | 0.001 | 0.001 | 0.003 | 0.002 | 0.002 | 0.001 | 0.002 |
| Basset Hound | 0 | 0.874 | 0.002 | 0.003 | 0.01 | 0.003 | 0.002 | 0.003 | 0.003 | 0.006 | 0.005 | 0.009 | 0.007 | 0.008 | 0.011 | 0.004 |
| Basset Hound | 0 | 0.787 | 0.003 | 0.007 | 0.012 | 0.002 | 0.006 | 0.008 | 0.008 | 0.004 | 0.004 | 0.004 | 0.002 | 0.006 | 0.001 | 0.016 |
| Basset Hound | 0 | 0.535 | 0.011 | 0.005 | 0.008 | 0.001 | 0.011 | 0.006 | 0.055 | 0.006 | 0.011 | 0.006 | 0.001 | 0.168 | 0.002 | 0.004 |
| Basset Hound | 0 | 0.914 | 0.005 | 0.003 | 0.005 | 0.002 | 0.001 | 0.003 | 0.002 | 0.001 | 0.001 | 0.002 | 0.007 | 0.002 | 0.002 | 0.003 |
| Basset Hound | (1) | 0.878 | 0.008 | 0.004 | 0.004 | 0.002 | 0.01 | 0.01 | 0.003 | 0.008 | 0.004 | 0.002 | 0.002 | 0.002 | 0.002 | 0.005 |
| Basset Hound | 0 | 0.891 | 0.005 | 0.001 | 0.002 | 0.003 | 0.001 | 0.004 | 0.002 | 0.001 | 0.004 | 0.002 | 0.006 | 0.005 | 0.002 | 0.002 |
| Basset Hound | (2) | 0.782 | 0.053 | 0.001 | 0.007 | 0.001 | 0.011 | 0.004 | 0.006 | 0.004 | 0.004 | 0.003 | 0.001 | 0.015 | 0.002 | 0.008 |
| Basset Hound | (6) | 0.861 | 0.006 | 0.007 | 0.002 | 0.001 | 0.004 | 0.004 | 0.006 | 0.011 | 0.002 | 0.005 | 0.004 | 0.003 | 0.002 | 0.003 |
| Basset Hound | 0 | 0.9 | 0.003 | 0.005 | 0.004 | 0.003 | 0.003 | 0.003 | 0.002 | 0.005 | 0.001 | 0.01 | 0.002 | 0.003 | 0.004 | 0.003 |
| Basset Hound | 0 | 0.936 | 0.002 | 0.011 | 0.003 | 0.003 | 0.002 | 0.001 | 0.002 | 0.001 | 0.001 | 0.005 | 0.002 | 0.003 | 0.001 | 0.002 |
| Basset Hound | (1) | 0.898 | 0.004 | 0.022 | 0.004 | 0.002 | 0.002 | 0.002 | 0.005 | 0.001 | 0.004 | 0.005 | 0.01 | 0.003 | 0.001 | 0.002 |
| Basset Hound | (1) | 0.935 | 0.004 | 0.003 | 0.002 | 0.001 | 0.002 | 0.004 | 0.003 | 0.001 | 0.001 | 0.001 | 0.002 | 0.003 | 0.002 | 0.002 |
| Basset Hound | (2) | 0.84 | 0.008 | 0.012 | 0.005 | 0.005 | 0.024 | 0.003 | 0.005 | 0.004 | 0.002 | 0.008 | 0.002 | 0.009 | 0.004 | 0.004 |
| Basset Hound | 0 | 0.938 | 0.002 | 0.003 | 0.003 | 0.002 | 0.002 | 0.001 | 0.002 | 0.002 | 0.001 | 0.003 | 0.002 | 0.003 | 0.002 | 0.001 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Basset Hound | | 0.001 | 0.001 | 0.001 | 0.001 | 0.001 | 0.001 | 0.001 | 0.001 | 0.003 | 0.001 | 0.001 | 0.001 | 0.001 | 0.001 | 0.001 |
| Basset Hound | | 0.001 | 0.001 | 0.002 | 0.001 | 0.001 | 0.001 | 0.001 | 0.001 | 0.002 | 0.001 | 0.001 | 0.001 | 0.001 | 0.001 | 0.001 |
| Basset Hound | | 0.001 | 0.002 | 0.004 | 0.001 | 0.002 | 0.001 | 0.005 | 0.003 | 0.002 | 0.001 | 0.001 | 0.002 | 0.001 | 0.002 | 0.001 |
| Basset Hound | | 0.003 | 0.001 | 0.001 | 0.002 | 0.001 | 0.001 | 0.001 | 0.001 | 0.002 | 0.002 | 0.002 | 0.001 | 0.001 | 0.001 | 0.002 |
| Basset Hound | | 0.001 | 0.003 | 0.003 | 0.007 | 0.002 | 0.003 | 0.012 | 0.008 | 0.007 | 0.001 | 0.004 | 0.002 | 0.001 | 0.004 | 0.006 |
| Basset Hound | | 0.001 | 0.002 | 0.002 | 0.008 | 0.002 | 0.002 | 0.004 | 0.002 | 0.003 | 0.003 | 0.002 | 0.003 | 0.001 | 0.001 | 0.002 |
| Basset Hound | | 0.001 | 0.002 | 0.001 | 0.003 | 0.002 | 0.003 | 0.003 | 0.003 | 0.003 | 0.001 | 0.001 | 0.002 | 0.001 | 0.001 | 0.003 |
| Basset Hound | | 0.002 | 0.016 | 0.014 | 0.002 | 0.003 | 0.003 | 0.03 | 0.004 | 0.005 | 0.005 | 0.01 | 0.005 | 0.005 | 0.003 | 0.01 |
| Basset Hound | | 0.001 | 0.001 | 0.002 | 0.001 | 0.001 | 0.002 | 0.002 | 0.001 | 0.003 | 0.003 | 0.002 | 0.002 | 0.002 | 0.001 | 0.001 |
| Basset Hound | | 0.001 | 0.009 | 0.005 | 0.004 | 0.003 | 0.001 | 0.017 | 0.007 | 0.002 | 0.004 | 0.002 | 0.004 | 0.004 | 0.001 | 0.006 |
| Basset Hound | | 0.002 | 0.003 | 0.001 | 0.002 | 0.001 | 0.002 | 0.003 | 0.002 | 0.006 | 0.001 | 0.002 | 0.002 | 0.001 | 0.003 | 0.002 |
| Basset Hound | | 0.001 | 0.001 | 0.004 | 0.002 | 0.002 | 0.002 | 0.001 | 0.002 | 0.003 | 0.001 | 0.002 | 0.003 | 0.002 | 0.002 | 0.002 |
| Basset Hound | | 0.001 | 0.001 | 0.001 | 0.003 | 0.001 | 0.003 | 0.001 | 0.001 | 0.002 | 0.001 | 0.003 | 0.002 | 0.001 | 0.001 | 0.004 |
| Basset Hound | | 0.002 | 0.001 | 0.002 | 0.002 | 0.001 | 0.002 | 0.003 | 0.002 | 0.002 | 0.001 | 0.002 | 0.002 | 0.001 | 0.001 | 0.004 |
| Basset Hound | | 0.003 | 0.001 | 0.003 | 0.005 | 0.002 | 0.006 | 0.012 | 0.004 | 0.004 | 0.006 | 0.002 | 0.004 | 0.002 | 0.001 | 0.004 |
| Basset Hound | | 0.004 | 0.001 | 0.002 | 0.012 | 0.002 | 0.005 | 0.005 | 0.003 | 0.008 | 0.002 | 0.002 | 0.002 | 0.002 | 0.003 | 0.007 |
| Basset Hound | | 0.002 | 0.004 | 0.007 | 0.002 | 0.002 | 0.006 | 0.001 | 0.007 | 0.004 | 0.007 | 0.009 | 0.002 | 0.015 | 0.002 | 0.007 |
| Basset Hound | | 0.001 | 0.004 | 0.007 | 0.001 | 0.001 | 0.005 | 0.001 | 0.002 | 0.002 | 0.013 | 0.006 | 0.005 | 0.001 | 0.001 | 0.005 |
| Basset Hound | | 0.001 | 0.006 | 0.022 | 0.001 | 0.002 | 0.002 | 0.001 | 0.003 | 0.003 | 0.021 | 0.025 | 0.01 | 0.014 | 0.005 | 0.004 |
| Basset Hound | | 0.002 | 0.002 | 0.002 | 0.002 | 0.001 | 0.004 | 0.019 | 0.007 | 0.009 | 0.002 | 0.013 | 0.008 | 0.003 | 0.009 | 0.006 |
| Basset Hound | | 0.007 | 0.005 | 0.003 | 0.003 | 0.008 | 0.004 | 0.007 | 0.001 | 0.002 | 0.004 | 0.002 | 0.004 | 0.002 | 0.003 | 0.002 |
| Basset Hound | | 0.001 | 0.015 | 0.003 | 0.002 | 0.005 | 0.002 | 0.004 | 0.002 | 0.007 | 0.008 | 0.002 | 0.056 | 0.003 | 0.003 | 0.004 |
| Basset Hound | | 0.001 | 0.008 | 0.006 | 0.002 | 0.002 | 0.001 | 0.004 | 0.003 | 0.004 | 0.013 | 0.003 | 0.027 | 0.005 | 0.004 | 0.015 |
| Basset Hound | | 0.002 | 0.001 | 0.002 | 0.002 | 0.002 | 0.001 | 0.002 | 0.001 | 0.002 | 0.003 | 0.002 | 0.009 | 0.002 | 0.003 | 0.001 |
| Basset Hound | | 0.002 | 0.006 | 0.002 | 0.005 | 0.002 | 0.015 | 0.002 | 0.005 | 0.002 | 0.001 | 0.002 | 0.003 | 0.002 | 0.003 | 0.002 |
| Basset Hound | | 0.014 | 0.024 | 0.002 | 0.005 | 0.004 | 0.011 | 0.007 | 0.007 | 0.011 | 0.017 | 0.001 | 0.012 | 0.004 | 0.005 | 0.005 |
| Basset Hound | | 0.002 | 0.026 | 0.002 | 0.008 | 0.001 | 0.002 | 0.004 | 0.006 | 0.002 | 0.047 | 0.007 | 0.04 | 0.009 | 0.004 | 0.009 |
| Basset Hound | | 0.001 | 0.002 | 0.002 | 0.001 | 0.004 | 0.007 | 0.009 | 0.002 | 0.003 | 0.002 | 0.002 | 0.005 | 0.001 | 0.003 | 0.004 |
| Basset Hound | | 0.001 | 0.005 | 0.003 | 0.002 | 0.001 | 0.002 | 0.003 | 0.001 | 0.003 | 0.004 | 0.002 | 0.001 | 0.003 | 0.002 | 0.021 |
| Basset Hound | | 0.002 | 0.004 | 0.002 | 0.002 | 0.002 | 0.002 | 0.002 | 0.007 | 0.001 | 0.002 | 0.002 | 0.031 | 0.002 | 0.005 | 0.001 |
| Basset Hound | | 0.001 | 0.007 | 0.018 | 0.006 | 0.003 | 0.01 | 0.005 | 0.009 | 0.003 | 0.006 | 0.006 | 0.004 | 0.005 | 0.01 | 0.007 |
| Basset Hound | | 0.001 | 0.003 | 0.006 | 0.005 | 0.002 | 0.002 | 0.003 | 0.004 | 0.004 | 0.002 | 0.003 | 0.015 | 0.003 | 0.008 | 0.02 |
| Basset Hound | | 0.001 | 0.006 | 0.007 | 0.004 | 0.001 | 0.002 | 0.002 | 0.003 | 0.007 | 0.001 | 0.001 | 0.005 | 0.002 | 0.003 | 0.004 |
| Basset Hound | | 0.001 | 0.001 | 0.002 | 0.002 | 0.002 | 0.002 | 0.001 | 0.001 | 0.002 | 0.001 | 0.001 | 0.002 | 0.002 | 0.001 | 0.003 |
| Basset Hound | | 0.001 | 0.001 | 0.004 | 0.002 | 0.003 | 0.002 | 0.002 | 0.002 | 0.002 | 0.002 | 0.001 | 0.002 | 0.004 | 0.003 | 0.005 |
| Basset Hound | | 0.002 | 0.002 | 0.002 | 0.002 | 0.001 | 0.003 | 0.002 | 0.003 | 0.002 | 0.001 | 0.001 | 0.003 | 0.002 | 0.002 | 0.006 |
| Basset Hound | | 0.003 | 0.003 | 0.004 | 0.011 | 0.006 | 0.004 | 0.002 | 0.008 | 0.004 | 0.003 | 0.003 | 0.002 | 0.004 | 0.002 | 0.008 |
| Basset Hound | | 0.001 | 0.001 | 0.004 | 0.003 | 0.003 | 0.003 | 0.001 | 0.001 | 0.002 | 0.001 | 0.001 | 0.002 | 0.006 | 0.002 | 0.002 |
| | % | Cluster | | | | | | | | | | | | | | |
| | missing | Assignment | | | | | | | | | | | | | | |
| Breed | data | 1 | 2 | 3 | 4 | 5 | 6 | 7 | 8 | 9 | 10 | 11 | 12 | 13 | 14 | 15 |
| Beagle | 0 | 0.002 | 0.004 | 0.01 | 0.006 | 0.003 | 0.004 | 0.007 | 0.006 | 0.019 | 0.002 | 0.012 | 0.006 | 0.004 | 0.006 | 0.007 |
| Beagle | (1) | 0.011 | 0.01 | 0.003 | 0.002 | 0.002 | 0.002 | 0.004 | 0.016 | 0.002 | 0.007 | 0.004 | 0.002 | 0.002 | 0.008 | 0.003 |
| Beagle | 0 | 0.059 | 0.015 | 0.004 | 0.002 | 0.003 | 0.007 | 0.009 | 0.017 | 0.002 | 0.005 | 0.009 | 0.002 | 0.004 | 0.005 | 0.005 |
| Beagle | 0 | 0.009 | 0.005 | 0.004 | 0.003 | 0.006 | 0.006 | 0.009 | 0.005 | 0.001 | 0.003 | 0.01 | 0.002 | 0.006 | 0.004 | 0.003 |
| Beagle | 0 | 0.008 | 0.008 | 0.011 | 0.013 | 0.001 | 0.004 | 0.004 | 0.03 | 0.002 | 0.011 | 0.006 | 0.004 | 0.022 | 0.002 | 0.011 |
| Beagle | 0 | 0.003 | 0.001 | 0.001 | 0.001 | 0.004 | 0.003 | 0.004 | 0.002 | 0.003 | 0.001 | 0.002 | 0.001 | 0.002 | 0.002 | 0.003 |
| Beagle | (1) | 0.002 | 0.002 | 0.003 | 0.002 | 0.004 | 0.003 | 0.001 | 0.002 | 0.001 | 0.001 | 0.002 | 0.001 | 0.002 | 0.001 | 0.003 |
| Beagle | 0 | 0.002 | 0.002 | 0.015 | 0.007 | 0.002 | 0.004 | 0.006 | 0.006 | 0.003 | 0.005 | 0.008 | 0.016 | 0.023 | 0.005 | 0.003 |
| Beagle | 0 | 0.009 | 0.002 | 0.002 | 0.002 | 0.003 | 0.002 | 0.004 | 0.002 | 0.005 | 0.001 | 0.002 | 0.005 | 0.003 | 0.002 | 0.006 |
| Beagle | 0 | 0.003 | 0.003 | 0.003 | 0.003 | 0.004 | 0.008 | 0.003 | 0.002 | 0.005 | 0.002 | 0.015 | 0.001 | 0.01 | 0.005 | 0.009 |
| Beagle | 0 | 0.002 | 0.002 | 0.003 | 0.002 | 0.002 | 0.001 | 0.002 | 0.01 | 0.002 | 0.003 | 0.004 | 0.004 | 0.003 | 0.005 | 0.006 |
| Beagle | 0 | 0.003 | 0.002 | 0.002 | 0.001 | 0.001 | 0.002 | 0.004 | 0.002 | 0.007 | 0.014 | 0.003 | 0.005 | 0.003 | 0.002 | 0.002 |
| Beagle | 0 | 0.01 | 0.004 | 0.006 | 0.005 | 0.011 | 0.007 | 0.005 | 0.017 | 0.001 | 0.002 | 0.008 | 0.004 | 0.008 | 0.004 | 0.002 |
| Beagle | 0 | 0.002 | 0.004 | 0.003 | 0.004 | 0.001 | 0.002 | 0.001 | 0.004 | 0.001 | 0.004 | 0.001 | 0.001 | 0.002 | 0.002 | 0.002 |
| Beagle | (1) | 0.003 | 0.002 | 0.001 | 0.001 | 0.001 | 0.001 | 0.002 | 0.002 | 0.002 | 0.003 | 0.003 | 0.002 | 0.001 | 0.003 | 0.001 |
| Beagle | 0 | 0.002 | 0.002 | 0.001 | 0.001 | 0.001 | 0.002 | 0.002 | 0.003 | 0.002 | 0.005 | 0.003 | 0.002 | 0.002 | 0.005 | 0.001 |
| Beagle | 0 | 0.015 | 0.002 | 0.013 | 0.003 | 0.004 | 0.003 | 0.011 | 0.009 | 0.022 | 0.007 | 0.009 | 0.004 | 0.003 | 0.004 | 0.003 |
| Beagle | (1) | 0.004 | 0.004 | 0.003 | 0.023 | 0.023 | 0.012 | 0.005 | 0.01 | 0.005 | 0.005 | 0.003 | 0.002 | 0.004 | 0.002 | 0.011 |
| Beagle | 0 | 0.003 | 0.004 | 0.003 | 0.006 | 0.091 | 0.004 | 0.004 | 0.006 | 0.004 | 0.003 | 0.003 | 0.002 | 0.002 | 0.006 | 0.062 |
| Beagle | 0 | 0.002 | 0.004 | 0.003 | 0.002 | 0.003 | 0.003 | 0.002 | 0.004 | 0.001 | 0.003 | 0.002 | 0.002 | 0.003 | 0.005 | 0.003 |
| Beagle | 0 | 0.004 | 0.001 | 0.001 | 0.001 | 0.002 | 0.003 | 0.002 | 0.002 | 0.001 | 0.002 | 0.001 | 0.002 | 0.002 | 0.002 | 0.002 |
| Beagle | (2) | 0.007 | 0.002 | 0.002 | 0.001 | 0.002 | 0.001 | 0.001 | 0.002 | 0.001 | 0.013 | 0.002 | 0.001 | 0.001 | 0.004 | 0.002 |
| Beagle | 0 | 0.003 | 0.002 | 0.003 | 0.001 | 0.001 | 0.002 | 0.001 | 0.003 | 0.001 | 0.003 | 0.003 | 0.001 | 0.001 | 0.002 | 0.001 |
| Beagle | (5) | 0.001 | 0.001 | 0.001 | 0.002 | 0.001 | 0.002 | 0.001 | 0.002 | 0.001 | 0.002 | 0.002 | 0.001 | 0.001 | 0.002 | 0.001 |
| Beagle | (7) | 0.005 | 0.004 | 0.021 | 0.004 | 0.014 | 0.002 | 0.003 | 0.003 | 0.001 | 0.003 | 0.003 | 0.008 | 0.004 | 0.004 | 0.009 |
| Beagle | (1) | 0.005 | 0.004 | 0.004 | 0.005 | 0.013 | 0.011 | 0.006 | 0.002 | 0.002 | 0.025 | 0.018 | 0.002 | 0.005 | 0.009 | 0.005 |
| Beagle | 0 | 0.007 | 0.004 | 0.001 | 0.003 | 0.001 | 0.003 | 0.021 | 0.002 | 0.014 | 0.005 | 0.002 | 0.004 | 0.007 | 0.001 | 0.003 |
| Beagle | 0 | 0.002 | 0.002 | 0.002 | 0.001 | 0.001 | 0.004 | 0.003 | 0.006 | 0.002 | 0.001 | 0.001 | 0.005 | 0.003 | 0.004 | 0.003 |
| Beagle | 0 | 0.002 | 0.012 | 0.003 | 0.002 | 0.001 | 0.006 | 0.006 | 0.005 | 0.002 | 0.002 | 0.003 | 0.002 | 0.021 | 0.015 | 0.003 |
| Beagle | (1) | 0.002 | 0.002 | 0.002 | 0.003 | 0.001 | 0.002 | 0.001 | 0.002 | 0.002 | 0.001 | 0.002 | 0.002 | 0.004 | 0.002 | 0.001 |
| Beagle | 0 | 0.006 | 0.002 | 0.004 | 0.003 | 0.005 | 0.003 | 0.002 | 0.003 | 0.002 | 0.002 | 0.009 | 0.002 | 0.004 | 0.001 | 0.004 |
| Beagle | 0 | 0.004 | 0.012 | 0.002 | 0.007 | 0.002 | 0.004 | 0.015 | 0.005 | 0.004 | 0.049 | 0.002 | 0.014 | 0.006 | 0.006 | 0.01 |
| Beagle | 0 | 0.011 | 0.005 | 0.003 | 0.009 | 0.003 | 0.021 | 0.003 | 0.002 | 0.016 | 0.003 | 0.004 | 0.001 | 0.003 | 0.004 | 0.013 |
| Beagle | (1) | 0.003 | 0.002 | 0.001 | 0.003 | 0.002 | 0.003 | 0.003 | 0.003 | 0.006 | 0.002 | 0.001 | 0.003 | 0.003 | 0.004 | 0.005 |
| Beagle | (3) | 0.004 | 0.005 | 0.012 | 0.014 | 0.012 | 0.004 | 0.001 | 0.002 | 0.006 | 0.003 | 0.014 | 0.003 | 0.003 | 0.01 | 0.01 |
| Beagle | 0 | 0.003 | 0.02 | 0.012 | 0.007 | 0.002 | 0.004 | 0.006 | 0.009 | 0.006 | 0.003 | 0.001 | 0.008 | 0.02 | 0.002 | 0.002 |
| Beagle | 0 | 0.003 | 0.002 | 0.002 | 0.002 | 0.045 | 0.008 | 0.006 | 0.005 | 0.003 | 0.002 | 0.005 | 0.002 | 0.002 | 0.011 | 0.004 |
| Beagle | (1) | 0.006 | 0.01 | 0.004 | 0.002 | 0.001 | 0.004 | 0.008 | 0.007 | 0.004 | 0.01 | 0.003 | 0.004 | 0.003 | 0.005 | 0.003 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Beagle | | 0.002 | 0.004 | 0.007 | 0.008 | 0.003 | 0.806 | 0.016 | 0.003 | 0.01 | 0.003 | 0.002 | 0.003 | 0.027 | 0.001 | 0.006 |
| Beagle | | 0.002 | 0.043 | 0.017 | 0.005 | 0.002 | 0.809 | 0.004 | 0.002 | 0.003 | 0.015 | 0.003 | 0.005 | 0.002 | 0.002 | 0.007 |
| Beagle | | 0.006 | 0.003 | 0.002 | 0.003 | 0.005 | 0.765 | 0.002 | 0.004 | 0.002 | 0.008 | 0.007 | 0.011 | 0.002 | 0.021 | 0.01 |
| Beagle | | 0.004 | 0.004 | 0.002 | 0.006 | 0.006 | 0.854 | 0.002 | 0.002 | 0.002 | 0.006 | 0.011 | 0.006 | 0.011 | 0.005 | 0.003 |
| Beagle | | 0.001 | 0.008 | 0.004 | 0.002 | 0.002 | 0.785 | 0.008 | 0.004 | 0.003 | 0.003 | 0.003 | 0.004 | 0.007 | 0.026 | 0.002 |
| Beagle | | 0.002 | 0.002 | 0.003 | 0.002 | 0.003 | 0.929 | 0.003 | 0.002 | 0.004 | 0.002 | 0.001 | 0.002 | 0.002 | 0.002 | 0.011 |
| Beagle | | 0.001 | 0.003 | 0.002 | 0.002 | 0.003 | 0.94 | 0.003 | 0.002 | 0.002 | 0.001 | 0.002 | 0.001 | 0.002 | 0.002 | 0.005 |
| Beagle | | 0.001 | 0.019 | 0.014 | 0.043 | 0.004 | 0.767 | 0.009 | 0.005 | 0.002 | 0.008 | 0.002 | 0.003 | 0.012 | 0.002 | 0.006 |
| Beagle | | 0.001 | 0.001 | 0.003 | 0.006 | 0.002 | 0.89 | 0.003 | 0.01 | 0.006 | 0.003 | 0.004 | 0.004 | 0.004 | 0.007 | 0.009 |
| Beagle | | 0.001 | 0.004 | 0.004 | 0.004 | 0.002 | 0.859 | 0.009 | 0.007 | 0.006 | 0.003 | 0.003 | 0.005 | 0.006 | 0.004 | 0.006 |
| Beagle | | 0.001 | 0.006 | 0.005 | 0.002 | 0.003 | 0.9 | 0.002 | 0.002 | 0.004 | 0.003 | 0.002 | 0.007 | 0.003 | 0.002 | 0.004 |
| Beagle | | 0.009 | 0.01 | 0.004 | 0.004 | 0.001 | 0.887 | 0.002 | 0.008 | 0.001 | 0.002 | 0.006 | 0.008 | 0.002 | 0.002 | 0.002 |
| Beagle | | 0.002 | 0.004 | 0.003 | 0.028 | 0.011 | 0.825 | 0.003 | 0.003 | 0.002 | 0.001 | 0.002 | 0.004 | 0.012 | 0.002 | 0.004 |
| Beagle | | 0.001 | 0.001 | 0.002 | 0.003 | 0.002 | 0.932 | 0.003 | 0.001 | 0.003 | 0.003 | 0.003 | 0.001 | 0.001 | 0.008 | 0.002 |
| Beagle | | 0.001 | 0.002 | 0.003 | 0.002 | 0.002 | 0.946 | 0.004 | 0.002 | 0.002 | 0.002 | 0.001 | 0.002 | 0.001 | 0.002 | 0.002 |
| Beagle | | 0.001 | 0.004 | 0.002 | 0.002 | 0.002 | 0.938 | 0.002 | 0.004 | 0.002 | 0.002 | 0.002 | 0.002 | 0.002 | 0.002 | 0.002 |
| Beagle | | 0.002 | 0.002 | 0.004 | 0.005 | 0.003 | 0.829 | 0.003 | 0.004 | 0.006 | 0.008 | 0.002 | 0.011 | 0.006 | 0.001 | 0.002 |
| Beagle | | 0.006 | 0.002 | 0.002 | 0.002 | 0.004 | 0.811 | 0.002 | 0.003 | 0.004 | 0.016 | 0.007 | 0.006 | 0.003 | 0.004 | 0.013 |
| Beagle | | 0.003 | 0.002 | 0.004 | 0.002 | 0.003 | 0.746 | 0.002 | 0.002 | 0.004 | 0.006 | 0.001 | 0.003 | 0.002 | 0.002 | 0.014 |
| Beagle | | 0.002 | 0.004 | 0.003 | 0.001 | 0.002 | 0.918 | 0.002 | 0.002 | 0.004 | 0.002 | 0.006 | 0.002 | 0.002 | 0.007 | 0.002 |
| Beagle | | 0.007 | 0.005 | 0.001 | 0.006 | 0.002 | 0.933 | 0.002 | 0.002 | 0.003 | 0.002 | 0.002 | 0.001 | 0.001 | 0.001 | 0.002 |
| Beagle | | 0.003 | 0.001 | 0.004 | 0.003 | 0.003 | 0.915 | 0.004 | 0.001 | 0.002 | 0.003 | 0.004 | 0.002 | 0.001 | 0.002 | 0.007 |
| Beagle | | 0.001 | 0.002 | 0.002 | 0.002 | 0.001 | 0.943 | 0.001 | 0.002 | 0.002 | 0.004 | 0.004 | 0.002 | 0.002 | 0.002 | 0.003 |
| Beagle | | 0.001 | 0.002 | 0.002 | 0.002 | 0.001 | 0.952 | 0.002 | 0.002 | 0.001 | 0.001 | 0.002 | 0.002 | 0.001 | 0.005 | 0.002 |
| Beagle | | 0.001 | 0.002 | 0.003 | 0.003 | 0.001 | 0.873 | 0.011 | 0.003 | 0.002 | 0.001 | 0.002 | 0.001 | 0.002 | 0.003 | 0.005 |
| Beagle | | 0.003 | 0.004 | 0.003 | 0.011 | 0.006 | 0.765 | 0.003 | 0.047 | 0.006 | 0.014 | 0.004 | 0.006 | 0.003 | 0.009 | 0.002 |
| Beagle | | 0.004 | 0.004 | 0.004 | 0.002 | 0.002 | 0.777 | 0.013 | 0.012 | 0.002 | 0.001 | 0.006 | 0.087 | 0.004 | 0.002 | 0.002 |
| Beagle | | 0.001 | 0.001 | 0.002 | 0.003 | 0.001 | 0.929 | 0.002 | 0.002 | 0.003 | 0.006 | 0.002 | 0.001 | 0.002 | 0.001 | 0.003 |
| Beagle | | 0.002 | 0.002 | 0.003 | 0.002 | 0.001 | 0.848 | 0.003 | 0.004 | 0.002 | 0.009 | 0.025 | 0.003 | 0.002 | 0.002 | 0.006 |
| Beagle | | 0.002 | 0.002 | 0.002 | 0.002 | 0.002 | 0.946 | 0.003 | 0.003 | 0.002 | 0.002 | 0.001 | 0.002 | 0.002 | 0.001 | 0.002 |
| Beagle | | 0.001 | 0.002 | 0.002 | 0.003 | 0.002 | 0.9 | 0.002 | 0.013 | 0.007 | 0.002 | 0.002 | 0.003 | 0.004 | 0.002 | 0.005 |
| Beagle | | 0.001 | 0.003 | 0.003 | 0.006 | 0.004 | 0.775 | 0.004 | 0.005 | 0.008 | 0.003 | 0.017 | 0.012 | 0.003 | 0.008 | 0.006 |
| Beagle | | 0.001 | 0.001 | 0.002 | 0.048 | 0.002 | 0.789 | 0.006 | 0.004 | 0.01 | 0.002 | 0.005 | 0.002 | 0.005 | 0.016 | 0.003 |
| Beagle | | 0.002 | 0.002 | 0.002 | 0.004 | 0.002 | 0.913 | 0.004 | 0.005 | 0.003 | 0.001 | 0.004 | 0.002 | 0.003 | 0.005 | 0.003 |
| Beagle | | 0.002 | 0.003 | 0.004 | 0.011 | 0.003 | 0.843 | 0.007 | 0.002 | 0.007 | 0.002 | 0.001 | 0.002 | 0.005 | 0.001 | 0.004 |
| Beagle | | 0.009 | 0.002 | 0.002 | 0.002 | 0.002 | 0.829 | 0.003 | 0.001 | 0.003 | 0.006 | 0.004 | 0.006 | 0.002 | 0.002 | 0.022 |
| Beagle | | 0.002 | 0.003 | 0.003 | 0.003 | 0.002 | 0.819 | 0.006 | 0.004 | 0.022 | 0.002 | 0.005 | 0.002 | 0.013 | 0.007 | 0.007 |
| Beagle | | 0.001 | 0.009 | 0.006 | 0.004 | 0.003 | 0.812 | 0.066 | 0.002 | 0.004 | 0.002 | 0.004 | 0.002 | 0.003 | 0.002 | 0.006 |
| | % | Cluster | | | | | | | | | | | | | | |
| | missing | Assignment | | | | | | | | | | | | | | |
| Breed | data | 1 | 2 | 3 | 4 | 5 | 6 | 7 | 8 | 9 | 10 | 11 | 12 | 13 | 14 | 15 |
| Belgian | 0 | 0.002 | 0.003 | 0.006 | 0.003 | 0.001 | 0.003 | 0.002 | 0.004 | .002 | 0.005 | 0.004 | 0.002 | 0.002 | 0.002 | 0.002 |
| Tervuren | | | | | | | | | | | | | | | | |
| Belgian | 0 | 0.015 | 0.003 | 0.004 | 0.005 | 0.003 | 0.01 | 0.003 | 0.007 | 0.002 | 0.002 | 0.015 | 0.002 | 0.005 | 0.002 | 0.003 |
| Tervuren | | | | | | | | | | | | | | | | |
| Belgian | (1) | 0.003 | 0.003 | 0.006 | 0.006 | 0.001 | 0.002 | 0.003 | 0.004 | 0.002 | 0.003 | 0.002 | 0.002 | 0.001 | 0.002 | 0.001 |
| Tervuren | | | | | | | | | | | | | | | | |
| Belgian | 0 | 0.004 | 0.002 | 0.004 | 0.006 | 0.001 | 0.005 | 0.051 | 0.007 | 0.001 | 0.011 | 0.002 | 0.011 | 0.012 | 0.002 | 0.002 |
| Tervuren | | | | | | | | | | | | | | | | |
| Belgian | 0 | 0.006 | 0.003 | 0.001 | 0.001 | 0.001 | 0.002 | 0.001 | 0.002 | 0.001 | 0.003 | 0.005 | 0.001 | 0.002 | 0.003 | 0.003 |
| Tervuren | | | | | | | | | | | | | | | | |
| Belgian | (2) | 0.003 | 0.005 | 0.003 | 0.004 | 0.003 | 0.006 | 0.004 | 0.002 | 0.004 | 0.003 | 0.005 | 0.017 | 0.009 | 0.004 | 0.003 |
| Tervuren | | | | | | | | | | | | | | | | |
| Belgian | (1) | 0.005 | 0.033 | 0.01 | 0.003 | 0.003 | 0.005 | 0.002 | 0.003 | 0.008 | 0.002 | 0.003 | 0.001 | 0.003 | 0.002 | 0.003 |
| Tervuren | | | | | | | | | | | | | | | | |
| Belgian | 0 | 0.002 | 0.002 | 0.004 | 0.005 | 0.001 | 0.018 | 0.003 | 0.004 | 0.002 | 0.003 | 0.009 | 0.002 | 0.014 | 0.004 | 0.002 |
| Tervuren | | | | | | | | | | | | | | | | |
| Belgian | (1) | 0.004 | 0.004 | 0.003 | 0.003 | 0.002 | 0.002 | 0.002 | 0.002 | 0.001 | 0.001 | 0.002 | 0.006 | 0.003 | 0.004 | 0.001 |
| Tervuren | | | | | | | | | | | | | | | | |
| Belgian | 0 | 0.083 | 0.003 | 0.001 | 0.002 | 0.003 | 0.004 | 0.003 | 0.004 | 0.004 | 0.001 | 0.004 | 0.002 | 0.003 | 0.003 | 0.002 |
| Tervuren | | | | | | | | | | | | | | | | |
| Belgian | (1) | 0.002 | 0.003 | 0.002 | 0.001 | 0.002 | 0.003 | 0.003 | 0.004 | 0.001 | 0.003 | 0.003 | 0.002 | 0.002 | 0.002 | 0.003 |
| Tervuren | | | | | | | | | | | | | | | | |
| Belgian | (3) | 0.007 | 0.004 | 0.003 | 0.006 | 0.002 | 0.006 | 0.004 | 0.013 | 0.001 | 0.002 | 0.009 | 0.002 | 0.004 | 0.003 | 0.002 |
| Tervuren | | | | | | | | | | | | | | | | |
| Belgian | (1) | 0.005 | 0.007 | 0.004 | 0.003 | 0.002 | 0.003 | 0.007 | 0.004 | 0.003 | 0.002 | 0.005 | 0.002 | 0.002 | 0.002 | 0.002 |
| Tervuren | | | | | | | | | | | | | | | | |
| Belgian | 0 | 0.006 | 0.003 | 0.003 | 0.003 | 0.007 | 0.006 | 0.004 | 0.009 | 0.005 | 0.001 | 0.003 | 0.019 | 0.003 | 0.004 | 0.004 |
| Tervuren | | | | | | | | | | | | | | | | |
| Belgian | 0 | 0.004 | 0.001 | 0.002 | 0.003 | 0.013 | 0.006 | 0.003 | 0.002 | 0.004 | 0.001 | 0.005 | 0.009 | 0.002 | 0.003 | 0.002 |
| Tervuren | | | | | | | | | | | | | | | | |
| Belgian | 0 | 0.005 | 0.002 | 0.001 | 0.001 | 0.003 | 0.002 | 0.004 | 0.004 | 0.007 | 0.001 | 0.004 | 0.004 | 0.002 | 0.002 | 0.001 |
| Tervuren | | | | | | | | | | | | | | | | |
| Belgian | 0 | 0.002 | 0.011 | 0.002 | 0.001 | 0.002 | 0.004 | 0.005 | 0.013 | 0.111 | 0.04 | 0.003 | 0.004 | 0.064 | 0.004 | 0.001 |
| Tervuren | | | | | | | | | | | | | | | | |
| Belgian | 0 | 0.002 | 0.001 | 0.001 | 0.001 | 0.002 | 0.002 | 0.002 | 0.003 | 0.006 | 0.004 | 0.004 | 0.006 | 0.002 | 0.004 | 0.001 |
| Tervuren | | | | | | | | | | | | | | | | |
| Belgian | 0 | 0.002 | 0.001 | 0.002 | 0.001 | 0.001 | 0.002 | 0.001 | 0.002 | 0.001 | 0.001 | 0.012 | 0.001 | 0.002 | 0.003 | 0.002 |
| Tervuren | | | | | | | | | | | | | | | | |
| Belgian | (1) | 0.006 | 0.002 | 0.002 | 0.002 | 0.002 | 0.006 | 0.002 | 0.006 | 0.003 | 0.002 | 0.009 | 0.004 | 0.005 | 0.016 | 0.004 |
| Tervuren | | | | | | | | | | | | | | | | |
| Belgian | 0 | 0.002 | 0.006 | 0.015 | 0.004 | 0.001 | 0.004 | 0.006 | 0.006 | 0.009 | 0.011 | 0.002 | 0.002 | 0.02 | 0.002 | 0.002 |
| Tervuren | | | | | | | | | | | | | | | | |
| Belgian | 0 | 0.014 | 0.024 | 0.002 | 0.002 | 0.003 | 0.009 | 0.008 | 0.003 | 0.006 | 0.012 | 0.007 | 0.002 | 0.003 | 0.005 | 0.006 |
| Tervuren | | | | | | | | | | | | | | | | |
| Belgian | 0 | 0.009 | 0.032 | 0.003 | 0.004 | 0.001 | 0.009 | 0.008 | 0.006 | 0.003 | 0.005 | 0.006 | 0.002 | 0.004 | 0.005 | 0.007 |
| Tervuren | | | | | | | | | | | | | | | | |
| Belgian | 0 | 0.002 | 0.002 | 0.003 | 0.004 | 0.001 | 0.002 | 0.002 | 0.002 | 0.002 | 0.037 | 0.01 | 0.004 | 0.002 | 0.003 | 0.006 |
| Tervuren | | | | | | | | | | | | | | | | |
| Belgian | (1) | 0.004 | 0.004 | 0.003 | 0.002 | 0.001 | 0.002 | 0.005 | 0.004 | 0.003 | 0.002 | 0.002 | 0.001 | 0.002 | 0.002 | 0.002 |
| Tervuren | | | | | | | | | | | | | | | | |
| Belgian | 0 | 0.003 | 0.007 | 0.002 | 0.003 | 0.003 | 0.001 | 0.002 | 0.002 | 0.002 | 0.002 | 0.001 | 0.004 | 0.002 | 0.002 | 0.001 |
| Tervuren | | | | | | | | | | | | | | | | |
| Belgian | (1) | 0.002 | 0.009 | 0.002 | 0.003 | 0.004 | 0.003 | 0.002 | 0.002 | 0.004 | 0.003 | 0.018 | 0.002 | 0.006 | 0.002 | 0.002 |
| Tervuren | | | | | | | | | | | | | | | | |
| Belgian | 0 | 0.001 | 0.021 | 0.008 | 0.003 | 0.004 | 0.002 | 0.002 | 0.004 | 0.002 | 0.004 | 0.006 | 0.003 | 0.003 | 0.034 | 0.006 |
| Tervuren | | | | | | | | | | | | | | | | |
| Belgian | 0 | 0.004 | 0.03 | 0.003 | 0.002 | 0.002 | 0.002 | 0.006 | 0.016 | 0.01 | 0.002 | 0.007 | 0.006 | 0.004 | 0.008 | 0.003 |
| Tervuren | | | | | | | | | | | | | | | | |
| Belgian | (4) | 0.001 | 0.003 | 0.006 | 0.002 | 0.004 | 0.002 | 0.001 | 0.003 | 0.001 | 0.006 | 0.004 | 0.002 | 0.003 | 0.006 | 0.005 |
| Tervuren | | | | | | | | | | | | | | | | |
| Belgian | (3) | 0.002 | 0.012 | 0.002 | 0.001 | 0.001 | 0.003 | 0.002 | 0.003 | 0.002 | 0.005 | 0.003 | 0.002 | 0.008 | 0.005 | 0.002 |
| Tervuren | | | | | | | | | | | | | | | | |
| Belgian | (1) | 0.002 | 0.005 | 0.016 | 0.002 | 0.003 | 0.003 | 0.003 | 0.003 | 0.002 | 0.006 | 0.003 | 0.002 | 0.003 | 0.004 | 0.003 |
| Tervuren | | | | | | | | | | | | | | | | |
| Belgian | 0 | 0.003 | 0.003 | 0.014 | 0.006 | 0.002 | 0.002 | 0.002 | 0.005 | 0.002 | 0.003 | 0.003 | 0.004 | 0.003 | 0.003 | 0.002 |
| Tervuren | | | | | | | | | | | | | | | | |
| Belgian | 0 | 0.003 | 0.003 | 0.001 | 0.002 | 0.001 | 0.003 | 0.007 | 0.01 | 0.007 | 0.005 | 0.002 | 0.003 | 0.002 | 0.002 | 0.004 |
| Tervuren | | | | | | | | | | | | | | | | |
| Belgian | 0 | 0.011 | 0.031 | 0.004 | 0.002 | 0.001 | 0.009 | 0.002 | 0.005 | 0.001 | 0.004 | 0.005 | 0.002 | 0.002 | 0.004 | 0.074 |
| Tervuren | | | | | | | | | | | | | | | | |
| Belgian | 0 | 0.013 | 0.01 | 0.002 | 0.004 | 0.001 | 0.016 | 0.01 | 0.005 | 0.003 | 0.006 | 0.004 | 0.007 | 0.005 | 0.003 | 0.003 |
| Tervuren | | | | | | | | | | | | | | | | |
| Belgian | 0 | 0.015 | 0.003 | 0.004 | 0.002 | 0.002 | 0.002 | 0.007 | 0.014 | 0.013 | 0.002 | 0.004 | 0.002 | 0.026 | 0.002 | 0.001 |
| Tervuren | | | | | | | | | | | | | | | | |
| Belgian | 0 | 0.002 | 0.003 | 0.004 | 0.004 | 0.001 | 0.002 | 0.003 | 0.003 | 0.001 | 0.005 | 0.004 | 0.001 | 0.003 | 0.002 | 0.001 |
| Tervuren | | | | | | | | | | | | | | | | |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Belgian | | 0.003 | 0.005 | 0.911 | 0.004 | 0.003 | 0.004 | 0.001 | 0.003 | 0.002 | 0.002 | 0.008 | 0.002 | 0.003 | 0.003 | 0.004 |
| Tervuren | | | | | | | | | | | | | | | | |
| Belgian | | 0.005 | 0.024 | 0.835 | 0.015 | 0.003 | 0.005 | 0.004 | 0.005 | 0.006 | 0.003 | 0.002 | 0.004 | 0.004 | 0.002 | 0.001 |
| Tervuren | | | | | | | | | | | | | | | | |
| Belgian | | 0.003 | 0.005 | 0.914 | 0.003 | 0.001 | 0.003 | 0.001 | 0.005 | 0.003 | 0.002 | 0.004 | 0.003 | 0.006 | 0.005 | 0.001 |
| Tervuren | | | | | | | | | | | | | | | | |
| Belgian | | 0.002 | 0.004 | 0.781 | 0.003 | 0.021 | 0.003 | 0.004 | 0.008 | 0.005 | 0.027 | 0.008 | 0.006 | 0.002 | 0.001 | 0.003 |
| Tervuren | | | | | | | | | | | | | | | | |
| Belgian | | 0.001 | 0.002 | 0.942 | 0.001 | 0.002 | 0.002 | 0.002 | 0.001 | 0.002 | 0.001 | 0.001 | 0.001 | 0.002 | 0.003 | 0.001 |
| Tervuren | | | | | | | | | | | | | | | | |
| Belgian | | 0.001 | 0.003 | 0.838 | 0.007 | 0.002 | 0.002 | 0.041 | 0.003 | 0.003 | 0.001 | 0.004 | 0.001 | 0.004 | 0.002 | 0.018 |
| Tervuren | | | | | | | | | | | | | | | | |
| Belgian | | 0.003 | 0.003 | 0.827 | 0.004 | 0.005 | 0.01 | 0.005 | 0.003 | 0.005 | 0.009 | 0.005 | 0.003 | 0.003 | 0.002 | 0.028 |
| Tervuren | | | | | | | | | | | | | | | | |
| Belgian | | 0.001 | 0.005 | 0.883 | 0.003 | 0.002 | 0.003 | 0.005 | 0.001 | 0.002 | 0.002 | 0.002 | 0.003 | 0.007 | 0.003 | 0.004 |
| Tervuren | | | | | | | | | | | | | | | | |
| Belgian | | 0.001 | 0.003 | 0.925 | 0.002 | 0.002 | 0.003 | 0.004 | 0.001 | 0.003 | 0.001 | 0.001 | 0.001 | 0.002 | 0.002 | 0.007 |
| Tervuren | | | | | | | | | | | | | | | | |
| Belgian | | 0.004 | 0.002 | 0.842 | 0.002 | 0.002 | 0.002 | 0.002 | 0.001 | 0.002 | 0.003 | 0.003 | 0.003 | 0.002 | 0.004 | 0.003 |
| Tervuren | | | | | | | | | | | | | | | | |
| Belgian | | 0.002 | 0.002 | 0.908 | 0.004 | 0.003 | 0.003 | 0.003 | 0.007 | 0.004 | 0.002 | 0.006 | 0.006 | 0.003 | 0.002 | 0.01 |
| Tervuren | | | | | | | | | | | | | | | | |
| Belgian | | 0.002 | 0.007 | 0.845 | 0.009 | 0.006 | 0.01 | 0.005 | 0.003 | 0.006 | 0.002 | 0.005 | 0.005 | 0.022 | 0.004 | 0.003 |
| Tervuren | | | | | | | | | | | | | | | | |
| Belgian | | 0.001 | 0.015 | 0.846 | 0.006 | 0.003 | 0.007 | 0.002 | 0.04 | 0.003 | 0.002 | 0.004 | 0.002 | 0.003 | 0.001 | 0.01 |
| Tervuren | | | | | | | | | | | | | | | | |
| Belgian | | 0.001 | 0.004 | 0.856 | 0.003 | 0.014 | 0.003 | 0.006 | 0.003 | 0.017 | 0.001 | 0.002 | 0.001 | 0.005 | 0.003 | 0.002 |
| Tervuren | | | | | | | | | | | | | | | | |
| Belgian | | 0.002 | 0.005 | 0.88 | 0.006 | 0.009 | 0.001 | 0.002 | 0.005 | 0.012 | 0.001 | 0.002 | 0.002 | 0.005 | 0.004 | 0.004 |
| Tervuren | | | | | | | | | | | | | | | | |
| Belgian | | 0.001 | 0.002 | 0.921 | 0.004 | 0.001 | 0.003 | 0.002 | 0.001 | 0.005 | 0.005 | 0.004 | 0.002 | 0.002 | 0.001 | 0.003 |
| Tervuren | | | | | | | | | | | | | | | | |
| Belgian | | 0.002 | 0.003 | 0.673 | 0.002 | 0.012 | 0.004 | 0.002 | 0.005 | 0.001 | 0.008 | 0.004 | 0.011 | 0.003 | 0.002 | 0.001 |
| Tervuren | | | | | | | | | | | | | | | | |
| Belgian | | 0.002 | 0.009 | 0.908 | 0.003 | 0.012 | 0.001 | 0.003 | 0.002 | 0.008 | 0.002 | 0.001 | 0.001 | 0.002 | 0.001 | 0.001 |
| Tervuren | | | | | | | | | | | | | | | | |
| Belgian | | 0.001 | 0.006 | 0.938 | 0.002 | 0.002 | 0.001 | 0.002 | 0.002 | 0.002 | 0.002 | 0.002 | 0.003 | 0.001 | 0.002 | 0.001 |
| Tervuren | | | | | | | | | | | | | | | | |
| Belgian | | 0.002 | 0.002 | 0.851 | 0.011 | 0.001 | 0.005 | 0.003 | 0.005 | 0.01 | 0.022 | 0.002 | 0.007 | 0.001 | 0.003 | 0.004 |
| Tervuren | | | | | | | | | | | | | | | | |
| Belgian | | 0.001 | 0.002 | 0.83 | 0.002 | 0.004 | 0.002 | 0.024 | 0.003 | 0.001 | 0.004 | 0.006 | 0.015 | 0.003 | 0.001 | 0.007 |
| Tervuren | | | | | | | | | | | | | | | | |
| Belgian | | 0.005 | 0.01 | 0.824 | 0.01 | 0.006 | 0.003 | 0.009 | 0.003 | 0.003 | 0.002 | 0.002 | 0.003 | 0.012 | 0.002 | 0.002 |
| Tervuren | | | | | | | | | | | | | | | | |
| Belgian | | 0.002 | 0.002 | 0.825 | 0.005 | 0.009 | 0.005 | 0.005 | 0.007 | 0.005 | 0.001 | 0.008 | 0.003 | 0.008 | 0.002 | 0.007 |
| Tervuren | | | | | | | | | | | | | | | | |
| Belgian | | 0.001 | 0.003 | 0.882 | 0.004 | 0.008 | 0.002 | 0.002 | 0.002 | 0.003 | 0.001 | 0.002 | 0.002 | 0.004 | 0.002 | 0.002 |
| Tervuren | | | | | | | | | | | | | | | | |
| Belgian | | 0.001 | 0.007 | 0.911 | 0.002 | 0.002 | 0.006 | 0.002 | 0.002 | 0.002 | 0.003 | 0.008 | 0.002 | 0.003 | 0.002 | 0.008 |
| Tervuren | | | | | | | | | | | | | | | | |
| Belgian | | 0.001 | 0.005 | 0.911 | 0.002 | 0.004 | 0.006 | 0.011 | 0.002 | 0.002 | 0.003 | 0.003 | 0.002 | 0.001 | 0.002 | 0.008 |
| Tervuren | | | | | | | | | | | | | | | | |
| Belgian | | 0.002 | 0.003 | 0.904 | 0.004 | 0.002 | 0.002 | 0.002 | 0.001 | 0.002 | 0.002 | 0.003 | 0.002 | 0.002 | 0.003 | 0.003 |
| Tervuren | | | | | | | | | | | | | | | | |
| Belgian | | 0.002 | 0.003 | 0.834 | 0.007 | 0.001 | 0.006 | 0.002 | 0.005 | 0.004 | 0.016 | 0.002 | 0.002 | 0.003 | 0.006 | 0.002 |
| Tervuren | | | | | | | | | | | | | | | | |
| Belgian | | 0.001 | 0.003 | 0.823 | 0.003 | 0.002 | 0.017 | 0.004 | 0.004 | 0.004 | 0.005 | 0.005 | 0.003 | 0.005 | 0.004 | 0.009 |
| Tervuren | | | | | | | | | | | | | | | | |
| Belgian | | 0.004 | 0.004 | 0.899 | 0.008 | 0.001 | 0.004 | 0.004 | 0.003 | 0.002 | 0.006 | 0.002 | 0.002 | 0.003 | 0.008 | 0.001 |
| Tervuren | | | | | | | | | | | | | | | | |
| Belgian | | 0.002 | 0.005 | 0.906 | 0.001 | 0.002 | 0.005 | 0.004 | 0.003 | 0.003 | 0.004 | 0.002 | 0.002 | 0.002 | 0.003 | 0.002 |
| Tervuren | | | | | | | | | | | | | | | | |
| Belgian | | 0.002 | 0.003 | 0.877 | 0.005 | 0.002 | 0.004 | 0.002 | 0.012 | 0.003 | 0.009 | 0.004 | 0.002 | 0.003 | 0.008 | 0.002 |
| Tervuren | | | | | | | | | | | | | | | | |
| Belgian | | 0.002 | 0.012 | 0.884 | 0.008 | 0.001 | 0.004 | 0.002 | 0.002 | 0.004 | 0.006 | 0.003 | 0.004 | 0.004 | 0.003 | 0.006 |
| Tervuren | | | | | | | | | | | | | | | | |
| Belgian | | 0.001 | 0.003 | 0.902 | 0.002 | 0.004 | 0.003 | 0.009 | 0.002 | 0.002 | 0.009 | 0.002 | 0.002 | 0.002 | 0.002 | 0.003 |
| Tervuren | | | | | | | | | | | | | | | | |
| Belgian | | 0.004 | 0.005 | 0.779 | 0.003 | 0.007 | 0.005 | 0.008 | 0.003 | 0.006 | 0.001 | 0.007 | 0.002 | 0.005 | 0.004 | 0.004 |
| Tervuren | | | | | | | | | | | | | | | | |
| Belgian | | 0.009 | 0.01 | 0.803 | 0.035 | 0.004 | 0.004 | 0.007 | 0.004 | 0.004 | 0.003 | 0.002 | 0.004 | 0.008 | 0.009 | 0.004 |
| Tervuren | | | | | | | | | | | | | | | | |
| Belgian | | 0.003 | 0.004 | 0.824 | 0.002 | 0.003 | 0.003 | 0.005 | 0.001 | 0.007 | 0.012 | 0.014 | 0.01 | 0.003 | 0.002 | 0.007 |
| Tervuren | | | | | | | | | | | | | | | | |
| Belgian | | 0.005 | 0.008 | 0.918 | 0.004 | 0.003 | 0.004 | 0.001 | 0.003 | 0.004 | 0.003 | 0.002 | 0.003 | 0.002 | 0.001 | 0.003 |
| Tervuren | | | | | | | | | | | | | | | | |
| | % | Cluster | | | | | | | | | | | | | | |
| | missing | Assignment | | | | | | | | | | | | | | |
| Breed | data | 1 | 2 | 3 | 4 | 5 | 6 | 7 | 8 | 9 | 10 | 11 | 12 | 13 | 14 | 15 |
| Bernese | (3) | 0.004 | 0.004 | 0.002 | 0.002 | 0.004 | 0.003 | 0.002 | 0.005 | 0.002 | 0.001 | 0.005 | 0.003 | 0.002 | 0.001 | 0.005 |
| Mountain | | | | | | | | | | | | | | | | |
| Bernese | 0 | 0.002 | 0.002 | 0.001 | 0.001 | 0.001 | 0.001 | 0.002 | 0.003 | 0.001 | 0.001 | 0.001 | 0.001 | 0.001 | 0.001 | 0.002 |
| Mountain | | | | | | | | | | | | | | | | |
| Bernese | 0 | 0.001 | 0.002 | 0.001 | 0.001 | 0.001 | 0.001 | 0.001 | 0.001 | 0.001 | 0.002 | 0.001 | 0.001 | 0.001 | 0.001 | 0.001 |
| Mountain | | | | | | | | | | | | | | | | |
| Bernese | 0 | 0.002 | 0.005 | 0.003 | 0.002 | 0.001 | 0.004 | 0.004 | 0.002 | 0.001 | 0.004 | 0.003 | 0.002 | 0.008 | 0.006 | 0.002 |
| Mountain | | | | | | | | | | | | | | | | |
| Bernese | (1) | 0.002 | 0.002 | 0.001 | 0.002 | 0.002 | 0.001 | 0.002 | 0.002 | 0.002 | 0.002 | 0.003 | 0.001 | 0.001 | 0.003 | 0.002 |
| Mountain | | | | | | | | | | | | | | | | |
| Bernese | 0 | 0.003 | 0.002 | 0.002 | 0.002 | 0.001 | 0.002 | 0.002 | 0.002 | 0.002 | 0.003 | 0.001 | 0.002 | 0.002 | 0.001 | 0.002 |
| Mountain | | | | | | | | | | | | | | | | |
| Bernese | (4) | 0.002 | 0.003 | 0.003 | 0.002 | 0.001 | 0.002 | 0.002 | 0.002 | 0.001 | 0.005 | 0.002 | 0.001 | 0.002 | 0.003 | 0.006 |
| Mountain | | | | | | | | | | | | | | | | |
| Bernese | (2) | 0.001 | 0.001 | 0.001 | 0.002 | 0.002 | 0.002 | 0.001 | 0.002 | 0.002 | 0.001 | 0.004 | 0.001 | 0.001 | 0.001 | 0.001 |
| Mountain | | | | | | | | | | | | | | | | |
| Bernese | (3) | 0.002 | 0.003 | 0.003 | 0.002 | 0.003 | 0.003 | 0.003 | 0.004 | 0.003 | 0.001 | 0.013 | 0.002 | 0.004 | 0.002 | 0.003 |
| Mountain | | | | | | | | | | | | | | | | |
| Bernese | (39) | 0.003 | 0.007 | 0.003 | 0.007 | 0.002 | 0.002 | 0.007 | 0.026 | 0.002 | 0.002 | 0.01 | 0.006 | 0.008 | 0.004 | 0.002 |
| Mountain | | | | | | | | | | | | | | | | |
| Bernese | 0 | 0.003 | 0.002 | 0.002 | 0.006 | 0.004 | 0.004 | 0.005 | 0.003 | 0.002 | 0.005 | 0.006 | 0.005 | 0.006 | 0.003 | 0.002 |
| Mountain | | | | | | | | | | | | | | | | |
| Bernese | 0 | 0.003 | 0.001 | 0.002 | 0.002 | 0.001 | 0.001 | 0.002 | 0.003 | 0.001 | 0.002 | 0.002 | 0.001 | 0.003 | 0.002 | 0.001 |
| Mountain | | | | | | | | | | | | | | | | |
| Bernese | (2) | 0.002 | 0.002 | 0.001 | 0.002 | 0.001 | 0.002 | 0.002 | 0.002 | 0.003 | 0.002 | 0.002 | 0.002 | 0.001 | 0.005 | 0.003 |
| Mountain | | | | | | | | | | | | | | | | |
| Bernese | 0 | 0.003 | 0.012 | 0.001 | 0.001 | 0.005 | 0.008 | 0.007 | 0.002 | 0.002 | 0.002 | 0.003 | 0.001 | 0.002 | 0.029 | 0.002 |
| Mountain | | | | | | | | | | | | | | | | |
| Bernese | (2) | 0.001 | 0.002 | 0.002 | 0.001 | 0.001 | 0.001 | 0.001 | 0.001 | 0.002 | 0.002 | 0.002 | 0.001 | 0.001 | 0.002 | 0.002 |
| Mountain | | | | | | | | | | | | | | | | |
| Bernese | (2) | 0.002 | 0.003 | 0.005 | 0.005 | 0.004 | 0.002 | 0.002 | 0.005 | 0.001 | 0.001 | 0.006 | 0.01 | 0.006 | 0.005 | 0.003 |
| Mountain | | | | | | | | | | | | | | | | |
| Bernese | (1) | 0.001 | 0.004 | 0.002 | 0.001 | 0.001 | 0.004 | 0.002 | 0.002 | 0.002 | 0.003 | 0.003 | 0.001 | 0.006 | 0.005 | 0.003 |
| Mountain | | | | | | | | | | | | | | | | |
| Bernese | (1) | 0.004 | 0.002 | 0.002 | 0.004 | 0.001 | 0.004 | 0.001 | 0.001 | 0.001 | 0.001 | 0.002 | 0.002 | 0.002 | 0.005 | 0.002 |
| Mountain | | | | | | | | | | | | | | | | |
| Bernese | 0 | 0.001 | 0.001 | 0.001 | 0.001 | 0.001 | 0.001 | 0.001 | 0.001 | 0.001 | 0.001 | 0.001 | 0.001 | 0.001 | 0.001 | 0.002 |
| Mountain | | | | | | | | | | | | | | | | |
| Bernese | (4) | 0.003 | 0.002 | 0.011 | 0.006 | 0.002 | 0.002 | 0.007 | 0.007 | 0.007 | 0.003 | 0.008 | 0.002 | 0.003 | 0.002 | 0.001 |
| Mountain | | | | | | | | | | | | | | | | |
| Bernese | 0 | 0.001 | 0.002 | 0.002 | 0.001 | 0.001 | 0.001 | 0.001 | 0.001 | 0.001 | 0.001 | 0.001 | 0.001 | 0.001 | 0.001 | 0.001 |
| Mountain | | | | | | | | | | | | | | | | |
| Bernese | 0 | 0.002 | 0.004 | 0.008 | 0.007 | 0.001 | 0.006 | 0.002 | 0.001 | 0.002 | 0.008 | 0.007 | 0.001 | 0.002 | 0.002 | 0.002 |
| Mountain | | | | | | | | | | | | | | | | |
| Bernese | 0 | 0.01 | 0.003 | 0.003 | 0.004 | 0.002 | 0.002 | 0.003 | 0.019 | 0.001 | 0.003 | 0.008 | 0.01 | 0.004 | 0.003 | 0.009 |
| Mountain | | | | | | | | | | | | | | | | |
| Bernese | 0 | 0.002 | 0.002 | 0.002 | 0.002 | 0.002 | 0.002 | 0.002 | 0.003 | 0.001 | 0.004 | 0.002 | 0.001 | 0.002 | 0.001 | 0.002 |
| Mountain | | | | | | | | | | | | | | | | |
| Bernese | (1) | 0.001 | 0.002 | 0.001 | 0.002 | 0.001 | 0.002 | 0.001 | 0.002 | 0.001 | 0.002 | 0.001 | 0.001 | 0.003 | 0.001 | 0.002 |
| Mountain | | | | | | | | | | | | | | | | |
| Bernese | (1) | 0.002 | 0.002 | 0.003 | 0.002 | 0.001 | 0.001 | 0.001 | 0.002 | 0.001 | 0.002 | 0.003 | 0.001 | 0.001 | 0.001 | 0.002 |
| Mountain | | | | | | | | | | | | | | | | |
| Bernese | (1) | 0.003 | 0.002 | 0.001 | 0.001 | 0.001 | 0.001 | 0.001 | 0.001 | 0.001 | 0.002 | 0.001 | 0.001 | 0.001 | 0.001 | 0.002 |
| Mountain | | | | | | | | | | | | | | | | |
| Bernese | (3) | 0.003 | 0.001 | 0.001 | 0.002 | 0.001 | 0.002 | 0.003 | 0.002 | 0.003 | 0.002 | 0.002 | 0.001 | 0.001 | 0.002 | 0.003 |
| Mountain | | | | | | | | | | | | | | | | |
| Bernese | (2) | 0.001 | 0.001 | 0.001 | 0.001 | 0.001 | 0.001 | 0.001 | 0.003 | 0.001 | 0.001 | 0.002 | 0.001 | 0.003 | 0.001 | 0.001 |
| Mountain | | | | | | | | | | | | | | | | |
| Bernese | 0 | 0.005 | 0.002 | 0.002 | 0.001 | 0.009 | 0.002 | 0.008 | 0.003 | 0.004 | 0.002 | 0.003 | 0.002 | 0.004 | 0.003 | 0.005 |
| Mountain | | | | | | | | | | | | | | | | |
| Bernese | 0 | 0.003 | 0.003 | 0.002 | 0.002 | 0.001 | 0.003 | 0.002 | 0.003 | 0.002 | 0.002 | 0.003 | 0.001 | 0.002 | 0.002 | 0.002 |
| Mountain | | | | | | | | | | | | | | | | |
| Bernese | (2) | 0.001 | 0.002 | 0.002 | 0.002 | 0.002 | 0.002 | 0.001 | 0.002 | 0.003 | 0.001 | 0.001 | 0.002 | 0.002 | 0.001 | 0.002 |
| Mountain | | | | | | | | | | | | | | | | |
| Bernese | (18) | 0.001 | 0.011 | 0.017 | 0.004 | 0.002 | 0.008 | 0.003 | 0.004 | 0.002 | 0.009 | 0.006 | 0.001 | 0.007 | 0.006 | 0.008 |
| Mountain | | | | | | | | | | | | | | | | |
| Bernese | (3) | 0.002 | 0.004 | 0.002 | 0.003 | 0.003 | 0.002 | 0.003 | 0.002 | 0.004 | 0.002 | 0.003 | 0.011 | 0.001 | 0.005 | 0.005 |
| Mountain | | | | | | | | | | | | | | | | |
| Bernese | (1) | 0.027 | 0.004 | 0.005 | 0.003 | 0.002 | 0.002 | 0.005 | 0.003 | 0.001 | 0.004 | 0.003 | 0.013 | 0.002 | 0.002 | 0.002 |
| Mountain | | | | | | | | | | | | | | | | |
| Bernese | 0 | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Mountain | | | | | | | | | | | | | | | | |
| Bernese | (6) | 0.003 | 0.004 | 0.007 | 0.002 | 0.002 | 0.002 | 0.002 | 0.003 | 0.002 | 0.004 | 0.004 | 0.003 | 0.001 | 0.004 | 0.001 |
| Mountain | | | | | | | | | | | | | | | | |
| Bernese | (12) | 0.003 | 0.002 | 0.001 | 0.002 | 0.002 | 0.002 | 0.011 | 0.022 | 0.005 | 0.001 | 0.018 | 0.002 | 0.003 | 0.003 | 0.003 |
| Mountain | | | | | | | | | | | | | | | | |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Bernese | | 0.002 | 0.002 | 0.004 | 0.006 | 0.002 | 0.002 | 0.002 | 0.002 | 0.003 | 0.002 | 0.003 | 0.002 | 0.004 | 0.916 | 0.002 |
| Mountain | | | | | | | | | | | | | | | | |
| Bernese | | 0.001 | 0.002 | 0.002 | 0.002 | 0.002 | 0.002 | 0.001 | 0.002 | 0.002 | 0.002 | 0.002 | 0.002 | 0.002 | 0.951 | 0.001 |
| Mountain | | | | | | | | | | | | | | | | |
| Bernese | | 0.001 | 0.001 | 0.001 | 0.001 | 0.001 | 0.002 | 0.001 | 0.001 | 0.001 | 0.001 | 0.001 | 0.002 | 0.001 | 0.968 | 0.001 |
| Mountain | | | | | | | | | | | | | | | | |
| Bernese | | 0.003 | 0.004 | 0.002 | 0.002 | 0.002 | 0.004 | 0.002 | 0.003 | 0.003 | 0.003 | 0.01 | 0.002 | 0.005 | 0.906 | 0.001 |
| Mountain | | | | | | | | | | | | | | | | |
| Bernese | | 0.001 | 0.003 | 0.003 | 0.002 | 0.002 | 0.001 | 0.001 | 0.002 | 0.004 | 0.004 | 0.002 | 0.003 | 0.002 | 0.944 | 0.002 |
| Mountain | | | | | | | | | | | | | | | | |
| Bernese | | 0.001 | 0.001 | 0.003 | 0.003 | 0.001 | 0.001 | 0.003 | 0.004 | 0.002 | 0.003 | 0.004 | 0.002 | 0.002 | 0.943 | 0.001 |
| Mountain | | | | | | | | | | | | | | | | |
| Bernese | | 0.001 | 0.003 | 0.003 | 0.003 | 0.002 | 0.003 | 0.006 | 0.003 | 0.005 | 0.002 | 0.001 | 0.002 | 0.003 | 0.922 | 0.003 |
| Mountain | | | | | | | | | | | | | | | | |
| Bernese | | 0.002 | 0.001 | 0.002 | 0.002 | 0.003 | 0.001 | 0.002 | 0.002 | 0.003 | 0.004 | 0.002 | 0.001 | 0.003 | 0.947 | 0.002 |
| Mountain | | | | | | | | | | | | | | | | |
| Bernese | | 0.001 | 0.005 | 0.008 | 0.002 | 0.002 | 0.001 | 0.003 | 0.001 | 0.002 | 0.002 | 0.004 | 0.002 | 0.005 | 0.905 | 0.005 |
| Mountain | | | | | | | | | | | | | | | | |
| Bernese | | 0.002 | 0.048 | 0.01 | 0.002 | 0.007 | 0.002 | 0.003 | 0.006 | 0.003 | 0.014 | 0.02 | 0.055 | 0.003 | 0.728 | 0.004 |
| Mountain | | | | | | | | | | | | | | | | |
| Bernese | | 0.001 | 0.005 | 0.002 | 0.004 | 0.002 | 0.001 | 0.007 | 0.01 | 0.002 | 0.002 | 0.002 | 0.005 | 0.003 | 0.89 | 0.005 |
| Mountain | | | | | | | | | | | | | | | | |
| Bernese | | 0.002 | 0.002 | 0.002 | 0.002 | 0.003 | 0.003 | 0.003 | 0.005 | 0.005 | 0.004 | 0.006 | 0.005 | 0.002 | 0.924 | 0.001 |
| Mountain | | | | | | | | | | | | | | | | |
| Bernese | | 0.001 | 0.002 | 0.002 | 0.001 | 0.001 | 0.001 | 0.001 | 0.001 | 0.002 | 0.001 | 0.001 | 0.001 | 0.002 | 0.946 | 0.003 |
| Mountain | | | | | | | | | | | | | | | | |
| Bernese | | 0.008 | 0.001 | 0.001 | 0.003 | 0.001 | 0.004 | 0.001 | 0.001 | 0.003 | 0.001 | 0.005 | 0.001 | 0.002 | 0.885 | 0.002 |
| Mountain | | | | | | | | | | | | | | | | |
| Bernese | | 0.001 | 0.003 | 0.001 | 0.001 | 0.001 | 0.002 | 0.002 | 0.002 | 0.003 | 0.001 | 0.001 | 0.002 | 0.001 | 0.954 | 0.004 |
| Mountain | | | | | | | | | | | | | | | | |
| Bernese | | 0.001 | 0.002 | 0.004 | 0.004 | 0.001 | 0.001 | 0.01 | 0.003 | 0.002 | 0.002 | 0.008 | 0.004 | 0.009 | 0.886 | 0.004 |
| Mountain | | | | | | | | | | | | | | | | |
| Bernese | | 0.001 | 0.002 | 0.002 | 0.001 | 0.002 | 0.002 | 0.002 | 0.006 | 0.003 | 0.004 | 0.002 | 0.005 | 0.003 | 0.925 | 0.002 |
| Mountain | | | | | | | | | | | | | | | | |
| Bernese | | 0.001 | 0.001 | 0.002 | 0.001 | 0.004 | 0.001 | 0.003 | 0.001 | 0.007 | 0.001 | 0.001 | 0.003 | 0.001 | 0.935 | 0.002 |
| Mountain | | | | | | | | | | | | | | | | |
| Bernese | | 0.001 | 0.001 | 0.002 | 0.001 | 0.001 | 0.001 | 0.002 | 0.001 | 0.003 | 0.001 | 0.001 | 0.001 | 0.001 | 0.963 | 0.001 |
| Mountain | | | | | | | | | | | | | | | | |
| Bernese | | 0.002 | 0.003 | 0.004 | 0.003 | 0.004 | 0.003 | 0.002 | 0.002 | 0.002 | 0.003 | 0.003 | 0.003 | 0.009 | 0.887 | 0.003 |
| Mountain | | | | | | | | | | | | | | | | |
| Bernese | | 0.001 | 0.001 | 0.001 | 0.001 | 0.001 | 0.001 | 0.002 | 0.002 | 0.001 | 0.002 | 0.001 | 0.001 | 0.001 | 0.966 | 0.001 |
| Mountain | | | | | | | | | | | | | | | | |
| Bernese | | 0.002 | 0.002 | 0.005 | 0.002 | 0.001 | 0.001 | 0.002 | 0.003 | 0.002 | 0.003 | 0.011 | 0.002 | 0.002 | 0.895 | 0.012 |
| Mountain | | | | | | | | | | | | | | | | |
| Bernese | | 0.001 | 0.002 | 0.01 | 0.004 | 0.003 | 0.003 | 0.016 | 0.006 | 0.005 | 0.014 | 0.018 | 0.01 | 0.003 | 0.815 | 0.006 |
| Mountain | | | | | | | | | | | | | | | | |
| Bernese | | 0.001 | 0.003 | 0.003 | 0.003 | 0.001 | 0.001 | 0.002 | 0.002 | 0.004 | 0.003 | 0.001 | 0.003 | 0.001 | 0.94 | 0.002 |
| Mountain | | | | | | | | | | | | | | | | |
| Bernese | | 0.001 | 0.003 | 0.002 | 0.001 | 0.001 | 0.002 | 0.002 | 0.003 | 0.002 | 0.002 | 0.002 | 0.003 | 0.002 | 0.949 | 0.001 |
| Mountain | | | | | | | | | | | | | | | | |
| Bernese | | 0.001 | 0.005 | 0.002 | 0.002 | 0.002 | 0.001 | 0.002 | 0.001 | 0.002 | 0.002 | 0.002 | 0.001 | 0.002 | 0.951 | 0.001 |
| Mountain | | | | | | | | | | | | | | | | |
| Bernese | | 0.001 | 0.001 | 0.002 | 0.001 | 0.001 | 0.002 | 0.001 | 0.001 | 0.003 | 0.001 | 0.002 | 0.001 | 0.001 | 0.959 | 0.002 |
| Mountain | | | | | | | | | | | | | | | | |
| Bernese | | 0.002 | 0.002 | 0.002 | 0.001 | 0.001 | 0.001 | 0.002 | 0.003 | 0.001 | 0.003 | 0.002 | 0.002 | 0.001 | 0.943 | 0.006 |
| Mountain | | | | | | | | | | | | | | | | |
| Bernese | | 0.001 | 0.003 | 0.002 | 0.001 | 0.002 | 0.002 | 0.002 | 0.007 | 0.003 | 0.003 | 0.003 | 0.008 | 0.001 | 0.938 | 0.002 |
| Mountain | | | | | | | | | | | | | | | | |
| Bernese | | 0.002 | 0.002 | 0.004 | 0.003 | 0.005 | 0.003 | 0.002 | 0.008 | 0.004 | 0.003 | 0.003 | 0.023 | 0.002 | 0.879 | 0.003 |
| Mountain | | | | | | | | | | | | | | | | |
| Bernese | | 0.002 | 0.007 | 0.002 | 0.001 | 0.002 | 0.002 | 0.003 | 0.003 | 0.002 | 0.002 | 0.002 | 0.003 | 0.002 | 0.93 | 0.004 |
| Mountain | | | | | | | | | | | | | | | | |
| Bernese | | 0.001 | 0.001 | 0.001 | 0.002 | 0.001 | 0.001 | 0.002 | 0.003 | 0.001 | 0.001 | 0.001 | 0.002 | 0.002 | 0.954 | 0.002 |
| Mountain | | | | | | | | | | | | | | | | |
| Bernese | | 0.001 | 0.005 | 0.004 | 0.004 | 0.003 | 0.01 | 0.003 | 0.005 | 0.003 | 0.011 | 0.008 | 0.012 | 0.011 | 0.83 | 0.003 |
| Mountain | | | | | | | | | | | | | | | | |
| Bernese | | 0.001 | 0.002 | 0.004 | 0.002 | 0.001 | 0.001 | 0.001 | 0.002 | 0.002 | 0.001 | 0.002 | 0.002 | 0.002 | 0.92 | 0.003 |
| Mountain | | | | | | | | | | | | | | | | |
| Bernese | | 0.002 | 0.002 | 0.006 | 0.004 | 0.002 | 0.003 | 0.001 | 0.126 | 0.003 | 0.001 | 0.004 | 0.005 | 0.002 | 0.76 | 0.003 |
| Mountain | | | | | | | | | | | | | | | | |
| Bernese | | 0.001 | 0.001 | 0.001 | 0.001 | 0.001 | 0.001 | 0.001 | 0.001 | 0.001 | 0.001 | 0.001 | 0.002 | 0.001 | 0.966 | 0.002 |
| Mountain | | | | | | | | | | | | | | | | |
| Bernese | | 0.001 | 0.001 | 0.002 | 0.001 | 0.003 | 0.002 | 0.002 | 0.002 | 0.002 | 0.003 | 0.001 | 0.001 | 0.003 | 0.93 | 0.002 |
| Mountain | | | | | | | | | | | | | | | | |
| Bernese | | 0.002 | 0.011 | 0.008 | 0.002 | 0.005 | 0.002 | 0.004 | 0.005 | 0.005 | 0.005 | 0.012 | 0.006 | 0.017 | 0.833 | 0.003 |
| Mountain | | | | | | | | | | | | | | | | |
| | % | Cluster | | | | | | | | | | | | | | |
| | missing | Assignment | | | | | | | | | | | | | | |
| Breed | data | 1 | 2 | 3 | 4 | 5 | 6 | 7 | 8 | 9 | 10 | 11 | 12 | 13 | 14 | 15 |
| Borzoi | (4) | 0.002 | 0.002 | 0.002 | 0.002 | 0.001 | 0.003 | 0.001 | 0.003 | 0.002 | 0.001 | 0.006 | 0.001 | 0.002 | 0.002 | 0.004 |
| Borzoi | 0 | 0.002 | 0.005 | 0.002 | 0.003 | 0.001 | 0.004 | 0.004 | 0.002 | 0.003 | 0.001 | 0.008 | 0.002 | 0.003 | 0.004 | 0.004 |
| Borzoi | (1) | 0.001 | 0.005 | 0.002 | 0.002 | 0.001 | 0.007 | 0.004 | 0.004 | 0.004 | 0.003 | 0.006 | 0.002 | 0.002 | 0.005 | 0.002 |
| Borzoi | (1) | 0.002 | 0.002 | 0.006 | 0.007 | 0.003 | 0.004 | 0.003 | 0.004 | 0.002 | 0.002 | 0.011 | 0.002 | 0.003 | 0.002 | 0.001 |
| Borzoi | 0 | 0.003 | 0.011 | 0.005 | 0.019 | 0.004 | 0.082 | 0.026 | 0.004 | 0.003 | 0.006 | 0.003 | 0.005 | 0.006 | 0.005 | 0.002 |
| Borzoi | (3) | 0.005 | 0.002 | 0.002 | 0.003 | 0.005 | 0.009 | 0.005 | 0.002 | 0.002 | 0.006 | 0.003 | 0.006 | 0.004 | 0.004 | 0.002 |
| Borzoi | (4) | 0.059 | 0.004 | 0.016 | 0.051 | 0.017 | 0.006 | 0.003 | 0.007 | 0.003 | 0.002 | 0.015 | 0.005 | 0.012 | 0.014 | 0.002 |
| Borzoi | (33) | 0.02 | 0.018 | 0.037 | 0.031 | 0.019 | 0.023 | 0.003 | 0.005 | 0.006 | 0.003 | 0.06 | 0.204 | 0.016 | 0.008 | 0.004 |
| Borzoi | (20) | 0.003 | 0.003 | 0.004 | 0.003 | 0.01 | 0.003 | 0.002 | 0.007 | 0.002 | 0.004 | 0.005 | 0.019 | 0.002 | 0.005 | 0.005 |
| Borzoi | (1) | 0.023 | 0.019 | 0.013 | 0.006 | 0.002 | 0.014 | 0.017 | 0.01 | 0.004 | 0.003 | 0.076 | 0.004 | 0.018 | 0.004 | 0.002 |
| Borzoi | (2) | 0.013 | 0.023 | 0.015 | 0.009 | 0.002 | 0.044 | 0.008 | 0.008 | 0.015 | 0.008 | 0.029 | 0.003 | 0.002 | 0.002 | 0.001 |
| Borzoi | (30) | 0.005 | 0.006 | 0.021 | 0.071 | 0.01 | 0.001 | 0.002 | 0.007 | 0.002 | 0.001 | 0.009 | 0.004 | 0.003 | 0.002 | 0.005 |
| Borzoi | (2) | 0.04 | 0.032 | 0.031 | 0.034 | 0.018 | 0.003 | 0.002 | 0.004 | 0.002 | 0.005 | 0.006 | 0.002 | 0.006 | 0.002 | 0.011 |
| Borzoi | (1) | 0.004 | 0.007 | 0.004 | 0.005 | 0.004 | 0.003 | 0.004 | 0.005 | 0.003 | 0.003 | 0.003 | 0.044 | 0.018 | 0.014 | 0.002 |
| Borzoi | (4) | 0.009 | 0.007 | 0.011 | 0.061 | 0.005 | 0.003 | 0.002 | 0.004 | 0.016 | 0.001 | 0.002 | 0.004 | 0.002 | 0.015 | 0.004 |
| Borzoi | (1) | 0.006 | 0.026 | 0.002 | 0.009 | 0.004 | 0.014 | 0.002 | 0.002 | 0.021 | 0.001 | 0.002 | 0.002 | 0.002 | 0.013 | 0.002 |
| Borzoi | 0 | 0.007 | 0.002 | 0.018 | 0.018 | 0.002 | 0.007 | 0.001 | 0.002 | 0.001 | 0.002 | 0.004 | 0.002 | 0.004 | 0.005 | 0.002 |
| Borzoi | (9) | 0.002 | 0.002 | 0.005 | 0.004 | 0.004 | 0.002 | 0.001 | 0.002 | 0.002 | 0.003 | 0.003 | 0.001 | 0.002 | 0.002 | 0.001 |
| Borzoi | (29) | 0.004 | 0.004 | 0.002 | 0.002 | 0.002 | 0.005 | 0.006 | 0.003 | 0.004 | 0.004 | 0.007 | 0.002 | 0.006 | 0.003 | 0.005 |
| Borzoi | (1) | 0.001 | 0.001 | 0.001 | 0.001 | 0.002 | 0.002 | 0.001 | 0.002 | 0.002 | 0.001 | 0.001 | 0.001 | 0.001 | 0.001 | 0.001 |
| Borzoi | (1) | 0.001 | 0.002 | 0.002 | 0.001 | 0.002 | 0.003 | 0.003 | 0.001 | 0.002 | 0.003 | 0.001 | 0.002 | 0.001 | 0.002 | 0.004 |
| Borzoi | 0 | 0.002 | 0.001 | 0.003 | 0.002 | 0.002 | 0.006 | 0.002 | 0.002 | 0.009 | 0.001 | 0.003 | 0.001 | 0.005 | 0.001 | 0.002 |
| Borzoi | 0 | 0.002 | 0.001 | 0.002 | 0.002 | 0.001 | 0.002 | 0.003 | 0.001 | 0.004 | 0.001 | 0.004 | 0.001 | 0.003 | 0.001 | 0.001 |
| Borzoi | (2) | 0.001 | 0.001 | 0.001 | 0.001 | 0.001 | 0.004 | 0.004 | 0.002 | 0.004 | 0.001 | 0.002 | 0.001 | 0.002 | 0.001 | 0.001 |
| Borzoi | 0 | 0.002 | 0.001 | 0.001 | 0.001 | 0.002 | 0.003 | 0.003 | 0.002 | 0.008 | 0.001 | 0.002 | 0.001 | 0.002 | 0.001 | 0.001 |
| Borzoi | (2) | 0.002 | 0.004 | 0.002 | 0.003 | 0.001 | 0.003 | 0.002 | 0.002 | 0.001 | 0.001 | 0.006 | 0.001 | 0.004 | 0.002 | 0.002 |
| Borzoi | (2) | 0.001 | 0.005 | 0.013 | 0.002 | 0.002 | 0.002 | 0.002 | 0.002 | 0.002 | 0.002 | 0.005 | 0.002 | 0.001 | 0.01 | 0.003 |
| Borzoi | 0 | 0.009 | 0.017 | 0.033 | 0.023 | 0.045 | 0.003 | 0.004 | 0.027 | 0.006 | 0.003 | 0.012 | 0.003 | 0.004 | 0.017 | 0.002 |
| Borzoi | 0 | 0.006 | 0.012 | 0.005 | 0.004 | 0.003 | 0.003 | 0.001 | 0.003 | 0.002 | 0.006 | 0.003 | 0.003 | 0.002 | 0.006 | 0.004 |
| Borzoi | 0 | 0.01 | 0.014 | 0.021 | 0.003 | 0.003 | 0.004 | 0.002 | 0.005 | 0.003 | 0.006 | 0.002 | 0.002 | 0.002 | 0.004 | 0.006 |
| Borzoi | 0 | 0.014 | 0.011 | 0.045 | 0.01 | 0.004 | 0.004 | 0.002 | 0.008 | 0.004 | 0.004 | 0.008 | 0.003 | 0.005 | 0.003 | 0.003 |
| Borzoi | 0 | 0.003 | 0.02 | 0.006 | 0.012 | 0.004 | 0.005 | 0.104 | 0.008 | 0.003 | 0.001 | 0.003 | 0.002 | 0.013 | 0.003 | 0.007 |
| Borzoi | 0 | 0.001 | 0.001 | 0.001 | 0.001 | 0.002 | 0.001 | 0.001 | 0.001 | 0.002 | 0.003 | 0.001 | 0.001 | 0.001 | 0.003 | 0.002 |
| Borzoi | 0 | 0.003 | 0.003 | 0.001 | 0.001 | 0.003 | 0.002 | 0.002 | 0.001 | 0.002 | 0.002 | 0.004 | 0.002 | 0.002 | 0.003 | 0.002 |
| Borzoi | 0 | 0.003 | 0.007 | 0.001 | 0.002 | 0.001 | 0.002 | 0.004 | 0.004 | 0.003 | 0.001 | 0.002 | 0.001 | 0.003 | 0.002 | 0.003 |
| Borzoi | (2) | 0.002 | 0.003 | 0.002 | 0.003 | 0.007 | 0.002 | 0.003 | 0.004 | 0.004 | 0.003 | 0.002 | 0.004 | 0.002 | 0.003 | 0.003 |
| Borzoi | (3) | 0.002 | 0.002 | 0.001 | 0.002 | 0.003 | 0.002 | 0.002 | 0.002 | 0.001 | 0.001 | 0.001 | 0.001 | 0.001 | 0.001 | 0.003 |
| Borzoi | (2) | 0.004 | 0.006 | 0.005 | 0.009 | 0.002 | 0.005 | 0.003 | 0.002 | 0.002 | 0.003 | 0.011 | 0.005 | 0.004 | 0.005 | 0.012 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Borzoi | | 0.001 | 0.007 | 0.005 | 0.006 | 0.002 | 0.002 | 0.005 | 0.003 | 0.004 | 0.002 | 0.002 | 0.001 | 0.92 | 0.001 | 0.003 |
| Borzoi | | 0.001 | 0.01 | 0.01 | 0.006 | 0.002 | 0.005 | 0.005 | 0.003 | 0.003 | 0.002 | 0.003 | 0.001 | 0.895 | 0.002 | 0.003 |
| Borzoi | | 0.001 | 0.011 | 0.01 | 0.004 | 0.002 | 0.003 | 0.007 | 0.002 | 0.002 | 0.002 | 0.003 | 0.003 | 0.896 | 0.001 | 0.003 |
| Borzoi | | 0.003 | 0.004 | 0.004 | 0.078 | 0.003 | 0.003 | 0.003 | 0.002 | 0.003 | 0.002 | 0.001 | 0.001 | 0.833 | 0.002 | 0.002 |
| Borzoi | | 0.006 | 0.004 | 0.01 | 0.08 | 0.003 | 0.005 | 0.029 | 0.013 | 0.005 | 0.005 | 0.002 | 0.005 | 0.639 | 0.004 | 0.01 |
| Borzoi | | 0.003 | 0.001 | 0.003 | 0.001 | 0.008 | 0.008 | 0.001 | 0.002 | 0.001 | 0.002 | 0.001 | 0.002 | 0.901 | 0.001 | 0.001 |
| Borzoi | | 0.016 | 0.013 | 0.023 | 0.01 | 0.006 | 0.026 | 0.003 | 0.003 | 0.029 | 0.003 | 0.002 | 0.005 | 0.638 | 0.004 | 0.002 |
| Borzoi | | 0.002 | 0.004 | 0.016 | 0.019 | 0.004 | 0.004 | 0.039 | 0.006 | 0.119 | 0.002 | 0.003 | 0.003 | 0.31 | 0.006 | 0.004 |
| Borzoi | | 0.003 | 0.002 | 0.002 | 0.002 | 0.003 | 0.002 | 0.004 | 0.003 | 0.003 | 0.001 | 0.009 | 0.004 | 0.878 | 0.004 | 0.003 |
| Borzoi | | 0.006 | 0.013 | 0.007 | 0.007 | 0.022 | 0.008 | 0.006 | 0.009 | 0.016 | 0.004 | 0.003 | 0.01 | 0.663 | 0.005 | 0.005 |
| Borzoi | | 0.038 | 0.004 | 0.011 | 0.006 | 0.005 | 0.002 | 0.004 | 0.005 | 0.004 | 0.004 | 0.007 | 0.007 | 0.71 | 0.002 | 0.009 |
| Borzoi | | 0.004 | 0.003 | 0.004 | 0.002 | 0.002 | 0.003 | 0.005 | 0.003 | 0.002 | 0.011 | 0.003 | 0.013 | 0.776 | 0.014 | 0.005 |
| Borzoi | | 0.004 | 0.003 | 0.007 | 0.004 | 0.002 | 0.006 | 0.004 | 0.003 | 0.006 | 0.003 | 0.002 | 0.004 | 0.743 | 0.002 | 0.011 |
| Borzoi | | 0.023 | 0.006 | 0.007 | 0.004 | 0.001 | 0.014 | 0.002 | 0.003 | 0.002 | 0.006 | 0.001 | 0.007 | 0.796 | 0.002 | 0.002 |
| Borzoi | | 0.002 | 0.002 | 0.011 | 0.005 | 0.002 | 0.004 | 0.002 | 0.002 | 0.015 | 0.001 | 0.001 | 0.001 | 0.8 | 0.001 | 0.003 |
| Borzoi | | 0.003 | 0.001 | 0.003 | 0.003 | 0.002 | 0.003 | 0.002 | 0.003 | 0.017 | 0.001 | 0.001 | 0.001 | 0.847 | 0.001 | 0.003 |
| Borzoi | | 0.001 | 0.002 | 0.003 | 0.003 | 0.008 | 0.001 | 0.004 | 0.002 | 0.008 | 0.002 | 0.003 | 0.003 | 0.874 | 0.002 | 0.008 |
| Borzoi | | 0.001 | 0.002 | 0.003 | 0.004 | 0.005 | 0.003 | 0.001 | 0.002 | 0.002 | 0.002 | 0.003 | 0.003 | 0.929 | 0.002 | 0.002 |
| Borzoi | | 0.002 | 0.008 | 0.004 | 0.003 | 0.003 | 0.004 | 0.002 | 0.007 | 0.002 | 0.003 | 0.006 | 0.025 | 0.863 | 0.003 | 0.005 |
| Borzoi | | 0.001 | 0.002 | 0.001 | 0.002 | 0.002 | 0.001 | 0.001 | 0.001 | 0.001 | 0.001 | 0.001 | 0.002 | 0.961 | 0.001 | 0.001 |
| Borzoi | | 0.001 | 0.003 | 0.002 | 0.001 | 0.003 | 0.002 | 0.002 | 0.001 | 0.002 | 0.001 | 0.001 | 0.001 | 0.94 | 0.003 | 0.005 |
| Borzoi | | 0.001 | 0.002 | 0.002 | 0.003 | 0.002 | 0.001 | 0.002 | 0.002 | 0.003 | 0.001 | 0.002 | 0.002 | 0.928 | 0.003 | 0.002 |
| Borzoi | | 0.001 | 0.002 | 0.003 | 0.001 | 0.003 | 0.001 | 0.002 | 0.002 | 0.001 | 0.002 | 0.003 | 0.003 | 0.946 | 0.001 | 0.002 |
| Borzoi | | 0.001 | 0.003 | 0.002 | 0.002 | 0.002 | 0.001 | 0.002 | 0.001 | 0.001 | 0.002 | 0.004 | 0.001 | 0.948 | 0.001 | 0.002 |
| Borzoi | | 0.001 | 0.001 | 0.001 | 0.002 | 0.002 | 0.001 | 0.002 | 0.002 | 0.003 | 0.001 | 0.002 | 0.002 | 0.945 | 0.002 | 0.003 |
| Borzoi | | 0.001 | 0.005 | 0.004 | 0.002 | 0.001 | 0.002 | 0.003 | 0.002 | 0.001 | 0.002 | 0.002 | 0.002 | 0.934 | 0.002 | 0.001 |
| Borzoi | | 0.001 | 0.008 | 0.009 | 0.002 | 0.004 | 0.005 | 0.003 | 0.003 | 0.002 | 0.003 | 0.003 | 0.001 | 0.898 | 0.003 | 0.001 |
| Borzoi | | 0.005 | 0.011 | 0.007 | 0.003 | 0.004 | 0.008 | 0.002 | 0.002 | 0.03 | 0.015 | 0.003 | 0.006 | 0.679 | 0.009 | 0.008 |
| Borzoi | | 0.002 | 0.002 | 0.015 | 0.006 | 0.002 | 0.003 | 0.012 | 0.02 | 0.008 | 0.005 | 0.004 | 0.003 | 0.806 | 0.002 | 0.046 |
| Borzoi | | 0.002 | 0.003 | 0.007 | 0.012 | 0.002 | 0.004 | 0.005 | 0.004 | 0.004 | 0.006 | 0.003 | 0.002 | 0.841 | 0.003 | 0.014 |
| Borzoi | | 0.003 | 0.003 | 0.01 | 0.006 | 0.008 | 0.006 | 0.012 | 0.002 | 0.003 | 0.018 | 0.006 | 0.009 | 0.772 | 0.004 | 0.011 |
| Borzoi | | 0.003 | 0.006 | 0.006 | 0.004 | 0.015 | 0.005 | 0.018 | 0.01 | 0.003 | 0.027 | 0.002 | 0.005 | 0.69 | 0.002 | 0.012 |
| Borzoi | | 0.001 | 0.002 | 0.002 | 0.002 | 0.001 | 0.002 | 0.001 | 0.002 | 0.002 | 0.002 | 0.002 | 0.001 | 0.951 | 0.001 | 0.004 |
| Borzoi | | 0.002 | 0.004 | 0.005 | 0.002 | 0.002 | 0.001 | 0.002 | 0.002 | 0.006 | 0.002 | 0.001 | 0.002 | 0.935 | 0.001 | 0.001 |
| Borzoi | | 0.001 | 0.003 | 0.014 | 0.002 | 0.003 | 0.001 | 0.177 | 0.002 | 0.003 | 0.002 | 0.002 | 0.002 | 0.741 | 0.002 | 0.007 |
| Borzoi | | 0.002 | 0.002 | 0.002 | 0.004 | 0.01 | 0.003 | 0.004 | 0.002 | 0.005 | 0.002 | 0.001 | 0.001 | 0.913 | 0.003 | 0.001 |
| Borzoi | | 0.001 | 0.002 | 0.003 | 0.005 | 0.001 | 0.002 | 0.001 | 0.002 | 0.003 | 0.001 | 0.002 | 0.001 | 0.942 | 0.003 | 0.002 |
| Borzoi | | 0.002 | 0.002 | 0.003 | 0.017 | 0.003 | 0.002 | 0.006 | 0.005 | 0.005 | 0.001 | 0.002 | 0.002 | 0.867 | 0.003 | 0.003 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Chihuahua | 0 | 0.024 | 0.006 | 0.003 | 0.004 | 0.005 | 0.609 | 0.005 | 0.003 | 0.007 | 0.006 | 0.019 | 0.018 | 0.003 | 0.01 | 0.045 |
| Chihuahua | (15) | 0.013 | 0.007 | 0.008 | 0.008 | 0.003 | 0.702 | 0.012 | 0.015 | 0.005 | 0.006 | 0.004 | 0.015 | 0.006 | 0.011 | 0.045 |
| Chihuahua | 0 | 0.025 | 0.003 | 0.003 | 0.011 | 0.001 | 0.694 | 0.005 | 0.008 | 0.007 | 0.003 | 0.005 | 0.002 | 0.004 | 0.01 | 0.003 |
| Chihuahua | 0 | 0.151 | 0.006 | 0.003 | 0.002 | 0.001 | 0.71 | 0.003 | 0.003 | 0.004 | 0.004 | 0.008 | 0.004 | 0.004 | 0.006 | 0.004 |
| Chihuahua | 0 | 0.009 | 0.002 | 0.004 | 0.011 | 0.005 | 0.748 | 0.007 | 0.005 | 0.003 | 0.004 | 0.003 | 0.009 | 0.002 | 0.002 | 0.012 |
| Chihuahua | 0 | 0.004 | 0.007 | 0.042 | 0.005 | 0.054 | 0.768 | 0.006 | 0.016 | 0.001 | 0.004 | 0.009 | 0.003 | 0.003 | 0.015 | 0.007 |
| Chihuahua | 0 | 0.011 | 0.021 | 0.013 | 0.003 | 0.003 | 0.591 | 0.009 | 0.016 | 0.003 | 0.001 | 0.05 | 0.121 | 0.011 | 0.006 | 0.005 |
| Chihuahua | 0 | 0.001 | 0.001 | 0.001 | 0.001 | 0.001 | 0.958 | 0.002 | 0.001 | 0.001 | 0.001 | 0.001 | 0.001 | 0.001 | 0.001 | 0.002 |
| Chihuahua | 0 | 0.011 | 0.007 | 0.003 | 0.007 | 0.003 | 0.858 | 0.002 | 0.003 | 0.002 | 0.001 | 0.006 | 0.004 | 0.011 | 0.003 | 0.002 |
| Chihuahua | (2) | 0.002 | 0.004 | 0.002 | 0.004 | 0.002 | 0.794 | 0.006 | 0.004 | 0.003 | 0.003 | 0.004 | 0.002 | 0.017 | 0.007 | 0.002 |
| Chihuahua | 0 | 0.002 | 0.002 | 0.002 | 0.002 | 0.002 | 0.936 | 0.003 | 0.001 | 0.001 | 0.002 | 0.002 | 0.001 | 0.002 | 0.001 | 0.003 |
| Chihuahua | (25) | 0.001 | 0.002 | 0.001 | 0.002 | 0.001 | 0.932 | 0.002 | 0.002 | 0.002 | 0.004 | 0.002 | 0.001 | 0.004 | 0.001 | 0.002 |
| Chihuahua | 0 | 0.015 | 0.015 | 0.032 | 0.007 | 0.005 | 0.727 | 0.006 | 0.014 | 0.061 | 0.013 | 0.002 | 0.004 | 0.002 | 0.008 | 0.008 |
| Chihuahua | 0 | 0.004 | 0.013 | 0.035 | 0.004 | 0.002 | 0.637 | 0.04 | 0.046 | 0.002 | 0.002 | 0.004 | 0.004 | 0.017 | 0.003 | 0.008 |
| Chihuahua | 0 | 0.002 | 0.008 | 0.005 | 0.002 | 0.002 | 0.588 | 0.014 | 0.044 | 0.001 | 0.003 | 0.005 | 0.006 | 0.137 | 0.003 | 0.004 |
| Chihuahua | 0 | 0.019 | 0.006 | 0.014 | 0.023 | 0.007 | 0.755 | 0.003 | 0.007 | 0.003 | 0.003 | 0.014 | 0.025 | 0.005 | 0.007 | 0.004 |
| Chihuahua | 0 | 0.003 | 0.005 | 0.065 | 0.004 | 0.016 | 0.483 | 0.18 | 0.154 | 0.002 | 0.003 | 0.007 | 0.007 | 0.005 | 0.012 | 0.004 |
| Chihuahua | (11) | 0.002 | 0.002 | 0.001 | 0.002 | 0.001 | 0.921 | 0.003 | 0.002 | 0.003 | 0.002 | 0.003 | 0.002 | 0.005 | 0.002 | 0.001 |
| Chihuahua | 0 | 0.008 | 0.02 | 0.049 | 0.006 | 0.008 | 0.785 | 0.017 | 0.01 | 0.004 | 0.011 | 0.004 | 0.004 | 0.003 | 0.003 | 0.008 |
| Chihuahua | (12) | 0.016 | 0.003 | 0.006 | 0.018 | 0.004 | 0.778 | 0.005 | 0.01 | 0.011 | 0.006 | 0.008 | 0.007 | 0.003 | 0.015 | 0.013 |
| Chihuahua | 0 | 0.003 | 0.002 | 0.005 | 0.008 | 0.023 | 0.699 | 0.01 | 0.007 | 0.011 | 0.004 | 0.008 | 0.004 | 0.005 | 0.004 | 0.02 |
| Chihuahua | (1) | 0.002 | 0.004 | 0.002 | 0.016 | 0.002 | 0.177 | 0.016 | 0.01 | 0.01 | 0.221 | 0.003 | 0.002 | 0.002 | 0.005 | 0.003 |
| Chihuahua | 0 | 0.003 | 0.017 | 0.004 | 0.004 | 0.042 | 0.178 | 0.009 | 0.002 | 0.035 | 0.231 | 0.036 | 0.002 | 0.008 | 0.044 | 0.008 |
| Chihuahua | 0 | 0.003 | 0.017 | 0.002 | 0.002 | 0.001 | 0.398 | 0.005 | 0.007 | 0.062 | 0.066 | 0.003 | 0.003 | 0.003 | 0.016 | 0.002 |
| Chihuahua | 0 | 0.004 | 0.092 | 0.031 | 0.024 | 0.004 | 0.225 | 0.016 | 0.002 | 0.018 | 0.204 | 0.008 | 0.007 | 0.009 | 0.055 | 0.004 |
| Chihuahua | (23) | 0.008 | 0.004 | 0.005 | 0.006 | 0.002 | 0.473 | 0.007 | 0.007 | 0.004 | 0.009 | 0.006 | 0.008 | 0.198 | 0.004 | 0.003 |
| Chihuahua | 0 | 0.003 | 0.003 | 0.005 | 0.002 | 0.002 | 0.688 | 0.003 | 0.006 | 0.003 | 0.003 | 0.004 | 0.005 | 0.011 | 0.002 | 0.012 |
| Chihuahua | 0 | 0.007 | 0.009 | 0.022 | 0.006 | 0.002 | 0.682 | 0.011 | 0.014 | 0.006 | 0.002 | 0.003 | 0.005 | 0.054 | 0.003 | 0.003 |
| Chihuahua | (4) | 0.009 | 0.009 | 0.009 | 0.008 | 0.005 | 0.664 | 0.003 | 0.016 | 0.002 | 0.003 | 0.003 | 0.005 | 0.008 | 0.002 | 0.003 |
| Chihuahua | 0 | 0.001 | 0.008 | 0.002 | 0.002 | 0.002 | 0.769 | 0.005 | 0.007 | 0.003 | 0.001 | 0.003 | 0.004 | 0.008 | 0.004 | 0.005 |
| Chihuahua | 0 | 0.002 | 0.014 | 0.143 | 0.004 | 0.004 | 0.633 | 0.013 | 0.007 | 0.001 | 0.004 | 0.007 | 0.036 | 0.006 | 0.013 | 0.004 |
| Chihuahua | (6) | 0.003 | 0.002 | 0.003 | 0.003 | 0.001 | 0.886 | 0.005 | 0.003 | 0.004 | 0.002 | 0.003 | 0.002 | 0.002 | 0.002 | 0.003 |
| Chihuahua | 0 | 0.005 | 0.004 | 0.003 | 0.004 | 0.002 | 0.877 | 0.002 | 0.003 | 0.002 | 0.012 | 0.004 | 0.003 | 0.003 | 0.002 | 0.004 |
| Chihuahua | (29) | 0.004 | 0.014 | 0.015 | 0.004 | 0.002 | 0.84 | 0.003 | 0.003 | 0.005 | 0.017 | 0.004 | 0.003 | 0.011 | 0.007 | 0.004 |
| Chihuahua | 0 | 0.003 | 0.012 | 0.003 | 0.007 | 0.082 | 0.675 | 0.003 | 0.004 | 0.004 | 0.019 | 0.005 | 0.004 | 0.013 | 0.006 | 0.005 |
| Chihuahua | (13) | 0.004 | 0.003 | 0.002 | 0.003 | 0.002 | 0.909 | 0.005 | 0.002 | 0.005 | 0.014 | 0.004 | 0.002 | 0.002 | 0.002 | 0.002 |
| Chihuahua | 0 | 0.021 | 0.009 | 0.002 | 0.051 | 0.007 | 0.743 | 0.005 | 0.02 | 0.007 | 0.014 | 0.003 | 0.002 | 0.002 | 0.002 | 0.004 |
| Chihuahua | (21) | 0.034 | 0.008 | 0.012 | 0.075 | 0.003 | 0.187 | 0.022 | 0.05 | 0.011 | 0.006 | 0.019 | 0.068 | 0.01 | 0.008 | 0.002 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Chihuahua | | 0.004 | 0.005 | 0.007 | 0.015 | 0.005 | 0.013 | 0.009 | 0.007 | 0.148 | 0.002 | 0.004 | 0.004 | 0.004 | 0.002 | 0.003 |
| Chihuahua | | 0.005 | 0.012 | 0.005 | 0.01 | 0.009 | 0.005 | 0.003 | 0.012 | 0.035 | 0.005 | 0.002 | 0.011 | 0.006 | 0.003 | 0.015 |
| Chihuahua | | 0.002 | 0.006 | 0.025 | 0.053 | 0.003 | 0.002 | 0.002 | 0.014 | 0.012 | 0.061 | 0.001 | 0.022 | 0.003 | 0.007 | 0.002 |
| Chihuahua | | 0.003 | 0.003 | 0.007 | 0.008 | 0.002 | 0.005 | 0.003 | 0.004 | 0.025 | 0.003 | 0.003 | 0.006 | 0.004 | 0.008 | 0.002 |
| Chihuahua | | 0.053 | 0.002 | 0.005 | 0.005 | 0.013 | 0.004 | 0.003 | 0.018 | 0.006 | 0.005 | 0.009 | 0.004 | 0.034 | 0.003 | 0.008 |
| Chihuahua | | 0.002 | 0.003 | 0.004 | 0.003 | 0.002 | 0.005 | 0.005 | 0.003 | 0.003 | 0.001 | 0.001 | 0.003 | 0.013 | 0.003 | 0.005 |
| Chihuahua | | 0.01 | 0.016 | 0.017 | 0.008 | 0.006 | 0.003 | 0.002 | 0.013 | 0.004 | 0.003 | 0.008 | 0.009 | 0.029 | 0.003 | 0.007 |
| Chihuahua | | 0.001 | 0.001 | 0.001 | 0.003 | 0.002 | 0.002 | 0.001 | 0.001 | 0.001 | 0.002 | 0.001 | 0.001 | 0.002 | 0.002 | 0.002 |
| Chihuahua | | 0.004 | 0.004 | 0.003 | 0.005 | 0.005 | 0.002 | 0.002 | 0.009 | 0.006 | 0.006 | 0.003 | 0.004 | 0.014 | 0.005 | 0.005 |
| Chihuahua | | 0.002 | 0.041 | 0.003 | 0.009 | 0.007 | 0.011 | 0.007 | 0.007 | 0.004 | 0.004 | 0.022 | 0.011 | 0.005 | 0.009 | 0.002 |
| Chihuahua | | 0.002 | 0.002 | 0.002 | 0.002 | 0.004 | 0.002 | 0.002 | 0.003 | 0.003 | 0.002 | 0.002 | 0.003 | 0.004 | 0.005 | 0.002 |
| Chihuahua | | 0.001 | 0.007 | 0.002 | 0.002 | 0.005 | 0.002 | 0.004 | 0.002 | 0.004 | 0.002 | 0.001 | 0.002 | 0.002 | 0.002 | 0.003 |
| Chihuahua | | 0.009 | 0.002 | 0.003 | 0.004 | 0.005 | 0.006 | 0.007 | 0.004 | 0.01 | 0.002 | 0.013 | 0.002 | 0.005 | 0.002 | 0.007 |
| Chihuahua | | 0.008 | 0.028 | 0.019 | 0.006 | 0.002 | 0.003 | 0.009 | 0.027 | 0.006 | 0.003 | 0.006 | 0.005 | 0.003 | 0.002 | 0.052 |
| Chihuahua | | 0.004 | 0.086 | 0.004 | 0.002 | 0.002 | 0.005 | 0.004 | 0.016 | 0.007 | 0.008 | 0.003 | 0.021 | 0.004 | 0.007 | 0.005 |
| Chihuahua | | 0.002 | 0.004 | 0.004 | 0.014 | 0.004 | 0.003 | 0.002 | 0.006 | 0.009 | 0.006 | 0.006 | 0.004 | 0.027 | 0.005 | 0.007 |
| Chihuahua | | 0.005 | 0.001 | 0.004 | 0.005 | 0.002 | 0.002 | 0.003 | 0.005 | 0.003 | 0.002 | 0.001 | 0.003 | 0.009 | 0.002 | 0.002 |
| Chihuahua | | 0.001 | 0.004 | 0.004 | 0.002 | 0.004 | 0.002 | 0.005 | 0.003 | 0.003 | 0.002 | 0.004 | 0.004 | 0.003 | 0.001 | 0.007 |
| Chihuahua | | 0.002 | 0.002 | 0.005 | 0.004 | 0.002 | 0.009 | 0.01 | 0.004 | 0.003 | 0.001 | 0.003 | 0.001 | 0.002 | 0.002 | 0.01 |
| Chihuahua | | 0.005 | 0.014 | 0.004 | 0.003 | 0.002 | 0.005 | 0.01 | 0.006 | 0.011 | 0.002 | 0.003 | 0.007 | 0.016 | 0.006 | 0.003 |
| Chihuahua | | 0.015 | 0.003 | 0.004 | 0.004 | 0.002 | 0.004 | 0.003 | 0.004 | 0.008 | 0.003 | 0.051 | 0.057 | 0.013 | 0.006 | 0.011 |
| Chihuahua | | 0.14 | 0.006 | 0.104 | 0.01 | 0.002 | 0.004 | 0.024 | 0.076 | 0.005 | 0.009 | 0.016 | 0.001 | 0.003 | 0.013 | 0.113 |
| Chihuahua | | 0.005 | 0.012 | 0.026 | 0.006 | 0.007 | 0.003 | 0.01 | 0.099 | 0.051 | 0.004 | 0.017 | 0.001 | 0.023 | 0.106 | 0.008 |
| Chihuahua | | 0.011 | 0.005 | 0.224 | 0.002 | 0.003 | 0.015 | 0.037 | 0.003 | 0.004 | 0.003 | 0.055 | 0.002 | 0.006 | 0.005 | 0.035 |
| Chihuahua | | 0.006 | 0.002 | 0.012 | 0.005 | 0.002 | 0.012 | 0.007 | 0.01 | 0.005 | 0.001 | 0.018 | 0.002 | 0.189 | 0.011 | 0.015 |
| Chihuahua | | 0.004 | 0.011 | 0.02 | 0.009 | 0.066 | 0.014 | 0.023 | 0.006 | 0.019 | 0.019 | 0.012 | 0.027 | 0.004 | 0.004 | 0.019 |
| Chihuahua | | 0.004 | 0.005 | 0.099 | 0.022 | 0.058 | 0.006 | 0.004 | 0.002 | 0.012 | 0.007 | 0.004 | 0.005 | 0.006 | 0.006 | 0.006 |
| Chihuahua | | 0.046 | 0.004 | 0.026 | 0.008 | 0.011 | 0.006 | 0.005 | 0.003 | 0.023 | 0.011 | 0.004 | 0.009 | 0.004 | 0.003 | 0.007 |
| Chihuahua | | 0.017 | 0.007 | 0.108 | 0.01 | 0.02 | 0.032 | 0.007 | 0.002 | 0.016 | 0.002 | 0.003 | 0.002 | 0.014 | 0.005 | 0.003 |
| Chihuahua | | 0.007 | 0.092 | 0.007 | 0.004 | 0.015 | 0.004 | 0.005 | 0.003 | 0.003 | 0.003 | 0.004 | 0.01 | 0.003 | 0.002 | 0.013 |
| Chihuahua | | 0.004 | 0.003 | 0.059 | 0.006 | 0.003 | 0.002 | 0.002 | 0.003 | 0.002 | 0.003 | 0.003 | 0.002 | 0.008 | 0.004 | 0.004 |
| Chihuahua | | 0.003 | 0.008 | 0.007 | 0.01 | 0.002 | 0.006 | 0.004 | 0.002 | 0.002 | 0.005 | 0.009 | 0.002 | 0.002 | 0.002 | 0.011 |
| Chihuahua | | 0.002 | 0.002 | 0.004 | 0.004 | 0.002 | 0.006 | 0.002 | 0.01 | 0.003 | 0.005 | 0.021 | 0.001 | 0.002 | 0.002 | 0.003 |
| Chihuahua | | 0.002 | 0.002 | 0.005 | 0.005 | 0.004 | 0.003 | 0.004 | 0.003 | 0.01 | 0.004 | 0.007 | 0.003 | 0.001 | 0.003 | 0.007 |
| Chihuahua | | 0.004 | 0.004 | 0.006 | 0.043 | 0.003 | 0.012 | 0.049 | 0.002 | 0.008 | 0.011 | 0.003 | 0.002 | 0.004 | 0.003 | 0.003 |
| Chihuahua | | 0.001 | 0.001 | 0.002 | 0.002 | 0.002 | 0.002 | 0.002 | 0.003 | 0.002 | 0.001 | 0.011 | 0.001 | 0.002 | 0.003 | 0.003 |
| Chihuahua | | 0.003 | 0.008 | 0.007 | 0.004 | 0.006 | 0.003 | 0.017 | 0.007 | 0.004 | 0.005 | 0.004 | 0.005 | 0.024 | 0.006 | 0.005 |
| Chihuahua | | 0.006 | 0.101 | 0.005 | 0.036 | 0.007 | 0.018 | 0.126 | 0.016 | 0.048 | 0.007 | 0.054 | 0.011 | 0.031 | 0.01 | 0.01 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Chinese Shar-Pei | 0 | 0.003 | 0.005 | 0.002 | 0.002 | 0.002 | 0.002 | 0.002 | 0.002 | 0.003 | 0.001 | 0.002 | 0.001 | 0.001 | 0.005 | 0.915 |
| Chinese Shar-Pei | 0 | 0.006 | 0.01 | 0.002 | 0.003 | 0.005 | 0.004 | 0.001 | 0.002 | 0.001 | 0.001 | 0.004 | 0.002 | 0.001 | 0.003 | 0.861 |
| Chinese Shar-Pei | 0 | 0.005 | 0.004 | 0.004 | 0.011 | 0.003 | 0.016 | 0.004 | 0.007 | 0.002 | 0.006 | 0.008 | 0.001 | 0.005 | 0.013 | 0.806 |
| Chinese Shar-Pei | (1) | 0.003 | 0.007 | 0.002 | 0.003 | 0.002 | 0.001 | 0.001 | 0.002 | 0.001 | 0.004 | 0.001 | 0.001 | 0.004 | 0.003 | 0.931 |
| Chinese Shar-Pei | (2) | 0.003 | 0.003 | 0.002 | 0.003 | 0.002 | 0.004 | 0.003 | 0.003 | 0.002 | 0.005 | 0.002 | 0.001 | 0.002 | 0.003 | 0.915 |
| Chinese Shar-Pei | 0 | 0.002 | 0.002 | 0.001 | 0.003 | 0.001 | 0.004 | 0.002 | 0.002 | 0.001 | 0.002 | 0.005 | 0.001 | 0.003 | 0.004 | 0.939 |
| Chinese Shar-Pei | (1) | 0.004 | 0.006 | 0.003 | 0.108 | 0.003 | 0.003 | 0.004 | 0.003 | 0.002 | 0.005 | 0.001 | 0.002 | 0.002 | 0.002 | 0.762 |
| Chinese Shar-Pei | 0 | 0.019 | 0.048 | 0.002 | 0.005 | 0.02 | 0.004 | 0.008 | 0.036 | 0.006 | 0.005 | 0.005 | 0.028 | 0.005 | 0.005 | 0.717 |
| Chinese Shar-Pei | 0 | 0.002 | 0.004 | 0.002 | 0.004 | 0.002 | 0.01 | 0.005 | 0.002 | 0.002 | 0.002 | 0.005 | 0.002 | 0.006 | 0.014 | 0.878 |
| Chinese Shar-Pei | (1) | 0.017 | 0.02 | 0.009 | 0.012 | 0.008 | 0.013 | 0.003 | 0.003 | 0.008 | 0.001 | 0.004 | 0.003 | 0.005 | 0.005 | 0.746 |
| Chinese Shar-Pei | 0 | 0.017 | 0.012 | 0.007 | 0.021 | 0.008 | 0.01 | 0.002 | 0.002 | 0.002 | 0.001 | 0.009 | 0.005 | 0.013 | 0.003 | 0.79 |
| Chinese Shar-Pei | (8) | 0.004 | 0.013 | 0.007 | 0.02 | 0.001 | 0.009 | 0.016 | 0.002 | 0.002 | 0.001 | 0.005 | 0.002 | 0.08 | 0.009 | 0.768 |
| Chinese Shar-Pei | 0 | 0.005 | 0.008 | 0.001 | 0.016 | 0.004 | 0.004 | 0.023 | 0.001 | 0.001 | 0.002 | 0.001 | 0.008 | 0.004 | 0.051 | 0.809 |
| Chinese Shar-Pei | (2) | 0.007 | 0.004 | 0.002 | 0.003 | 0.001 | 0.007 | 0.015 | 0.005 | 0.001 | 0.005 | 0.004 | 0.018 | 0.004 | 0.006 | 0.858 |
| Chinese Shar-Pei | 0 | 0.004 | 0.004 | 0.002 | 0.006 | 0.008 | 0.009 | 0.006 | 0.001 | 0.001 | 0.002 | 0.001 | 0.01 | 0.002 | 0.049 | 0.776 |
| Chinese Shar-Pei | (1) | 0.005 | 0.002 | 0.001 | 0.006 | 0.02 | 0.002 | 0.002 | 0.001 | 0.002 | 0.003 | 0.003 | 0.002 | 0.004 | 0.011 | 0.674 |
| Chinese Shar-Pei | 0 | 0.009 | 0.01 | 0.002 | 0.003 | 0.001 | 0.004 | 0.011 | 0.014 | 0.003 | 0.003 | 0.002 | 0.013 | 0.002 | 0.003 | 0.785 |
| Chinese Shar-Pei | (1) | 0.003 | 0.006 | 0.003 | 0.003 | 0.001 | 0.002 | 0.006 | 0.006 | 0.004 | 0.008 | 0.001 | 0.002 | 0.002 | 0.011 | 0.839 |
| Chinese Shar-Pei | 0 | 0.021 | 0.005 | 0.003 | 0.003 | 0.004 | 0.005 | 0.002 | 0.002 | 0.002 | 0.003 | 0.002 | 0.002 | 0.003 | 0.004 | 0.845 |
| Chinese Shar-Pei | (7) | 0.01 | 0.03 | 0.003 | 0.012 | 0.002 | 0.003 | 0.012 | 0.004 | 0.002 | 0.006 | 0.002 | 0.008 | 0.004 | 0.04 | 0.774 |
| Chinese Shar-Pei | 0 | 0.003 | 0.011 | 0.001 | 0.008 | 0.001 | 0.002 | 0.012 | 0.005 | 0.004 | 0.003 | 0.011 | 0.003 | 0.006 | 0.018 | 0.736 |
| Chinese Shar-Pei | (3) | 0.002 | 0.006 | 0.001 | 0.002 | 0.001 | 0.002 | 0.005 | 0.002 | 0.016 | 0.007 | 0.002 | 0.002 | 0.01 | 0.003 | 0.843 |
| Chinese Shar-Pei | (1) | 0.008 | 0.006 | 0.001 | 0.003 | 0.049 | 0.005 | 0.008 | 0.002 | 0.006 | 0.002 | 0.003 | 0.002 | 0.011 | 0.003 | 0.828 |
| Chinese Shar-Pei | (6) | 0.002 | 0.002 | 0.003 | 0.004 | 0.005 | 0.002 | 0.002 | 0.002 | 0.002 | 0.005 | 0.002 | 0.002 | 0.001 | 0.002 | 0.907 |
| Chinese Shar-Pei | (7) | 0.003 | 0.013 | 0.002 | 0.012 | 0.004 | 0.006 | 0.007 | 0.004 | 0.014 | 0.006 | 0.013 | 0.007 | 0.028 | 0.009 | 0.695 |
| Chinese Shar-Pei | (5) | 0.005 | 0.008 | 0.003 | 0.005 | 0.004 | 0.019 | 0.017 | 0.004 | 0.086 | 0.002 | 0.01 | 0.002 | 0.005 | 0.006 | 0.741 |
| Chinese Shar-Pei | (4) | 0.004 | 0.015 | 0.002 | 0.003 | 0.003 | 0.019 | 0.004 | 0.003 | 0.005 | 0.004 | 0.003 | 0.006 | 0.019 | 0.01 | 0.661 |
| Chinese Shar-Pei | (2) | 0.017 | 0.004 | 0.005 | 0.065 | 0.002 | 0.006 | 0.003 | 0.005 | 0.009 | 0.003 | 0.006 | 0.002 | 0.005 | 0.013 | 0.763 |
| Chinese Shar-Pei | 0 | 0.003 | 0.071 | 0.004 | 0.009 | 0.002 | 0.002 | 0.003 | 0.002 | 0.001 | 0.003 | 0.002 | 0.001 | 0.004 | 0.117 | 0.737 |
| Chinese Shar-Pei | (6) | 0.008 | 0.156 | 0.006 | 0.003 | 0.012 | 0.002 | 0.002 | 0.004 | 0.005 | 0.005 | 0.01 | 0.002 | 0.007 | 0.004 | 0.699 |
| Chinese Shar-Pei | (3) | 0.035 | 0.009 | 0.003 | 0.004 | 0.002 | 0.002 | 0.003 | 0.003 | 0.004 | 0.002 | 0.005 | 0.002 | 0.003 | 0.005 | 0.802 |
| Chinese Shar-Pei | (5) | 0.003 | 0.003 | 0.008 | 0.003 | 0.043 | 0.003 | 0.002 | 0.005 | 0.003 | 0.001 | 0.002 | 0.002 | 0.003 | 0.001 | 0.864 |
| Chinese Shar-Pei | (7) | 0.004 | 0.011 | 0.003 | 0.008 | 0.023 | 0.005 | 0.002 | 0.006 | 0.006 | 0.002 | 0.004 | 0.021 | 0.005 | 0.003 | 0.686 |
| Chinese Shar-Pei | (1) | 0.008 | 0.033 | 0.003 | 0.004 | 0.006 | 0.005 | 0.002 | 0.004 | 0.002 | 0.001 | 0.003 | 0.003 | 0.002 | 0.003 | 0.695 |
| Chinese Shar-Pei | (5) | 0.003 | 0.019 | 0.003 | 0.002 | 0.002 | 0.002 | 0.003 | 0.002 | 0.001 | 0.004 | 0.003 | 0.003 | 0.002 | 0.008 | 0.891 |
| Chinese Shar-Pei | (1) | 0.004 | 0.102 | 0.002 | 0.005 | 0.002 | 0.007 | 0.005 | 0.001 | 0.003 | 0.001 | 0.002 | 0.003 | 0.005 | 0.011 | 0.752 |
| Chinese Shar-Pei | (3) | 0.003 | 0.005 | 0.002 | 0.003 | 0.001 | 0.005 | 0.002 | 0.002 | 0.003 | 0.002 | 0.002 | 0.003 | 0.004 | 0.003 | 0.9 |
| Chinese Shar-Pei | (1) | 0.002 | 0.003 | 0.002 | 0.004 | 0.001 | 0.007 | 0.003 | 0.003 | 0.006 | 0.002 | 0.001 | 0.002 | 0.005 | 0.002 | 0.896 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Chinese Shar-Pei | | 0.003 | 0.004 | 0.002 | 0.003 | 0.014 | 0.003 | 0.002 | 0.004 | 0.007 | 0.002 | 0.001 | 0.001 | 0.003 | 0.001 | 0.003 |
| Chinese Shar-Pei | | 0.002 | 0.002 | 0.005 | 0.003 | 0.001 | 0.002 | 0.002 | 0.001 | 0.063 | 0.002 | 0.001 | 0.001 | 0.002 | 0.004 | 0.002 |
| Chinese Shar-Pei | | 0.004 | 0.003 | 0.005 | 0.005 | 0.002 | 0.035 | 0.006 | 0.003 | 0.01 | 0.003 | 0.003 | 0.003 | 0.009 | 0.01 | 0.003 |
| Chinese Shar-Pei | | 0.001 | 0.002 | 0.003 | 0.001 | 0.002 | 0.006 | 0.003 | 0.002 | 0.001 | 0.002 | 0.001 | 0.001 | 0.002 | 0.004 | 0.002 |
| Chinese Shar-Pei | | 0.001 | 0.001 | 0.003 | 0.002 | 0.002 | 0.005 | 0.002 | 0.003 | 0.003 | 0.004 | 0.001 | 0.002 | 0.007 | 0.008 | 0.002 |
| Chinese Shar-Pei | | 0.002 | 0.002 | 0.001 | 0.001 | 0.001 | 0.002 | 0.004 | 0.001 | 0.002 | 0.002 | 0.001 | 0.002 | 0.002 | 0.002 | 0.002 |
| Chinese Shar-Pei | | 0.006 | 0.006 | 0.003 | 0.003 | 0.004 | 0.011 | 0.003 | 0.006 | 0.004 | 0.001 | 0.001 | 0.003 | 0.004 | 0.03 | 0.003 |
| Chinese Shar-Pei | | 0.005 | 0.002 | 0.013 | 0.009 | 0.004 | 0.003 | 0.001 | 0.006 | 0.006 | 0.012 | 0.001 | 0.002 | 0.002 | 0.015 | 0.005 |
| Chinese Shar-Pei | | 0.003 | 0.005 | 0.005 | 0.003 | 0.006 | 0.003 | 0.004 | 0.006 | 0.007 | 0.002 | 0.004 | 0.002 | 0.005 | 0.003 | 0.002 |
| Chinese Shar-Pei | | 0.004 | 0.002 | 0.017 | 0.01 | 0.002 | 0.003 | 0.008 | 0.002 | 0.069 | 0.004 | 0.001 | 0.001 | 0.003 | 0.009 | 0.007 |
| Chinese Shar-Pei | | 0.001 | 0.003 | 0.011 | 0.003 | 0.002 | 0.003 | 0.003 | 0.002 | 0.052 | 0.003 | 0.001 | 0.001 | 0.002 | 0.006 | 0.004 |
| Chinese Shar-Pei | | 0.002 | 0.003 | 0.002 | 0.002 | 0.002 | 0.003 | 0.005 | 0.005 | 0.003 | 0.002 | 0.003 | 0.011 | 0.009 | 0.002 | 0.005 |
| Chinese Shar-Pei | | 0.004 | 0.001 | 0.002 | 0.004 | 0.002 | 0.004 | 0.003 | 0.013 | 0.01 | 0.002 | 0.006 | 0.001 | 0.003 | 0.003 | 0.005 |
| Chinese Shar-Pei | | 0.002 | 0.004 | 0.005 | 0.002 | 0.001 | 0.002 | 0.003 | 0.004 | 0.019 | 0.005 | 0.003 | 0.002 | 0.003 | 0.002 | 0.005 |
| Chinese Shar-Pei | | 0.009 | 0.003 | 0.001 | 0.004 | 0.01 | 0.002 | 0.003 | 0.006 | 0.042 | 0.001 | 0.002 | 0.002 | 0.027 | 0.006 | 0.002 |
| Chinese Shar-Pei | | 0.002 | 0.001 | 0.004 | 0.004 | 0.007 | 0.004 | 0.005 | 0.003 | 0.212 | 0.002 | 0.002 | 0.002 | 0.002 | 0.006 | 0.002 |
| Chinese Shar-Pei | | 0.003 | 0.013 | 0.002 | 0.002 | 0.001 | 0.004 | 0.003 | 0.017 | 0.018 | 0.002 | 0.008 | 0.002 | 0.002 | 0.052 | 0.008 |
| Chinese Shar-Pei | | 0.005 | 0.012 | 0.003 | 0.002 | 0.005 | 0.003 | 0.006 | 0.008 | 0.017 | 0.003 | 0.003 | 0.002 | 0.003 | 0.028 | 0.003 |
| Chinese Shar-Pei | | 0.002 | 0.002 | 0.002 | 0.002 | 0.002 | 0.004 | 0.004 | 0.002 | 0.054 | 0.004 | 0.002 | 0.004 | 0.001 | 0.004 | 0.006 |
| Chinese Shar-Pei | | 0.002 | 0.002 | 0.011 | 0.003 | 0.002 | 0.006 | 0.01 | 0.007 | 0.009 | 0.002 | 0.004 | 0.003 | 0.003 | 0.009 | 0.016 |
| Chinese Shar-Pei | | 0.004 | 0.014 | 0.04 | 0.003 | 0.002 | 0.002 | 0.003 | 0.005 | 0.018 | 0.003 | 0.005 | 0.008 | 0.005 | 0.064 | 0.001 |
| Chinese Shar-Pei | | 0.002 | 0.004 | 0.004 | 0.001 | 0.048 | 0.002 | 0.002 | 0.011 | 0.004 | 0.001 | 0.006 | 0.002 | 0.002 | 0.004 | 0.002 |
| Chinese Shar-Pei | | 0.002 | 0.004 | 0.002 | 0.003 | 0.005 | 0.004 | 0.002 | 0.004 | 0.012 | 0.004 | 0.002 | 0.011 | 0.002 | 0.006 | 0.003 |
| Chinese Shar-Pei | | 0.006 | 0.002 | 0.003 | 0.004 | 0.002 | 0.007 | 0.002 | 0.002 | 0.009 | 0.001 | 0.002 | 0.002 | 0.011 | 0.003 | 0.001 |
| Chinese Shar-Pei | | 0.002 | 0.002 | 0.036 | 0.006 | 0.002 | 0.001 | 0.009 | 0.025 | 0.006 | 0.016 | 0.003 | 0.039 | 0.015 | 0.012 | 0.006 |
| Chinese Shar-Pei | | 0.002 | 0.004 | 0.009 | 0.004 | 0.003 | 0.001 | 0.003 | 0.01 | 0.004 | 0.01 | 0.002 | 0.004 | 0.008 | 0.003 | 0.016 |
| Chinese Shar-Pei | | 0.002 | 0.003 | 0.013 | 0.002 | 0.006 | 0.004 | 0.003 | 0.02 | 0.083 | 0.012 | 0.002 | 0.006 | 0.003 | 0.007 | 0.072 |
| Chinese Shar-Pei | | 0.002 | 0.004 | 0.01 | 0.006 | 0.002 | 0.008 | 0.003 | 0.002 | 0.018 | 0.003 | 0.003 | 0.015 | 0.004 | 0.01 | 0.002 |
| Chinese Shar-Pei | | 0.003 | 0.002 | 0.003 | 0.001 | 0.001 | 0.002 | 0.002 | 0.002 | 0.007 | 0.002 | 0.001 | 0.001 | 0.002 | 0.008 | 0.003 |
| Chinese Shar-Pei | | 0.002 | 0.004 | 0.005 | 0.004 | 0.005 | 0.002 | 0.001 | 0.004 | 0.004 | 0.007 | 0.002 | 0.017 | 0.006 | 0.009 | 0.005 |
| Chinese Shar-Pei | | 0.004 | 0.007 | 0.007 | 0.003 | 0.005 | 0.002 | 0.003 | 0.004 | 0.053 | 0.003 | 0.002 | 0.01 | 0.004 | 0.003 | 0.004 |
| Chinese Shar-Pei | | 0.003 | 0.002 | 0.004 | 0.006 | 0.002 | 0.004 | 0.003 | 0.002 | 0.003 | 0.003 | 0.002 | 0.003 | 0.003 | 0.012 | 0.002 |
| Chinese Shar-Pei | | 0.003 | 0.009 | 0.005 | 0.024 | 0.001 | 0.005 | 0.006 | 0.003 | 0.016 | 0.001 | 0.002 | 0.005 | 0.005 | 0.123 | 0.004 |
| Chinese Shar-Pei | | 0.001 | 0.001 | 0.003 | 0.003 | 0.001 | 0.003 | 0.002 | 0.002 | 0.131 | 0.002 | 0.002 | 0.001 | 0.002 | 0.013 | 0.058 |
| Chinese Shar-Pei | | 0.002 | 0.002 | 0.005 | 0.005 | 0.001 | 0.003 | 0.002 | 0.002 | 0.006 | 0.002 | 0.002 | 0.001 | 0.002 | 0.003 | 0.012 |
| Chinese Shar-Pei | | 0.003 | 0.003 | 0.003 | 0.003 | 0.002 | 0.002 | 0.003 | 0.002 | 0.06 | 0.001 | 0.002 | 0.001 | 0.004 | 0.005 | 0.003 |
| Chinese Shar-Pei | | 0.001 | 0.002 | 0.006 | 0.001 | 0.003 | 0.002 | 0.003 | 0.003 | 0.011 | 0.004 | 0.001 | 9.004 | 0.002 | 0.014 | 0.004 |
| Chinese Shar-Pei | | 0.001 | 0.007 | 0.006 | 0.003 | 0.003 | 0.002 | 0.003 | 0.006 | 0.01 | 0.003 | 0.003 | 0.003 | 0.002 | 0.006 | 0.002 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Cocker Spaniel | 0 | 0.003 | 0.002 | 0.003 | 0.002 | 0.001 | 0.003 | 0.005 | 0.004 | 0.002 | 0.003 | 0.01 | 0.002 | 0.009 | 0.002 | 0.001 |
| Cocker Spaniel | (2) | 0.006 | 0.021 | 0.012 | 0.007 | 0.005 | 0.002 | 0.004 | 0.006 | 0.003 | 0.001 | 0.002 | 0.003 | 0.001 | 0.003 | 0.008 |
| Cocker Spaniel | 0 | 0.003 | 0.001 | 0.002 | 0.001 | 0.001 | 0.016 | 0.01 | 0.016 | 0.002 | 0.004 | 0.004 | 0.009 | 0.005 | 0.002 | 0.002 |
| Cocker Spaniel | (1) | 0.003 | 0.003 | 0.002 | 0.001 | 0.002 | 0.005 | 0.007 | 0.009 | 0.046 | 0.002 | 0.003 | 0.001 | 0.031 | 0.001 | 0.002 |
| Cocker Spaniel | 0 | 0.002 | 0.003 | 0.007 | 0.003 | 0.002 | 0.011 | 0.004 | 0.002 | 0.002 | 0.002 | 0.003 | 0.003 | 0.006 | 0.002 | 0.002 |
| Cocker Spaniel | 0 | 0.002 | 0.001 | 0.002 | 0.003 | 0.001 | 0.003 | 0.003 | 0.002 | 0.003 | 0.003 | 0.002 | 0.001 | 0.008 | 0.001 | 0.001 |
| Cocker Spaniel | (1) | 0.003 | 0.002 | 0.008 | 0.002 | 0.002 | 0.032 | 0.002 | 0.002 | 0.001 | 0.011 | 0.002 | 0.002 | 0.002 | 0.001 | 0.007 |
| Cocker Spaniel | 0 | 0.003 | 0.002 | 0.003 | 0.002 | 0.005 | 0.001 | 0.002 | 0.002 | 0.002 | 0.009 | 0.002 | 0.002 | 0.004 | 0.003 | 0.003 |
| Cocker Spaniel | 0 | 0.001 | 0.001 | 0.001 | 0.001 | 0.001 | 0.001 | 0.002 | 0.001 | 0.001 | 0.001 | 0.001 | 0.001 | 0.001 | 0.001 | 0.001 |
| Cocker Spaniel | (9) | 0.001 | 0.001 | 0.001 | 0.002 | 0.001 | 0.001 | 0.001 | 0.001 | 0.001 | 0.002 | 0.001 | 0.001 | 0.002 | 0.001 | 0.001 |
| Cocker Spaniel | 0 | 0.004 | 0.002 | 0.001 | 0.001 | 0.001 | 0.007 | 0.072 | 0.003 | 0.006 | 0.002 | 0.003 | 0.002 | 0.004 | 0.001 | 0.002 |
| Cocker Spaniel | 0 | 0.003 | 0.002 | 0.003 | 0.004 | 0.002 | 0.013 | 0.001 | 0.004 | 0.002 | 0.002 | 0.004 | 0.002 | 0.002 | 0.004 | 0.003 |
| Cocker Spaniel | (4) | 0.004 | 0.005 | 0.002 | 0.002 | 0.003 | 0.005 | 0.016 | 0.003 | 0.009 | 0.002 | 0.004 | 0.002 | 0.004 | 0.001 | 0.007 |
| Cocker Spaniel | 0 | 0.002 | 0.013 | 0.001 | 0.001 | 0.003 | 0.007 | 0.005 | 0.007 | 0.004 | 0.003 | 0.003 | 0.003 | 0.001 | 0.01 | 0.007 |
| Cocker Spaniel | 0 | 0.001 | 0.001 | 0.001 | 0.001 | 0.001 | 0.002 | 0.002 | 0.001 | 0.001 | 0.002 | 0.001 | 0.001 | 0.001 | 0.001 | 0.001 |
| Cocker Spaniel | (2) | 0.002 | 0.001 | 0.002 | 0.001 | 0.006 | 0.003 | 0.001 | 0.003 | 0.001 | 0.004 | 0.002 | 0.001 | 0.002 | 0.002 | 0.006 |
| Cocker Spaniel | (13) | 0.001 | 0.004 | 0.002 | 0.002 | 0.001 | 0.003 | 0.002 | 0.003 | 0.001 | 0.031 | 0.001 | 0.003 | 0.001 | 0.005 | 0.003 |
| Cocker Spaniel | (37) | 0.002 | 0.002 | 0.033 | 0.004 | 0.002 | 0.017 | 0.01 | 0.005 | 0.002 | 0.014 | 0.002 | 0.012 | 0.004 | 0.002 | 0.003 |
| Cocker Spaniel | 0 | 0.002 | 0.002 | 0.002 | 0.002 | 0.002 | 0.006 | 0.002 | 0.003 | 0.002 | 0.001 | 0.002 | 0.002 | 0.002 | 0.002 | 0.002 |
| Cocker Spaniel | 0 | 0.003 | 0.01 | 0.005 | 0.007 | 0.004 | 0.003 | 0.007 | 0.006 | 0.011 | 0.007 | 0.004 | 0.004 | 0.001 | 0.013 | 0.004 |
| Cocker Spaniel | 0 | 0.003 | 0.005 | 0.009 | 0.01 | 0.001 | 0.007 | 0.002 | 0.002 | 0.002 | 0.003 | 0.004 | 0.012 | 0.002 | 0.02 | 0.006 |
| Cocker Spaniel | (4) | 0.003 | 0.003 | 0.005 | 0.006 | 0.002 | 0.02 | 0.004 | 0.002 | 0.012 | 0.004 | 0.004 | 0.002 | 0.024 | 0.001 | 0.003 |
| Cocker Spaniel | 0 | 0.001 | 0.012 | 0.002 | 0.003 | 0.001 | 0.003 | 0.009 | 0.004 | 0.004 | 0.011 | 0.001 | 0.003 | 0.006 | 0.002 | 0.002 |
| Cocker Spaniel | 0 | 0.001 | 0.001 | 0.001 | 0.001 | 0.001 | 0.002 | 0.001 | 0.002 | 0.001 | 0.002 | 0.002 | 0.002 | 0.002 | 0.001 | 0.001 |
| Cocker Spaniel | 0 | 0.001 | 0.002 | 0.001 | 0.002 | 0.001 | 0.003 | 0.005 | 0.004 | 0.002 | 0.002 | 0.002 | 0.005 | 0.001 | 0.005 | 0.002 |
| Cocker Spaniel | 0 | 0.002 | 0.001 | 0.002 | 0.001 | 0.001 | 0.002 | 0.001 | 0.001 | 0.001 | 0.001 | 0.001 | 0.001 | 0.001 | 0.002 | 0.001 |
| Cocker Spaniel | 0 | 0.004 | 0.001 | 0.009 | 0.011 | 0.001 | 0.002 | 0.003 | 0.003 | 0.002 | 0.005 | 0.001 | 0.001 | 0.004 | 0.001 | 0.003 |
| Cocker Spaniel | (2) | 0.001 | 0.002 | 0.001 | 0.001 | 0.001 | 0.001 | 0.001 | 0.001 | 0.001 | 0.001 | 0.001 | 0.001 | 0.003 | 0.001 | 0.001 |
| Cocker Spaniel | 0 | 0.001 | 0.001 | 0.002 | 0.002 | 0.001 | 0.001 | 0.001 | 0.002 | 0.001 | 0.001 | 0.002 | 0.001 | 0.002 | 0.001 | 0.001 |
| Cocker Spaniel | 0 | 0.001 | 0.002 | 0.001 | 0.001 | 0.001 | 0.001 | 0.001 | 0.001 | 0.001 | 0.001 | 0.002 | 0.001 | 0.002 | 0.001 | 0.001 |
| Cocker Spaniel | 0 | 0.001 | 0.003 | 0.002 | 0.001 | 0.002 | 0.003 | 0.004 | 0.006 | 0.002 | 0.002 | 0.002 | 0.007 | 0.001 | 0.002 | 0.004 |
| Cocker Spaniel | 0 | 0.002 | 0.003 | 0.002 | 0.003 | 0.002 | 0.004 | 0.003 | 0.002 | 0.006 | 0.002 | 0.003 | 0.002 | 0.001 | 0.002 | 0.003 |
| Cocker Spaniel | 0 | 0.001 | 0.001 | 0.001 | 0.001 | 0.001 | 0.002 | 0.001 | 0.001 | 0.001 | 0.002 | 0.001 | 0.001 | 0.001 | 0.002 | 0.001 |
| Cocker Spaniel | 0 | 0.001 | 0.003 | 0.004 | 0.001 | 0.002 | 0.004 | 0.001 | 0.003 | 0.002 | 0.016 | 0.003 | 0.005 | 0.001 | 0.005 | 0.005 |
| Cocker Spaniel | 0 | 0.012 | 0.011 | 0.018 | 0.022 | 0.039 | 0.027 | 0.013 | 0.004 | 0.027 | 0.003 | 0.022 | 0.005 | 0.008 | 0.003 | 0.008 |
| Cocker Spaniel | 0 | 0.001 | 0.001 | 0.001 | 0.001 | 0.001 | 0.001 | 0.002 | 0.003 | 0.001 | 0.001 | 0.001 | 0.001 | 0.001 | 0.001 | 0.001 |
| Cocker Spaniel | 0 | 0.001 | 0.009 | 0.002 | 0.002 | 0.003 | 0.002 | 0.005 | 0.004 | 0.001 | 0.002 | 0.001 | 0.001 | 0.003 | 0.001 | 0.002 |
| Cocker Spaniel | 0 | 0.003 | 0.002 | 0.002 | 0.003 | 0.002 | 0.003 | 0.002 | 0.001 | 0.001 | 0.003 | 0.002 | 0.001 | 0.001 | 0.002 | 0.002 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Cocker Spaniel | | 0.001 | 0.005 | 0.003 | 0.002 | 0.008 | 0.001 | 0.001 | 0.002 | 0.002 | 0.009 | 0.897 | 0.013 | 0.002 | 0.002 | 0.001 |
| Cocker Spaniel | | 0.007 | 0.001 | 0.003 | 0.005 | 0.001 | 0.004 | 0.002 | 0.002 | 0.009 | 0.001 | 0.845 | 0.001 | 0.002 | 0.012 | 0.02 |
| Cocker Spaniel | | 0.002 | 0.003 | 0.006 | 0.002 | 0.003 | 0.001 | 0.001 | 0.001 | 0.002 | 0.01 | 0.876 | 0.004 | 0.003 | 0.001 | 0.003 |
| Cocker Spaniel | | 0.003 | 0.003 | 0.002 | 0.002 | 0.003 | 0.002 | 0.001 | 0.003 | 0.005 | 0.003 | 0.839 | 0.005 | 0.006 | 0.002 | 0.002 |
| Cocker Spaniel | | 0.005 | 0.043 | 0.004 | 0.001 | 0.006 | 0.01 | 0.003 | 0.013 | 0.003 | 0.002 | 0.842 | 0.002 | 0.004 | 0.004 | 0.003 |
| Cocker Spaniel | | 0.001 | 0.001 | 0.003 | 0.001 | 0.002 | 0.002 | 0.002 | 0.002 | 0.002 | 0.002 | 0.935 | 0.008 | 0.002 | 0.001 | 0.002 |
| Cocker Spaniel | | 0.075 | 0.003 | 0.006 | 0.003 | 0.015 | 0.003 | 0.001 | 0.003 | 0.004 | 0.001 | 0.746 | 0.001 | 0.049 | 0.003 | 0.005 |
| Cocker Spaniel | | 0.001 | 0.003 | 0.001 | 0.001 | 0.002 | 0.006 | 0.002 | 0.009 | 0.002 | 0.004 | 0.911 | 0.003 | 0.003 | 0.004 | 0.003 |
| Cocker Spaniel | | 0.001 | 0.001 | 0.002 | 0.001 | 0.001 | 0.001 | 0.001 | 0.002 | 0.001 | 0.002 | 0.967 | 0.001 | 0.001 | 0.002 | 0.001 |
| Cocker Spaniel | | 0.001 | 0.002 | 0.001 | 0.001 | 0.002 | 0.001 | 0.001 | 0.003 | 0.002 | 0.002 | 0.957 | 0.002 | 0.002 | 0.002 | 0.001 |
| Cocker Spaniel | | 0.008 | 0.003 | 0.008 | 0.002 | 0.003 | 0.001 | 0.002 | 0.01 | 0.001 | 0.003 | 0.839 | 0.002 | 0.002 | 0.001 | 0.002 |
| Cocker Spaniel | | 0.002 | 0.002 | 0.004 | 0.003 | 0.004 | 0.002 | 0.003 | 0.002 | 0.006 | 0.002 | 0.909 | 0.001 | 0.002 | 0.003 | 0.002 |
| Cocker Spaniel | | 0.002 | 0.001 | 0.002 | 0.003 | 0.002 | 0.007 | 0.002 | 0.002 | 0.004 | 0.001 | 0.876 | 0.004 | 0.019 | 0.007 | 0.001 |
| Cocker Spaniel | | 0.002 | 0.001 | 0.003 | 0.006 | 0.001 | 0.008 | 0.003 | 0.004 | 0.009 | 0.003 | 0.882 | 0.001 | 0.002 | 0.002 | 0.003 |
| Cocker Spaniel | | 0.002 | 0.003 | 0.001 | 0.001 | 0.001 | 0.001 | 0.001 | 0.002 | 0.001 | 0.002 | 0.962 | 0.002 | 0.001 | 0.002 | 0.002 |
| Cocker Spaniel | | 0.002 | 0.001 | 0.002 | 0.003 | 0.003 | 0.004 | 0.002 | 0.002 | 0.003 | 0.001 | 0.923 | 0.002 | 0.001 | 0.002 | 0.011 |
| Cocker Spaniel | | 0.001 | 0.006 | 0.007 | 0.002 | 0.005 | 0.004 | 0.007 | 0.002 | 0.002 | 0.005 | 0.882 | 0.001 | 0.001 | 0.002 | 0.011 |
| Cocker Spaniel | | 0.008 | 0.04 | 0.002 | 0.003 | 0.003 | 0.002 | 0.018 | 0.005 | 0.002 | 0.002 | 0.78 | 0.005 | 0.003 | 0.008 | 0.007 |
| Cocker Spaniel | | 0.002 | 0.004 | 0.004 | 0.003 | 0.002 | 0.004 | 0.003 | 0.006 | 0.003 | 0.001 | 0.918 | 0.002 | 0.003 | 0.012 | 0.002 |
| Cocker Spaniel | | 0.003 | 0.002 | 0.005 | 0.011 | 0.001 | 0.006 | 0.005 | 0.009 | 0.004 | 0.002 | 0.742 | 0.001 | 0.005 | 0.004 | 0.11 |
| Cocker Spaniel | | 0.002 | 0.002 | 0.002 | 0.006 | 0.001 | 0.003 | 0.005 | 0.002 | 0.029 | 0.001 | 0.833 | 0.001 | 0.002 | 0.004 | 0.018 |
| Cocker Spaniel | | 0.001 | 0.002 | 0.006 | 0.006 | 0.006 | 0.004 | 0.002 | 0.003 | 0.003 | 0.001 | 0.856 | 0.004 | 0.004 | 0.004 | 0.002 |
| Cocker Spaniel | | 0.001 | 0.009 | 0.01 | 0.003 | 0.003 | 0.002 | 0.002 | 0.006 | 0.002 | 0.003 | 0.882 | 0.006 | 0.002 | 0.002 | 0.003 |
| Cocker Spaniel | | 0.001 | 0.001 | 0.001 | 0.001 | 0.001 | 0.001 | 0.002 | 0.002 | 0.001 | 0.001 | 0.964 | 0.001 | 0.001 | 0.001 | 0.001 |
| Cocker Spaniel | | 0.001 | 0.001 | 0.002 | 0.004 | 0.001 | 0.003 | 0.002 | 0.002 | 0.004 | 0.003 | 0.932 | 0.001 | 0.003 | 0.002 | 0.001 |
| Cocker Spaniel | | 0.001 | 0.001 | 0.001 | 0.001 | 0.002 | 0.001 | 0.003 | 0.001 | 0.001 | 0.001 | 0.963 | 0.001 | 0.001 | 0.001 | 0.002 |
| Cocker Spaniel | | 0.003 | 0.002 | 0.002 | 0.003 | 0.002 | 0.002 | 0.001 | 0.011 | 0.001 | 0.006 | 0.904 | 0.002 | 0.004 | 0.001 | 0.005 |
| Cocker Spaniel | | 0.001 | 0.002 | 0.001 | 0.001 | 0.001 | 0.001 | 0.002 | 0.001 | 0.001 | 0.001 | 0.966 | 0.002 | 0.001 | 0.002 | 0.001 |
| Cocker Spaniel | | 0.001 | 0.001 | 0.001 | 0.001 | 0.001 | 0.001 | 0.001 | 0.002 | 0.001 | 0.001 | 0.962 | 0.002 | 0.001 | 0.003 | 0.001 |
| Cocker Spaniel | | 0.001 | 0.001 | 0.001 | 0.001 | 0.002 | 0.002 | 0.001 | 0.001 | 0.001 | 0.001 | 0.963 | 0.001 | 0.001 | 0.003 | 0.001 |
| Cocker Spaniel | | 0.002 | 0.001 | 0.002 | 0.004 | 0.001 | 0.002 | 0.003 | 0.002 | 0.003 | 0.003 | 0.928 | 0.001 | 0.002 | 0.002 | 0.003 |
| Cocker Spaniel | | 0.003 | 0.001 | 0.002 | 0.002 | 0.004 | 0.002 | 0.016 | 0.005 | 0.007 | 0.001 | 0.895 | 0.003 | 0.009 | 0.006 | 0.003 |
| Cocker Spaniel | | 0.001 | 0.001 | 0.001 | 0.001 | 0.001 | 0.001 | 0.001 | 0.001 | 0.001 | 0.001 | 0.97 | 0.001 | 0.001 | 0.001 | 0.001 |
| Cocker Spaniel | | 0.001 | 0.002 | 0.007 | 0.019 | 0.004 | 0.006 | 0.013 | 0.002 | 0.003 | 0.003 | 0.851 | 0.001 | 0.002 | 0.001 | 0.028 |
| Cocker Spaniel | | 0.021 | 0.006 | 0.034 | 0.018 | 0.017 | 0.008 | 0.005 | 0.007 | 0.185 | 0.003 | 0.413 | 0.002 | 0.009 | 0.046 | 0.005 |
| Cocker Spaniel | | 0.001 | 0.002 | 0.001 | 0.002 | 0.001 | 0.002 | 0.001 | 0.002 | 0.001 | 0.002 | 0.958 | 0.003 | 0.002 | 0.001 | 0.001 |
| Cocker Spaniel | | 0.001 | 0.003 | 0.005 | 0.001 | 0.019 | 0.014 | 0.004 | 0.002 | 0.003 | 0.003 | 0.879 | 0.003 | 0.001 | 0.002 | 0.022 |
| Cocker Spaniel | | 0.001 | 0.001 | 0.002 | 0.003 | 0.001 | 0.003 | 0.001 | 0.001 | 0.002 | 0.001 | 0.941 | 0.001 | 0.002 | 0.004 | 0.005 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Dachshund | (3) | 0.007 | 0.006 | 0.002 | 0.001 | 0.001 | 0.002 | 0.009 | 0.838 | 0.008 | 0.002 | 0.006 | 0.005 | 0.003 | 0.001 | 0.001 |
| Dachshund | (5) | 0.008 | 0.004 | 0.008 | 0.005 | 0.003 | 0.006 | 0.002 | 0.856 | 0.004 | 0.003 | 0.003 | 0.003 | 0.011 | 0.004 | 0.002 |
| Dachshund | 0 | 0.005 | 0.009 | 0.008 | 0.002 | 0.007 | 0.01 | 0.009 | 0.816 | 0.005 | 0.005 | 0.004 | 0.007 | 0.007 | 0.003 | 0.006 |
| Dachshund | 0 | 0.016 | 0.006 | 0.006 | 0.011 | 0.001 | 0.007 | 0.012 | 0.086 | 0.024 | 0.001 | 0.005 | 0.011 | 0.005 | 0.004 | 0.001 |
| Dachshund | 0 | 0.005 | 0.004 | 0.013 | 0.005 | 0.001 | 0.003 | 0.006 | 0.013 | 0.006 | 0.003 | 0.189 | 0.011 | 0.003 | 0.008 | 0.002 |
| Dachshund | (6) | 0.087 | 0.003 | 0.005 | 0.004 | 0.008 | 0.027 | 0.009 | 0.594 | 0.003 | 0.005 | 0.01 | 0.004 | 0.005 | 0.003 | 0.058 |
| Dachshund | 0 | 0.003 | 0.006 | 0.003 | 0.005 | 0.001 | 0.002 | 0.02 | 0.843 | 0.001 | 0.003 | 0.002 | 0.005 | 0.005 | 0.002 | 0.002 |
| Dachshund | 0 | 0.003 | 0.003 | 0.006 | 0.004 | 0.001 | 0.003 | 0.034 | 0.768 | 0.004 | 0.002 | 0.004 | 0.027 | 0.007 | 0.006 | 0.006 |
| Dachshund | (2) | 0.004 | 0.003 | 0.002 | 0.002 | 0.001 | 0.002 | 0.111 | 0.649 | 0.002 | 0.002 | 0.002 | 0.023 | 0.006 | 0.002 | 0.001 |
| Dachshund | (1) | 0.008 | 0.01 | 0.01 | 0.002 | 0.028 | 0.005 | 0.006 | 0.034 | 0.02 | 0.002 | 0.044 | 0.002 | 0.002 | 0.021 | 0.006 |
| Dachshund | 0 | 0.005 | 0.029 | 0.004 | 0.017 | 0.001 | 0.003 | 0.005 | 0.723 | 0.003 | 0.006 | 0.004 | 0.006 | 0.005 | 0.01 | 0.003 |
| Dachshund | 0 | 0.003 | 0.006 | 0.002 | 0.004 | 0.002 | 0.002 | 0.006 | 0.513 | 0.006 | 0.002 | 0.007 | 0.006 | 0.003 | 0.004 | 0.001 |
| Dachshund | 0 | 0.004 | 0.003 | 0.004 | 0.008 | 0.002 | 0.002 | 0.027 | 0.4 | 0.002 | 0.002 | 0.005 | 0.011 | 0.007 | 0.011 | 0.001 |
| Dachshund | 0 | 0.002 | 0.002 | 0.001 | 0.001 | 0.001 | 0.002 | 0.002 | 0.003 | 0.001 | 0.003 | 0.002 | 0.005 | 0.001 | 0.002 | 0.001 |
| Dachshund | 0 | 0.001 | 0.001 | 0.001 | 0.001 | 0.001 | 0.001 | 0.001 | 0.001 | 0.001 | 0.001 | 0.001 | 0.001 | 0.002 | 0.001 | 0.001 |
| Dachshund | 0 | 0.001 | 0.001 | 0.001 | 0.001 | 0.001 | 0.001 | 0.001 | 0.002 | 0.001 | 0.001 | 0.001 | 0.001 | 0.001 | 0.001 | 0.001 |
| Dachshund | (52) | 0.016 | 0.006 | 0.005 | 0.007 | 0.01 | 0.005 | 0.007 | 0.039 | 0.005 | 0.005 | 0.034 | 0.002 | 0.005 | 0.006 | 0.01 |
| Dachshund | (19) | 0.005 | 0.003 | 0.58 | 0.004 | 0.01 | 0.004 | 0.004 | 0.23 | 0.004 | 0.003 | 0.007 | 0.004 | 0.004 | 0.014 | 0.005 |
| Dachshund | 0 | 0.002 | 0.002 | 0.002 | 0.001 | 0.001 | 0.002 | 0.006 | 0.933 | 0.005 | 0.002 | 0.001 | 0.003 | 0.002 | 0.002 | 0.001 |
| Dachshund | 0 | 0.004 | 0.068 | 0.004 | 0.002 | 0.002 | 0.006 | 0.022 | 0.747 | 0.007 | 0.028 | 0.002 | 0.009 | 0.01 | 0.002 | 0.004 |
| Dachshund | 0 | 0.003 | 0.002 | 0.003 | 0.002 | 0.003 | 0.001 | 0.013 | 0.898 | 0.001 | 0.003 | 0.002 | 0.002 | 0.002 | 0.002 | 0.004 |
| Dachshund | 0 | 0.026 | 0.026 | 0.057 | 0.016 | 0.005 | 0.016 | 0.006 | 0.543 | 0.004 | 0.011 | 0.004 | 0.001 | 0.005 | 0.004 | 0.063 |
| Dachshund | 0 | 0.002 | 0.002 | 0.006 | 0.002 | 0.001 | 0.006 | 0.001 | 0.004 | 0.001 | 0.002 | 0.002 | 0.004 | 0.004 | 0.001 | 0.002 |
| Dachshund | (5) | 0.004 | 0.006 | 0.03 | 0.04 | 0.018 | 0.007 | 0.013 | 0.578 | 0.002 | 0.001 | 0.006 | 0.006 | 0.003 | 0.005 | 0.011 |
| Dachshund | (2) | 0.011 | 0.002 | 0.005 | 0.007 | 0.084 | 0.023 | 0.019 | 0.012 | 0.008 | 0.032 | 0.101 | 0.004 | 0.008 | 0.012 | 0.024 |
| Dachshund | 0 | 0.04 | 0.018 | 0.008 | 0.004 | 0.152 | 0.008 | 0.006 | 0.251 | 0.017 | 0.003 | 0.019 | 0.003 | 0.003 | 0.007 | 0.007 |
| Dachshund | 0 | 0.015 | 0.008 | 0.003 | 0.005 | 0.009 | 0.005 | 0.01 | 0.794 | 0.002 | 0.004 | 0.005 | 0.004 | 0.002 | 0.009 | 0.002 |
| Dachshund | 0 | 0.006 | 0.011 | 0.031 | 0.002 | 0.009 | 0.009 | 0.003 | 0.395 | 0.015 | 0.002 | 0.035 | 0.004 | 0.005 | 0.006 | 0.02 |
| Dachshund | 0 | 0.002 | 0.002 | 0.001 | 0.002 | 0.001 | 0.003 | 0.004 | 0.003 | 0.015 | 0.001 | 0.002 | 0.001 | 0.002 | 0.005 | 0.002 |
| Dachshund | 0 | 0.26 | 0.006 | 0.005 | 0.002 | 0.025 | 0.002 | 0.003 | 0.532 | 0.01 | 0.001 | 0.019 | 0.009 | 0.003 | 0.012 | 0.004 |
| Dachshund | 0 | 0.004 | 0.004 | 0.029 | 0.014 | 0.005 | 0.008 | 0.003 | 0.64 | 0.003 | 0.002 | 0.005 | 0.002 | 0.005 | 0.003 | 0.004 |
| Dachshund | 0 | 0.016 | 0.003 | 0.027 | 0.002 | 0.009 | 0.003 | 0.014 | 0.497 | 0.005 | 0.003 | 0.006 | 0.018 | 0.007 | 0.002 | 0.011 |
| Dachshund | 0 | 0.007 | 0.006 | 0.003 | 0.011 | 0.002 | 0.01 | 0.013 | 0.557 | 0.108 | 0.026 | 0.019 | 0.002 | 0.003 | 0.005 | 0.008 |
| Dachshund | (1) | 0.005 | 0.004 | 0.005 | 0.002 | 0.002 | 0.027 | 0.007 | 0.61 | 0.008 | 0.003 | 0.005 | 0.003 | 0.004 | 0.006 | 0.013 |
| Dachshund | (2) | 0.004 | 0.003 | 0.005 | 0.007 | 0.023 | 0.005 | 0.055 | 0.623 | 0.01 | 0.005 | 0.015 | 0.037 | 0.003 | 0.038 | 0.032 |
| Dachshund | 0 | 0.09 | 0.011 | 0.022 | 0.033 | 0.006 | 0.008 | 0.008 | 0.08 | 0.003 | 0.002 | 0.165 | 0.003 | 0.012 | 0.003 | 0.002 |
| Dachshund | (1) | 0.008 | 0.083 | 0.004 | 0.017 | 0.003 | 0.003 | 0.005 | 0.115 | 0.023 | 0.005 | 0.006 | 0.094 | 0.032 | 0.016 | 0.007 |
| Dachshund | (3) | 0.007 | 0.009 | 0.003 | 0.01 | 0.007 | 0.015 | 0.005 | 0.472 | 0.003 | 0.012 | 0.041 | 0.022 | 0.012 | 0.007 | 0.003 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Dachshund | | 0.011 | 0.003 | 0.009 | 0.002 | 0.002 | 0.003 | 0.011 | 0.006 | 0.003 | 0.015 | 0.009 | 0.026 | 0.002 | 0.005 | 0.002 |
| Dachshund | | 0.002 | 0.004 | 0.004 | 0.003 | 0.008 | 0.004 | 0.004 | 0.002 | 0.002 | 0.032 | 0.004 | 0.004 | 0.002 | 0.002 | 0.002 |
| Dachshund | | 0.002 | 0.005 | 0.003 | 0.002 | 0.009 | 0.011 | 0.003 | 0.005 | 0.003 | 0.011 | 0.003 | 0.005 | 0.008 | 0.004 | 0.025 |
| Dachshund | | 0.002 | 0.003 | 0.011 | 0.008 | 0.002 | 0.002 | 0.021 | 0.003 | 0.003 | 0.708 | 0.01 | 0.014 | 0.006 | 0.003 | 0.009 |
| Dachshund | | 0.001 | 0.008 | 0.029 | 0.003 | 0.003 | 0.002 | 0.021 | 0.003 | 0.004 | 0.635 | 0.002 | 0.005 | 0.002 | 0.001 | 0.01 |
| Dachshund | | 0.026 | 0.002 | 0.01 | 0.004 | 0.025 | 0.005 | 0.005 | 0.007 | 0.007 | 0.011 | 0.011 | 0.01 | 0.004 | 0.022 | 0.024 |
| Dachshund | | 0.002 | 0.003 | 0.003 | 0.003 | 0.001 | 0.003 | 0.002 | 0.005 | 0.002 | 0.059 | 0.006 | 0.002 | 0.004 | 0.002 | 0.001 |
| Dachshund | | 0.001 | 0.01 | 0.003 | 0.005 | 0.002 | 0.002 | 0.006 | 0.016 | 0.009 | 0.049 | 0.004 | 0.008 | 0.002 | 0.001 | 0.002 |
| Dachshund | | 0.001 | 0.002 | 0.004 | 0.002 | 0.001 | 0.002 | 0.002 | 0.005 | 0.003 | 0.153 | 0.004 | 0.002 | 0.003 | 0.002 | 0.003 |
| Dachshund | | 0.003 | 0.002 | 0.007 | 0.003 | 0.001 | 0.399 | 0.003 | 0.002 | 0.011 | 0.321 | 0.001 | 0.005 | 0.026 | 0.011 | 0.004 |
| Dachshund | | 0.003 | 0.002 | 0.016 | 0.004 | 0.001 | 0.023 | 0.003 | 0.004 | 0.003 | 0.005 | 0.008 | 0.083 | 0.002 | 0.004 | 0.016 |
| Dachshund | | 0.001 | 0.018 | 0.008 | 0.003 | 0.001 | 0.003 | 0.004 | 0.002 | 0.005 | 0.366 | 0.003 | 0.007 | 0.006 | 0.004 | 0.004 |
| Dachshund | | 0.001 | 0.016 | 0.004 | 0.011 | 0.001 | 0.016 | 0.006 | 0.004 | 0.004 | 0.417 | 0.006 | 0.004 | 0.011 | 0.004 | 0.004 |
| Dachshund | | 0.001 | 0.002 | 0.001 | 0.001 | 0.001 | 0.001 | 0.001 | 0.002 | 0.001 | 0.954 | 0.001 | 0.002 | 0.001 | 0.001 | 0.001 |
| Dachshund | | 0.001 | 0.001 | 0.001 | 0.001 | 0.001 | 0.001 | 0.001 | 0.001 | 0.001 | 0.971 | 0.001 | 0.002 | 0.001 | 0.001 | 0.001 |
| Dachshund | | 0.001 | 0.002 | 0.001 | 0.001 | 0.001 | 0.001 | 0.001 | 0.001 | 0.001 | 0.971 | 0.001 | 0.001 | 0.001 | 0.001 | 0.001 |
| Dachshund | | 0.009 | 0.003 | 0.006 | 0.008 | 0.005 | 0.009 | 0.008 | 0.004 | 0.006 | 0.729 | 0.008 | 0.008 | 0.009 | 0.019 | 0.007 |
| Dachshund | | 0.006 | 0.01 | 0.004 | 0.002 | 0.002 | 0.022 | 0.005 | 0.005 | 0.003 | 0.03 | 0.002 | 0.012 | 0.003 | 0.009 | 0.005 |
| Dachshund | | 0.004 | 0.002 | 0.002 | 0.001 | 0.002 | 0.002 | 0.001 | 0.001 | 0.002 | 0.005 | 0.005 | 0.002 | 0.002 | 0.001 | 0.002 |
| Dachshund | | 0.003 | 0.004 | 0.004 | 0.006 | 0.002 | 0.003 | 0.006 | 0.01 | 0.002 | 0.003 | 0.004 | 0.003 | 0.029 | 0.002 | 0.003 |
| Dachshund | | 0.002 | 0.005 | 0.002 | 0.002 | 0.002 | 0.005 | 0.002 | 0.002 | 0.003 | 0.01 | 0.002 | 0.002 | 0.005 | 0.009 | 0.004 |
| Dachshund | | 0.003 | 0.016 | 0.004 | 0.02 | 0.002 | 0.019 | 0.02 | 0.008 | 0.004 | 0.002 | 0.003 | 0.032 | 0.054 | 0.014 | 0.009 |
| Dachshund | | 0.003 | 0.005 | 0.002 | 0.002 | 0.002 | 0.004 | 0.002 | 0.002 | 0.002 | 0.926 | 0.002 | 0.003 | 0.001 | 0.004 | 0.001 |
| Dachshund | | 0.003 | 0.043 | 0.079 | 0.015 | 0.026 | 0.004 | 0.006 | 0.015 | 0.038 | 0.007 | 0.002 | 0.002 | 0.014 | 0.012 | 0.004 |
| Dachshund | | 0.02 | 0.003 | 0.002 | 0.003 | 0.004 | 0.031 | 0.006 | 0.003 | 0.068 | 0.405 | 0.006 | 0.072 | 0.008 | 0.012 | 0.004 |
| Dachshund | | 0.005 | 0.003 | 0.004 | 0.008 | 0.002 | 0.025 | 0.008 | 0.005 | 0.017 | 0.326 | 0.003 | 0.013 | 0.011 | 0.004 | 0.02 |
| Dachshund | | 0.003 | 0.004 | 0.006 | 0.005 | 0.002 | 0.041 | 0.011 | 0.007 | 0.004 | 0.005 | 0.002 | 0.003 | 0.005 | 0.006 | 0.02 |
| Dachshund | | 0.007 | 0.004 | 0.014 | 0.098 | 0.007 | 0.051 | 0.022 | 0.003 | 0.02 | 0.188 | 0.004 | 0.008 | 0.007 | 0.006 | 0.007 |
| Dachshund | | 0.004 | 0.002 | 0.012 | 0.001 | 0.001 | 0.002 | 0.002 | 0.003 | 0.006 | 0.906 | 0.002 | 0.007 | 0.002 | 0.003 | 0.003 |
| Dachshund | | 0.005 | 0.002 | 0.009 | 0.004 | 0.002 | 0.009 | 0.003 | 0.004 | 0.032 | 0.004 | 0.002 | 0.002 | 0.006 | 0.015 | 0.006 |
| Dachshund | | 0.009 | 0.003 | 0.108 | 0.013 | 0.008 | 0.021 | 0.002 | 0.043 | 0.011 | 0.002 | 0.019 | 0.005 | 0.005 | 0.012 | 0.005 |
| Dachshund | | 0.006 | 0.009 | 0.021 | 0.058 | 0.002 | 0.004 | 0.005 | 0.014 | 0.004 | 0.001 | 0.004 | 0.017 | 0.001 | 0.153 | 0.075 |
| Dachshund | | 0.017 | 0.02 | 0.04 | 0.019 | 0.013 | 0.004 | 0.007 | 0.002 | 0.049 | 0.027 | 0.006 | 0.006 | 0.002 | 0.002 | 0.004 |
| Dachshund | | 0.003 | 0.013 | 0.047 | 0.014 | 0.032 | 0.002 | 0.005 | 0.007 | 0.008 | 0.075 | 0.003 | 0.007 | 0.021 | 0.003 | 0.055 |
| Dachshund | | 0.002 | 0.008 | 0.012 | 0.005 | 0.004 | 0.005 | 0.003 | 0.004 | 0.035 | 0.004 | 0.006 | 0.028 | 0.005 | 0.007 | 0.008 |
| Dachshund | | 0.003 | 0.009 | 0.012 | 0.015 | 0.003 | 0.004 | 0.004 | 0.008 | 0.002 | 0.45 | 0.006 | 0.008 | 0.023 | 0.002 | 0.003 |
| Dachshund | | 0.035 | 0.011 | 0.127 | 0.006 | 0.014 | 0.01 | 0.07 | 0.013 | 0.018 | 0.004 | 0.005 | 0.163 | 0.035 | 0.056 | 0.008 |
| Dachshund | | 0.008 | 0.061 | 0.069 | 0.003 | 0.109 | 0.006 | 0.008 | 0.002 | 0.007 | 0.032 | 0.037 | 0.009 | 0.007 | 0.008 | 0.005 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Doberman | (1) | 0.003 | 0.001 | 0.003 | 0.001 | 0.002 | 0.002 | 0.003 | 0.006 | 0.001 | 0.002 | 0.003 | 0.916 | 0.002 | 0.003 | 0.005 |
| Pinscher | | | | | | | | | | | | | | | | |
| Doberman | 0 | 0.005 | 0.003 | 0.002 | 0.001 | 0.005 | 0.003 | 0.004 | 0.011 | 0.002 | 0.001 | 0.002 | 0.911 | 0.005 | 0.002 | 0.002 |
| Pinscher | | | | | | | | | | | | | | | | |
| Doberman | 0 | 0.001 | 0.001 | 0.001 | 0.001 | 0.001 | 0.001 | 0.001 | 0.001 | 0.001 | 0.001 | 0.001 | 0.971 | 0.001 | 0.001 | 0.001 |
| Pinscher | | | | | | | | | | | | | | | | |
| Doberman | 0 | 0.002 | 0.005 | 0.001 | 0.002 | 0.013 | 0.001 | 0.002 | 0.007 | 0.002 | 0.003 | 0.002 | 0.862 | 0.002 | 0.035 | 0.003 |
| Pinscher | | | | | | | | | | | | | | | | |
| Doberman | 0 | 0.001 | 0.001 | 0.002 | 0.002 | 0.004 | 0.002 | 0.001 | 0.002 | 0.001 | 0.003 | 0.001 | 0.947 | 0.001 | 0.002 | 0.002 |
| Pinscher | | | | | | | | | | | | | | | | |
| Doberman | 0 | 0.001 | 0.001 | 0.001 | 0.001 | 0.001 | 0.002 | 0.001 | 0.002 | 0.002 | 0.002 | 0.002 | 0.962 | 0.001 | 0.002 | 0.001 |
| Pinscher | | | | | | | | | | | | | | | | |
| Doberman | (2) | 0.003 | 0.003 | 0.003 | 0.002 | 0.004 | 0.002 | 0.003 | 0.002 | 0.002 | 0.003 | 0.002 | 0.917 | 0.004 | 0.009 | 0.002 |
| Pinscher | | | | | | | | | | | | | | | | |
| Doberman | 0 | 0.003 | 0.001 | 0.001 | 0.002 | 0.001 | 0.002 | 0.002 | 0.003 | 0.001 | 0.002 | 0.002 | 0.943 | 0.001 | 0.002 | 0.001 |
| Pinscher | | | | | | | | | | | | | | | | |
| Doberman | 0 | 0.001 | 0.001 | 0.002 | 0.002 | 0.001 | 0.002 | 0.001 | 0.001 | 0.002 | 0.003 | 0.003 | 0.946 | 0.001 | 0.004 | 0.001 |
| Pinscher | | | | | | | | | | | | | | | | |
| Doberman | 0 | 0.003 | 0.002 | 0.004 | 0.004 | 0.001 | 0.002 | 0.003 | 0.004 | 0.002 | 0.001 | 0.006 | 0.916 | 0.006 | 0.002 | 0.002 |
| Pinscher | | | | | | | | | | | | | | | | |
| Doberman | 0 | 0.005 | 0.001 | 0.004 | 0.011 | 0.001 | 0.002 | 0.002 | 0.003 | 0.001 | 0.007 | 0.002 | 0.905 | 0.005 | 0.008 | 0.001 |
| Pinscher | | | | | | | | | | | | | | | | |
| Doberman | 0 | 0.003 | 0.001 | 0.001 | 0.016 | 0.006 | 0.002 | 0.003 | 0.002 | 0.002 | 0.001 | 0.001 | 0.866 | 0.002 | 0.002 | 0.002 |
| Pinscher | | | | | | | | | | | | | | | | |
| Doberman | 0 | 0.003 | 0.004 | 0.003 | 0.007 | 0.004 | 0.001 | 0.002 | 0.005 | 0.001 | 0.001 | 0.008 | 0.913 | 0.003 | 0.011 | 0.002 |
| Pinscher | | | | | | | | | | | | | | | | |
| Doberman | 0 | 0.003 | 0.003 | 0.003 | 0.003 | 0.002 | 0.002 | 0.005 | 0.004 | 0.002 | 0.001 | 0.002 | 0.909 | 0.006 | 0.004 | 0.004 |
| Pinscher | | | | | | | | | | | | | | | | |
| Doberman | (1) | 0.002 | 0.009 | 0.005 | 0.01 | 0.001 | 0.006 | 0.002 | 0.007 | 0.001 | 0.007 | 0.002 | 0.84 | 0.024 | 0.004 | 0.002 |
| Pinscher | | | | | | | | | | | | | | | | |
| Doberman | 0 | 0.003 | 0.002 | 0.003 | 0.005 | 0.001 | 0.002 | 0.002 | 0.007 | 0.001 | 0.001 | 0.002 | 0.921 | 0.006 | 0.002 | 0.001 |
| Pinscher | | | | | | | | | | | | | | | | |
| Doberman | 0 | 0.002 | 0.004 | 0.003 | 0.085 | 0.001 | 0.005 | 0.001 | 0.002 | 0.004 | 0.004 | 0.002 | 0.719 | 0.002 | 0.002 | 0.002 |
| Pinscher | | | | | | | | | | | | | | | | |
| Doberman | 0 | 0.002 | 0.003 | 0.002 | 0.002 | 0.001 | 0.004 | 0.002 | 0.002 | 0.004 | 0.003 | 0.001 | 0.919 | 0.003 | 0.001 | 0.002 |
| Pinscher | | | | | | | | | | | | | | | | |
| Doberman | (1) | 0.007 | 0.005 | 0.002 | 0.01 | 0.007 | 0.002 | 0.002 | 0.002 | 0.002 | 0.004 | 0.002 | 0.874 | 0.002 | 0.003 | 0.003 |
| Pinscher | | | | | | | | | | | | | | | | |
| Doberman | 0 | 0.001 | 0.001 | 0.001 | 0.001 | 0.004 | 0.001 | 0.001 | 0.001 | 0.001 | 0.001 | 0.003 | 0.961 | 0.001 | 0.001 | 0.002 |
| Pinscher | | | | | | | | | | | | | | | | |
| Doberman | 0 | 0.001 | 0.001 | 0.001 | 0.001 | 0.001 | 0.001 | 0.001 | 0.001 | 0.001 | 0.002 | 0.001 | 0.968 | 0.001 | 0.002 | 0.001 |
| Pinscher | | | | | | | | | | | | | | | | |
| Doberman | 0 | 0.004 | 0.001 | 0.002 | 0.004 | 0.002 | 0.002 | 0.006 | 0.003 | 0.001 | 0.003 | 0.003 | 0.87 | 0.007 | 0.005 | 0.002 |
| Pinscher | | | | | | | | | | | | | | | | |
| Doberman | 0 | 0.001 | 0.001 | 0.001 | 0.001 | 0.001 | 0.001 | 0.001 | 0.001 | 0.002 | 0.002 | 0.002 | 0.96 | 0.001 | 0.002 | 0.002 |
| Pinscher | | | | | | | | | | | | | | | | |
| Doberman | 0 | 0.003 | 0.004 | 0.002 | 0.001 | 0.005 | 0.004 | 0.002 | 0.003 | 0.002 | 0.001 | 0.001 | 0.925 | 0.002 | 0.001 | 0.004 |
| Pinscher | | | | | | | | | | | | | | | | |
| Doberman | 0 | 0.002 | 0.002 | 0.002 | 0.002 | 0.001 | 0.002 | 0.002 | 0.001 | 0.004 | 0.001 | 0.002 | 0.946 | 0.004 | 0.001 | 0.002 |
| Pinscher | | | | | | | | | | | | | | | | |
| Doberman | 0 | 0.002 | 0.001 | 0.001 | 0.001 | 0.001 | 0.002 | 0.002 | 0.001 | 0.001 | 0.002 | 0.002 | 0.955 | 0.002 | 0.003 | 0.001 |
| Pinscher | | | | | | | | | | | | | | | | |
| Doberman | 0 | 0.003 | 0.002 | 0.002 | 0.002 | 0.002 | 0.002 | 0.003 | 0.002 | 0.001 | 0.003 | 0.002 | 0.936 | 0.002 | 0.004 | 0.002 |
| Pinscher | | | | | | | | | | | | | | | | |
| Doberman | 0 | 0.001 | 0.001 | 0.001 | 0.002 | 0.001 | 0.001 | 0.001 | 0.001 | 0.001 | 0.001 | 0.001 | 0.968 | 0.001 | 0.001 | 0.001 |
| Pinscher | | | | | | | | | | | | | | | | |
| Doberman | 0 | 0.003 | 0.002 | 0.002 | 0.004 | 0.001 | 0.004 | 0.019 | 0.019 | 0.006 | 0.001 | 0.001 | 0.858 | 0.005 | 0.001 | 0.002 |
| Pinscher | | | | | | | | | | | | | | | | |
| Doberman | 0 | 0.006 | 0.002 | 0.002 | 0.018 | 0.02 | 0.005 | 0.004 | 0.006 | 0.002 | 0.002 | 0.03 | 0.818 | 0.008 | 0.004 | 0.012 |
| Pinscher | | | | | | | | | | | | | | | | |
| Doberman | 0 | 0.002 | 0.001 | 0.003 | 0.004 | 0.001 | 0.001 | 0.001 | 0.001 | 0.001 | 0.001 | 0.002 | 0.955 | 0.002 | 0.001 | 0.001 |
| Pinscher | | | | | | | | | | | | | | | | |
| Doberman | 0 | 0.004 | 0.002 | 0.003 | 0.002 | 0.001 | 0.003 | 0.002 | 0.006 | 0.001 | 0.004 | 0.002 | 0.925 | 0.002 | 0.002 | 0.002 |
| Pinscher | | | | | | | | | | | | | | | | |
| Doberman | 0 | 0.001 | 0.001 | 0.003 | 0.002 | 0.001 | 0.001 | 0.001 | 0.002 | 0.001 | 0.001 | 0.003 | 0.951 | 0.001 | 0.001 | 0.002 |
| Pinscher | | | | | | | | | | | | | | | | |
| Doberman | 0 | 0.007 | 0.001 | 0.002 | 0.002 | 0.001 | 0.003 | 0.002 | 0.008 | 0.004 | 0.001 | 0.009 | 0.899 | 0.004 | 0.003 | 0.001 |
| Pinscher | | | | | | | | | | | | | | | | |
| Doberman | 0 | 0.014 | 0.006 | 0.005 | 0.008 | 0.004 | 0.005 | 0.002 | 0.002 | 0.005 | 0.003 | 0.003 | 0.88 | 0.007 | 0.001 | 0.003 |
| Pinscher | | | | | | | | | | | | | | | | |
| Doberman | 0 | 0.003 | 0.002 | 0.001 | 0.002 | 0.001 | 0.002 | 0.003 | 0.002 | 0.006 | 0.001 | 0.002 | 0.925 | 0.003 | 0.002 | 0.002 |
| Pinscher | | | | | | | | | | | | | | | | |
| Doberman | 0 | 0.001 | 0.001 | 0.001 | 0.001 | 0.001 | 0.001 | 0.001 | 0.001 | 0.001 | 0.002 | 0.001 | 0.967 | 0.001 | 0.001 | 0.001 |
| Pinscher | | | | | | | | | | | | | | | | |
| Doberman | 0 | 0.002 | 0.002 | 0.002 | 0.002 | 0.002 | 0.003 | 0.006 | 0.004 | 0.002 | 0.005 | 0.003 | 0.903 | 0.003 | 0.003 | 0.002 |
| Pinscher | | | | | | | | | | | | | | | | |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Doberman | | 0.001 | 0.005 | 0.007 | 0.003 | 0.008 | 0.002 | 0.005 | 0.001 | 0.004 | 0.002 | 0.001 | 0.003 | 0.001 | 0.001 | 0.002 |
| Pinscher | | | | | | | | | | | | | | | | |
| Doberman | | 0.001 | 0.003 | 0.004 | 0.003 | 0.002 | 0.002 | 0.006 | 0.002 | 0.003 | 0.002 | 0.002 | 0.003 | 0.003 | 0.005 | 0.004 |
| Pinscher | | | | | | | | | | | | | | | | |
| Doberman | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Pinscher | | | | | | | | | | | | | | | | |
| Doberman | | 0.001 | 0.001 | 0.004 | 0.013 | 0.002 | 0.008 | 0.004 | 0.002 | 0.006 | 0.002 | 0.001 | 0.002 | 0.002 | 0.003 | 0.004 |
| Pinscher | | | | | | | | | | | | | | | | |
| Doberman | | 0.001 | 0.001 | 0.002 | 0.003 | 0.003 | 0.001 | 0.002 | 0.001 | 0.002 | 0.001 | 0.001 | 0.001 | 0.003 | 0.001 | 0.002 |
| Pinscher | | | | | | | | | | | | | | | | |
| Doberman | | 0.001 | 0.001 | 0.001 | 0.001 | 0.001 | 0.001 | 0.001 | 0.001 | 0.001 | 0.001 | 0.001 | 0.002 | 0.002 | 0.001 | 0.002 |
| Pinscher | | | | | | | | | | | | | | | | |
| Doberman | | 0.001 | 0.001 | 0.002 | 0.003 | 0.002 | 0.003 | 0.005 | 0.003 | 0.003 | 0.003 | 0.002 | 0.004 | 0.002 | 0.003 | 0.004 |
| Pinscher | | | | | | | | | | | | | | | | |
| Doberman | | 0.002 | 0.002 | 0.002 | 0.003 | 0.003 | 0.003 | 0.002 | 0.002 | 0.004 | 0.002 | 0.001 | 0.001 | 0.002 | 0.001 | 0.002 |
| Pinscher | | | | | | | | | | | | | | | | |
| Doberman | | 0.001 | 0.003 | 0.002 | 0.002 | 0.001 | 0.003 | 0.002 | 0.002 | 0.003 | 0.001 | 0.001 | 0.001 | 0.002 | 0.002 | 0.001 |
| Pinscher | | | | | | | | | | | | | | | | |
| Doberman | | 0.002 | 0.006 | 0.002 | 0.007 | 0.002 | 0.002 | 0.001 | 0.003 | 0.002 | 0.003 | 0.002 | 0.004 | 0.004 | 0.001 | 0.001 |
| Pinscher | | | | | | | | | | | | | | | | |
| Doberman | | 0.001 | 0.002 | 0.003 | 0.002 | 0.002 | 0.004 | 0.003 | 0.001 | 0.006 | 0.003 | 0.002 | 0.003 | 0.002 | 0.005 | 0.002 |
| Pinscher | | | | | | | | | | | | | | | | |
| Doberman | | 0.005 | 0.001 | 0.003 | 0.002 | 0.01 | 0.009 | 0.018 | 0.001 | 0.001 | 0.004 | 0.003 | 0.002 | 0.005 | 0.001 | 0.023 |
| Pinscher | | | | | | | | | | | | | | | | |
| Doberman | | 0.001 | 0.002 | 0.004 | 0.002 | 0.002 | 0.003 | 0.001 | 0.003 | 0.003 | 0.002 | 0.001 | 0.002 | 0.002 | 0.003 | 0.002 |
| Pinscher | | | | | | | | | | | | | | | | |
| Doberman | | 0.001 | 0.004 | 0.008 | 0.004 | 0.002 | 0.002 | 0.002 | 0.004 | 0.006 | 0.002 | 0.001 | 0.003 | 0.002 | 0.002 | 0.003 |
| Pinscher | | | | | | | | | | | | | | | | |
| Doberman | | 0.001 | 0.007 | 0.014 | 0.005 | 0.002 | 0.003 | 0.004 | 0.002 | 0.004 | 0.008 | 0.008 | 0.006 | 0.003 | 0.012 | 0.002 |
| Pinscher | | | | | | | | | | | | | | | | |
| Doberman | | 0.001 | 0.002 | 0.003 | 0.004 | 0.002 | 0.002 | 0.001 | 0.003 | 0.005 | 0.006 | 0.003 | 0.003 | 0.002 | 0.003 | 0.001 |
| Pinscher | | | | | | | | | | | | | | | | |
| Doberman | | 0.001 | 0.013 | 0.011 | 0.008 | 0.002 | 0.002 | 0.008 | 0.002 | 0.003 | 0.003 | 0.002 | 0.003 | 0.101 | 0.002 | 0.002 |
| Pinscher | | | | | | | | | | | | | | | | |
| Doberman | | 0.002 | 0.004 | 0.005 | 0.005 | 0.003 | 0.002 | 0.005 | 0.003 | 0.003 | 0.002 | 0.005 | 0.002 | 0.003 | 0.001 | 0.006 |
| Pinscher | | | | | | | | | | | | | | | | |
| Doberman | | 0.001 | 0.002 | 0.003 | 0.004 | 0.013 | 0.021 | 0.007 | 0.001 | 0.007 | 0.001 | 0.003 | 0.002 | 0.001 | 0.002 | 0.005 |
| Pinscher | | | | | | | | | | | | | | | | |
| Doberman | | 0.001 | 0.001 | 0.001 | 0.002 | 0.001 | 0.001 | 0.001 | 0.002 | 0.002 | 0.001 | 0.001 | 0.002 | 0.001 | 0.001 | 0.001 |
| Pinscher | | | | | | | | | | | | | | | | |
| Doberman | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Pinscher | | | | | | | | | | | | | | | | |
| Doberman | | 0.002 | 0.001 | 0.004 | 0.002 | 0.002 | 0.023 | 0.03 | 0.002 | 0.002 | 0.003 | 0.002 | 0.004 | 0.002 | 0.004 | 0.002 |
| Pinscher | | | | | | | | | | | | | | | | |
| Doberman | | 0.001 | 0.002 | 0.001 | 0.001 | 0.001 | 0.001 | 0.001 | 0.002 | 0.002 | 0.002 | 0.001 | 0.001 | 0.001 | 0.001 | 0.003 |
| Pinscher | | | | | | | | | | | | | | | | |
| Doberman | | 0.001 | 0.001 | 0.001 | 0.002 | 0.003 | 0.003 | 0.003 | 0.002 | 0.006 | 0.001 | 0.002 | 0.001 | 0.003 | 0.003 | 0.006 |
| Pinscher | | | | | | | | | | | | | | | | |
| Doberman | | 0.001 | 0.001 | 0.002 | 0.001 | 0.001 | 0.001 | 0.001 | 0.001 | 0.002 | 0.001 | 0.003 | 0.002 | 0.003 | 0.002 | 0.002 |
| Pinscher | | | | | | | | | | | | | | | | |
| Doberman | | 0.001 | 0.001 | 0.002 | 0.001 | 0.002 | 0.001 | 0.002 | 0.001 | 0.003 | 0.002 | 0.001 | 0.001 | 0.002 | 0.001 | 0.001 |
| Pinscher | | | | | | | | | | | | | | | | |
| Doberman | | 0.001 | 0.001 | 0.002 | 0.002 | 0.003 | 0.002 | 0.003 | 0.003 | 0.005 | 0.001 | 0.003 | 0.001 | 0.001 | 0.002 | 0.002 |
| Pinscher | | | | | | | | | | | | | | | | |
| Doberman | | 0.001 | 0.001 | 0.001 | 0.001 | 0.001 | 0.001 | 0.001 | 0.001 | 0.001 | 0.002 | 0.001 | 0.001 | 0.001 | 0.002 | 0.001 |
| Pinscher | | | | | | | | | | | | | | | | |
| Doberman | | 0.003 | 0.002 | 0.008 | 0.005 | 0.001 | 0.001 | 0.003 | 0.003 | 0.005 | 0.006 | 0.007 | 0.003 | 0.004 | 0.003 | 0.017 |
| Pinscher | | | | | | | | | | | | | | | | |
| Doberman | | 0.015 | 0.002 | 0.003 | 0.007 | 0.001 | 0.002 | 0.003 | 0.001 | 0.005 | 0.005 | 0.002 | 0.002 | 0.006 | 0.001 | 0.005 |
| Pinscher | | | | | | | | | | | | | | | | |
| Doberman | | 0.001 | 0.002 | 0.001 | 0.003 | 0.001 | 0.001 | 0.001 | 0.002 | 0.001 | 0.002 | 0.001 | 0.001 | 0.001 | 0.002 | 0.001 |
| Pinscher | | | | | | | | | | | | | | | | |
| Doberman | | 0.001 | 0.003 | 0.003 | 0.002 | 0.002 | 0.003 | 0.002 | 0.003 | 0.002 | 0.005 | 0.003 | 0.002 | 0.002 | 0.002 | 0.002 |
| Pinscher | | | | | | | | | | | | | | | | |
| Doberman | | 0.001 | 0.002 | 0.002 | 0.002 | 0.003 | 0.001 | 0.001 | 0.001 | 0.001 | 0.003 | 0.003 | 0.001 | 0.001 | 0.002 | 0.002 |
| Pinscher | | | | | | | | | | | | | | | | |
| Doberman | | 0.003 | 0.002 | 0.013 | 0.002 | 0.001 | 0.002 | 0.002 | 0.002 | 0.003 | 0.005 | 0.002 | 0.005 | 0.005 | 0.003 | 0.005 |
| Pinscher | | | | | | | | | | | | | | | | |
| Doberman | | 0.004 | 0.002 | 0.003 | 0.004 | 0.002 | 0.001 | 0.013 | 0.002 | 0.002 | 0.005 | 0.002 | 0.002 | 0.004 | 0.004 | 0.003 |
| Pinscher | | | | | | | | | | | | | | | | |
| Doberman | | 0.001 | 0.009 | 0.003 | 0.002 | 0.003 | 0.002 | 0.004 | 0.004 | 0.002 | 0.002 | 0.002 | 0.003 | 0.002 | 0.001 | 0.003 |
| Pinscher | | | | | | | | | | | | | | | | |
| Doberman | | 0.001 | 0.002 | 0.001 | 0.001 | 0.001 | 0.002 | 0.001 | 0.001 | 0.001 | 0.001 | 0.002 | 0.001 | 0.001 | 0.001 | 0.001 |
| Pinscher | | | | | | | | | | | | | | | | |
| Doberman | | 0.001 | 0.005 | 0.002 | 0.003 | 0.006 | 0.003 | 0.002 | 0.003 | 0.006 | 0.002 | 0.005 | 0.007 | 0.002 | 0.003 | 0.006 |
| Pinscher | | | | | | | | | | | | | | | | |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| German | (11) | 0.003 | 0.005 | 0.004 | 0.007 | 0.002 | 0.009 | 0.027 | 0.006 | 0.006 | 0.004 | 0.002 | 0.004 | 0.005 | 0.006 | 0.002 |
| Shepherd Dog | | | | | | | | | | | | | | | | |
| German | (12) | 0.001 | 0.003 | 0.001 | 0.002 | 0.001 | 0.003 | 0.003 | 0.005 | 0.002 | 0.001 | 0.001 | 0.005 | 0.002 | 0.002 | 0.002 |
| Shepherd Dog | | | | | | | | | | | | | | | | |
| German | (8) | 0.001 | 0.001 | 0.005 | 0.002 | 0.019 | 0.001 | 0.001 | 0.001 | 0.002 | 0.001 | 0.006 | 0.001 | 0.001 | 0.001 | 0.007 |
| Shepherd Dog | | | | | | | | | | | | | | | | |
| German | (2) | 0.001 | 0.003 | 0.005 | 0.001 | 0.002 | 0.002 | 0.004 | 0.004 | 0.003 | 0.003 | 0.002 | 0.003 | 0.002 | 0.001 | 0.002 |
| Shepherd Dog | | | | | | | | | | | | | | | | |
| German | (2) | 0.002 | 0.002 | 0.001 | 0.001 | 0.001 | 0.001 | 0.003 | 0.003 | 0.001 | 0.004 | 0.001 | 0.004 | 0.001 | 0.001 | 0.002 |
| Shepherd Dog | | | | | | | | | | | | | | | | |
| German | 0 | 0.002 | 0.002 | 0.001 | 0.006 | 0.001 | 0.008 | 0.007 | 0.007 | 0.005 | 0.002 | 0.003 | 0.001 | 0.018 | 0.002 | 0.003 |
| Shepherd Dog | | | | | | | | | | | | | | | | |
| German | (5) | 0.01 | 0.003 | 0.001 | 0.002 | 0.004 | 0.004 | 0.006 | 0.002 | 0.004 | 0.002 | 0.009 | 0.002 | 0.005 | 0.008 | 0.001 |
| Shepherd Dog | | | | | | | | | | | | | | | | |
| German | (3) | 0.001 | 0.001 | 0.002 | 0.001 | 0.001 | 0.002 | 0.001 | 0.001 | 0.001 | 0.001 | 0.001 | 0.001 | 0.002 | 0.001 | 0.001 |
| Shepherd Dog | | | | | | | | | | | | | | | | |
| German | (5) | 0.002 | 0.002 | 0.002 | 0.001 | 0.001 | 0.003 | 0.003 | 0.003 | 0.002 | 0.002 | 0.004 | 0.001 | 0.003 | 0.005 | 0.006 |
| Shepherd Dog | | | | | | | | | | | | | | | | |
| German | (1) | 0.02 | 0.003 | 0.002 | 0.003 | 0.001 | 0.003 | 0.004 | 0.043 | 0.003 | 0.005 | 0.004 | 0.009 | 0.005 | 0.004 | 0.002 |
| Shepherd Dog | | | | | | | | | | | | | | | | |
| German | 0 | 0.008 | 0.003 | 0.002 | 0.002 | 0.001 | 0.002 | 0.002 | 0.003 | 0.01 | 0.002 | 0.003 | 0.003 | 0.002 | 0.001 | 0.001 |
| Shepherd Dog | | | | | | | | | | | | | | | | |
| German | (1) | 0.002 | 0.001 | 0.002 | 0.001 | 0.001 | 0.002 | 0.001 | 0.001 | 0.001 | 0.002 | 0.001 | 0.001 | 0.003 | 0.001 | 0.002 |
| Shepherd Dog | | | | | | | | | | | | | | | | |
| German | (3) | 0.002 | 0.001 | 0.003 | 0.01 | 0.002 | 0.003 | 0.004 | 0.004 | 0.002 | 0.002 | 0.002 | 0.012 | 0.003 | 0.007 | 0.001 |
| Shepherd Dog | | | | | | | | | | | | | | | | |
| German | 0 | 0.004 | 0.002 | 0.002 | 0.002 | 0.009 | 0.004 | 0.002 | 0.001 | 0.004 | 0.003 | 0.001 | 0.002 | 0.005 | 0.002 | 0.005 |
| Shepherd Dog | | | | | | | | | | | | | | | | |
| German | (11) | 0.001 | 0.001 | 0.001 | 0.001 | 0.001 | 0.001 | 0.001 | 0.001 | 0.001 | 0.002 | 0.001 | 0.002 | 0.003 | 0.001 | 0.001 |
| Shepherd Dog | | | | | | | | | | | | | | | | |
| German | (5) | 0.001 | 0.001 | 0.001 | 0.001 | 0.001 | 0.001 | 0.001 | 0.001 | 0.001 | 0.001 | 0.002 | 0.001 | 0.001 | 0.001 | 0.001 |
| Shepherd Dog | | | | | | | | | | | | | | | | |
| German | (4) | 0.002 | 0.007 | 0.005 | 0.001 | 0.001 | 0.004 | 0.018 | 0.006 | 0.006 | 0.007 | 0.001 | 0.002 | 0.003 | 0.004 | 0.002 |
| Shepherd Dog | | | | | | | | | | | | | | | | |
| German | 0 | 0.002 | 0.001 | 0.001 | 0.002 | 0.002 | 0.001 | 0.002 | 0.002 | 0.003 | 0.005 | 0.002 | 0.002 | 0.001 | 0.001 | 0.002 |
| Shepherd Dog | | | | | | | | | | | | | | | | |
| German | 0 | 0.001 | 0.002 | 0.001 | 0.001 | 0.001 | 0.001 | 0.002 | 0.001 | 0.002 | 0.003 | 0.001 | 0.003 | 0.001 | 0.002 | 0.001 |
| Shepherd Dog | | | | | | | | | | | | | | | | |
| German | (1) | 0.003 | 0.002 | 0.001 | 0.002 | 0.001 | 0.003 | 0.001 | 0.002 | 0.001 | 0.002 | 0.002 | 0.003 | 0.004 | 0.001 | 0.001 |
| Shepherd Dog | | | | | | | | | | | | | | | | |
| German | 0 | 0.003 | 0.001 | 0.002 | 0.001 | 0.001 | 0.001 | 0.002 | 0.002 | 0.003 | 0.001 | 0.001 | 0.002 | 0.003 | 0.002 | 0.001 |
| Shepherd Dog | | | | | | | | | | | | | | | | |
| German | (11) | 0.007 | 0.002 | 0.002 | 0.002 | 0.002 | 0.005 | 0.006 | 0.003 | 0.002 | 0.017 | 0.002 | 0.054 | 0.006 | 0.002 | 0.004 |
| Shepherd Dog | | | | | | | | | | | | | | | | |
| German | (47) | 0.007 | 0.005 | 0.004 | 0.007 | 0.005 | 0.004 | 0.003 | 0.002 | 0.003 | 0.005 | 0.064 | 0.01 | 0.007 | 0.004 | 0.011 |
| Shepherd Dog | | | | | | | | | | | | | | | | |
| German | (5) | 0.001 | 0.002 | 0.001 | 0.001 | 0.001 | 0.001 | 0.001 | 0.001 | 0.001 | 0.001 | 0.001 | 0.002 | 0.001 | 0.001 | 0.007 |
| Shepherd Dog | | | | | | | | | | | | | | | | |
| German | (19) | 0.001 | 0.002 | 0.002 | 0.001 | 0.001 | 0.002 | 0.002 | 0.002 | 0.002 | 0.001 | 0.002 | 0.001 | 0.002 | 0.001 | 0.001 |
| Shepherd Dog | | | | | | | | | | | | | | | | |
| German | (4) | 0.002 | 0.002 | 0.002 | 0.002 | 0.001 | 0.005 | 0.005 | 0.009 | 0.002 | 0.002 | 0.003 | 0.003 | 0.004 | 0.001 | 0.001 |
| Shepherd Dog | | | | | | | | | | | | | | | | |
| German | (2) | 0.004 | 0.012 | 0.002 | 0.003 | 0.001 | 0.002 | 0.011 | 0.005 | 0.004 | 0.003 | 0.002 | 0.001 | 0.003 | 0.005 | 0.002 |
| Shepherd Dog | | | | | | | | | | | | | | | | |
| German | 0 | 0.009 | 0.011 | 0.002 | 0.003 | 0.017 | 0.003 | 0.004 | 0.004 | 0.003 | 0.004 | 0.004 | 0.001 | 0.005 | 0.005 | 0.005 |
| Shepherd Dog | | | | | | | | | | | | | | | | |
| German | (1) | 0.003 | 0.001 | 0.001 | 0.002 | 0.002 | 0.001 | 0.001 | 0.001 | 0.002 | 0.001 | 0.003 | 0.001 | 0.001 | 0.001 | 0.001 |
| Shepherd Dog | | | | | | | | | | | | | | | | |
| German | (2) | 0.002 | 0.003 | 0.019 | 0.006 | 0.007 | 0.005 | 0.002 | 0.007 | 0.003 | 0.002 | 0.003 | 0.004 | 0.006 | 0.002 | 0.002 |
| Shepherd Dog | | | | | | | | | | | | | | | | |
| German | (10) | 0.001 | 0.001 | 0.002 | 0.001 | 0.002 | 0.001 | 0.001 | 0.002 | 0.001 | 0.001 | 0.004 | 0.001 | 0.001 | 0.001 | 0.003 |
| Shepherd Dog | | | | | | | | | | | | | | | | |
| German | (2) | 0.001 | 0.002 | 0.002 | 0.003 | 0.001 | 0.001 | 0.002 | 0.006 | 0.002 | 0.006 | 0.002 | 0.009 | 0.003 | 0.002 | 0.001 |
| Shepherd Dog | | | | | | | | | | | | | | | | |
| German | (3) | 0.002 | 0.002 | 0.004 | 0.002 | 0.001 | 0.009 | 0.003 | 0.002 | 0.006 | 0.004 | 0.003 | 0.003 | 0.013 | 0.001 | 0.001 |
| Shepherd Dog | | | | | | | | | | | | | | | | |
| German | (1) | 0.002 | 0.002 | 0.001 | 0.001 | 0.001 | 0.003 | 0.002 | 0.001 | 0.002 | 0.002 | 0.001 | 0.002 | 0.005 | 0.001 | 0.001 |
| Shepherd Dog | | | | | | | | | | | | | | | | |
| German | 0 | 0.002 | 0.001 | 0.001 | 0.001 | 0.002 | 0.001 | 0.001 | 0.001 | 0.002 | 0.002 | 0.002 | 0.001 | 0.001 | 0.002 | 0.006 |
| Shepherd Dog | | | | | | | | | | | | | | | | |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| German | | 0.002 | 0.011 | 0.003 | 0.001 | 0.816 | 0.002 | 0.003 | 0.003 | 0.004 | 0.009 | 0.003 | 0.007 | 0.008 | 0.004 | 0.03 |
| Shepherd Dog | | | | | | | | | | | | | | | | |
| German | | 0.001 | 0.002 | 0.003 | 0.004 | 0.938 | 0.002 | 0.002 | 0.002 | 0.003 | 0.006 | 0.001 | 0.001 | 0.001 | 0.001 | 0.001 |
| Shepherd Dog | | | | | | | | | | | | | | | | |
| German | | 0.001 | 0.002 | 0.001 | 0.001 | 0.927 | 0.002 | 0.003 | 0.001 | 0.001 | 0.003 | 0.001 | 0.001 | 0.002 | 0.002 | 0.002 |
| Shepherd Dog | | | | | | | | | | | | | | | | |
| German | | 0.002 | 0.002 | 0.001 | 0.001 | 0.91 | 0.001 | 0.004 | 0.004 | 0.002 | 0.005 | 0.016 | 0.008 | 0.001 | 0.003 | 0.002 |
| Shepherd Dog | | | | | | | | | | | | | | | | |
| German | | 0.001 | 0.002 | 0.001 | 0.001 | 0.949 | 0.003 | 0.001 | 0.002 | 0.001 | 0.001 | 0.003 | 0.001 | 0.001 | 0.001 | 0.003 |
| Shepherd Dog | | | | | | | | | | | | | | | | |
| German | | 0.003 | 0.003 | 0.011 | 0.006 | 0.88 | 0.002 | 0.003 | 0.002 | 0.004 | 0.003 | 0.003 | 0.002 | 0.006 | 0.003 | 0.003 |
| Shepherd Dog | | | | | | | | | | | | | | | | |
| German | | 0.007 | 0.007 | 0.007 | 0.005 | 0.86 | 0.005 | 0.002 | 0.003 | 0.012 | 0.003 | 0.008 | 0.008 | 0.003 | 0.002 | 0.004 |
| Shepherd Dog | | | | | | | | | | | | | | | | |
| German | | 0.001 | 0.005 | 0.001 | 0.002 | 0.957 | 0.002 | 0.003 | 0.002 | 0.001 | 0.002 | 0.002 | 0.002 | 0.001 | 0.001 | 0.001 |
| Shepherd Dog | | | | | | | | | | | | | | | | |
| German | | 0.003 | 0.01 | 0.001 | 0.009 | 0.894 | 0.003 | 0.015 | 0.008 | 0.003 | 0.001 | 0.004 | 0.003 | 0.002 | 0.002 | 0.001 |
| Shepherd Dog | | | | | | | | | | | | | | | | |
| German | | 0.001 | 0.021 | 0.003 | 0.002 | 0.782 | 0.01 | 0.015 | 0.003 | 0.015 | 0.008 | 0.003 | 0.013 | 0.002 | 0.004 | 0.006 |
| Shepherd Dog | | | | | | | | | | | | | | | | |
| German | | 0.005 | 0.004 | 0.013 | 0.005 | 0.889 | 0.002 | 0.011 | 0.007 | 0.003 | 0.003 | 0.002 | 0.003 | 0.003 | 0.002 | 0.003 |
| Shepherd Dog | | | | | | | | | | | | | | | | |
| German | | 0.001 | 0.003 | 0.002 | 0.001 | 0.955 | 0.003 | 0.003 | 0.001 | 0.001 | 0.002 | 0.001 | 0.001 | 0.002 | 0.001 | 0.001 |
| Shepherd Dog | | | | | | | | | | | | | | | | |
| German | | 0.001 | 0.006 | 0.005 | 0.016 | 0.859 | 0.004 | 0.004 | 0.003 | 0.006 | 0.027 | 0.001 | 0.001 | 0.005 | 0.001 | 0.002 |
| Shepherd Dog | | | | | | | | | | | | | | | | |
| German | | 0.002 | 0.002 | 0.003 | 0.002 | 0.911 | 0.002 | 0.002 | 0.002 | 0.007 | 0.002 | 0.002 | 0.002 | 0.005 | 0.003 | 0.006 |
| Shepherd Dog | | | | | | | | | | | | | | | | |
| German | | 0.001 | 0.001 | 0.001 | 0.002 | 0.964 | 0.001 | 0.001 | 0.002 | 0.001 | 0.001 | 0.001 | 0.001 | 0.002 | 0.001 | 0.001 |
| Shepherd Dog | | | | | | | | | | | | | | | | |
| German | | 0.001 | 0.001 | 0.001 | 0.001 | 0.969 | 0.001 | 0.002 | 0.001 | 0.001 | 0.001 | 0.001 | 0.001 | 0.002 | 0.001 | 0.001 |
| Shepherd Dog | | | | | | | | | | | | | | | | |
| German | | 0.003 | 0.004 | 0.003 | 0.003 | 0.887 | 0.002 | 0.001 | 0.003 | 0.004 | 0.002 | 0.002 | 0.002 | 0.01 | 0.002 | 0.002 |
| Shepherd Dog | | | | | | | | | | | | | | | | |
| German | | 0.001 | 0.001 | 0.002 | 0.002 | 0.934 | 0.002 | 0.012 | 0.001 | 0.002 | 0.003 | 0.002 | 0.002 | 0.001 | 0.005 | 0.002 |
| Shepherd Dog | | | | | | | | | | | | | | | | |
| German | | 0.001 | 0.001 | 0.002 | 0.001 | 0.957 | 0.001 | 0.002 | 0.001 | 0.002 | 0.001 | 0.002 | 0.001 | 0.001 | 0.001 | 0.002 |
| Shepherd Dog | | | | | | | | | | | | | | | | |
| German | | 0.001 | 0.002 | 0.003 | 0.002 | 0.947 | 0.001 | 0.002 | 0.002 | 0.001 | 0.002 | 0.002 | 0.001 | 0.002 | 0.001 | 0.001 |
| Shepherd Dog | | | | | | | | | | | | | | | | |
| German | | 0.002 | 0.008 | 0.003 | 0.001 | 0.939 | 0.001 | 0.001 | 0.002 | 0.001 | 0.002 | 0.007 | 0.002 | 0.002 | 0.002 | 0.001 |
| Shepherd Dog | | | | | | | | | | | | | | | | |
| German | | 0.001 | 0.002 | 0.004 | 0.008 | 0.797 | 0.015 | 0.003 | 0.013 | 0.003 | 0.002 | 0.009 | 0.002 | 0.005 | 0.007 | 0.01 |
| Shepherd Dog | | | | | | | | | | | | | | | | |
| German | | 0.002 | 0.016 | 0.016 | 0.011 | 0.726 | 0.005 | 0.008 | 0.002 | 0.004 | 0.003 | 0.004 | 0.006 | 0.037 | 0.016 | 0.003 |
| Shepherd Dog | | | | | | | | | | | | | | | | |
| German | | 0.001 | 0.002 | 0.002 | 0.001 | 0.949 | 0.001 | 0.004 | 0.001 | 0.007 | 0.001 | 0.003 | 0.002 | 0.001 | 0.001 | 0.001 |
| Shepherd Dog | | | | | | | | | | | | | | | | |
| German | | 0.002 | 0.004 | 0.002 | 0.001 | 0.951 | 0.001 | 0.002 | 0.001 | 0.001 | 0.003 | 0.001 | 0.002 | 0.002 | 0.002 | 0.001 |
| Shepherd Dog | | | | | | | | | | | | | | | | |
| German | | 0.002 | 0.002 | 0.001 | 0.003 | 0.911 | 0.001 | 0.002 | 0.003 | 0.001 | 0.01 | 0.005 | 0.006 | 0.003 | 0.002 | 0.001 |
| Shepherd Dog | | | | | | | | | | | | | | | | |
| German | | 0.001 | 0.003 | 0.003 | 0.003 | 0.861 | 0.002 | 0.011 | 0.008 | 0.003 | 0.003 | 0.005 | 0.02 | 0.003 | 0.006 | 0.006 |
| Shepherd Dog | | | | | | | | | | | | | | | | |
| German | | 0.001 | 0.004 | 0.002 | 0.005 | 0.813 | 0.011 | 0.014 | 0.012 | 0.006 | 0.002 | 0.008 | 0.009 | 0.005 | 0.007 | 0.02 |
| Shepherd Dog | | | | | | | | | | | | | | | | |
| German | | 0.002 | 0.002 | 0.003 | 0.003 | 0.953 | 0.001 | 0.001 | 0.002 | 0.002 | 0.001 | 0.001 | 0.001 | 0.003 | 0.001 | 0.002 |
| Shepherd Dog | | | | | | | | | | | | | | | | |
| German | | 0.003 | 0.007 | 0.018 | 0.002 | 0.84 | 0.024 | 0.004 | 0.002 | 0.001 | 0.008 | 0.001 | 0.002 | 0.002 | 0.006 | 0.007 |
| Shepherd Dog | | | | | | | | | | | | | | | | |
| German | | 0.001 | 0.001 | 0.002 | 0.002 | 0.956 | 0.001 | 0.002 | 0.002 | 0.001 | 0.001 | 0.001 | 0.001 | 0.003 | 0.001 | 0.001 |
| Shepherd Dog | | | | | | | | | | | | | | | | |
| German | | 0.002 | 0.003 | 0.001 | 0.001 | 0.913 | 0.001 | 0.001 | 0.001 | 0.002 | 0.02 | 0.002 | 0.005 | 0.001 | 0.001 | 0.001 |
| Shepherd Dog | | | | | | | | | | | | | | | | |
| German | | 0.002 | 0.002 | 0.002 | 0.002 | 0.912 | 0.001 | 0.002 | 0.003 | 0.001 | 0.003 | 0.004 | 0.005 | 0.004 | 0.001 | 0.001 |
| Shepherd Dog | | | | | | | | | | | | | | | | |
| German | | 0.001 | 0.002 | 0.002 | 0.003 | 0.944 | 0.002 | 0.002 | 0.003 | 0.001 | 0.001 | 0.002 | 0.002 | 0.004 | 0.002 | 0.001 |
| Shepherd Dog | | | | | | | | | | | | | | | | |
| German | | 0.002 | 0.002 | 0.004 | 0.002 | 0.941 | 0.001 | 0.005 | 0.002 | 0.004 | 0.001 | 0.001 | 0.002 | 0.001 | 0.002 | 0.002 |
| Shepherd Dog | | | | | | | | | | | | | | | | |
| German | | 0.001 | 0.001 | 0.001 | 0.001 | 0.967 | 0.001 | 0.002 | 0.001 | 0.001 | 0.001 | 0.001 | 0.002 | 0.001 | 0.001 | 0.001 |
| Shepherd Dog | | | | | | | | | | | | | | | | |
| German | | 0.001 | 0.002 | 0.003 | 0.003 | 0.945 | 0.001 | 0.001 | 0.002 | 0.004 | 0.006 | 0.001 | 0.001 | 0.002 | 0.001 | 0.001 |
| Shepherd Dog | | | | | | | | | | | | | | | | |
| German | | 0.001 | 0.002 | 0.002 | 0.002 | 0.929 | 0.003 | 0.004 | 0.004 | 0.002 | 0.002 | 0.002 | 0.001 | 0.005 | 0.003 | 0.001 |
| Shepherd Dog | | | | | | | | | | | | | | | | |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| German | 0 | 0.01 | 0.012 | 0.026 | 0.023 | 0.002 | 0.004 | 0.537 | 0.046 | 0.002 | 0.004 | 0.054 | 0.001 | 0.01 | 0.009 | 0.005 |
| Shorthaired | | | | | | | | | | | | | | | | |
| Pointer | | | | | | | | | | | | | | | | |
| German | 0 | 0.028 | 0.009 | 0.007 | 0.011 | 0.002 | 0.006 | 0.657 | 0.056 | 0.004 | 0.004 | 0.029 | 0.006 | 0.008 | 0.014 | 0.004 |
| Shorthaired | | | | | | | | | | | | | | | | |
| Pointer | | | | | | | | | | | | | | | | |
| German | 0 | 0.005 | 0.005 | 0.006 | 0.022 | 0.002 | 0.013 | 0.691 | 0.008 | 0.031 | 0.066 | 0.003 | 0.013 | 0.005 | 0.011 | 0.002 |
| Shorthaired | | | | | | | | | | | | | | | | |
| Pointer | | | | | | | | | | | | | | | | |
| German | (3) | 0.001 | 0.003 | 0.003 | 0.002 | 0.001 | 0.002 | 0.815 | 0.007 | 0.003 | 0.005 | 0.002 | 0.019 | 0.003 | 0.002 | 0.002 |
| Shorthaired | | | | | | | | | | | | | | | | |
| Pointer | | | | | | | | | | | | | | | | |
| German | 0 | 0.001 | 0.002 | 0.001 | 0.002 | 0.002 | 0.018 | 0.896 | 0.003 | 0.005 | 0.002 | 0.001 | 0.001 | 0.001 | 0.002 | 0.004 |
| Shorthaired | | | | | | | | | | | | | | | | |
| Pointer | | | | | | | | | | | | | | | | |
| German | (3) | 0.001 | 0.005 | 0.002 | 0.001 | 0.001 | 0.003 | 0.524 | 0.002 | 0.005 | 0.008 | 0.002 | 0.003 | 0.005 | 0.003 | 0.002 |
| Shorthaired | | | | | | | | | | | | | | | | |
| Pointer | | | | | | | | | | | | | | | | |
| German | 0 | 0.003 | 0.004 | 0.002 | 0.001 | 0.002 | 0.013 | 0.732 | 0.019 | 0.002 | 0.004 | 0.003 | 0.003 | 0.164 | 0.002 | 0.002 |
| Shorthaired | | | | | | | | | | | | | | | | |
| Pointer | | | | | | | | | | | | | | | | |
| German | 0 | 0.017 | 0.009 | 0.004 | 0.002 | 0.004 | 0.002 | 0.663 | 0.027 | 0.026 | 0.076 | 0.032 | 0.019 | 0.004 | 0.005 | 0.002 |
| Shorthaired | | | | | | | | | | | | | | | | |
| Pointer | | | | | | | | | | | | | | | | |
| German | (1) | 0.004 | 0.003 | 0.004 | 0.002 | 0.004 | 0.003 | 0.419 | 0.279 | 0.002 | 0.002 | 0.024 | 0.002 | 0.002 | 0.037 | 0.007 |
| Shorthaired | | | | | | | | | | | | | | | | |
| Pointer | | | | | | | | | | | | | | | | |
| German | 0 | 0.002 | 0.003 | 0.001 | 0.001 | 0.001 | 0.003 | 0.935 | 0.003 | 0.002 | 0.006 | 0.001 | 0.003 | 0.002 | 0.003 | 0.002 |
| Shorthaired | | | | | | | | | | | | | | | | |
| Pointer | | | | | | | | | | | | | | | | |
| German | 0 | 0.003 | 0.007 | 0.004 | 0.002 | 0.001 | 0.002 | 0.81 | 0.005 | 0.002 | 0.007 | 0.002 | 0.008 | 0.016 | 0.003 | 0.003 |
| Shorthaired | | | | | | | | | | | | | | | | |
| Pointer | | | | | | | | | | | | | | | | |
| German | (1) | 0.003 | 0.004 | 0.002 | 0.004 | 0.001 | 0.069 | 0.808 | 0.013 | 0.014 | 0.002 | 0.003 | 0.001 | 0.012 | 0.002 | 0.003 |
| Shorthaired | | | | | | | | | | | | | | | | |
| Pointer | | | | | | | | | | | | | | | | |
| German | 0 | 0.01 | 0.023 | 0.004 | 0.004 | 0.002 | 0.012 | 0.844 | 0.002 | 0.006 | 0.002 | 0.003 | 0.002 | 0.004 | 0.002 | 0.004 |
| Shorthaired | | | | | | | | | | | | | | | | |
| Pointer | | | | | | | | | | | | | | | | |
| German | 0 | 0.004 | 0.005 | 0.003 | 0.003 | 0.003 | 0.001 | 0.877 | 0.004 | 0.002 | 0.007 | 0.002 | 0.001 | 0.003 | 0.012 | 0.002 |
| Shorthaired | | | | | | | | | | | | | | | | |
| Pointer | | | | | | | | | | | | | | | | |
| German | 0 | 0.008 | 0.007 | 0.048 | 0.005 | 0.035 | 0.005 | 0.755 | 0.022 | 0.002 | 0.003 | 0.005 | 0.002 | 0.002 | 0.007 | 0.015 |
| Shorthaired | | | | | | | | | | | | | | | | |
| Pointer | | | | | | | | | | | | | | | | |
| German | 0 | 0.018 | 0.024 | 0.003 | 0.01 | 0.004 | 0.137 | 0.625 | 0.034 | 0.007 | 0.002 | 0.003 | 0.002 | 0.041 | 0.002 | 0.002 |
| Shorthaired | | | | | | | | | | | | | | | | |
| Pointer | | | | | | | | | | | | | | | | |
| German | 0 | 0.002 | 0.003 | 0.002 | 0.002 | 0.002 | 0.006 | 0.399 | 0.331 | 0.014 | 0.005 | 0.004 | 0.005 | 0.003 | 0.003 | 0.014 |
| Shorthaired | | | | | | | | | | | | | | | | |
| Pointer | | | | | | | | | | | | | | | | |
| German | 0 | 0.012 | 0.002 | 0.009 | 0.003 | 0.003 | 0.004 | 0.852 | 0.017 | 0.002 | 0.002 | 0.02 | 0.006 | 0.004 | 0.004 | 0.004 |
| Shorthaired | | | | | | | | | | | | | | | | |
| Pointer | | | | | | | | | | | | | | | | |
| German | 0 | 0.001 | 0.003 | 0.003 | 0.003 | 0.001 | 0.004 | 0.684 | 0.007 | 0.005 | 0.026 | 0.015 | 0.002 | 0.006 | 0.053 | 0.002 |
| Shorthaired | | | | | | | | | | | | | | | | |
| Pointer | | | | | | | | | | | | | | | | |
| German | 0 | 0.002 | 0.003 | 0.001 | 0.002 | 0.001 | 0.003 | 0.903 | 0.003 | 0.005 | 0.017 | 0.001 | 0.006 | 0.003 | 0.003 | 0.001 |
| Shorthaired | | | | | | | | | | | | | | | | |
| Pointer | | | | | | | | | | | | | | | | |
| German | 0 | 0.002 | 0.004 | 0.003 | 0.009 | 0.002 | 0.03 | 0.848 | 0.003 | 0.006 | 0.001 | 0.002 | 0.003 | 0.002 | 0.003 | 0.002 |
| Shorthaired | | | | | | | | | | | | | | | | |
| Pointer | | | | | | | | | | | | | | | | |
| German | 0 | 0.003 | 0.005 | 0.004 | 0.002 | 0.001 | 0.015 | 0.822 | 0.003 | 0.013 | 0.008 | 0.012 | 0.001 | 0.01 | 0.025 | 0.004 |
| Shorthaired | | | | | | | | | | | | | | | | |
| Pointer | | | | | | | | | | | | | | | | |
| German | (2) | 0.011 | 0.004 | 0.002 | 0.002 | 0.002 | 0.002 | 0.885 | 0.004 | 0.003 | 0.006 | 0.001 | 0.017 | 0.003 | 0.002 | 0.008 |
| Shorthaired | | | | | | | | | | | | | | | | |
| Pointer | | | | | | | | | | | | | | | | |
| German | 0 | 0.002 | 0.004 | 0.002 | 0.002 | 0.001 | 0.002 | 0.899 | 0.01 | 0.004 | 0.002 | 0.003 | 0.006 | 0.005 | 0.002 | 0.002 |
| Shorthaired | | | | | | | | | | | | | | | | |
| Pointer | | | | | | | | | | | | | | | | |
| German | (3) | 0.003 | 0.016 | 0.006 | 0.004 | 0.002 | 0.003 | 0.852 | 0.003 | 0.003 | 0.013 | 0.004 | 0.002 | 0.007 | 0.005 | 0.004 |
| Shorthaired | | | | | | | | | | | | | | | | |
| Pointer | | | | | | | | | | | | | | | | |
| German | 0 | 0.008 | 0.002 | 0.009 | 0.003 | 0.003 | 0.005 | 0.839 | 0.003 | 0.009 | 0.009 | 0.007 | 0.002 | 0.011 | 0.002 | 0.006 |
| Shorthaired | | | | | | | | | | | | | | | | |
| Pointer | | | | | | | | | | | | | | | | |
| German | 0 | 0.003 | 0.008 | 0.001 | 0.002 | 0.001 | 0.002 | 0.861 | 0.005 | 0.006 | 0.005 | 0.003 | 0.001 | 0.005 | 0.003 | 0.003 |
| Shorthaired | | | | | | | | | | | | | | | | |
| Pointer | | | | | | | | | | | | | | | | |
| German | 0 | 0.004 | 0.01 | 0.002 | 0.002 | 0.001 | 0.012 | 0.857 | 0.005 | 0.002 | 0.005 | 0.003 | 0.001 | 0.008 | 0.003 | 0.002 |
| Shorthaired | | | | | | | | | | | | | | | | |
| Pointer | | | | | | | | | | | | | | | | |
| German | (4) | 0.034 | 0.014 | 0.015 | 0.005 | 0.002 | 0.003 | 0.767 | 0.012 | 0.002 | 0.011 | 0.003 | 0.015 | 0.013 | 0.003 | 0.003 |
| Shorthaired | | | | | | | | | | | | | | | | |
| Pointer | | | | | | | | | | | | | | | | |
| German | 0 | 0.007 | 0.007 | 0.002 | 0.002 | 0.005 | 0.006 | 0.534 | 0.037 | 0.006 | 0.003 | 0.003 | 0.005 | 0.004 | 0.011 | 0.007 |
| Shorthaired | | | | | | | | | | | | | | | | |
| Pointer | | | | | | | | | | | | | | | | |
| German | (3) | 0.004 | 0.002 | 0.002 | 0.004 | 0.005 | 0.016 | 0.633 | 0.103 | 0.01 | 0.007 | 0.004 | 0.002 | 0.035 | 0.002 | 0.002 |
| Shorthaired | | | | | | | | | | | | | | | | |
| Pointer | | | | | | | | | | | | | | | | |
| German | (15) | 0.033 | 0.004 | 0.003 | 0.01 | 0.005 | 0.118 | 0.44 | 0.026 | 0.004 | 0.003 | 0.002 | 0.006 | 0.004 | 0.004 | 0.003 |
| Shorthaired | | | | | | | | | | | | | | | | |
| Pointer | | | | | | | | | | | | | | | | |
| German | 0 | 0.009 | 0.006 | 0.013 | 0.002 | 0.003 | 0.002 | 0.668 | 0.06 | 0.018 | 0.012 | 0.008 | 0.002 | 0.007 | 0.004 | 0.003 |
| Shorthaired | | | | | | | | | | | | | | | | |
| Pointer | | | | | | | | | | | | | | | | |
| German | 0 | 0.002 | 0.001 | 0.004 | 0.002 | 0.006 | 0.004 | 0.901 | 0.003 | 0.002 | 0.002 | 0.017 | 0.001 | 0.004 | 0.002 | 0.003 |
| Shorthaired | | | | | | | | | | | | | | | | |
| Pointer | | | | | | | | | | | | | | | | |
| German | 0 | 0.003 | 0.001 | 0.001 | 0.002 | 0.001 | 0.002 | 0.898 | 0.004 | 0.005 | 0.001 | 0.002 | 0.028 | 0.001 | 0.002 | 0.001 |
| Shorthaired | | | | | | | | | | | | | | | | |
| Pointer | | | | | | | | | | | | | | | | |
| German | 0 | 0.002 | 0.005 | 0.002 | 0.004 | 0.003 | 0.008 | 0.46 | 0.206 | 0.012 | 0.005 | 0.002 | 0.012 | 0.007 | 0.004 | 0.004 |
| Shorthaired | | | | | | | | | | | | | | | | |
| Pointer | | | | | | | | | | | | | | | | |
| German | (1) | 0.004 | 0.002 | 0.002 | 0.002 | 0.001 | 0.006 | 0.86 | 0.008 | 0.003 | 0.003 | 0.003 | 0.003 | 0.005 | 0.006 | 0.001 |
| Shorthaired | | | | | | | | | | | | | | | | |
| Pointer | | | | | | | | | | | | | | | | |
| German | 0 | 0.002 | 0.003 | 0.002 | 0.003 | 0.002 | 0.004 | 0.897 | 0.003 | 0.002 | 0.001 | 0.014 | 0.007 | 0.002 | 0.004 | 0.002 |
| Shorthaired | | | | | | | | | | | | | | | | |
| Pointer | | | | | | | | | | | | | | | | |
| | | Cluster Assignment | | | | | | | | | | | | | | |
| Breed | | 16 | 17 | 18 | 19 | 20 | 21 | 22 | 23 | 24 | 25 | 26 | 27 | 28 | 29 | 30 |
| German | | 0.003 | 0.006 | 0.008 | 0.007 | 0.002 | 0.021 | 0.064 | 0.046 | 0.003 | 0.003 | 0.004 | 0.039 | 0.003 | 0.01 | 0.034 |
| Shorthaired | | | | | | | | | | | | | | | | |
| Pointer | | | | | | | | | | | | | | | | |
| German | | 0.001 | 0.023 | 0.049 | 0.004 | 0.007 | 0.013 | 0.01 | 0.008 | 0.012 | 0.004 | 0.002 | 0.004 | 0.009 | 0.002 | 0.008 |
| Shorthaired | | | | | | | | | | | | | | | | |
| Pointer | | | | | | | | | | | | | | | | |
| German | | 0.003 | 0.004 | 0.016 | 0.003 | 0.006 | 0.013 | 0.022 | 0.007 | 0.01 | 0.003 | 0.007 | 0.004 | 0.012 | 0.002 | 0.005 |
| Shorthaired | | | | | | | | | | | | | | | | |
| Pointer | | | | | | | | | | | | | | | | |
| German | | 0.001 | 0.004 | 0.004 | 0.001 | 0.001 | 0.002 | 0.007 | 0.004 | 0.002 | 0.003 | 0.077 | 0.007 | 0.002 | 0.003 | 0.01 |
| Shorthaired | | | | | | | | | | | | | | | | |
| Pointer | | | | | | | | | | | | | | | | |
| German | | 0.001 | 0.002 | 0.005 | 0.005 | 0.005 | 0.003 | 0.003 | 0.01 | 0.005 | 0.003 | 0.006 | 0.001 | 0.002 | 0.004 | 0.004 |
| Shorthaired | | | | | | | | | | | | | | | | |
| Pointer | | | | | | | | | | | | | | | | |
| German | | 0.001 | 0.002 | 0.003 | 0.002 | 0.005 | 0.002 | 0.399 | 0.003 | 0.001 | 0.002 | 0.004 | 0.002 | 0.002 | 0.002 | 0.004 |
| Shorthaired | | | | | | | | | | | | | | | | |
| Pointer | | | | | | | | | | | | | | | | |
| German | | 0.002 | 0.005 | 0.002 | 0.003 | 0.002 | 0.006 | 0.002 | 0.003 | 0.002 | 0.003 | 0.002 | 0.006 | 0.002 | 0.003 | 0.002 |
| Shorthaired | | | | | | | | | | | | | | | | |
| Pointer | | | | | | | | | | | | | | | | |
| German | | 0.006 | 0.004 | 0.014 | 0.004 | 0.005 | 0.012 | 0.003 | 0.002 | 0.003 | 0.002 | 0.008 | 0.003 | 0.038 | 0.002 | 0.003 |
| Shorthaired | | | | | | | | | | | | | | | | |
| Pointer | | | | | | | | | | | | | | | | |
| German | | 0.006 | 0.004 | 0.007 | 0.009 | 0.002 | 0.002 | 0.003 | 0.085 | 0.021 | 0.005 | 0.02 | 0.007 | 0.005 | 0.006 | 0.023 |
| Shorthaired | | | | | | | | | | | | | | | | |
| Pointer | | | | | | | | | | | | | | | | |
| German | | 0.001 | 0.002 | 0.003 | 0.002 | 0.003 | 0.001 | 0.003 | 0.004 | 0.002 | 0.002 | 0.001 | 0.003 | 0.002 | 0.001 | 0.002 |
| Shorthaired | | | | | | | | | | | | | | | | |
| Pointer | | | | | | | | | | | | | | | | |
| German | | 0.009 | 0.031 | 0.004 | 0.002 | 0.014 | 0.008 | 0.02 | 0.001 | 0.003 | 0.014 | 0.006 | 0.005 | 0.002 | 0.002 | 0.002 |
| Shorthaired | | | | | | | | | | | | | | | | |
| Pointer | | | | | | | | | | | | | | | | |
| German | | 0.003 | 0.002 | 0.002 | 0.002 | 0.002 | 0.007 | 0.002 | 0.008 | 0.003 | 0.002 | 0.004 | 0.002 | 0.008 | 0.006 | 0.003 |
| Shorthaired | | | | | | | | | | | | | | | | |
| Pointer | | | | | | | | | | | | | | | | |
| German | | 0.003 | 0.005 | 0.004 | 0.004 | 0.009 | 0.013 | 0.004 | 0.002 | 0.005 | 0.002 | 0.001 | 0.003 | 0.01 | 0.003 | 0.007 |
| Shorthaired | | | | | | | | | | | | | | | | |
| Pointer | | | | | | | | | | | | | | | | |
| German | | 0.002 | 0.003 | 0.003 | 0.001 | 0.003 | 0.003 | 0.009 | 0.018 | 0.002 | 0.002 | 0.005 | 0.006 | 0.002 | 0.009 | 0.004 |
| Shorthaired | | | | | | | | | | | | | | | | |
| Pointer | | | | | | | | | | | | | | | | |
| German | | 0.003 | 0.005 | 0.005 | 0.007 | 0.005 | 0.014 | 0.007 | 0.007 | 0.004 | 0.002 | 0.006 | 0.005 | 0.003 | 0.003 | 0.004 |
| Shorthaired | | | | | | | | | | | | | | | | |
| Pointer | | | | | | | | | | | | | | | | |
| German | | 0.011 | 0.002 | 0.001 | 0.013 | 0.001 | 0.013 | 0.002 | 0.006 | 0.002 | 0.005 | 0.002 | 0.003 | 0.013 | 0.003 | 0.005 |
| Shorthaired | | | | | | | | | | | | | | | | |
| Pointer | | | | | | | | | | | | | | | | |
| German | | 0.004 | 0.003 | 0.034 | 0.002 | 0.087 | 0.008 | 0.008 | 0.005 | 0.005 | 0.007 | 0.014 | 0.007 | 0.007 | 0.003 | 0.012 |
| Shorthaired | | | | | | | | | | | | | | | | |
| Pointer | | | | | | | | | | | | | | | | |
| German | | 0.003 | 0.002 | 0.003 | 0.009 | 0.002 | 0.003 | 0.002 | 0.002 | 0.003 | 0.005 | 0.005 | 0.004 | 0.007 | 0.002 | 0.004 |
| Shorthaired | | | | | | | | | | | | | | | | |
| Pointer | | | | | | | | | | | | | | | | |
| German | | 0.006 | 0.012 | 0.088 | 0.003 | 0.021 | 0.003 | 0.005 | 0.005 | 0.002 | 0.008 | 0.008 | 0.005 | 0.008 | 0.005 | 0.005 |
| Shorthaired | | | | | | | | | | | | | | | | |
| Pointer | | | | | | | | | | | | | | | | |
| German | | 0.001 | 0.002 | 0.002 | 0.001 | 0.002 | 0.001 | 0.002 | 0.006 | 0.001 | 0.008 | 0.01 | 0.004 | 0.002 | 0.001 | 0.003 |
| Shorthaired | | | | | | | | | | | | | | | | |
| Pointer | | | | | | | | | | | | | | | | |
| German | | 0.003 | 0.004 | 0.005 | 0.002 | 0.003 | 0.001 | 0.002 | 0.004 | 0.005 | 0.002 | 0.038 | 0.002 | 0.003 | 0.002 | 0.006 |
| Shorthaired | | | | | | | | | | | | | | | | |
| Pointer | | | | | | | | | | | | | | | | |
| German | | 0.002 | 0.005 | 0.011 | 0.003 | 0.003 | 0.011 | 0.003 | 0.006 | 0.007 | 0.004 | 0.007 | 0.002 | 0.003 | 0.002 | 0.002 |
| Shorthaired | | | | | | | | | | | | | | | | |
| Pointer | | | | | | | | | | | | | | | | |
| German | | 0.013 | 0.003 | 0.002 | 0.001 | 0.002 | 0.002 | 0.001 | 0.005 | 0.003 | 0.003 | 0.005 | 0.003 | 0.001 | 0.001 | 0.003 |
| Shorthaired | | | | | | | | | | | | | | | | |
| Pointer | | | | | | | | | | | | | | | | |
| German | | 0.001 | 0.003 | 0.004 | 0.001 | 0.001 | 0.002 | 0.002 | 0.002 | 0.002 | 0.004 | 0.015 | 0.004 | 0.002 | 0.007 | 0.004 |
| Shorthaired | | | | | | | | | | | | | | | | |
| Pointer | | | | | | | | | | | | | | | | |
| German | | 0.001 | 0.006 | 0.002 | 0.002 | 0.003 | 0.006 | 0.013 | 0.004 | 0.003 | 0.008 | 0.001 | 0.015 | 0.003 | 0.004 | 0.002 |
| Shorthaired | | | | | | | | | | | | | | | | |
| Pointer | | | | | | | | | | | | | | | | |
| German | | 0.001 | 0.004 | 0.002 | 0.002 | 0.009 | 0.004 | 0.003 | 0.003 | 0.004 | 0.003 | 0.004 | 0.01 | 0.006 | 0.002 | 0.026 |
| Shorthaired | | | | | | | | | | | | | | | | |
| Pointer | | | | | | | | | | | | | | | | |
| German | | 0.001 | 0.003 | 0.006 | 0.001 | 0.002 | 0.046 | 0.008 | 0.003 | 0.002 | 0.003 | 0.002 | 0.007 | 0.003 | 0.001 | 0.003 |
| Shorthaired | | | | | | | | | | | | | | | | |
| Pointer | | | | | | | | | | | | | | | | |
| German | | 0.001 | 0.006 | 0.003 | 0.002 | 0.003 | 0.001 | 0.008 | 0.018 | 0.002 | 0.018 | 0.005 | 0.007 | 0.002 | 0.001 | 0.004 |
| Shorthaired | | | | | | | | | | | | | | | | |
| Pointer | | | | | | | | | | | | | | | | |
| German | | 0.004 | 0.002 | 0.004 | 0.017 | 0.01 | 0.01 | 0.005 | 0.003 | 0.003 | 0.002 | 0.019 | 0.001 | 0.003 | 0.005 | 0.009 |
| Shorthaired | | | | | | | | | | | | | | | | |
| Pointer | | | | | | | | | | | | | | | | |
| German | | 0.011 | 0.003 | 0.011 | 0.004 | 0.003 | 0.019 | 0.003 | 0.004 | 0.006 | 0.005 | 0.209 | 0.003 | 0.004 | 0.004 | 0.076 |
| Shorthaired | | | | | | | | | | | | | | | | |
| Pointer | | | | | | | | | | | | | | | | |
| German | | 0.01 | 0.009 | 0.009 | 0.017 | 0.005 | 0.027 | 0.004 | 0.005 | 0.002 | 0.003 | 0.014 | 0.025 | 0.021 | 0.005 | 0.011 |
| Shorthaired | | | | | | | | | | | | | | | | |
| Pointer | | | | | | | | | | | | | | | | |
| German | | 0.006 | 0.005 | 0.042 | 0.035 | 0.002 | 0.009 | 0.008 | 0.005 | 0.008 | 0.002 | 0.089 | 0.002 | 0.04 | 0.043 | 0.038 |
| Shorthaired | | | | | | | | | | | | | | | | |
| Pointer | | | | | | | | | | | | | | | | |
| German | | 0.009 | 0.004 | 0.005 | 0.003 | 0.004 | 0.018 | 0.015 | 0.002 | 0.003 | 0.003 | 0.053 | 0.016 | 0.033 | 0.002 | 0.011 |
| Shorthaired | | | | | | | | | | | | | | | | |
| Pointer | | | | | | | | | | | | | | | | |
| German | | 0.002 | 0.003 | 0.002 | 0.003 | 0.003 | 0.003 | 0.003 | 0.002 | 0.004 | 0.004 | 0.001 | 0.009 | 0.002 | 0.002 | 0.003 |
| Shorthaired | | | | | | | | | | | | | | | | |
| Pointer | | | | | | | | | | | | | | | | |
| German | | 0.008 | 0.002 | 0.005 | 0.001 | 0.009 | 0.001 | 0.001 | 0.002 | 0.002 | 0.004 | 0.002 | 0.002 | 0.002 | 0.001 | 0.001 |
| Shorthaired | | | | | | | | | | | | | | | | |
| Pointer | | | | | | | | | | | | | | | | |
| German | | 0.002 | 0.005 | 0.013 | 0.004 | 0.058 | 0.004 | 0.013 | 0.019 | 0.012 | 0.075 | 0.026 | 0.015 | 0.008 | 0.002 | 0.01 |
| Shorthaired | | | | | | | | | | | | | | | | |
| Pointer | | | | | | | | | | | | | | | | |
| German | | 0.002 | 0.002 | 0.003 | 0.003 | 0.002 | 0.003 | 0.006 | 0.005 | 0.002 | 0.006 | 0.041 | 0.005 | 0.007 | 0.003 | 0.002 |
| Shorthaired | | | | | | | | | | | | | | | | |
| Pointer | | | | | | | | | | | | | | | | |
| German | | 0.001 | 0.002 | 0.003 | 0.002 | 0.002 | 0.001 | 0.002 | 0.002 | 0.004 | 0.002 | 0.003 | 0.002 | 0.022 | 0.002 | 0.002 |
| Shorthaired | | | | | | | | | | | | | | | | |
| Pointer | | | | | | | | | | | | | | | | |
| | % | | | | | | | | | | | | | | | |
| | missing | Cluster assignment | | | | | | | | | | | | | | |
| Breed | data | 1 | 2 | 3 | 4 | 5 | 6 | 7 | 8 | 9 | 10 | 11 | 12 | 13 | 14 | 15 |
| Golden Retriever | 0 | 0.002 | 0.008 | 0.002 | 0.005 | 0.003 | 0.002 | 0.004 | 0.001 | 0.001 | 0.003 | 0.002 | 0.002 | 0.002 | 0.002 | 0.001 |
| Golden Retriever | 0 | 0.002 | 0.004 | 0.001 | 0.002 | 0.003 | 0.002 | 0.004 | 0.002 | 0.002 | 0.008 | 0.002 | 0.002 | 0.003 | 0.003 | 0.003 |
| Golden Retriever | 0 | 0.028 | 0.004 | 0.01 | 0.004 | 0.005 | 0.002 | 0.003 | 0.003 | 0.002 | 0.002 | 0.003 | 0.002 | 0.003 | 0.003 | 0.003 |
| Golden Retriever | (1) | 0.006 | 0.003 | 0.002 | 0.003 | 0.001 | 0.002 | 0.002 | 0.002 | 0.001 | 0.005 | 0.002 | 0.001 | 0.003 | 0.004 | 0.002 |
| Golden Retriever | (1) | 0.034 | 0.006 | 0.004 | 0.003 | 0.002 | 0.003 | 0.002 | 0.004 | 0.002 | 0.002 | 0.003 | 0.001 | 0.003 | 0.002 | 0.009 |
| Golden Retriever | 0 | 0.007 | 0.002 | 0.002 | 0.001 | 0.001 | 0.004 | 0.002 | 0.003 | 0.002 | 0.002 | 0.001 | 0.001 | 0.005 | 0.002 | 0.006 |
| Golden Retriever | 0 | 0.002 | 0.001 | 0.002 | 0.002 | 0.002 | 0.002 | 0.003 | 0.005 | 0.001 | 0.001 | 0.001 | 0.001 | 0.001 | 0.001 | 0.002 |
| Golden Retriever | 0 | 0.004 | 0.002 | 0.014 | 0.005 | 0.001 | 0.002 | 0.004 | 0.006 | 0.004 | 0.004 | 0.002 | 0.002 | 0.008 | 0.003 | 0.004 |
| Golden Retriever | 0 | 0.01 | 0.003 | 0.003 | 0.074 | 0.025 | 0.014 | 0.003 | 0.005 | 0.006 | 0.003 | 0.006 | 0.004 | 0.004 | 0.02 | 0.066 |
| Golden Retriever | (1) | 0.002 | 0.002 | 0.001 | 0.001 | 0.002 | 0.002 | 0.002 | 0.002 | 0.002 | 0.001 | 0.001 | 0.002 | 0.001 | 0.002 | 0.002 |
| Golden Retriever | 0 | 0.023 | 0.006 | 0.025 | 0.016 | 0.003 | 0.017 | 0.005 | 0.003 | 0.002 | 0.01 | 0.005 | 0.003 | 0.014 | 0.014 | 0.016 |
| Golden Retriever | (1) | 0.011 | 0.007 | 0.01 | 0.016 | 0.001 | 0.003 | 0.002 | 0.002 | 0.002 | 0.007 | 0.002 | 0.004 | 0.016 | 0.002 | 0.002 |
| Golden Retriever | 0 | 0.012 | 0.007 | 0.004 | 0.011 | 0.002 | 0.022 | 0.009 | 0.003 | 0.019 | 0.003 | 0.003 | 0.003 | 0.022 | 0.003 | 0.002 |
| Golden Retriever | 0 | 0.003 | 0.002 | 0.002 | 0.002 | 0.009 | 0.004 | 0.002 | 0.005 | 0.002 | 0.001 | 0.004 | 0.002 | 0.004 | 0.003 | 0.003 |
| Golden Retriever | 0 | 0.008 | 0.005 | 0.002 | 0.001 | 0.005 | 0.009 | 0.015 | 0.005 | 0.016 | 0.002 | 0.01 | 0.002 | 0.002 | 0.002 | 0.01 |
| Golden Retriever | 0 | 0.002 | 0.02 | 0.002 | 0.006 | 0.001 | 0.004 | 0.01 | 0.008 | 0.001 | 0.002 | 0.003 | 0.004 | 0.003 | 0.002 | 0.002 |
| Golden Retriever | 0 | 0.004 | 0.009 | 0.002 | 0.004 | 0.004 | 0.005 | 0.013 | 0.02 | 0.002 | 0.002 | 0.001 | 0.006 | 0.011 | 0.001 | 0.003 |
| Golden Retriever | 0 | 0.002 | 0.003 | 0.002 | 0.002 | 0.001 | 0.001 | 0.002 | 0.003 | 0.002 | 0.001 | 0.002 | 0.002 | 0.001 | 0.002 | 0.001 |
| Golden Retriever | 0 | 0.001 | 0.001 | 0.001 | 0.001 | 0.001 | 0.002 | 0.002 | 0.003 | 0.002 | 0.001 | 0.001 | 0.001 | 0.002 | 0.001 | 0.001 |
| Golden Retriever | 0 | 0.001 | 0.001 | 0.001 | 0.001 | 0.001 | 0.002 | 0.002 | 0.002 | 0.001 | 0.001 | 0.001 | 0.002 | 0.001 | 0.002 | 0.001 |
| Golden Retriever | 0 | 0.041 | 0.007 | 0.003 | 0.003 | 0.001 | 0.002 | 0.01 | 0.005 | 0.002 | 0.007 | 0.005 | 0.003 | 0.006 | 0.003 | 0.01 |
| Golden Retriever | 0 | 0.002 | 0.006 | 0.003 | 0.006 | 0.002 | 0.023 | 0.004 | 0.003 | 0.003 | 0.002 | 0.002 | 0.005 | 0.002 | 0.004 | 0.023 |
| Golden Retriever | (7) | 0.003 | 0.013 | 0.003 | 0.006 | 0.002 | 0.051 | 0.009 | 0.004 | 0.024 | 0.002 | 0.021 | 0.002 | 0.019 | 0.005 | 0.002 |
| Golden Retriever | (8) | 0.003 | 0.007 | 0.001 | 0.002 | 0.005 | 0.002 | 0.003 | 0.003 | 0.002 | 0.004 | 0.002 | 0.003 | 0.006 | 0.003 | 0.006 |
| Golden Retriever | (4) | 0.003 | 0.001 | 0.002 | 0.004 | 0.001 | 0.002 | 0.001 | 0.002 | 0.001 | 0.001 | 0.002 | 0.002 | 0.001 | 0.001 | 0.002 |
| Golden Retriever | (2) | 0.002 | 0.002 | 0.005 | 0.003 | 0.001 | 0.005 | 0.003 | 0.003 | 0.001 | 0.002 | 0.003 | 0.002 | 0.001 | 0.001 | 0.001 |
| Golden Retriever | (5) | 0.002 | 0.003 | 0.004 | 0.002 | 0.001 | 0.001 | 0.001 | 0.002 | 0.003 | 0.002 | 0.001 | 0.002 | 0.004 | 0.002 | 0.001 |
| Golden Retriever | (1) | 0.001 | 0.002 | 0.002 | 0.001 | 0.001 | 0.001 | 0.002 | 0.002 | 0.001 | 0.002 | 0.001 | 0.001 | 0.001 | 0.001 | 0.002 |
| Golden Retriever | (1) | 0.002 | 0.003 | 0.004 | 0.002 | 0.001 | 0.003 | 0.004 | 0.002 | 0.003 | 0.002 | 0.003 | 0.002 | 0.001 | 0.001 | 0.001 |
| Golden Retriever | (1) | 0.003 | 0.002 | 0.004 | 0.007 | 0.003 | 0.001 | 0.001 | 0.002 | 0.001 | 0.003 | 0.001 | 0.003 | 0.002 | 0.001 | 0.004 |
| Golden Retriever | (2) | 0.004 | 0.006 | 0.002 | 0.004 | 0.001 | 0.003 | 0.015 | 0.013 | 0.001 | 0.002 | 0.004 | 0.003 | 0.005 | 0.008 | 0.007 |
| Golden Retriever | (3) | 0.016 | 0.015 | 0.002 | 0.001 | 0.001 | 0.003 | 0.003 | 0.005 | 0.003 | 0.002 | 0.003 | 0.008 | 0.003 | 0.005 | 0.002 |
| Golden Retriever | (1) | 0.004 | 0.007 | 0.002 | 0.003 | 0.004 | 0.004 | 0.003 | 0.008 | 0.013 | 0.004 | 0.011 | 0.003 | 0.002 | 0.01 | 0.002 |
| Golden Retriever | (1) | 0.003 | 0.002 | 0.009 | 0.012 | 0.003 | 0.004 | 0.002 | 0.006 | 0.003 | 0.001 | 0.002 | 0.001 | 0.002 | 0.001 | 0.006 |
| Golden Retriever | 0 | 0.005 | 0.011 | 0.021 | 0.02 | 0.006 | 0.002 | 0.005 | 0.043 | 0.001 | 0.018 | 0.013 | 0.006 | 0.002 | 0.006 | 0.01 |
| Golden Retriever | 0 | 0.013 | 0.009 | 0.002 | 0.002 | 0.003 | 0.022 | 0.015 | 0.002 | 0.001 | 0.002 | 0.005 | 0.003 | 0.011 | 0.002 | 0.003 |
| Golden Retriever | (1) | 0.039 | 0.002 | 0.001 | 0.002 | 0.003 | 0.008 | 0.008 | 0.005 | 0.003 | 0.001 | 0.008 | 0.003 | 0.003 | 0.002 | 0.01 |
| Golden Retriever | (1) | 0.003 | 0.002 | 0.001 | 0.003 | 0.004 | 0.003 | 0.002 | 0.002 | 0.002 | 0.003 | 0.003 | 0.001 | 0.001 | 0.001 | 0.001 |
| | | Cluster Assignment | | | | | | | | | | | | | | |
| Breed | | 16 | 17 | 18 | 19 | 20 | 21 | 22 | 23 | 24 | 25 | 26 | 27 | 28 | 29 | 30 |
| Golden Retriever | | 0.001 | 0.003 | 0.002 | 0.002 | 0.002 | 0.001 | 0.006 | 0.004 | 0.003 | 0.002 | 0.001 | 0.007 | 0.001 | 0.002 | 0.922 |
| Golden Retriever | | 0.001 | 0.001 | 0.003 | 0.005 | 0.003 | 0.006 | 0.001 | 0.008 | 0.002 | 0.002 | 0.005 | 0.007 | 0.002 | 0.008 | 0.901 |
| Golden Retriever | | 0.001 | 0.002 | 0.002 | 0.002 | 0.003 | 0.004 | 0.004 | 0.002 | 0.009 | 0.006 | 0.001 | 0.003 | 0.001 | 0.003 | 0.88 |
| Golden Retriever | | 0.001 | 0.004 | 0.003 | 0.002 | 0.002 | 0.005 | 0.003 | 0.002 | 0.008 | 0.003 | 0.002 | 0.004 | 0.002 | 0.002 | 0.92 |
| Golden Retriever | | 0.001 | 0.002 | 0.003 | 0.002 | 0.002 | 0.004 | 0.006 | 0.002 | 0.042 | 0.003 | 0.002 | 0.001 | 0.002 | 0.002 | 0.846 |
| Golden Retriever | | 0.002 | 0.002 | 0.002 | 0.002 | 0.001 | 0.003 | 0.003 | 0.001 | 0.005 | 0.003 | 0.002 | 0.001 | 0.001 | 0.001 | 0.927 |
| Golden Retriever | | 0.001 | 0.001 | 0.002 | 0.002 | 0.001 | 0.003 | 0.003 | 0.002 | 0.002 | 0.002 | 0.002 | 0.001 | 0.003 | 0.002 | 0.945 |
| Golden Retriever | | 0.002 | 0.003 | 0.006 | 0.003 | 0.003 | 0.003 | 0.005 | 0.004 | 0.002 | 0.002 | 0.005 | 0.013 | 0.006 | 0.003 | 0.873 |
| Golden Retriever | | 0.001 | 0.002 | 0.002 | 0.005 | 0.001 | 0.003 | 0.002 | 0.009 | 0.014 | 0.002 | 0.002 | 0.003 | 0.003 | 0.02 | 0.683 |
| Golden Retriever | | 0.001 | 0.002 | 0.002 | 0.003 | 0.001 | 0.001 | 0.001 | 0.002 | 0.002 | 0.001 | 0.001 | 0.001 | 0.001 | 0.001 | 0.953 |
| Golden Retriever | | 0.003 | 0.015 | 0.005 | 0.005 | 0.006 | 0.008 | 0.005 | 0.004 | 0.01 | 0.007 | 0.001 | 0.012 | 0.004 | 0.004 | 0.749 |
| Golden Retriever | | 0.002 | 0.003 | 0.004 | 0.002 | 0.004 | 0.007 | 0.007 | 0.003 | 0.005 | 0.002 | 0.006 | 0.011 | 0.004 | 0.003 | 0.851 |
| Golden Retriever | | 0.003 | 0.005 | 0.015 | 0.005 | 0.003 | 0.006 | 0.005 | 0.009 | 0.004 | 0.004 | 0.014 | 0.007 | 0.003 | 0.007 | 0.785 |
| Golden Retriever | | 0.002 | 0.007 | 0.005 | 0.004 | 0.002 | 0.007 | 0.003 | 0.002 | 0.003 | 0.002 | 0.003 | 0.003 | 0.002 | 0.006 | 0.9 |
| Golden Retriever | | 0.004 | 0.003 | 0.004 | 0.003 | 0.004 | 0.005 | 0.002 | 0.001 | 0.002 | 0.001 | 0.001 | 0.002 | 0.004 | 0.002 | 0.869 |
| Golden Retriever | | 0.003 | 0.002 | 0.006 | 0.002 | 0.001 | 0.001 | 0.011 | 0.013 | 0.002 | 0.003 | 0.009 | 0.003 | 0.002 | 0.003 | 0.871 |
| Golden Retriever | | 0.004 | 0.002 | 0.007 | 0.022 | 0.003 | 0.002 | 0.016 | 0.004 | 0.004 | 0.007 | 0.005 | 0.003 | 0.005 | 0.007 | 0.822 |
| Golden Retriever | | 0.001 | 0.002 | 0.003 | 0.004 | 0.001 | 0.001 | 0.004 | 0.002 | 0.002 | 0.001 | 0.001 | 0.001 | 0.002 | 0.003 | 0.943 |
| Golden Retriever | | 0.001 | 0.005 | 0.002 | 0.001 | 0.002 | 0.004 | 0.002 | 0.002 | 0.001 | 0.002 | 0.001 | 0.004 | 0.001 | 0.006 | 0.945 |
| Golden Retriever | | 0.001 | 0.001 | 0.001 | 0.001 | 0.001 | 0.002 | 0.001 | 0.001 | 0.002 | 0.002 | 0.002 | 0.001 | 0.001 | 0.001 | 0.961 |
| Golden Retriever | | 0.001 | 0.001 | 0.006 | 0.002 | 0.004 | 0.001 | 0.005 | 0.004 | 0.005 | 0.021 | 0.002 | 0.005 | 0.003 | 0.006 | 0.824 |
| Golden Retriever | | 0.001 | 0.001 | 0.003 | 0.007 | 0.001 | 0.003 | 0.002 | 0.005 | 0.015 | 0.005 | 0.001 | 0.001 | 0.001 | 0.005 | 0.86 |
| Golden Retriever | | 0.023 | 0.003 | 0.038 | 0.005 | 0.003 | 0.004 | 0.018 | 0.005 | 0.002 | 0.032 | 0.021 | 0.003 | 0.021 | 0.002 | 0.65 |
| Golden Retriever | | 0.002 | 0.004 | 0.087 | 0.009 | 0.037 | 0.012 | 0.017 | 0.002 | 0.003 | 0.002 | 0.011 | 0.002 | 0.002 | 0.004 | 0.753 |
| Golden Retriever | | 0.001 | 0.002 | 0.001 | 0.001 | 0.002 | 0.001 | 0.003 | 0.005 | 0.002 | 0.002 | 0.001 | 0.002 | 0.001 | 0.001 | 0.95 |
| Golden Retriever | | 0.002 | 0.002 | 0.002 | 0.004 | 0.002 | 0.002 | 0.001 | 0.003 | 0.001 | 0.003 | 0.003 | 0.001 | 0.001 | 0.001 | 0.935 |
| Golden Retriever | | 0.001 | 0.002 | 0.003 | 0.002 | 0.001 | 0.002 | 0.002 | 0.002 | 0.002 | 0.003 | 0.004 | 0.005 | 0.002 | 0.001 | 0.938 |
| Golden Retriever | | 0.001 | 0.002 | 0.001 | 0.001 | 0.002 | 0.001 | 0.003 | 0.002 | 0.001 | 0.001 | 0.001 | 0.002 | 0.001 | 0.001 | 0.957 |
| Golden Retriever | | 0.001 | 0.007 | 0.008 | 0.002 | 0.006 | 0.001 | 0.003 | 0.003 | 0.001 | 0.002 | 0.001 | 0.001 | 0.002 | 0.001 | 0.929 |
| Golden Retriever | | 0.001 | 0.001 | 0.001 | 0.003 | 0.001 | 0.001 | 0.002 | 0.002 | 0.001 | 0.001 | 0.002 | 0.001 | 0.001 | 0.003 | 0.943 |
| Golden Retriever | | 0.032 | 0.289 | 0.02 | 0.003 | 0.002 | 0.006 | 0.004 | 0.019 | 0.004 | 0.014 | 0.021 | 0.005 | 0.002 | 0.003 | 0.498 |
| Golden Retriever | | 0.003 | 0.102 | 0.097 | 0.004 | 0.002 | 0.007 | 0.002 | 0.006 | 0.013 | 0.002 | 0.003 | 0.002 | 0.002 | 0.002 | 0.683 |
| Golden Retriever | | 0.001 | 0.025 | 0.008 | 0.003 | 0.001 | 0.004 | 0.007 | 0.003 | 0.004 | 0.01 | 0.007 | 0.009 | 0.004 | 0.012 | 0.821 |
| Golden Retriever | | 0.002 | 0.004 | 0.007 | 0.035 | 0.007 | 0.005 | 0.003 | 0.006 | 0.003 | 0.015 | 0.004 | 0.002 | 0.024 | 0.003 | 0.822 |
| Golden Retriever | | 0.002 | 0.004 | 0.016 | 0.006 | 0.007 | 0.005 | 0.002 | 0.009 | 0.004 | 0.112 | 0.011 | 0.011 | 0.006 | 0.023 | 0.61 |
| Golden Retriever | | 0.006 | 0.001 | 0.001 | 0.003 | 0.002 | 0.004 | 0.001 | 0.001 | 0.002 | 0.001 | 0.009 | 0.001 | 0.002 | 0.002 | 0.867 |
| Golden Retriever | | 0.007 | 0.001 | 0.002 | 0.002 | 0.005 | 0.003 | 0.014 | 0.002 | 0.009 | 0.004 | 0.002 | 0.003 | 0.003 | 0.001 | 0.844 |
| Golden Retriever | | 0.001 | 0.001 | 0.001 | 0.003 | 0.001 | 0.003 | 0.004 | 0.002 | 0.001 | 0.002 | 0.004 | 0.002 | 0.002 | 0.002 | 0.938 |
| | % | | | | | | | | | | | | | | | |
| | missing | Cluster assignment | | | | | | | | | | | | | | |
| Breed | data | 1 | 2 | 3 | 4 | 5 | 6 | 7 | 8 | 9 | 10 | 11 | 12 | 13 | 14 | 15 |
| Labrador | 0 | 0.001 | 0.001 | 0.002 | 0.001 | 0.001 | 0.002 | 0.002 | 0.003 | 0.002 | 0.002 | 0.001 | 0.001 | 0.001 | 0.002 | 0.001 |
| Retriever | | | | | | | | | | | | | | | | |
| Labrador | 0 | 0.001 | 0.002 | 0.002 | 0.002 | 0.001 | 0.002 | 0.002 | 0.001 | 0.002 | 0.002 | 0.002 | 0.001 | 0.002 | 0.002 | 0.001 |
| Retriever | | | | | | | | | | | | | | | | |
| Labrador | 0 | 0.001 | 0.001 | 0.001 | 0.002 | 0.001 | 0.001 | 0.002 | 0.001 | 0.001 | 0.001 | 0.001 | 0.001 | 0.001 | 0.001 | 0.001 |
| Retriever | | | | | | | | | | | | | | | | |
| Labrador | 0 | 0.002 | 0.002 | 0.004 | 0.008 | 0.004 | 0.002 | 0.009 | 0.002 | 0.008 | 0.004 | 0.004 | 0.002 | 0.002 | 0.004 | 0.003 |
| Retriever | | | | | | | | | | | | | | | | |
| Labrador | 0 | 0.001 | 0.003 | 0.003 | 0.003 | 0.001 | 0.004 | 0.022 | 0.003 | 0.011 | 0.004 | 0.004 | 0.001 | 0.003 | 0.011 | 0.011 |
| Retriever | | | | | | | | | | | | | | | | |
| Labrador | 0 | 0.011 | 0.007 | 0.055 | 0.087 | 0.003 | 0.005 | 0.004 | 0.009 | 0.002 | 0.023 | 0.013 | 0.053 | 0.003 | 0.002 | 0.009 |
| Retriever | | | | | | | | | | | | | | | | |
| Labrador | 0 | 0.002 | 0.005 | 0.001 | 0.002 | 0.001 | 0.015 | 0.075 | 0.003 | 0.005 | 0.003 | 0.002 | 0.003 | 0.009 | 0.011 | 0.001 |
| Retriever | | | | | | | | | | | | | | | | |
| Labrador | (2) | 0.001 | 0.002 | 0.014 | 0.006 | 0.003 | 0.001 | 0.003 | 0.005 | 0.006 | 0.005 | 0.003 | 0.005 | 0.002 | 0.003 | 0.003 |
| Retriever | | | | | | | | | | | | | | | | |
| Labrador | 0 | 0.002 | 0.002 | 0.002 | 0.003 | 0.001 | 0.003 | 0.014 | 0.003 | 0.002 | 0.002 | 0.01 | 0.001 | 0.003 | 0.001 | 0.001 |
| Retriever | | | | | | | | | | | | | | | | |
| Labrador | (3) | 0.002 | 0.002 | 0.004 | 0.002 | 0.002 | 0.01 | 0.006 | 0.004 | 0.008 | 0.008 | 0.01 | 0.002 | 0.004 | 0.003 | 0.003 |
| Retriever | | | | | | | | | | | | | | | | |
| Labrador | 0 | 0.004 | 0.002 | 0.004 | 0.005 | 0.001 | 0.002 | 0.002 | 0.038 | 0.004 | 0.002 | 0.03 | 0.002 | 0.002 | 0.005 | 0.003 |
| Retriever | | | | | | | | | | | | | | | | |
| Labrador | 0 | 0.002 | 0.004 | 0.002 | 0.003 | 0.002 | 0.002 | 0.003 | 0.002 | 0.005 | 0.004 | 0.002 | 0.001 | 0.004 | 0.001 | 0.002 |
| Retriever | | | | | | | | | | | | | | | | |
| Labrador | 0 | 0.009 | 0.006 | 0.002 | 0.002 | 0.001 | 0.005 | 0.003 | 0.068 | 0.01 | 0.004 | 0.002 | 0.004 | 0.008 | 0.004 | 0.004 |
| Retriever | | | | | | | | | | | | | | | | |
| Labrador | 0 | 0.01 | 0.003 | 0.002 | 0.004 | 0.002 | 0.005 | 0.004 | 0.023 | 0.025 | 0.002 | 0.025 | 0.01 | 0.004 | 0.003 | 0.004 |
| Retriever | | | | | | | | | | | | | | | | |
| Labrador | 0 | 0.016 | 0.056 | 0.005 | 0.012 | 0.003 | 0.009 | 0.124 | 0.011 | 0.003 | 0.003 | 0.003 | 0.016 | 0.003 | 0.041 | 0.009 |
| Retriever | | | | | | | | | | | | | | | | |
| Labrador | 0 | 0.002 | 0.061 | 0.002 | 0.002 | 0.001 | 0.016 | 0.005 | 0.003 | 0.022 | 0.004 | 0.003 | 0.004 | 0.008 | 0.004 | 0.002 |
| Retriever | | | | | | | | | | | | | | | | |
| Labrador | 0 | 0.002 | 0.003 | 0.005 | 0.004 | 0.007 | 0.003 | 0.003 | 0.006 | 0.002 | 0.014 | 0.004 | 0.004 | 0.006 | 0.008 | 0.003 |
| Retriever | | | | | | | | | | | | | | | | |
| Labrador | 0 | 0.002 | 0.001 | 0.002 | 0.002 | 0.002 | 0.002 | 0.002 | 0.006 | 0.001 | 0.005 | 0.003 | 0.002 | 0.001 | 0.004 | 0.002 |
| Retriever | | | | | | | | | | | | | | | | |
| Labrador | 0 | 0.003 | 0.003 | 0.002 | 0.003 | 0.002 | 0.002 | 0.004 | 0.007 | 0.001 | 0.005 | 0.002 | 0.009 | 0.004 | 0.004 | 0.002 |
| Retriever | | | | | | | | | | | | | | | | |
| Labrador | (1) | 0.001 | 0.008 | 0.002 | 0.004 | 0.002 | 0.005 | 0.002 | 0.002 | 0.009 | 0.012 | 0.003 | 0.003 | 0.005 | 0.001 | 0.002 |
| Retriever | | | | | | | | | | | | | | | | |
| Labrador | (1) | 0.005 | 0.005 | 0.002 | 0.005 | 0.003 | 0.027 | 0.034 | 0.009 | 0.014 | 0.005 | 0.009 | 0.004 | 0.04 | 0.014 | 0.015 |
| Retriever | | | | | | | | | | | | | | | | |
| Labrador | 0 | 0.001 | 0.007 | 0.018 | 0.013 | 0.002 | 0.002 | 0.003 | 0.009 | 0.001 | 0.005 | 0.002 | 0.003 | 0.002 | 0.001 | 0.005 |
| Retriever | | | | | | | | | | | | | | | | |
| Labrador | 0 | 0.003 | 0.003 | 0.005 | 0.012 | 0.002 | 0.002 | 0.002 | 0.006 | 0.002 | 0.008 | 0.002 | 0.003 | 0.002 | 0.004 | 0.005 |
| Retriever | | | | | | | | | | | | | | | | |
| Labrador | 0 | 0.008 | 0.008 | 0.014 | 0.015 | 0.002 | 0.003 | 0.003 | 0.011 | 0.003 | 0.004 | 0.031 | 0.004 | 0.003 | 0.01 | 0.005 |
| Retriever | | | | | | | | | | | | | | | | |
| Labrador | 0 | 0.009 | 0.002 | 0.005 | 0.009 | 0.004 | 0.002 | 0.003 | 0.003 | 0.002 | 0.002 | 0.003 | 0.137 | 0.004 | 0.006 | 0.004 |
| Retriever | | | | | | | | | | | | | | | | |
| Labrador | 0 | 0.005 | 0.002 | 0.002 | 0.006 | 0.002 | 0.002 | 0.005 | 0.002 | 0.002 | 0.003 | 0.003 | 0.033 | 0.005 | 0.004 | 0.002 |
| Retriever | | | | | | | | | | | | | | | | |
| Labrador | 0 | 0.003 | 0.007 | 0.001 | 0.002 | 0.002 | 0.003 | 0.014 | 0.006 | 0.002 | 0.003 | 0.001 | 0.006 | 0.006 | 0.002 | 0.001 |
| Retriever | | | | | | | | | | | | | | | | |
| Labrador | 0 | 0.001 | 0.001 | 0.001 | 0.001 | 0.001 | 0.001 | 0.002 | 0.002 | 0.001 | 0.001 | 0.001 | 0.001 | 0.002 | 0.001 | 0.001 |
| Retriever | | | | | | | | | | | | | | | | |
| Labrador | 0 | 0.001 | 0.002 | 0.002 | 0.001 | 0.001 | 0.002 | 0.003 | 0.001 | 0.012 | 0.001 | 0.001 | 0.001 | 0.001 | 0.002 | 0.002 |
| Retriever | | | | | | | | | | | | | | | | |
| Labrador | 0 | 0.006 | 0.002 | 0.019 | 0.013 | 0.008 | 0.009 | 0.001 | 0.005 | 0.007 | 0.01 | 0.013 | 0.002 | 0.016 | 0.004 | 0.007 |
| Retriever | | | | | | | | | | | | | | | | |
| Labrador | 0 | 0.003 | 0.008 | 0.003 | 0.002 | 0.001 | 0.003 | 0.001 | 0.004 | 0.001 | 0.002 | 0.003 | 0.012 | 0.001 | 0.008 | 0.008 |
| Retriever | | | | | | | | | | | | | | | | |
| Labrador | 0 | 0.001 | 0.001 | 0.001 | 0.002 | 0.001 | 0.001 | 0.004 | 0.005 | 0.003 | 0.004 | 0.002 | 0.004 | 0.001 | 0.002 | 0.001 |
| Retriever | | | | | | | | | | | | | | | | |
| Labrador | 0 | 0.006 | 0.004 | 0.004 | 0.007 | 0.001 | 0.002 | 0.003 | 0.003 | 0.001 | 0.022 | 0.003 | 0.003 | 0.002 | 0.011 | 0.003 |
| Retriever | | | | | | | | | | | | | | | | |
| Labrador | 0 | 0.016 | 0.003 | 0.01 | 0.009 | 0.005 | 0.016 | 0.004 | 0.005 | 0.013 | 0.003 | 0.007 | 0.002 | 0.003 | 0.004 | 0.003 |
| Retriever | | | | | | | | | | | | | | | | |
| Labrador | 0 | 0.002 | 0.004 | 0.004 | 0.002 | 0.001 | 0.002 | 0.002 | 0.002 | 0.001 | 0.003 | 0.002 | 0.003 | 0.001 | 0.004 | 0.004 |
| Retriever | | | | | | | | | | | | | | | | |
| Labrador | 0 | 0.002 | 0.002 | 0.001 | 0.001 | 0.001 | 0.002 | 0.005 | 0.005 | 0.003 | 0.002 | 0.001 | 0.001 | 0.002 | 0.001 | 0.002 |
| Retriever | | | | | | | | | | | | | | | | |
| Labrador | 0 | 0.005 | 0.012 | 0.003 | 0.003 | 0.003 | 0.008 | 0.008 | 0.076 | 0.009 | 0.004 | 0.002 | 0.007 | 0.026 | 0.002 | 0.028 |
| Retriever | | | | | | | | | | | | | | | | |
| | | Cluster Assignment | | | | | | | | | | | | | | |
| Breed | | 16 | 17 | 18 | 19 | 20 | 21 | 22 | 23 | 24 | 25 | 26 | 27 | 28 | 29 | 30 |
| Labrador | | 0.001 | 0.002 | 0.001 | 0.001 | 0.002 | 0.001 | 0.001 | 0.955 | 0.001 | 0.001 | 0.002 | 0.001 | 0.002 | 0.003 | 0.002 |
| Retriever | | | | | | | | | | | | | | | | |
| Labrador | | 0.001 | 0.004 | 0.002 | 0.002 | 0.001 | 0.001 | 0.001 | 0.953 | 0.001 | 0.001 | 0.003 | 0.001 | 0.002 | 0.002 | 0.001 |
| Retriever | | | | | | | | | | | | | | | | |
| Labrador | | 0.001 | 0.001 | 0.001 | 0.001 | 0.001 | 0.001 | 0.001 | 0.963 | 0.001 | 0.002 | 0.002 | 0.001 | 0.001 | 0.001 | 0.002 |
| Retriever | | | | | | | | | | | | | | | | |
| Labrador | | 0.001 | 0.003 | 0.005 | 0.002 | 0.045 | 0.016 | 0.012 | 0.792 | 0.003 | 0.011 | 0.025 | 0.005 | 0.012 | 0.003 | 0.004 |
| Retriever | | | | | | | | | | | | | | | | |
| Labrador | | 0.002 | 0.004 | 0.006 | 0.002 | 0.002 | 0.005 | 0.002 | 0.856 | 0.002 | 0.006 | 0.004 | 0.013 | 0.002 | 0.003 | 0.005 |
| Retriever | | | | | | | | | | | | | | | | |
| Labrador | | 0.004 | 0.135 | 0.169 | 0.003 | 0.002 | 0.033 | 0.004 | 0.185 | 0.004 | 0.003 | 0.045 | 0.003 | 0.01 | 0.103 | 0.008 |
| Retriever | | | | | | | | | | | | | | | | |
| Labrador | | 0.003 | 0.003 | 0.002 | 0.009 | 0.003 | 0.002 | 0.003 | 0.743 | 0.002 | 0.004 | 0.064 | 0.001 | 0.017 | 0.001 | 0.005 |
| Retriever | | | | | | | | | | | | | | | | |
| Labrador | | 0.002 | 0.002 | 0.002 | 0.006 | 0.002 | 0.002 | 0.002 | 0.882 | 0.003 | 0.008 | 0.008 | 0.012 | 0.005 | 0.003 | 0.001 |
| Retriever | | | | | | | | | | | | | | | | |
| Labrador | | 0.001 | 0.024 | 0.004 | 0.001 | 0.003 | 0.001 | 0.002 | 0.892 | 0.002 | 0.002 | 0.005 | 0.004 | 0.003 | 0.003 | 0.002 |
| Retriever | | | | | | | | | | | | | | | | |
| Labrador | | 0.002 | 0.044 | 0.01 | 0.007 | 0.002 | 0.001 | 0.004 | 0.821 | 0.003 | 0.006 | 0.006 | 0.011 | 0.004 | 0.003 | 0.004 |
| Retriever | | | | | | | | | | | | | | | | |
| Labrador | | 0.005 | 0.023 | 0.018 | 0.025 | 0.005 | 0.007 | 0.002 | 0.736 | 0.002 | 0.004 | 0.027 | 0.003 | 0.008 | 0.001 | 0.024 |
| Retriever | | | | | | | | | | | | | | | | |
| Labrador | | 0.001 | 0.003 | 0.005 | 0.002 | 0.007 | 0.001 | 0.002 | 0.924 | 0.001 | 0.002 | 0.002 | 0.003 | 0.001 | 0.002 | 0.002 |
| Retriever | | | | | | | | | | | | | | | | |
| Labrador | | 0.003 | 0.002 | 0.006 | 0.016 | 0.007 | 0.008 | 0.003 | 0.725 | 0.005 | 0.041 | 0.006 | 0.002 | 0.002 | 0.002 | 0.04 |
| Retriever | | | | | | | | | | | | | | | | |
| Labrador | | 0.002 | 0.003 | 0.011 | 0.015 | 0.016 | 0.004 | 0.004 | 0.743 | 0.017 | 0.024 | 0.015 | 0.003 | 0.005 | 0.006 | 0.005 |
| Retriever | | | | | | | | | | | | | | | | |
| Labrador | | 0.009 | 0.003 | 0.003 | 0.011 | 0.031 | 0.013 | 0.062 | 0.44 | 0.02 | 0.055 | 0.002 | 0.001 | 0.015 | 0.017 | 0.006 |
| Retriever | | | | | | | | | | | | | | | | |
| Labrador | | 0.004 | 0.008 | 0.016 | 0.002 | 0.013 | 0.004 | 0.005 | 0.762 | 0.005 | 0.004 | 0.005 | 0.004 | 0.016 | 0.002 | 0.011 |
| Retriever | | | | | | | | | | | | | | | | |
| Labrador | | 0.006 | 0.049 | 0.006 | 0.006 | 0.003 | 0.003 | 0.003 | 0.813 | 0.006 | 0.001 | 0.006 | 0.004 | 0.007 | 0.01 | 0.003 |
| Retriever | | | | | | | | | | | | | | | | |
| Labrador | | 0.002 | 0.003 | 0.002 | 0.003 | 0.002 | 0.003 | 0.002 | 0.885 | 0.002 | 0.002 | 0.002 | 0.006 | 0.002 | 0.049 | 0.002 |
| Retriever | | | | | | | | | | | | | | | | |
| Labrador | | 0.002 | 0.007 | 0.004 | 0.004 | 0.006 | 0.002 | 0.007 | 0.879 | 0.003 | 0.01 | 0.001 | 0.007 | 0.008 | 0.008 | 0.001 |
| Retriever | | | | | | | | | | | | | | | | |
| Labrador | | 0.001 | 0.004 | 0.002 | 0.004 | 0.002 | 0.006 | 0.004 | 0.897 | 0.002 | 0.001 | 0.002 | 0.002 | 0.003 | 0.004 | 0.005 |
| Retriever | | | | | | | | | | | | | | | | |
| Labrador | | 0.003 | 0.004 | 0.052 | 0.006 | 0.002 | 0.007 | 0.015 | 0.668 | 0.003 | 0.016 | 0.008 | 0.003 | 0.004 | 0.002 | 0.016 |
| Retriever | | | | | | | | | | | | | | | | |
| Labrador | | 0.002 | 0.029 | 0.006 | 0.003 | 0.005 | 0.014 | 0.012 | 0.83 | 0.003 | 0.003 | 0.002 | 0.006 | 0.002 | 0.007 | 0.004 |
| Retriever | | | | | | | | | | | | | | | | |
| Labrador | | 0.002 | 0.012 | 0.002 | 0.003 | 0.003 | 0.015 | 0.003 | 0.875 | 0.003 | 0.003 | 0.002 | 0.004 | 0.002 | 0.01 | 0.002 |
| Retriever | | | | | | | | | | | | | | | | |
| Labrador | | 0.001 | 0.009 | 0.005 | 0.013 | 0.001 | 0.002 | 0.003 | 0.797 | 0.002 | 0.004 | 0.005 | 0.004 | 0.023 | 0.005 | 0.003 |
| Retriever | | | | | | | | | | | | | | | | |
| Labrador | | 0.002 | 0.002 | 0.003 | 0.005 | 0.018 | 0.007 | 0.005 | 0.7 | 0.029 | 0.009 | 0.005 | 0.004 | 0.007 | 0.003 | 0.005 |
| Retriever | | | | | | | | | | | | | | | | |
| Labrador | | 0.001 | 0.002 | 0.003 | 0.002 | 0.004 | 0.01 | 0.004 | 0.874 | 0.003 | 0.005 | 0.006 | 0.003 | 0.002 | 0.002 | 0.004 |
| Retriever | | | | | | | | | | | | | | | | |
| Labrador | | 0.001 | 0.001 | 0.006 | 0.003 | 0.003 | 0.004 | 0.009 | 0.883 | 0.003 | 0.003 | 0.005 | 0.002 | 0.002 | 0.007 | 0.007 |
| Retriever | | | | | | | | | | | | | | | | |
| Labrador | | 0.001 | 0.001 | 0.001 | 0.002 | 0.001 | 0.002 | 0.002 | 0.964 | 0.001 | 0.001 | 0.002 | 0.001 | 0.001 | 0.002 | 0.001 |
| Retriever | | | | | | | | | | | | | | | | |
| Labrador | | 0.001 | 0.001 | 0.002 | 0.002 | 0.001 | 0.002 | 0.004 | 0.941 | 0.001 | 0.001 | 0.001 | 0.001 | 0.002 | 0.003 | 0.001 |
| Retriever | | | | | | | | | | | | | | | | |
| Labrador | | 0.003 | 0.007 | 0.007 | 0.053 | 0.008 | 0.018 | 0.002 | 0.719 | 0.012 | 0.008 | 0.009 | 0.02 | 0.002 | 0.002 | 0.011 |
| Retriever | | | | | | | | | | | | | | | | |
| Labrador | | 0.001 | 0.001 | 0.002 | 0.002 | 0.001 | 0.001 | 0.002 | 0.902 | 0.002 | 0.002 | 0.007 | 0.003 | 0.002 | 0.004 | 0.005 |
| Retriever | | | | | | | | | | | | | | | | |
| Labrador | | 0.001 | 0.003 | 0.002 | 0.001 | 0.002 | 0.001 | 0.003 | 0.932 | 0.002 | 0.004 | 0.005 | 0.003 | 0.002 | 0.002 | 0.003 |
| Retriever | | | | | | | | | | | | | | | | |
| Labrador | | 0.002 | 0.003 | 0.004 | 0.004 | 0.001 | 0.005 | 0.006 | 0.857 | 0.016 | 0.002 | 0.002 | 0.002 | 0.003 | 0.013 | 0.004 |
| Retriever | | | | | | | | | | | | | | | | |
| Labrador | | 0.001 | 0.004 | 0.012 | 0.052 | 0.014 | 0.017 | 0.003 | 0.696 | 0.043 | 0.003 | 0.002 | 0.003 | 0.013 | 0.024 | 0.009 |
| Retriever | | | | | | | | | | | | | | | | |
| Labrador | | 0.001 | 0.008 | 0.008 | 0.002 | 0.001 | 0.002 | 0.002 | 0.916 | 0.004 | 0.002 | 0.002 | 0.004 | 0.001 | 0.003 | 0.005 |
| Retriever | | | | | | | | | | | | | | | | |
| Labrador | | 0.001 | 0.003 | 0.002 | 0.002 | 0.001 | 0.003 | 0.003 | 0.932 | 0.001 | 0.002 | 0.003 | 0.003 | 0.003 | 0.005 | 0.003 |
| Retriever | | | | | | | | | | | | | | | | |
| Labrador | | 0.057 | 0.005 | 0.002 | 0.008 | 0.001 | 0.02 | 0.004 | 0.623 | 0.003 | 0.004 | 0.032 | 0.027 | 0.002 | 0.005 | 0.009 |
| Retriever | | | | | | | | | | | | | | | | |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Mastiff | (4) | 0.001 | 0.002 | 0.003 | 0.001 | 0.001 | 0.002 | 0.001 | 0.004 | 0.001 | 0.002 | 0.001 | 0.001 | 0.002 | 0.003 | 0.002 |
| Mastiff | 0 | 0.005 | 0.003 | 0.013 | 0.009 | 0.002 | 0.005 | 0.004 | 0.012 | 0.006 | 0.002 | 0.002 | 0.006 | 0.002 | 0.003 | 0.002 |
| Mastiff | 0 | 0.001 | 0.001 | 0.003 | 0.001 | 0.001 | 0.001 | 0.001 | 0.002 | 0.001 | 0.002 | 0.002 | 0.001 | 0.002 | 0.002 | 0.001 |
| Mastiff | 0 | 0.001 | 0.001 | 0.001 | 0.001 | 0.001 | 0.001 | 0.002 | 0.001 | 0.001 | 0.001 | 0.001 | 0.001 | 0.001 | 0.002 | 0.001 |
| Mastiff | 0 | 0.001 | 0.003 | 0.002 | 0.003 | 0.002 | 0.004 | 0.003 | 0.005 | 0.001 | 0.004 | 0.002 | 0.001 | 0.003 | 0.001 | 0.001 |
| Mastiff | (14) | 0.005 | 0.002 | 0.003 | 0.003 | 0.001 | 0.002 | 0.002 | 0.001 | 0.002 | 0.002 | 0.002 | 0.001 | 0.002 | 0.002 | 0.002 |
| Mastiff | 0 | 0.058 | 0.009 | 0.005 | 0.009 | 0.002 | 0.005 | 0.001 | 0.002 | 0.001 | 0.004 | 0.006 | 0.001 | 0.005 | 0.003 | 0.015 |
| Mastiff | 0 | 0.002 | 0.002 | 0.001 | 0.001 | 0.001 | 0.002 | 0.002 | 0.002 | 0.002 | 0.002 | 0.002 | 0.001 | 0.007 | 0.002 | 0.001 |
| Mastiff | 0 | 0.003 | 0.002 | 0.001 | 0.001 | 0.001 | 0.002 | 0.002 | 0.002 | 0.002 | 0.003 | 0.001 | 0.001 | 0.002 | 0.002 | 0.001 |
| Mastiff | 0 | 0.002 | 0.002 | 0.002 | 0.002 | 0.001 | 0.001 | 0.001 | 0.004 | 0.001 | 0.002 | 0.002 | 0.002 | 0.003 | 0.003 | 0.001 |
| Mastiff | 0 | 0.001 | 0.003 | 0.002 | 0.002 | 0.001 | 0.002 | 0.014 | 0.008 | 0.008 | 0.002 | 0.011 | 0.004 | 0.003 | 0.002 | 0.002 |
| Mastiff | 0 | 0.004 | 0.003 | 0.003 | 0.002 | 0.006 | 0.003 | 0.002 | 0.003 | 0.002 | 0.002 | 0.003 | 0.004 | 0.024 | 0.003 | 0.005 |
| Mastiff | 0 | 0.004 | 0.003 | 0.002 | 0.001 | 0.005 | 0.002 | 0.003 | 0.003 | 0.001 | 0.002 | 0.003 | 0.012 | 0.005 | 0.003 | 0.002 |
| Mastiff | 0 | 0.003 | 0.003 | 0.005 | 0.002 | 0.003 | 0.001 | 0.002 | 0.003 | 0.001 | 0.002 | 0.003 | 0.006 | 0.004 | 0.002 | 0.002 |
| Mastiff | (1) | 0.003 | 0.002 | 0.004 | 0.001 | 0.011 | 0.002 | 0.001 | 0.002 | 0.003 | 0.003 | 0.002 | 0.003 | 0.002 | 0.009 | 0.015 |
| Mastiff | 0 | 0.009 | 0.003 | 0.003 | 0.005 | 0.003 | 0.003 | 0.016 | 0.009 | 0.003 | 0.002 | 0.002 | 0.01 | 0.008 | 0.005 | 0.002 |
| Mastiff | (33) | 0.012 | 0.004 | 0.009 | 0.004 | 0.001 | 0.01 | 0.008 | 0.014 | 0.003 | 0.002 | 0.005 | 0.002 | 0.008 | 0.001 | 0.004 |
| Mastiff | (5) | 0.006 | 0.009 | 0.004 | 0.006 | 0.004 | 0.004 | 0.005 | 0.004 | 0.002 | 0.001 | 0.009 | 0.004 | 0.002 | 0.005 | 0.004 |
| Mastiff | 0 | 0.002 | 0.001 | 0.002 | 0.002 | 0.002 | 0.004 | 0.004 | 0.004 | 0.002 | 0.001 | 0.002 | 0.001 | 0.003 | 0.005 | 0.001 |
| Mastiff | 0 | 0.176 | 0.003 | 0.003 | 0.002 | 0.001 | 0.003 | 0.005 | 0.003 | 0.003 | 0.002 | 0.002 | 0.002 | 0.002 | 0.001 | 0.002 |
| Mastiff | 0 | 0.002 | 0.001 | 0.001 | 0.002 | 0.001 | 0.001 | 0.001 | 0.002 | 0.002 | 0.001 | 0.002 | 0.001 | 0.002 | 0.002 | 0.002 |
| Mastiff | (4) | 0.044 | 0.01 | 0.006 | 0.002 | 0.007 | 0.005 | 0.008 | 0.006 | 0.005 | 0.002 | 0.007 | 0.003 | 0.003 | 0.002 | 0.005 |
| Mastiff | 0 | 0.016 | 0.002 | 0.039 | 0.007 | 0.005 | 0.003 | 0.004 | 0.005 | 0.002 | 0.002 | 0.019 | 0.003 | 0.005 | 0.002 | 0.008 |
| Mastiff | 0 | 0.004 | 0.002 | 0.001 | 0.002 | 0.001 | 0.002 | 0.002 | 0.002 | 0.001 | 0.002 | 0.002 | 0.001 | 0.002 | 0.007 | 0.002 |
| Mastiff | (4) | 0.002 | 0.002 | 0.003 | 0.001 | 0.001 | 0.001 | 0.001 | 0.002 | 0.002 | 0.002 | 0.003 | 0.002 | 0.002 | 0.001 | 0.002 |
| Mastiff | 0 | 0.001 | 0.001 | 0.001 | 0.002 | 0.001 | 0.005 | 0.02 | 0.021 | 0.005 | 0.004 | 0.011 | 0.001 | 0.004 | 0.065 | 0.002 |
| Mastiff | 0 | 0.026 | 0.011 | 0.003 | 0.002 | 0.007 | 0.003 | 0.032 | 0.01 | 0.018 | 0.008 | 0.004 | 0.002 | 0.003 | 0.005 | 0.007 |
| Mastiff | 0 | 0.002 | 0.001 | 0.002 | 0.002 | 0.001 | 0.001 | 0.002 | 0.002 | 0.001 | 0.001 | 0.002 | 0.001 | 0.002 | 0.002 | 0.001 |
| Mastiff | 0 | 0.005 | 0.006 | 0.004 | 0.003 | 0.004 | 0.003 | 0.006 | 0.01 | 0.006 | 0.017 | 0.01 | 0.002 | 0.003 | 0.006 | 0.006 |
| Mastiff | 0 | 0.005 | 0.002 | 0.013 | 0.004 | 0.005 | 0.002 | 0.004 | 0.003 | 0.009 | 0.009 | 0.005 | 0.001 | 0.005 | 0.004 | 0.006 |
| Mastiff | (1) | 0.002 | 0.002 | 0.004 | 0.003 | 0.004 | 0.003 | 0.002 | 0.003 | 0.003 | 0.008 | 0.002 | 0.001 | 0.005 | 0.003 | 0.004 |
| Mastiff | 0 | 0.002 | 0.002 | 0.001 | 0.002 | 0.001 | 0.002 | 0.003 | 0.004 | 0.002 | 0.005 | 0.002 | 0.001 | 0.008 | 0.001 | 0.002 |
| Mastiff | (6) | 0.002 | 0.002 | 0.001 | 0.001 | 0.001 | 0.002 | 0.002 | 0.002 | 0.001 | 0.002 | 0.004 | 0.001 | 0.003 | 0.003 | 0.002 |
| Mastiff | 0 | 0.006 | 0.001 | 0.001 | 0.002 | 0.001 | 0.001 | 0.002 | 0.004 | 0.003 | 0.002 | 0.004 | 0.002 | 0.006 | 0.001 | 0.002 |
| Mastiff | 0 | 0.002 | 0.003 | 0.001 | 0.002 | 0.001 | 0.001 | 0.002 | 0.002 | 0.078 | 0.007 | 0.002 | 0.002 | 0.006 | 0.003 | 0.001 |
| Mastiff | 0 | 0.011 | 0.006 | 0.003 | 0.004 | 0.003 | 0.004 | 0.002 | 0.003 | 0.01 | 0.014 | 0.009 | 0.001 | 0.002 | 0.004 | 0.004 |
| Mastiff | 0 | 0.002 | 0.011 | 0.003 | 0.003 | 0.002 | 0.002 | 0.004 | 0.003 | 0.006 | 0.002 | 0.008 | 0.002 | 0.003 | 0.004 | 0.004 |
| Mastiff | (2) | 0.005 | 0.002 | 0.002 | 0.002 | 0.001 | 0.002 | 0.003 | 0.01 | 0.002 | 0.002 | 0.007 | 0.001 | 0.005 | 0.005 | 0.004 |
| | | Cluster Assignment | | | | | | | | | | | | | | |
| Breed | | 16 | 17 | 18 | 19 | 20 | 21 | 22 | 23 | 24 | 25 | 26 | 27 | 28 | 29 | 30 |
| Mastiff | | 0.001 | 0.003 | 0.002 | 0.002 | 0.003 | 0.002 | 0.003 | 0.001 | 0.001 | 0.002 | 0.002 | 0.938 | 0.007 | 0.001 | 0.002 |
| Mastiff | | 0.001 | 0.003 | 0.003 | 0.002 | 0.002 | 0.002 | 0.003 | 0.004 | 0.002 | 0.006 | 0.002 | 0.882 | 0.007 | 0.003 | 0.003 |
| Mastiff | | 0.001 | 0.001 | 0.001 | 0.001 | 0.002 | 0.001 | 0.002 | 0.001 | 0.001 | 0.003 | 0.002 | 0.955 | 0.002 | 0.002 | 0.001 |
| Mastiff | | 0.001 | 0.002 | 0.001 | 0.001 | 0.001 | 0.001 | 0.001 | 0.001 | 0.001 | 0.001 | 0.001 | 0.967 | 0.001 | 0.001 | 0.001 |
| Mastiff | | 0.002 | 0.002 | 0.005 | 0.001 | 0.002 | 0.003 | 0.002 | 0.002 | 0.002 | 0.004 | 0.004 | 0.928 | 0.003 | 0.001 | 0.002 |
| Mastiff | | 0.001 | 0.003 | 0.002 | 0.002 | 0.002 | 0.001 | 0.003 | 0.003 | 0.003 | 0.002 | 0.002 | 0.938 | 0.002 | 0.003 | 0.003 |
| Mastiff | | 0.002 | 0.002 | 0.004 | 0.006 | 0.002 | 0.003 | 0.007 | 0.016 | 0.012 | 0.002 | 0.002 | 0.808 | 0.003 | 0.003 | 0.003 |
| Mastiff | | 0.003 | 0.002 | 0.002 | 0.001 | 0.003 | 0.002 | 0.002 | 0.002 | 0.001 | 0.004 | 0.004 | 0.941 | 0.003 | 0.001 | 0.001 |
| Mastiff | | 0.001 | 0.002 | 0.002 | 0.001 | 0.001 | 0.001 | 0.003 | 0.002 | 0.002 | 0.002 | 0.002 | 0.952 | 0.001 | 0.001 | 0.002 |
| Mastiff | | 0.001 | 0.002 | 0.002 | 0.001 | 0.001 | 0.001 | 0.002 | 0.001 | 0.002 | 0.004 | 0.002 | 0.945 | 0.001 | 0.004 | 0.003 |
| Mastiff | | 0.002 | 0.013 | 0.005 | 0.003 | 0.003 | 0.006 | 0.01 | 0.003 | 0.002 | 0.012 | 0.002 | 0.859 | 0.004 | 0.01 | 0.003 |
| Mastiff | | 0.004 | 0.004 | 0.004 | 0.007 | 0.002 | 0.005 | 0.004 | 0.002 | 0.014 | 0.005 | 0.003 | 0.857 | 0.003 | 0.002 | 0.014 |
| Mastiff | | 0.001 | 0.004 | 0.001 | 0.006 | 0.001 | 0.004 | 0.003 | 0.003 | 0.014 | 0.002 | 0.001 | 0.895 | 0.001 | 0.002 | 0.01 |
| Mastiff | | 0.002 | 0.008 | 0.003 | 0.003 | 0.001 | 0.002 | 0.002 | 0.001 | 0.004 | 0.002 | 0.001 | 0.917 | 0.001 | 0.005 | 0.005 |
| Mastiff | | 0.005 | 0.002 | 0.001 | 0.001 | 0.001 | 0.003 | 0.002 | 0.003 | 0.022 | 0.001 | 0.001 | 0.857 | 0.001 | 0.029 | 0.007 |
| Mastiff | | 0.016 | 0.016 | 0.008 | 0.001 | 0.002 | 0.004 | 0.014 | 0.006 | 0.002 | 0.013 | 0.006 | 0.812 | 0.003 | 0.002 | 0.013 |
| Mastiff | | 0.003 | 0.006 | 0.006 | 0.007 | 0.007 | 0.001 | 0.007 | 0.004 | 0.005 | 0.015 | 0.002 | 0.84 | 0.005 | 0.003 | 0.002 |
| Mastiff | | 0.002 | 0.004 | 0.009 | 0.003 | 0.003 | 0.002 | 0.003 | 0.005 | 0.022 | 0.001 | 0.002 | 0.846 | 0.003 | 0.022 | 0.002 |
| Mastiff | | 0.002 | 0.003 | 0.005 | 0.002 | 0.003 | 0.002 | 0.002 | 0.001 | 0.003 | 0.001 | 0.001 | 0.931 | 0.003 | 0.001 | 0.002 |
| Mastiff | | 0.002 | 0.001 | 0.002 | 0.001 | 0.001 | 0.003 | 0.001 | 0.002 | 0.002 | 0.003 | 0.001 | 0.762 | 0.005 | 0.001 | 0.002 |
| Mastiff | | 0.001 | 0.001 | 0.001 | 0.001 | 0.001 | 0.002 | 0.002 | 0.001 | 0.001 | 0.004 | 0.001 | 0.955 | 0.002 | 0.002 | 0.001 |
| Mastiff | | 0.008 | 0.019 | 0.009 | 0.005 | 0.01 | 0.009 | 0.005 | 0.004 | 0.025 | 0.006 | 0.002 | 0.764 | 0.003 | 0.001 | 0.016 |
| Mastiff | | 0.001 | 0.009 | 0.005 | 0.007 | 0.002 | 0.003 | 0.011 | 0.003 | 0.003 | 0.002 | 0.001 | 0.804 | 0.007 | 0.014 | 0.004 |
| Mastiff | | 0.001 | 0.005 | 0.002 | 0.001 | 0.002 | 0.001 | 0.005 | 0.002 | 0.005 | 0.005 | 0.004 | 0.924 | 0.002 | 0.007 | 0.003 |
| Mastiff | | 0.002 | 0.004 | 0.002 | 0.001 | 0.001 | 0.002 | 0.002 | 0.002 | 0.001 | 0.002 | 0.003 | 0.948 | 0.001 | 0.002 | 0.002 |
| Mastiff | | 0.002 | 0.017 | 0.015 | 0.001 | 0.003 | 0.003 | 0.006 | 0.004 | 0.005 | 0.005 | 0.005 | 0.772 | 0.013 | 0.003 | 0.002 |
| Mastiff | | 0.002 | 0.001 | 0.003 | 0.001 | 0.002 | 0.004 | 0.003 | 0.004 | 0.009 | 0.016 | 0.002 | 0.779 | 0.01 | 0.013 | 0.01 |
| Mastiff | | 0.003 | 0.005 | 0.001 | 0.001 | 0.001 | 0.001 | 0.001 | 0.002 | 0.001 | 0.002 | 0.002 | 0.954 | 0.001 | 0.001 | 0.001 |
| Mastiff | | 0.007 | 0.031 | 0.004 | 0.001 | 0.001 | 0.008 | 0.001 | 0.004 | 0.004 | 0.005 | 0.004 | 0.806 | 0.027 | 0.004 | 0.002 |
| Mastiff | | 0.002 | 0.003 | 0.001 | 0.002 | 0.004 | 0.006 | 0.002 | 0.005 | 0.003 | 0.002 | 0.002 | 0.834 | 0.049 | 0.005 | 0.002 |
| Mastiff | | 0.002 | 0.03 | 0.004 | 0.003 | 0.01 | 0.005 | 0.005 | 0.003 | 0.004 | 0.002 | 0.002 | 0.874 | 0.003 | 0.002 | 0.004 |
| Mastiff | | 0.001 | 0.005 | 0.002 | 0.001 | 0.002 | 0.003 | 0.002 | 0.002 | 0.001 | 0.004 | 0.004 | 0.931 | 0.003 | 0.001 | 0.002 |
| Mastiff | | 0.001 | 0.002 | 0.003 | 0.001 | 0.001 | 0.002 | 0.001 | 0.001 | 0.003 | 0.004 | 0.002 | 0.943 | 0.001 | 0.003 | 0.001 |
| Mastiff | | 0.001 | 0.003 | 0.002 | 0.001 | 0.002 | 0.001 | 0.002 | 0.001 | 0.001 | 0.003 | 0.004 | 0.937 | 0.001 | 0.002 | 0.002 |
| Mastiff | | 0.001 | 0.002 | 0.002 | 0.002 | 0.001 | 0.002 | 0.001 | 0.032 | 0.001 | 0.005 | 0.009 | 0.826 | 0.001 | 0.002 | 0.001 |
| Mastiff | | 0.004 | 0.001 | 0.002 | 0.004 | 0.003 | 0.002 | 0.002 | 0.002 | 0.004 | 0.01 | 0.001 | 0.877 | 0.002 | 0.002 | 0.002 |
| Mastiff | | 0.001 | 0.004 | 0.005 | 0.003 | 0.002 | 0.004 | 0.039 | 0.002 | 0.004 | 0.01 | 0.002 | 0.835 | 0.011 | 0.013 | 0.005 |
| Mastiff | | 0.001 | 0.004 | 0.013 | 0.001 | 0.003 | 0.004 | 0.022 | 0.006 | 0.002 | 0.004 | 0.006 | 0.873 | 0.001 | 0.002 | 0.004 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Miniature | 0 | 0.003 | 0.002 | 0.002 | 0.003 | 0.001 | 0.001 | 0.001 | 0.001 | 0.003 | 0.936 | 0.002 | 0.002 | 0.005 | 0.002 | 0.001 |
| Schnauzer | | | | | | | | | | | | | | | | |
| Miniature | (7) | 0.003 | 0.002 | 0.001 | 0.002 | 0.001 | 0.002 | 0.002 | 0.005 | 0.002 | 0.925 | 0.003 | 0.002 | 0.002 | 0.002 | 0.002 |
| Schnauzer | | | | | | | | | | | | | | | | |
| Miniature | (1) | 0.002 | 0.004 | 0.003 | 0.004 | 0.001 | 0.002 | 0.006 | 0.011 | 0.004 | 0.874 | 0.003 | 0.003 | 0.003 | 0.01 | 0.011 |
| Schnauzer | | | | | | | | | | | | | | | | |
| Miniature | 0 | 0.001 | 0.001 | 0.001 | 0.002 | 0.001 | 0.002 | 0.002 | 0.001 | 0.002 | 0.934 | 0.002 | 0.001 | 0.002 | 0.004 | 0.002 |
| Schnauzer | | | | | | | | | | | | | | | | |
| Miniature | 0 | 0.002 | 0.001 | 0.004 | 0.003 | 0.001 | 0.004 | 0.002 | 0.002 | 0.002 | 0.912 | 0.004 | 0.001 | 0.002 | 0.001 | 0.003 |
| Schnauzer | | | | | | | | | | | | | | | | |
| Miniature | (1) | 0.004 | 0.002 | 0.007 | 0.008 | 0.003 | 0.016 | 0.002 | 0.001 | 0.007 | 0.87 | 0.013 | 0.003 | 0.009 | 0.002 | 0.004 |
| Schnauzer | | | | | | | | | | | | | | | | |
| Miniature | 0 | 0.004 | 0.005 | 0.003 | 0.003 | 0.001 | 0.003 | 0.003 | 0.002 | 0.001 | 0.75 | 0.004 | 0.001 | 0.006 | 0.003 | 0.002 |
| Schnauzer | | | | | | | | | | | | | | | | |
| Miniature | 0 | 0.013 | 0.002 | 0.001 | 0.001 | 0.001 | 0.007 | 0.006 | 0.004 | 0.002 | 0.877 | 0.021 | 0.003 | 0.02 | 0.001 | 0.002 |
| Schnauzer | | | | | | | | | | | | | | | | |
| Miniature | 0 | 0.001 | 0.003 | 0.001 | 0.001 | 0.001 | 0.002 | 0.002 | 0.002 | 0.002 | 0.957 | 0.001 | 0.005 | 0.001 | 0.004 | 0.002 |
| Schnauzer | | | | | | | | | | | | | | | | |
| Miniature | 0 | 0.001 | 0.002 | 0.001 | 0.001 | 0.004 | 0.003 | 0.002 | 0.003 | 0.003 | 0.84 | 0.002 | 0.005 | 0.002 | 0.008 | 0.001 |
| Schnauzer | | | | | | | | | | | | | | | | |
| Miniature | (2) | 0.002 | 0.002 | 0.004 | 0.003 | 0.004 | 0.002 | 0.003 | 0.003 | 0.002 | 0.911 | 0.006 | 0.001 | 0.003 | 0.011 | 0.003 |
| Schnauzer | | | | | | | | | | | | | | | | |
| Miniature | (44) | 0.002 | 0.002 | 0.001 | 0.001 | 0.001 | 0.002 | 0.001 | 0.002 | 0.001 | 0.948 | 0.001 | 0.001 | 0.002 | 0.003 | 0.002 |
| Schnauzer | | | | | | | | | | | | | | | | |
| Miniature | 0 | 0.001 | 0.002 | 0.002 | 0.002 | 0.001 | 0.001 | 0.001 | 0.001 | 0.002 | 0.962 | 0.001 | 0.001 | 0.002 | 0.001 | 0.001 |
| Schnauzer | | | | | | | | | | | | | | | | |
| Miniature | 0 | 0.001 | 0.001 | 0.001 | 0.001 | 0.001 | 0.001 | 0.002 | 0.003 | 0.002 | 0.956 | 0.001 | 0.001 | 0.002 | 0.002 | 0.001 |
| Schnauzer | | | | | | | | | | | | | | | | |
| Miniature | 0 | 0.001 | 0.001 | 0.001 | 0.001 | 0.001 | 0.001 | 0.002 | 0.003 | 0.002 | 0.962 | 0.001 | 0.002 | 0.001 | 0.001 | 0.001 |
| Schnauzer | | | | | | | | | | | | | | | | |
| Miniature | 0 | 0.002 | 0.002 | 0.006 | 0.004 | 0.001 | 0.002 | 0.004 | 0.003 | 0.003 | 0.886 | 0.002 | 0.004 | 0.002 | 0.004 | 0.002 |
| Schnauzer | | | | | | | | | | | | | | | | |
| Miniature | 0 | 0.016 | 0.002 | 0.002 | 0.001 | 0.003 | 0.016 | 0.011 | 0.048 | 0.002 | 0.743 | 0.075 | 0.003 | 0.005 | 0.009 | 0.003 |
| Schnauzer | | | | | | | | | | | | | | | | |
| Miniature | 0 | 0.003 | 0.002 | 0.004 | 0.003 | 0.002 | 0.002 | 0.004 | 0.009 | 0.005 | 0.896 | 0.007 | 0.001 | 0.002 | 0.002 | 0.002 |
| Schnauzer | | | | | | | | | | | | | | | | |
| Miniature | 0 | 0.001 | 0.004 | 0.002 | 0.001 | 0.001 | 0.001 | 0.002 | 0.003 | 0.003 | 0.944 | 0.002 | 0.001 | 0.003 | 0.003 | 0.001 |
| Schnauzer | | | | | | | | | | | | | | | | |
| Miniature | 0 | 0.001 | 0.001 | 0.001 | 0.002 | 0.001 | 0.001 | 0.001 | 0.001 | 0.001 | 0.962 | 0.001 | 0.001 | 0.002 | 0.002 | 0.002 |
| Schnauzer | | | | | | | | | | | | | | | | |
| Miniature | 0 | 0.003 | 0.003 | 0.004 | 0.006 | 0.005 | 0.002 | 0.003 | 0.007 | 0.002 | 0.863 | 0.003 | 0.002 | 0.002 | 0.002 | 0.008 |
| Schnauzer | | | | | | | | | | | | | | | | |
| Miniature | (2) | 0.001 | 0.002 | 0.001 | 0.002 | 0.006 | 0.003 | 0.002 | 0.01 | 0.003 | 0.884 | 0.004 | 0.006 | 0.001 | 0.009 | 0.003 |
| Schnauzer | | | | | | | | | | | | | | | | |
| Miniature | 0 | 0.001 | 0.001 | 0.002 | 0.001 | 0.001 | 0.001 | 0.002 | 0.001 | 0.001 | 0.958 | 0.001 | 0.001 | 0.002 | 0.002 | 0.002 |
| Schnauzer | | | | | | | | | | | | | | | | |
| Miniature | 0 | 0.001 | 0.001 | 0.001 | 0.001 | 0 | 0.001 | 0.001 | 0.001 | 0.001 | 0.969 | 0.001 | 0.001 | 0.001 | 0.002 | 0.001 |
| Schnauzer | | | | | | | | | | | | | | | | |
| Miniature | (1) | 0.001 | 0.001 | 0.001 | 0.001 | 0 | 0.001 | 0.001 | 0.001 | 0.001 | 0.977 | 0.001 | 0.001 | 0.001 | 0.001 | 0.001 |
| Schnauzer | | | | | | | | | | | | | | | | |
| Miniature | 0 | 0.003 | 0.003 | 0.003 | 0.007 | 0.001 | 0.002 | 0.002 | 0.001 | 0.001 | 0.896 | 0.001 | 0.002 | 0.002 | 0.001 | 0.004 |
| Schnauzer | | | | | | | | | | | | | | | | |
| Miniature | 0 | 0.002 | 0.005 | 0.001 | 0.003 | 0.002 | 0.002 | 0.002 | 0.001 | 0.001 | 0.901 | 0.001 | 0.005 | 0.002 | 0.001 | 0.003 |
| Schnauzer | | | | | | | | | | | | | | | | |
| Miniature | 0 | 0.005 | 0.005 | 0.006 | 0.002 | 0.002 | 0.006 | 0.005 | 0.005 | 0.003 | 0.87 | 0.003 | 0.002 | 0.002 | 0.002 | 0.007 |
| Schnauzer | | | | | | | | | | | | | | | | |
| Miniature | 0 | 0.001 | 0.002 | 0.001 | 0.001 | 0.001 | 0.001 | 0.002 | 0.001 | 0.001 | 0.964 | 0.001 | 0.002 | 0.002 | 0.001 | 0.001 |
| Schnauzer | | | | | | | | | | | | | | | | |
| Miniature | (1) | 0.001 | 0.001 | 0.001 | 0.001 | 0.001 | 0.001 | 0.001 | 0.001 | 0.001 | 0.973 | 0.001 | 0.001 | 0.001 | 0.001 | 0.001 |
| Schnauzer | | | | | | | | | | | | | | | | |
| Miniature | 0 | 0.002 | 0.002 | 0.001 | 0.002 | 0.001 | 0.001 | 0.001 | 0.002 | 0.003 | 0.943 | 0.002 | 0.001 | 0.003 | 0.002 | 0.002 |
| Schnauzer | | | | | | | | | | | | | | | | |
| Miniature | 0 | 0.002 | 0.001 | 0.001 | 0.001 | 0.001 | 0.001 | 0.001 | 0.001 | 0.002 | 0.964 | 0.001 | 0.002 | 0.002 | 0.001 | 0.001 |
| Schnauzer | | | | | | | | | | | | | | | | |
| Miniature | 0 | 0.002 | 0.002 | 0.001 | 0.001 | 0.001 | 0.001 | 0.001 | 0.002 | 0.001 | 0.952 | 0.002 | 0.002 | 0.002 | 0.001 | 0.002 |
| Schnauzer | | | | | | | | | | | | | | | | |
| Miniature | 0 | 0.001 | 0.001 | 0.005 | 0.001 | 0.002 | 0.001 | 0.002 | 0.002 | 0.002 | 0.948 | 0.003 | 0.001 | 0.002 | 0.002 | 0.002 |
| Schnauzer | | | | | | | | | | | | | | | | |
| Miniature | 0 | 0.002 | 0.001 | 0.001 | 0.002 | 0.009 | 0.004 | 0.003 | 0.006 | 0.001 | 0.904 | 0.002 | 0.003 | 0.006 | 0.005 | 0.003 |
| Schnauzer | | | | | | | | | | | | | | | | |
| Miniature | 0 | 0.002 | 0.002 | 0.002 | 0.003 | 0.001 | 0.002 | 0.003 | 0.007 | 0.001 | 0.854 | 0.004 | 0.001 | 0.003 | 0.01 | 0.002 |
| Schnauzer | | | | | | | | | | | | | | | | |
| Miniature | 0 | 0.001 | 0.002 | 0.002 | 0.001 | 0.002 | 0.001 | 0.002 | 0.003 | 0.001 | 0.945 | 0.003 | 0.002 | 0.001 | 0.006 | 0.001 |
| Schnauzer | | | | | | | | | | | | | | | | |
| Miniature | (1) | 0.003 | 0.002 | 0.002 | 0.005 | 0.002 | 0.002 | 0.002 | 0.003 | 0.008 | 0.917 | 0.003 | 0.005 | 0.003 | 0.003 | 0.001 |
| Schnauzer | | | | | | | | | | | | | | | | |
| | | Cluster Assignment | | | | | | | | | | | | | | |
| Breed | | 16 | 17 | 18 | 19 | 20 | 21 | 22 | 23 | 24 | 25 | 26 | 27 | 28 | 29 | 30 |
| Miniature | | 0.001 | 0.002 | 0.002 | 0.005 | 0.002 | 0.002 | 0.002 | 0.006 | 0.001 | 0.001 | 0.003 | 0.001 | 0.002 | 0.001 | 0.001 |
| Schnauzer | | | | | | | | | | | | | | | | |
| Miniature | | 0.003 | 0.003 | 0.002 | 0.003 | 0.001 | 0.003 | 0.003 | 0.002 | 0.002 | 0.005 | 0.007 | 0.003 | 0.003 | 0.002 | 0.003 |
| Schnauzer | | | | | | | | | | | | | | | | |
| Miniature | | 0.001 | 0.001 | 0.002 | 0.008 | 0.002 | 0.008 | 0.004 | 0.006 | 0.005 | 0.006 | 0.002 | 0.002 | 0.002 | 0.002 | 0.008 |
| Schnauzer | | | | | | | | | | | | | | | | |
| Miniature | | 0.001 | 0.002 | 0.002 | 0.008 | 0.001 | 0.002 | 0.004 | 0.004 | 0.002 | 0.003 | 0.001 | 0.002 | 0.001 | 0.004 | 0.002 |
| Schnauzer | | | | | | | | | | | | | | | | |
| Miniature | | 0.001 | 0.002 | 0.004 | 0.006 | 0.006 | 0.002 | 0.002 | 0.002 | 0.003 | 0.011 | 0.002 | 0.001 | 0.006 | 0.003 | 0.004 |
| Schnauzer | | | | | | | | | | | | | | | | |
| Miniature | | 0.002 | 0.002 | 0.004 | 0.008 | 0.009 | 0.003 | 0.001 | 0.002 | 0.004 | 0.001 | 0.003 | 0.002 | 0.005 | 0.001 | 0.002 |
| Schnauzer | | | | | | | | | | | | | | | | |
| Miniature | | 0.002 | 0.006 | 0.011 | 0.009 | 0.005 | 0.003 | 0.002 | 0.005 | 0.008 | 0.142 | 0.005 | 0.003 | 0.003 | 0.003 | 0.002 |
| Schnauzer | | | | | | | | | | | | | | | | |
| Miniature | | 0.003 | 0.002 | 0.004 | 0.004 | 0.005 | 0.002 | 0.001 | 0.001 | 0.003 | 0.006 | 0.001 | 0.004 | 0.001 | 0.002 | 0.002 |
| Schnauzer | | | | | | | | | | | | | | | | |
| Miniature | | 0 | 0.001 | 0.001 | 0.002 | 0.001 | 0.002 | 0.001 | 0.001 | 0.002 | 0.002 | 0.001 | 0.001 | 0.001 | 0.001 | 0.001 |
| Schnauzer | | | | | | | | | | | | | | | | |
| Miniature | | 0.001 | 0.005 | 0.002 | 0.001 | 0.002 | 0.003 | 0.007 | 0.006 | 0.014 | 0.001 | 0.002 | 0.002 | 0.003 | 0.071 | 0.001 |
| Schnauzer | | | | | | | | | | | | | | | | |
| Miniature | | 0.001 | 0.003 | 0.002 | 0.003 | 0.001 | 0.004 | 0.003 | 0.004 | 0.003 | 0.002 | 0.002 | 0.003 | 0.001 | 0.006 | 0.002 |
| Schnauzer | | | | | | | | | | | | | | | | |
| Miniature | | 0.001 | 0.002 | 0.002 | 0.001 | 0.004 | 0.002 | 0.003 | 0.002 | 0.002 | 0.002 | 0.002 | 0.002 | 0.001 | 0.002 | 0.002 |
| Schnauzer | | | | | | | | | | | | | | | | |
| Miniature | | 0.001 | 0.001 | 0.001 | 0.001 | 0.001 | 0.001 | 0.002 | 0.002 | 0.001 | 0.002 | 0.001 | 0.001 | 0.002 | 0.001 | 0.001 |
| Schnauzer | | | | | | | | | | | | | | | | |
| Miniature | | 0.001 | 0.003 | 0.001 | 0.001 | 0.002 | 0.001 | 0.004 | 0.002 | 0.001 | 0.001 | 0.002 | 0.001 | 0.001 | 0.001 | 0.002 |
| Schnauzer | | | | | | | | | | | | | | | | |
| Miniature | | 0.001 | 0.001 | 0.002 | 0.001 | 0.002 | 0.001 | 0.003 | 0.002 | 0.001 | 0.001 | 0.002 | 0.001 | 0.001 | 0.001 | 0.001 |
| Schnauzer | | | | | | | | | | | | | | | | |
| Miniature | | 0.002 | 0.006 | 0.004 | 0.005 | 0.003 | 0.004 | 0.012 | 0.007 | 0.002 | 0.003 | 0.014 | 0.002 | 0.001 | 0.003 | 0.003 |
| Schnauzer | | | | | | | | | | | | | | | | |
| Miniature | | 0.004 | 0.008 | 0.01 | 0.003 | 0.002 | 0.003 | 0.001 | 0.002 | 0.004 | 0.003 | 0.002 | 0.007 | 0.003 | 0.008 | 0.001 |
| Schnauzer | | | | | | | | | | | | | | | | |
| Miniature | | 0.002 | 0.005 | 0.003 | 0.007 | 0.006 | 0.003 | 0.002 | 0.003 | 0.002 | 0.008 | 0.006 | 0.003 | 0.002 | 0.002 | 0.003 |
| Schnauzer | | | | | | | | | | | | | | | | |
| Miniature | | 0.001 | 0.004 | 0.002 | 0.001 | 0.002 | 0.001 | 0.003 | 0.003 | 0.001 | 0.003 | 0.001 | 0.003 | 0.002 | 0.001 | 0.003 |
| Schnauzer | | | | | | | | | | | | | | | | |
| Miniature | | 0.001 | 0.001 | 0.002 | 0.002 | 0.001 | 0.001 | 0.001 | 0.001 | 0.001 | 0.003 | 0.001 | 0.001 | 0.001 | 0.001 | 0.001 |
| Schnauzer | | | | | | | | | | | | | | | | |
| Miniature | | 0.002 | 0.001 | 0.002 | 0.01 | 0.004 | 0.003 | 0.003 | 0.003 | 0.002 | 0.002 | 0.002 | 0.003 | 0.002 | 0.044 | 0.003 |
| Schnauzer | | | | | | | | | | | | | | | | |
| Miniature | | 0.002 | 0.002 | 0.005 | 0.003 | 0.002 | 0.002 | 0.003 | 0.001 | 0.005 | 0.004 | 0.006 | 0.002 | 0.008 | 0.017 | 0.002 |
| Schnauzer | | | | | | | | | | | | | | | | |
| Miniature | | 0.001 | 0.001 | 0.001 | 0.002 | 0.001 | 0.002 | 0.002 | 0.001 | 0.001 | 0.001 | 0.002 | 0.002 | 0.001 | 0.003 | 0.001 |
| Schnauzer | | | | | | | | | | | | | | | | |
| Miniature | | 0.001 | 0.002 | 0.001 | 0.001 | 0.001 | 0.001 | 0.002 | 0.001 | 0.001 | 0.001 | 0.001 | 0.001 | 0.001 | 0.001 | 0.001 |
| Schnauzer | | | | | | | | | | | | | | | | |
| Miniature | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Schnauzer | | | | | | | | | | | | | | | | |
| Miniature | | 0.001 | 0.003 | 0.001 | 0.002 | 0.001 | 0.004 | 0.003 | 0.035 | 0.002 | 0.002 | 0.002 | 0.001 | 0.002 | 0.006 | 0.006 |
| Schnauzer | | | | | | | | | | | | | | | | |
| Miniature | | 0.001 | 0.001 | 0.002 | 0.001 | 0.006 | 0.013 | 0.015 | 0.006 | 0.003 | 0.001 | 0.003 | 0.001 | 0.001 | 0.004 | 0.012 |
| Schnauzer | | | | | | | | | | | | | | | | |
| Miniature | | 0.002 | 0.002 | 0.004 | 0.008 | 0.001 | 0.003 | 0.008 | 0.002 | 0.002 | 0.001 | 0.029 | 0.002 | 0.002 | 0.003 | 0.007 |
| Schnauzer | | | | | | | | | | | | | | | | |
| Miniature | | 0.001 | 0.001 | 0.001 | 0.001 | 0.001 | 0.001 | 0.002 | 0.001 | 0.001 | 0.002 | 0.001 | 0.001 | 0.001 | 0.001 | 0.001 |
| Schnauzer | | | | | | | | | | | | | | | | |
| Miniature | | 0.001 | 0.001 | 0.001 | 0.001 | 0.001 | 0.001 | 0.002 | 0.001 | 0.001 | 0.001 | 0.001 | 0.001 | 0.001 | 0.001 | 0.001 |
| Schnauzer | | | | | | | | | | | | | | | | |
| Miniature | | 0.001 | 0.002 | 0.002 | 0.002 | 0.002 | 0.001 | 0.003 | 0.004 | 0.001 | 0.001 | 0.003 | 0.002 | 0.002 | 0.003 | 0.001 |
| Schnauzer | | | | | | | | | | | | | | | | |
| Miniature | | 0.001 | 0.001 | 0.001 | 0.001 | 0.001 | 0.001 | 0.002 | 0.002 | 0.001 | 0.001 | 0.001 | 0.002 | 0.001 | 0.001 | 0.001 |
| Schnauzer | | | | | | | | | | | | | | | | |
| Miniature | | 0.001 | 0.002 | 0.002 | 0.002 | 0.001 | 0.001 | 0.001 | 0.001 | 0.001 | 0.004 | 0.003 | 0.002 | 0.001 | 0.002 | 0.001 |
| Schnauzer | | | | | | | | | | | | | | | | |
| Miniature | | 0.001 | 0.002 | 0.002 | 0.002 | 0.002 | 0.002 | 0.001 | 0.001 | 0.002 | 0.001 | 0.003 | 0.001 | 0.002 | 0.001 | 0.001 |
| Schnauzer | | | | | | | | | | | | | | | | |
| Miniature | | 0.002 | 0.002 | 0.001 | 0.001 | 0.001 | 0.001 | 0.002 | 0.002 | 0.003 | 0.001 | 0.021 | 0.002 | 0.004 | 0.004 | 0.003 |
| Schnauzer | | | | | | | | | | | | | | | | |
| Miniature | | 0.002 | 0.011 | 0.003 | 0.005 | 0.002 | 0.004 | 0.004 | 0.011 | 0.004 | 0.005 | 0.04 | 0.002 | 0.003 | 0.005 | 0.002 |
| Schnauzer | | | | | | | | | | | | | | | | |
| Miniature | | 0.001 | 0.005 | 0.002 | 0.002 | 0.001 | 0.002 | 0.004 | 0.002 | 0.001 | 0.001 | 0.002 | 0.001 | 0.001 | 0.002 | 0.001 |
| Schnauzer | | | | | | | | | | | | | | | | |
| Miniature | | 0.001 | 0.001 | 0.003 | 0.005 | 0.002 | 0.002 | 0.003 | 0.003 | 0.002 | 0.003 | 0.004 | 0.003 | 0.004 | 0.003 | 0.001 |
| Schnauzer | | | | | | | | | | | | | | | | |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Poodle | (5) | 0.004 | 0.006 | 0.002 | 0.002 | 0.022 | 0.007 | 0.003 | 0.005 | 0.002 | 0.001 | 0.006 | 0.002 | 0.004 | 0.005 | 0.004 |
| Poodle | (1) | 0.003 | 0.002 | 0.002 | 0.004 | 0.02 | 0.01 | 0.002 | 0.003 | 0.007 | 0.001 | 0.008 | 0.001 | 0.003 | 0.001 | 0.003 |
| Poodle | (1) | 0.005 | 0.008 | 0.003 | 0.004 | 0.009 | 0.022 | 0.004 | 0.008 | 0.003 | 0.002 | 0.01 | 0.004 | 0.017 | 0.005 | 0.004 |
| Poodle | 0 | 0.007 | 0.004 | 0.002 | 0.005 | 0.002 | 0.024 | 0.004 | 0.002 | 0.003 | 0.002 | 0.003 | 0.002 | 0.006 | 0.002 | 0.001 |
| Poodle | 0 | 0.003 | 0.002 | 0.002 | 0.002 | 0.002 | 0.006 | 0.003 | 0.001 | 0.002 | 0.002 | 0.005 | 0.002 | 0.004 | 0.001 | 0.002 |
| Poodle | 0 | 0.007 | 0.008 | 0.004 | 0.006 | 0.046 | 0.018 | 0.005 | 0.007 | 0.003 | 0.006 | 0.016 | 0.003 | 0.063 | 0.006 | 0.002 |
| Poodle | 0 | 0.002 | 0.002 | 0.001 | 0.002 | 0.002 | 0.002 | 0.001 | 0.001 | 0.001 | 0.004 | 0.003 | 0.001 | 0.001 | 0.002 | 0.003 |
| Poodle | (1) | 0.01 | 0.022 | 0.001 | 0.012 | 0.001 | 0.013 | 0.003 | 0.007 | 0.002 | 0.004 | 0.004 | 0.006 | 0.026 | 0.003 | 0.002 |
| Poodle | (6) | 0.001 | 0.01 | 0.001 | 0.005 | 0.002 | 0.004 | 0.002 | 0.004 | 0.112 | 0.008 | 0.002 | 0.003 | 0.011 | 0.003 | 0.001 |
| Poodle | (1) | 0.014 | 0.006 | 0.006 | 0.004 | 0.002 | 0.005 | 0.011 | 0.007 | 0.044 | 0.107 | 0.005 | 0.01 | 0.008 | 0.004 | 0.009 |
| Poodle | (1) | 0.002 | 0.008 | 0.002 | 0.014 | 0.005 | 0.002 | 0.007 | 0.004 | 0.104 | 0.021 | 0.002 | 0.009 | 0.007 | 0.002 | 0.002 |
| Poodle | (1) | 0.006 | 0.019 | 0.002 | 0.004 | 0.002 | 0.004 | 0.035 | 0.005 | 0.007 | 0.002 | 0.003 | 0.029 | 0.002 | 0.005 | 0.002 |
| Poodle | 0 | 0.005 | 0.003 | 0.003 | 0.004 | 0.002 | 0.002 | 0.003 | 0.002 | 0.002 | 0.001 | 0.006 | 0.003 | 0.002 | 0.002 | 0.002 |
| Poodle | 0 | 0.019 | 0.003 | 0.015 | 0.004 | 0.015 | 0.003 | 0.004 | 0.009 | 0.001 | 0.002 | 0.042 | 0.002 | 0.002 | 0.004 | 0.004 |
| Poodle | 0 | 0.002 | 0.001 | 0.003 | 0.001 | 0.002 | 0.001 | 0.003 | 0.004 | 0.004 | 0.002 | 0.001 | 0.001 | 0.001 | 0.001 | 0.003 |
| Poodle | (5) | 0.007 | 0.002 | 0.005 | 0.002 | 0.007 | 0.004 | 0.002 | 0.005 | 0.002 | 0.003 | 0.003 | 0.003 | 0.001 | 0.004 | 0.006 |
| Poodle | (8) | 0.003 | 0.001 | 0.003 | 0.004 | 0.003 | 0.005 | 0.002 | 0.005 | 0.002 | 0.004 | 0.002 | 0.004 | 0.002 | 0.001 | 0.002 |
| Poodle | (1) | 0.004 | 0.005 | 0.004 | 0.004 | 0.003 | 0.004 | 0.006 | 0.003 | 0.004 | 0.002 | 0.003 | 0.002 | 0.002 | 0.003 | 0.004 |
| Poodle | 0 | 0.004 | 0.011 | 0.121 | 0.031 | 0.008 | 0.014 | 0.01 | 0.015 | 0.023 | 0.012 | 0.008 | 0.068 | 0.029 | 0.003 | 0.005 |
| Poodle | 0 | 0.008 | 0.007 | 0.005 | 0.003 | 0.009 | 0.161 | 0.044 | 0.022 | 0.022 | 0.007 | 0.004 | 0.005 | 0.009 | 0.023 | 0.004 |
| Poodle | (1) | 0.002 | 0.012 | 0.005 | 0.001 | 0.002 | 0.048 | 0.044 | 0.003 | 0.108 | 0.002 | 0.001 | 0.003 | 0.007 | 0.002 | 0.003 |
| Poodle | 0 | 0.001 | 0.003 | 0.002 | 0.002 | 0.001 | 0.004 | 0.004 | 0.008 | 0.002 | 0.008 | 0.002 | 0.003 | 0.005 | 0.002 | 0.003 |
| Poodle | 0 | 0.004 | 0.004 | 0.003 | 0.003 | 0.002 | 0.003 | 0.002 | 0.002 | 0.002 | 0.003 | 0.005 | 0.005 | 0.002 | 0.002 | 0.002 |
| Poodle | (1) | 0.003 | 0.009 | 0.006 | 0.004 | 0.003 | 0.026 | 0.016 | 0.019 | 0.002 | 0.002 | 0.004 | 0.006 | 0.023 | 0.002 | 0.003 |
| Poodle | (3) | 0.005 | 0.004 | 0.005 | 0.004 | 0.001 | 0.005 | 0.016 | 0.017 | 0.002 | 0.009 | 0.003 | 0.002 | 0.002 | 0.006 | 0.005 |
| Poodle | 0 | 0.01 | 0.002 | 0.002 | 0.004 | 0.003 | 0.013 | 0.007 | 0.003 | 0.002 | 0.002 | 0.014 | 0.005 | 0.004 | 0.002 | 0.003 |
| Poodle | (1) | 0.003 | 0.002 | 0.004 | 0.003 | 0.002 | 0.006 | 0.003 | 0.004 | 0.003 | 0.003 | 0.006 | 0.001 | 0.004 | 0.002 | 0.003 |
| Poodle | 0 | 0.003 | 0.003 | 0.034 | 0.005 | 0.014 | 0.007 | 0.003 | 0.004 | 0.004 | 0.004 | 0.002 | 0.007 | 0.005 | 0.003 | 0.031 |
| Poodle | 0 | 0.007 | 0.005 | 0.005 | 0.003 | 0.005 | 0.003 | 0.014 | 0.004 | 0.004 | 0.007 | 0.003 | 0.003 | 0.006 | 0.008 | 0.005 |
| Poodle | 0 | 0.008 | 0.01 | 0.003 | 0.008 | 0.002 | 0.004 | 0.015 | 0.039 | 0.001 | 0.005 | 0.01 | 0.002 | 0.002 | 0.009 | 0.004 |
| Poodle | (2) | 0.003 | 0.003 | 0.004 | 0.008 | 0.003 | 0.003 | 0.005 | 0.004 | 0.005 | 0.003 | 0.003 | 0.003 | 0.001 | 0.002 | 0.006 |
| Poodle | (1) | 0.009 | 0.003 | 0.002 | 0.002 | 0.001 | 0.003 | 0.016 | 0.018 | 0.003 | 0.002 | 0.004 | 0.004 | 0.003 | 0.002 | 0.003 |
| Poodle | (6) | 0.007 | 0.004 | 0.002 | 0.004 | 0.005 | 0.002 | 0.003 | 0.004 | 0.002 | 0.002 | 0.006 | 0.002 | 0.002 | 0.005 | 0.007 |
| Poodle | (6) | 0.006 | 0.004 | 0.005 | 0.004 | 0.009 | 0.002 | 0.003 | 0.002 | 0.003 | 0.001 | 0.004 | 0.004 | 0.002 | 0.002 | 0.012 |
| Poodle | (5) | 0.005 | 0.002 | 0.002 | 0.001 | 0.002 | 0.001 | 0.001 | 0.002 | 0.001 | 0.003 | 0.002 | 0.001 | 0.002 | 0.002 | 0.002 |
| Poodle | (4) | 0.004 | 0.002 | 0.002 | 0.002 | 0.001 | 0.002 | 0.002 | 0.002 | 0.002 | 0.002 | 0.002 | 0.001 | 0.006 | 0.002 | 0.007 |
| Poodle | (5) | 0.002 | 0.001 | 0.001 | 0.001 | 0.001 | 0.001 | 0.001 | 0.001 | 0.002 | 0.001 | 0.001 | 0.001 | 0.002 | 0.002 | 0.002 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Poodle | | 0.875 | 0.002 | 0.003 | 0.004 | 0.009 | 0.002 | 0.003 | 0.003 | 0.005 | 0.002 | 0.004 | 0.004 | 0.004 | 0.003 | 0.002 |
| Poodle | | 0.883 | 0.003 | 0.002 | 0.007 | 0.003 | 0.002 | 0.002 | 0.004 | 0.003 | 0.003 | 0.003 | 0.002 | 0.005 | 0.002 | 0.005 |
| Poodle | | 0.826 | 0.003 | 0.008 | 0.001 | 0.006 | 0.002 | 0.002 | 0.002 | 0.005 | 0.001 | 0.001 | 0.005 | 0.008 | 0.015 | 0.004 |
| Poodle | | 0.893 | 0.005 | 0.005 | 0.004 | 0.003 | 0.002 | 0.002 | 0.002 | 0.004 | 0.001 | 0.002 | 0.002 | 0.002 | 0.003 | 0.002 |
| Poodle | | 0.935 | 0.002 | 0.002 | 0.003 | 0.002 | 0.001 | 0.001 | 0.002 | 0.002 | 0.002 | 0.002 | 0.002 | 0.002 | 0.002 | 0.002 |
| Poodle | | 0.727 | 0.003 | 0.011 | 0.003 | 0.007 | 0.003 | 0.003 | 0.002 | 0.002 | 0.003 | 0.003 | 0.007 | 0.008 | 0.014 | 0.004 |
| Poodle | | 0.944 | 0.002 | 0.002 | 0.002 | 0.003 | 0.002 | 0.002 | 0.001 | 0.003 | 0.001 | 0.001 | 0.003 | 0.001 | 0.002 | 0.001 |
| Poodle | | 0.692 | 0.02 | 0.068 | 0.004 | 0.003 | 0.004 | 0.005 | 0.018 | 0.011 | 0.007 | 0.004 | 0.017 | 0.008 | 0.018 | 0.006 |
| Poodle | | 0.727 | 0.004 | 0.004 | 0.004 | 0.006 | 0.002 | 0.002 | 0.033 | 0.002 | 0.007 | 0.026 | 0.004 | 0.007 | 0.003 | 0.002 |
| Poodle | | 0.644 | 0.005 | 0.039 | 0.003 | 0.002 | 0.003 | 0.006 | 0.003 | 0.007 | 0.002 | 0.004 | 0.012 | 0.012 | 0.002 | 0.013 |
| Poodle | | 0.684 | 0.007 | 0.013 | 0.009 | 0.013 | 0.001 | 0.002 | 0.038 | 0.006 | 0.008 | 0.006 | 0.006 | 0.008 | 0.007 | 0.003 |
| Poodle | | 0.808 | 0.004 | 0.003 | 0.003 | 0.003 | 0.007 | 0.012 | 0.007 | 0.01 | 0.002 | 0.003 | 0.001 | 0.003 | 0.002 | 0.004 |
| Poodle | | 0.924 | 0.001 | 0.002 | 0.002 | 0.002 | 0.003 | 0.002 | 0.002 | 0.003 | 0.003 | 0.005 | 0.003 | 0.003 | 0.003 | 0.002 |
| Poodle | | 0.826 | 0.003 | 0.003 | 0.002 | 0.004 | 0.011 | 0.001 | 0.001 | 0.004 | 0.002 | 0.002 | 0.002 | 0.002 | 0.002 | 0.006 |
| Poodle | | 0.924 | 0.004 | 0.002 | 0.002 | 0.002 | 0.012 | 0.006 | 0.002 | 0.002 | 0.002 | 0.004 | 0.002 | 0.002 | 0.001 | 0.002 |
| Poodle | | 0.877 | 0.005 | 0.006 | 0.003 | 0.003 | 0.01 | 0.004 | 0.002 | 0.005 | 0.002 | 0.01 | 0.002 | 0.004 | 0.007 | 0.004 |
| Poodle | | 0.903 | 0.002 | 0.001 | 0.001 | 0.002 | 0.005 | 0.002 | 0.003 | 0.002 | 0.008 | 0.007 | 0.003 | 0.002 | 0.006 | 0.009 |
| Poodle | | 0.893 | 0.004 | 0.003 | 0.005 | 0.003 | 0.004 | 0.003 | 0.002 | 0.004 | 0.006 | 0.004 | 0.006 | 0.002 | 0.003 | 0.005 |
| Poodle | | 0.389 | 0.002 | 0.005 | 0.02 | 0.004 | 0.035 | 0.011 | 0.055 | 0.003 | 0.01 | 0.091 | 0.002 | 0.003 | 0.002 | 0.006 |
| Poodle | | 0.549 | 0.005 | 0.006 | 0.003 | 0.034 | 0.003 | 0.006 | 0.003 | 0.006 | 0.004 | 0.015 | 0.004 | 0.013 | 0.003 | 0.011 |
| Poodle | | 0.688 | 0.004 | 0.014 | 0.006 | 0.005 | 0.006 | 0.004 | 0.005 | 0.008 | 0.002 | 0.002 | 0.001 | 0.004 | 0.002 | 0.006 |
| Poodle | | 0.892 | 0.012 | 0.003 | 0.005 | 0.003 | 0.005 | 0.005 | 0.003 | 0.001 | 0.004 | 0.006 | 0.002 | 0.004 | 0.001 | 0.002 |
| Poodle | | 0.929 | 0.002 | 0.002 | 0.002 | 0.001 | 0.001 | 0.004 | 0.002 | 0.002 | 0.001 | 0.003 | 0.003 | 0.003 | 0.001 | 0.001 |
| Poodle | | 0.703 | 0.011 | 0.011 | 0.015 | 0.004 | 0.082 | 0.006 | 0.002 | 0.014 | 0.002 | 0.003 | 0.002 | 0.005 | 0.009 | 0.004 |
| Poodle | | 0.587 | 0.033 | 0.004 | 0.006 | 0.088 | 0.009 | 0.002 | 0.004 | 0.003 | 0.028 | 0.004 | 0.137 | 0.002 | 0.003 | 0.005 |
| Poodle | | 0.861 | 0.003 | 0.003 | 0.004 | 0.002 | 0.008 | 0.003 | 0.016 | 0.003 | 0.001 | 0.007 | 0.002 | 0.003 | 0.002 | 0.006 |
| Poodle | | 0.892 | 0.004 | 0.003 | 0.004 | 0.002 | 0.003 | 0.003 | 0.004 | 0.001 | 0.003 | 0.015 | 0.002 | 0.002 | 0.001 | 0.009 |
| Poodle | | 0.809 | 0.001 | 0.002 | 0.003 | 0.006 | 0.004 | 0.001 | 0.025 | 0.003 | 0.006 | 0.004 | 0.002 | 0.002 | 0.003 | 0.002 |
| Poodle | | 0.839 | 0.007 | 0.004 | 0.002 | 0.003 | 0.01 | 0.004 | 0.006 | 0.002 | 0.008 | 0.001 | 0.006 | 0.022 | 0.001 | 0.004 |
| Poodle | | 0.619 | 0.026 | 0.058 | 0.007 | 0.005 | 0.006 | 0.002 | 0.007 | 0.01 | 0.058 | 0.005 | 0.008 | 0.057 | 0.003 | 0.006 |
| Poodle | | 0.874 | 0.003 | 0.002 | 0.025 | 0.003 | 0.002 | 0.004 | 0.004 | 0.003 | 0.005 | 0.003 | 0.002 | 0.006 | 0.006 | 0.004 |
| Poodle | | 0.844 | 0.004 | 0.002 | 0.004 | 0.001 | 0.002 | 0.002 | 0.003 | 0.009 | 0.029 | 0.006 | 0.008 | 0.001 | 0.003 | 0.005 |
| Poodle | | 0.881 | 0.003 | 0.006 | 0.004 | 0.001 | 0.002 | 0.002 | 0.002 | 0.004 | 0.002 | 0.001 | 0.031 | 0.002 | 0.002 | 0.002 |
| Poodle | | 0.835 | 0.002 | 0.006 | 0.003 | 0.004 | 0.006 | 0.017 | 0.002 | 0.007 | 0.009 | 0.002 | 0.029 | 0.006 | 0.003 | 0.006 |
| Poodle | | 0.592 | 0.007 | 0.009 | 0.015 | 0.007 | 0.003 | 0.002 | 0.014 | 0.129 | 0.005 | 0.002 | 0.012 | 0.003 | 0.006 | 0.007 |
| Poodle | | 0.938 | 0.002 | 0.002 | 0.002 | 0.002 | 0.004 | 0.001 | 0.003 | 0.001 | 0.003 | 0.001 | 0.005 | 0.002 | 0.001 | 0.002 |
| Poodle | | 0.924 | 0.001 | 0.002 | 0.002 | 0.002 | 0.005 | 0.002 | 0.001 | 0.002 | 0.003 | 0.002 | 0.009 | 0.002 | 0.002 | 0.002 |
| Poodle | | 0.945 | 0.003 | 0.001 | 0.002 | 0.002 | 0.003 | 0.001 | 0.003 | 0.003 | 0.002 | 0.003 | 0.003 | 0.003 | 0.004 | 0.001 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Pug | 0 | 0.001 | 0.002 | 0.002 | 0.002 | 0.001 | 0.001 | 0.002 | 0.002 | 0.001 | 0.002 | 0.005 | 0.001 | 0.002 | 0.001 | 0.001 |
| Pug | 0 | 0.005 | 0.003 | 0.001 | 0.002 | 0.004 | 0.002 | 0.003 | 0.003 | 0.007 | 0.004 | 0.003 | 0.002 | 0.001 | 0.005 | 0.009 |
| Pug | (1) | 0.001 | 0.002 | 0.002 | 0.001 | 0.001 | 0.001 | 0.001 | 0.002 | 0.001 | 0.001 | 0.002 | 0.001 | 0.002 | 0.002 | 0.001 |
| Pug | 0 | 0.003 | 0.011 | 0.007 | 0.005 | 0.001 | 0.002 | 0.002 | 0.003 | 0.003 | 0.002 | 0.003 | 0.007 | 0.003 | 0.002 | 0.002 |
| Pug | 0 | 0.001 | 0.003 | 0.004 | 0.003 | 0.001 | 0.002 | 0.001 | 0.001 | 0.001 | 0.002 | 0.002 | 0.002 | 0.002 | 0.002 | 0.002 |
| Pug | 0 | 0.001 | 0.005 | 0.002 | 0.002 | 0.002 | 0.002 | 0.002 | 0.001 | 0.001 | 0.002 | 0.001 | 0.002 | 0.001 | 0.002 | 0.002 |
| Pug | 0 | 0.002 | 0.004 | 0.002 | 0.002 | 0.002 | 0.002 | 0.003 | 0.003 | 0.001 | 0.002 | 0.002 | 0.001 | 0.002 | 0.002 | 0.002 |
| Pug | 0 | 0.001 | 0.003 | 0.001 | 0.001 | 0.001 | 0.002 | 0.002 | 0.002 | 0.001 | 0.006 | 0.002 | 0.001 | 0.001 | 0.001 | 0.002 |
| Pug | 0 | 0.001 | 0.002 | 0.001 | 0.001 | 0.001 | 0.002 | 0.002 | 0.001 | 0.001 | 0.001 | 0.001 | 0.004 | 0.002 | 0.001 | 0.001 |
| Pug | 0 | 0.002 | 0.002 | 0.002 | 0.001 | 0.001 | 0.003 | 0.005 | 0.002 | 0.005 | 0.003 | 0.001 | 0.005 | 0.003 | 0.001 | 0.001 |
| Pug | 0 | 0.002 | 0.006 | 0.002 | 0.003 | 0.001 | 0.002 | 0.003 | 0.004 | 0.003 | 0.002 | 0.002 | 0.01 | 0.005 | 0.002 | 0.001 |
| Pug | 0 | 0.001 | 0.004 | 0.001 | 0.001 | 0.001 | 0.002 | 0.002 | 0.003 | 0.001 | 0.001 | 0.001 | 0.001 | 0.001 | 0.001 | 0.002 |
| Pug | 0 | 0.004 | 0.009 | 0.006 | 0.004 | 0.001 | 0.006 | 0.015 | 0.037 | 0.002 | 0.004 | 0.008 | 0.002 | 0.004 | 0.004 | 0.002 |
| Pug | 0 | 0.001 | 0.014 | 0.005 | 0.004 | 0.001 | 0.004 | 0.006 | 0.005 | 0.006 | 0.002 | 0.001 | 0.004 | 0.002 | 0.001 | 0.009 |
| Pug | 0 | 0.001 | 0.003 | 0.001 | 0.001 | 0.001 | 0.002 | 0.006 | 0.002 | 0.001 | 0.001 | 0.002 | 0.001 | 0.002 | 0.005 | 0.001 |
| Pug | 0 | 0.001 | 0.002 | 0.001 | 0.002 | 0.001 | 0.001 | 0.001 | 0.001 | 0.001 | 0.007 | 0.001 | 0.002 | 0.002 | 0.002 | 0.001 |
| Pug | 0 | 0.001 | 0.002 | 0.001 | 0.001 | 0.002 | 0.002 | 0.002 | 0.002 | 0.001 | 0.001 | 0.001 | 0.001 | 0.001 | 0.001 | 0.001 |
| Pug | 0 | 0.001 | 0.001 | 0.002 | 0.002 | 0.001 | 0.001 | 0.001 | 0.001 | 0.001 | 0.001 | 0.001 | 0.001 | 0.001 | 0.001 | 0.001 |
| Pug | 0 | 0.001 | 0.001 | 0.003 | 0.003 | 0.001 | 0.001 | 0.001 | 0.001 | 0.002 | 0.001 | 0.003 | 0.001 | 0.003 | 0.003 | 0.002 |
| Pug | 0 | 0.002 | 0.001 | 0.006 | 0.006 | 0.001 | 0.002 | 0.002 | 0.001 | 0.002 | 0.002 | 0.007 | 0.001 | 0.006 | 0.003 | 0.003 |
| Pug | 0 | 0.006 | 0.003 | 0.004 | 0.004 | 0.002 | 0.004 | 0.006 | 0.006 | 0.009 | 0.003 | 0.003 | 0.001 | 0.013 | 0.002 | 0.002 |
| Pug | 0 | 0.002 | 0.002 | 0.003 | 0.004 | 0.001 | 0.001 | 0.002 | 0.002 | 0.002 | 0.004 | 0.004 | 0.002 | 0.005 | 0.006 | 0.003 |
| Pug | 0 | 0.009 | 0.003 | 0.007 | 0.004 | 0.002 | 0.003 | 0.003 | 0.002 | 0.005 | 0.003 | 0.005 | 0.001 | 0.005 | 0.008 | 0.011 |
| Pug | 0 | 0.001 | 0.001 | 0.004 | 0.005 | 0.003 | 0.001 | 0.002 | 0.003 | 0.001 | 0.003 | 0.002 | 0.01 | 0.004 | 0.002 | 0.002 |
| Pug | (1) | 0.001 | 0.002 | 0.001 | 0.001 | 0.001 | 0.001 | 0.001 | 0.001 | 0.001 | 0.001 | 0.001 | 0.003 | 0.001 | 0.001 | 0.001 |
| Pug | 0 | 0.001 | 0.001 | 0.002 | 0.001 | 0.002 | 0.002 | 0.001 | 0.002 | 0.001 | 0.003 | 0.002 | 0.002 | 0.001 | 0.002 | 0.001 |
| Pug | 0 | 0.001 | 0.001 | 0.001 | 0.001 | 0.002 | 0.001 | 0.001 | 0.001 | 0.001 | 0.001 | 0.001 | 0.001 | 0.001 | 0.001 | 0.001 |
| Pug | 0 | 0.003 | 0.002 | 0.002 | 0.016 | 0.011 | 0.004 | 0.009 | 0.003 | 0.002 | 0.009 | 0.003 | 0.001 | 0.004 | 0.003 | 0.002 |
| Pug | 0 | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Pug | 0 | 0.001 | 0.002 | 0.002 | 0.001 | 0.002 | 0.002 | 0.001 | 0.001 | 0.002 | 0.003 | 0.002 | 0.001 | 0.001 | 0.004 | 0.002 |
| Pug | 0 | 0.001 | 0.003 | 0.003 | 0.002 | 0.002 | 0.002 | 0.002 | 0.001 | 0.001 | 0.006 | 0.001 | 0.002 | 0.002 | 0.004 | 0.003 |
| Pug | 0 | 0.001 | 0.001 | 0.003 | 0.002 | 0.001 | 0.001 | 0.001 | 0.002 | 0.003 | 0.001 | 0.004 | 0.001 | 0.001 | 0.002 | 0.002 |
| Pug | 0 | 0.003 | 0.001 | 0.002 | 0.002 | 0.002 | 0.002 | 0.002 | 0.003 | 0.019 | 0.002 | 0.002 | 0.002 | 0.004 | 0.002 | 0.001 |
| Pug | 0 | 0.001 | 0.001 | 0.002 | 0.002 | 0.002 | 0.001 | 0.002 | 0.002 | 0.001 | 0.004 | 0.002 | 0.003 | 0.001 | 0.001 | 0.002 |
| Pug | 0 | 0.001 | 0.001 | 0.001 | 0.001 | 0.004 | 0.002 | 0.001 | 0.001 | 0.001 | 0.002 | 0.002 | 0.002 | 0.001 | 0.002 | 0.001 |
| Pug | 0 | 0.002 | 0.005 | 0.002 | 0.001 | 0.001 | 0.008 | 0.013 | 0.001 | 0.004 | 0.001 | 0.004 | 0.004 | 0.005 | 0.005 | 0.002 |
| Pug | (1) | 0.002 | 0.001 | 0.001 | 0.002 | 0.001 | 0.002 | 0.002 | 0.001 | 0.001 | 0.003 | 0.002 | 0.001 | 0.002 | 0.001 | 0.002 |
| Pug | 0 | 0.002 | 0.002 | 0.001 | 0.001 | 0.001 | 0.001 | 0.001 | 0.003 | 0.002 | 0.002 | 0.001 | 0.001 | 0.001 | 0.002 | 0.001 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Pug | | 0.001 | 0.004 | 0.003 | 0.001 | 0.002 | 0.002 | 0.948 | 0.001 | 0.001 | 0.003 | 0.001 | 0.002 | 0.002 | 0.002 | 0.001 |
| Pug | | 0.001 | 0.004 | 0.002 | 0.002 | 0.004 | 0.002 | 0.87 | 0.003 | 0.009 | 0.002 | 0.004 | 0.004 | 0.002 | 0.001 | 0.035 |
| Pug | | 0.001 | 0.002 | 0.001 | 0.002 | 0.001 | 0.002 | 0.959 | 0.001 | 0.001 | 0.001 | 0.001 | 0.001 | 0.001 | 0.002 | 0.001 |
| Pug | | 0.001 | 0.005 | 0.004 | 0.004 | 0.002 | 0.002 | 0.906 | 0.003 | 0.002 | 0.002 | 0.001 | 0.003 | 0.002 | 0.005 | 0.002 |
| Pug | | 0.001 | 0.002 | 0.002 | 0.008 | 0.002 | 0.003 | 0.933 | 0.002 | 0.004 | 0.001 | 0.001 | 0.002 | 0.001 | 0.005 | 0.002 |
| Pug | | 0.001 | 0.002 | 0.002 | 0.002 | 0.001 | 0.001 | 0.945 | 0.002 | 0.002 | 0.001 | 0.001 | 0.002 | 0.002 | 0.002 | 0.005 |
| Pug | | 0.001 | 0.002 | 0.003 | 0.001 | 0.003 | 0.003 | 0.936 | 0.002 | 0.002 | 0.003 | 0.002 | 0.003 | 0.002 | 0.002 | 0.005 |
| Pug | | 0.001 | 0.004 | 0.002 | 0.003 | 0.002 | 0.001 | 0.941 | 0.004 | 0.001 | 0.002 | 0.003 | 0.003 | 0.002 | 0.003 | 0.001 |
| Pug | | 0.001 | 0.001 | 0.002 | 0.003 | 0.002 | 0.001 | 0.953 | 0.002 | 0.002 | 0.001 | 0.001 | 0.001 | 0.002 | 0.001 | 0.001 |
| Pug | | 0.001 | 0.003 | 0.003 | 0.004 | 0.004 | 0.003 | 0.916 | 0.007 | 0.001 | 0.001 | 0.002 | 0.003 | 0.003 | 0.006 | 0.004 |
| Pug | | 0.002 | 0.003 | 0.002 | 0.002 | 0.004 | 0.001 | 0.885 | 0.002 | 0.001 | 0.015 | 0.007 | 0.022 | 0.001 | 0.002 | 0.004 |
| Pug | | 0.001 | 0.002 | 0.001 | 0.002 | 0.001 | 0.002 | 0.955 | 0.002 | 0.002 | 0.001 | 0.001 | 0.001 | 0.001 | 0.002 | 0.002 |
| Pug | | 0.004 | 0.006 | 0.009 | 0.005 | 0.002 | 0.004 | 0.809 | 0.01 | 0.003 | 0.011 | 0.014 | 0.004 | 0.004 | 0.002 | 0.004 |
| Pug | | 0.003 | 0.017 | 0.005 | 0.002 | 0.001 | 0.004 | 0.877 | 0.007 | 0.002 | 0.005 | 0.002 | 0.002 | 0.001 | 0.002 | 0.002 |
| Pug | | 0.002 | 0.002 | 0.001 | 0.002 | 0.002 | 0.002 | 0.948 | 0.001 | 0.002 | 0.001 | 0.002 | 0.001 | 0.001 | 0.001 | 0.002 |
| Pug | | 0.001 | 0.002 | 0.004 | 0.002 | 0.002 | 0.002 | 0.947 | 0.002 | 0.001 | 0.001 | 0.001 | 0.001 | 0.001 | 0.005 | 0.002 |
| Pug | | 0.001 | 0.002 | 0.002 | 0.001 | 0.002 | 0.001 | 0.96 | 0.002 | 0.001 | 0.001 | 0.002 | 0.001 | 0.002 | 0.001 | 0.002 |
| Pug | | 0.001 | 0.001 | 0.001 | 0.001 | 0.001 | 0.001 | 0.969 | 0.001 | 0.001 | 0.001 | 0.001 | 0.001 | 0.001 | 0.001 | 0.001 |
| Pug | | 0.001 | 0.001 | 0.002 | 0.001 | 0.001 | 0.001 | 0.954 | 0.001 | 0.002 | 0.002 | 0.001 | 0.002 | 0.001 | 0.001 | 0.001 |
| Pug | | 0.001 | 0.003 | 0.003 | 0.002 | 0.002 | 0.002 | 0.926 | 0.002 | 0.002 | 0.003 | 0.001 | 0.003 | 0.003 | 0.001 | 0.002 |
| Pug | | 0.001 | 0.004 | 0.003 | 0.009 | 0.009 | 0.004 | 0.859 | 0.011 | 0.011 | 0.005 | 0.003 | 0.004 | 0.002 | 0.004 | 0.003 |
| Pug | | 0.002 | 0.007 | 0.006 | 0.002 | 0.005 | 0.006 | 0.898 | 0.007 | 0.005 | 0.003 | 0.002 | 0.006 | 0.001 | 0.002 | 0.003 |
| Pug | | 0.002 | 0.004 | 0.004 | 0.003 | 0.001 | 0.002 | 0.866 | 0.002 | 0.004 | 0.011 | 0.004 | 0.01 | 0.002 | 0.004 | 0.011 |
| Pug | | 0.004 | 0.001 | 0.002 | 0.004 | 0.005 | 0.002 | 0.917 | 0.002 | 0.001 | 0.005 | 0.006 | 0.003 | 0.001 | 0.002 | 0.001 |
| Pug | | 0.001 | 0.002 | 0.002 | 0.002 | 0.002 | 0.001 | 0.962 | 0.001 | 0.001 | 0.001 | 0.001 | 0.001 | 0.001 | 0.003 | 0.001 |
| Pug | | 0.001 | 0.002 | 0.001 | 0.001 | 0.001 | 0.002 | 0.959 | 0.001 | 0.001 | 0.001 | 0.001 | 0.001 | 0.002 | 0.002 | 0.002 |
| Pug | | 0.001 | 0.001 | 0.002 | 0.001 | 0.002 | 0.002 | 0.966 | 0.001 | 0.002 | 0.001 | 0.001 | 0.001 | 0.001 | 0.001 | 0.002 |
| Pug | | 0.005 | 0.002 | 0.002 | 0.003 | 0.006 | 0.01 | 0.867 | 0.003 | 0.002 | 0.009 | 0.002 | 0.003 | 0.003 | 0.007 | 0.005 |
| Pug | | 0.001 | 0.001 | 0.001 | 0.001 | 0.002 | 0.001 | 0.969 | 0.001 | 0.001 | 0.001 | 0.001 | 0.001 | 0.001 | 0.002 | 0.001 |
| Pug | | 0.001 | 0.004 | 0.004 | 0.001 | 0.001 | 0.002 | 0.95 | 0.001 | 0.002 | 0.001 | 0.001 | 0.001 | 0.002 | 0.002 | 0.002 |
| Pug | | 0.001 | 0.004 | 0.004 | 0.004 | 0.001 | 0.003 | 0.931 | 0.001 | 0.002 | 0.002 | 0.002 | 0.001 | 0.002 | 0.004 | 0.002 |
| Pug | | 0.001 | 0.001 | 0.001 | 0.001 | 0.002 | 0.001 | 0.958 | 0.001 | 0.001 | 0.001 | 0.001 | 0.001 | 0.002 | 0.001 | 0.001 |
| Pug | | 0.001 | 0.002 | 0.002 | 0.001 | 0.016 | 0.002 | 0.901 | 0.003 | 0.003 | 0.007 | 0.003 | 0.004 | 0.002 | 0.001 | 0.004 |
| Pug | | 0.001 | 0.001 | 0.002 | 0.002 | 0.006 | 0.001 | 0.944 | 0.001 | 0.001 | 0.001 | 0.002 | 0.001 | 0.003 | 0.004 | 0.001 |
| Pug | | 0.001 | 0.001 | 0.001 | 0.002 | 0.001 | 0.002 | 0.958 | 0.001 | 0.001 | 0.001 | 0.001 | 0.001 | 0.003 | 0.001 | 0.001 |
| Pug | | 0.003 | 0.003 | 0.003 | 0.002 | 0.005 | 0.001 | 0.896 | 0.002 | 0.005 | 0.004 | 0.004 | 0.002 | 0.004 | 0.002 | 0.006 |
| Pug | | 0.001 | 0.001 | 0.001 | 0.002 | 0.005 | 0.002 | 0.947 | 0.002 | 0.002 | 0.004 | 0.001 | 0.002 | 0.001 | 0.003 | 0.001 |
| Pug | | 0.001 | 0.001 | 0.001 | 0.002 | 0.001 | 0.001 | 0.96 | 0.002 | 0.001 | 0.001 | 0.001 | 0.002 | 0.001 | 0.001 | 0.001 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Rottweiler | 0 | 0.005 | 0.001 | 0.005 | 0.003 | 0.001 | 0.003 | 0.003 | 0.003 | 0.001 | 0.002 | 0.912 | 0.005 | 0.004 | 0.001 | 0.003 |
| Rottweiler | (9) | 0.005 | 0.002 | 0.002 | 0.003 | 0.001 | 0.002 | 0.002 | 0.003 | 0.002 | 0.003 | 0.94 | 0.003 | 0.002 | 0.002 | 0.003 |
| Rottweiler | 0 | 0.001 | 0.001 | 0.002 | 0.001 | 0.002 | 0.001 | 0.002 | 0.002 | 0.001 | 0.001 | 0.929 | 0.003 | 0.002 | 0.002 | 0.001 |
| Rottweiler | 0 | 0.002 | 0.001 | 0.004 | 0.002 | 0.001 | 0.003 | 0.001 | 0.002 | 0.002 | 0.004 | 0.925 | 0.004 | 0.001 | 0.001 | 0.002 |
| Rottweiler | 0 | 0.002 | 0.001 | 0.002 | 0.003 | 0.002 | 0.002 | 0.002 | 0.003 | 0.002 | 0.001 | 0.924 | 0.001 | 0.001 | 0.002 | 0.001 |
| Rottweiler | 0 | 0.002 | 0.007 | 0.005 | 0.002 | 0.012 | 0.004 | 0.004 | 0.017 | 0.002 | 0.001 | 0.889 | 0.002 | 0.002 | 0.005 | 0.004 |
| Rottweiler | 0 | 0.002 | 0.003 | 0.016 | 0.004 | 0.003 | 0.002 | 0.003 | 0.003 | 0.001 | 0.002 | 0.906 | 0.001 | 0.004 | 0.003 | 0.002 |
| Rottweiler | (1) | 0.005 | 0.003 | 0.006 | 0.007 | 0.003 | 0.004 | 0.004 | 0.003 | 0.006 | 0.002 | 0.905 | 0.002 | 0.002 | 0.001 | 0.005 |
| Rottweiler | (4) | 0.001 | 0.001 | 0.003 | 0.001 | 0.001 | 0.001 | 0.001 | 0.001 | 0.001 | 0.001 | 0.965 | 0.001 | 0.001 | 0.002 | 0.001 |
| Rottweiler | 0 | 0.001 | 0.002 | 0.011 | 0.002 | 0.005 | 0.003 | 0.001 | 0.004 | 0.001 | 0.003 | 0.92 | 0.001 | 0.002 | 0.004 | 0.003 |
| Rottweiler | (1) | 0.005 | 0.001 | 0.004 | 0.002 | 0.001 | 0.005 | 0.002 | 0.003 | 0.002 | 0.004 | 0.892 | 0.014 | 0.005 | 0.003 | 0.002 |
| Rottweiler | 0 | 0.004 | 0.002 | 0.006 | 0.01 | 0.001 | 0.001 | 0.002 | 0.003 | 0.003 | 0.002 | 0.889 | 0.002 | 0.002 | 0.003 | 0.005 |
| Rottweiler | 0 | 0.005 | 0.002 | 0.004 | 0.009 | 0.001 | 0.003 | 0.002 | 0.002 | 0.004 | 0.004 | 0.894 | 0.004 | 0.003 | 0.011 | 0.002 |
| Rottweiler | 0 | 0.007 | 0.002 | 0.003 | 0.005 | 0.003 | 0.003 | 0.002 | 0.003 | 0.002 | 0.002 | 0.917 | 0.001 | 0.003 | 0.002 | 0.006 |
| Rottweiler | (8) | 0.003 | 0.005 | 0.004 | 0.006 | 0.002 | 0.003 | 0.006 | 0.014 | 0.003 | 0.003 | 0.854 | 0.004 | 0.004 | 0.003 | 0.003 |
| Rottweiler | 0 | 0.005 | 0.003 | 0.004 | 0.001 | 0.009 | 0.005 | 0.001 | 0.003 | 0.001 | 0.004 | 0.869 | 0.004 | 0.003 | 0.002 | 0.005 |
| Rottweiler | 0 | 0.001 | 0.012 | 0.002 | 0.003 | 0.003 | 0.054 | 0.003 | 0.002 | 0.003 | 0.002 | 0.851 | 0.004 | 0.004 | 0.003 | 0.002 |
| Rottweiler | 0 | 0.011 | 0.002 | 0.037 | 0.02 | 0.005 | 0.002 | 0.008 | 0.029 | 0.002 | 0.003 | 0.761 | 0.018 | 0.002 | 0.003 | 0.003 |
| Rottweiler | 0 | 0.004 | 0.002 | 0.006 | 0.012 | 0.003 | 0.002 | 0.005 | 0.017 | 0.002 | 0.003 | 0.825 | 0.01 | 0.003 | 0.005 | 0.002 |
| Rottweiler | 0 | 0.001 | 0.001 | 0.001 | 0.001 | 0.002 | 0.002 | 0.001 | 0.001 | 0.001 | 0.001 | 0.966 | 0.001 | 0.001 | 0.001 | 0.001 |
| Rottweiler | (2) | 0.002 | 0.001 | 0.003 | 0.001 | 0.004 | 0.002 | 0.002 | 0.002 | 0.002 | 0.006 | 0.917 | 0.001 | 0.001 | 0.003 | 0.005 |
| Rottweiler | 0 | 0.005 | 0.002 | 0.002 | 0.015 | 0.002 | 0.003 | 0.008 | 0.005 | 0.001 | 0.003 | 0.876 | 0.016 | 0.005 | 0.002 | 0.002 |
| Rottweiler | 0 | 0.011 | 0.006 | 0.009 | 0.005 | 0.002 | 0.012 | 0.002 | 0.01 | 0.023 | 0.001 | 0.828 | 0.002 | 0.004 | 0.004 | 0.002 |
| Rottweiler | 0 | 0.001 | 0.004 | 0.002 | 0.005 | 0.002 | 0.003 | 0.003 | 0.001 | 0.004 | 0.002 | 0.91 | 0.002 | 0.003 | 0.002 | 0.004 |
| Rottweiler | (3) | 0.001 | 0.001 | 0.001 | 0.001 | 0.001 | 0.001 | 0.002 | 0.001 | 0.002 | 0.001 | 0.961 | 0.001 | 0.001 | 0.003 | 0.001 |
| Rottweiler | (17) | 0.004 | 0.027 | 0.004 | 0.004 | 0.001 | 0.007 | 0.002 | 0.003 | 0.001 | 0.003 | 0.797 | 0.005 | 0.008 | 0.053 | 0.004 |
| Rottweiler | 0 | 0.001 | 0.002 | 0.002 | 0.001 | 0.001 | 0.002 | 0.004 | 0.003 | 0.002 | 0.002 | 0.938 | 0.002 | 0.001 | 0.008 | 0.001 |
| Rottweiler | 0 | 0.001 | 0.02 | 0.002 | 0.003 | 0.003 | 0.007 | 0.004 | 0.003 | 0.005 | 0.002 | 0.824 | 0.003 | 0.001 | 0.068 | 0.002 |
| Rottweiler | 0 | 0.003 | 0.003 | 0.003 | 0.001 | 0.001 | 0.001 | 0.003 | 0.004 | 0.002 | 0.002 | 0.943 | 0.002 | 0.002 | 0.002 | 0.001 |
| Rottweiler | 0 | 0.074 | 0.002 | 0.01 | 0.013 | 0.001 | 0.005 | 0.004 | 0.01 | 0.002 | 0.001 | 0.77 | 0.004 | 0.05 | 0.004 | 0.002 |
| Rottweiler | 0 | 0.003 | 0.002 | 0.002 | 0.002 | 0.002 | 0.002 | 0.009 | 0.003 | 0.002 | 0.001 | 0.876 | 0.002 | 0.002 | 0.003 | 0.002 |
| Rottweiler | 0 | 0.001 | 0.001 | 0.003 | 0.002 | 0.001 | 0.001 | 0.001 | 0.001 | 0.001 | 0.001 | 0.951 | 0.001 | 0.002 | 0.003 | 0.001 |
| Rottweiler | (1) | 0.001 | 0.002 | 0.001 | 0.001 | 0.003 | 0.001 | 0.001 | 0.002 | 0.001 | 0.001 | 0.964 | 0.001 | 0.001 | 0.001 | 0.001 |
| Rottweiler | 0 | 0.003 | 0.009 | 0.001 | 0.004 | 0.002 | 0.005 | 0.003 | 0.006 | 0.009 | 0.004 | 0.838 | 0.014 | 0.005 | 0.003 | 0.031 |
| Rottweiler | (2) | 0.002 | 0.002 | 0.003 | 0.002 | 0.001 | 0.006 | 0.004 | 0.002 | 0.003 | 0.002 | 0.93 | 0.001 | 0.003 | 0.004 | 0.006 |
| Rottweiler | (1) | 0.001 | 0.002 | 0.002 | 0.002 | 0.002 | 0.002 | 0.001 | 0.001 | 0.001 | 0.002 | 0.953 | 0.001 | 0.001 | 0.003 | 0.001 |
| Rottweiler | (1) | 0.003 | 0.003 | 0.003 | 0.003 | 0.004 | 0.001 | 0.002 | 0.001 | 0.001 | 0.001 | 0.93 | 0.001 | 0.002 | 0.002 | 0.002 |
| Rottweiler | (47) | 0.002 | 0.002 | 0.002 | 0.002 | 0.002 | 0.001 | 0.001 | 0.003 | 0.002 | 0.002 | 0.944 | 0.001 | 0.002 | 0.001 | 0.003 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Rottweiler | | 0.001 | 0.005 | 0.005 | 0.007 | 0.002 | 0.002 | 0.007 | 0.003 | 0.001 | 0.002 | 0.002 | 0.001 | 0.003 | 0.002 | 0.004 |
| Rottweiler | | 0.001 | 0.001 | 0.001 | 0.001 | 0.002 | 0.002 | 0.002 | 0.001 | 0.002 | 0.004 | 0.002 | 0.002 | 0.002 | 0.002 | 0.001 |
| Rottweiler | | 0.006 | 0.001 | 0.009 | 0.002 | 0.006 | 0.001 | 0.001 | 0.001 | 0.001 | 0.001 | 0.001 | 0.001 | 0.015 | 0.001 | 0.001 |
| Rottweiler | | 0.001 | 0.005 | 0.003 | 0.003 | 0.003 | 0.002 | 0.003 | 0.002 | 0.002 | 0.003 | 0.001 | 0.003 | 0.005 | 0.007 | 0.002 |
| Rottweiler | | 0.001 | 0.002 | 0.004 | 0.004 | 0.002 | 0.002 | 0.005 | 0.002 | 0.005 | 0.001 | 0.001 | 0.004 | 0.013 | 0.001 | 0.001 |
| Rottweiler | | 0.003 | 0.005 | 0.006 | 0.003 | 0.002 | 0.006 | 0.001 | 0.003 | 0.002 | 0.004 | 0.001 | 0.002 | 0.002 | 0.002 | 0.002 |
| Rottweiler | | 0.001 | 0.003 | 0.002 | 0.006 | 0.002 | 0.003 | 0.003 | 0.003 | 0.002 | 0.005 | 0.004 | 0.003 | 0.002 | 0.005 | 0.002 |
| Rottweiler | | 0.001 | 0.002 | 0.003 | 0.003 | 0.004 | 0.002 | 0.002 | 0.005 | 0.004 | 0.002 | 0.001 | 0.004 | 0.005 | 0.002 | 0.003 |
| Rottweiler | | 0.001 | 0.002 | 0.002 | 0.002 | 0.001 | 0.001 | 0.001 | 0.001 | 0.001 | 0.001 | 0.001 | 0.001 | 0.002 | 0.001 | 0.001 |
| Rottweiler | | 0.001 | 0.004 | 0.002 | 0.004 | 0.006 | 0.002 | 0.002 | 0.001 | 0.002 | 0.003 | 0.001 | 0.002 | 0.003 | 0.002 | 0.001 |
| Rottweiler | | 0.002 | 0.009 | 0.002 | 0.002 | 0.008 | 0.005 | 0.003 | 0.002 | 0.005 | 0.002 | 0.004 | 0.002 | 0.002 | 0.003 | 0.006 |
| Rottweiler | | 0.001 | 0.003 | 0.003 | 0.002 | 0.002 | 0.002 | 0.004 | 0.022 | 0.003 | 0.002 | 0.003 | 0.002 | 0.008 | 0.003 | 0.003 |
| Rottweiler | | 0.001 | 0.004 | 0.002 | 0.002 | 0.002 | 0.002 | 0.004 | 0.011 | 0.003 | 0.007 | 0.001 | 0.003 | 0.003 | 0.002 | 0.002 |
| Rottweiler | | 0.001 | 0.001 | 0.002 | 0.004 | 0.001 | 0.005 | 0.003 | 0.002 | 0.002 | 0.002 | 0.003 | 0.003 | 0.004 | 0.003 | 0.002 |
| Rottweiler | | 0.004 | 0.003 | 0.007 | 0.021 | 0.003 | 0.006 | 0.006 | 0.006 | 0.002 | 0.003 | 0.004 | 0.006 | 0.007 | 0.002 | 0.006 |
| Rottweiler | | 0.003 | 0.003 | 0.016 | 0.01 | 0.004 | 0.004 | 0.003 | 0.001 | 0.008 | 0.002 | 0.001 | 0.002 | 0.002 | 0.015 | 0.005 |
| Rottweiler | | 0.007 | 0.004 | 0.004 | 0.006 | 0.006 | 0.002 | 0.004 | 0.002 | 0.002 | 0.002 | 0.004 | 0.002 | 0.003 | 0.002 | 0.002 |
| Rottweiler | | 0.006 | 0.003 | 0.003 | 0.008 | 0.002 | 0.004 | 0.006 | 0.008 | 0.003 | 0.002 | 0.004 | 0.006 | 0.034 | 0.001 | 0.003 |
| Rottweiler | | 0.006 | 0.004 | 0.003 | 0.005 | 0.003 | 0.002 | 0.006 | 0.002 | 0.009 | 0.005 | 0.006 | 0.005 | 0.039 | 0.001 | 0.004 |
| Rottweiler | | 0.001 | 0.002 | 0.002 | 0.001 | 0.001 | 0.001 | 0.001 | 0.001 | 0.001 | 0.002 | 0.001 | 0.001 | 0.001 | 0.001 | 0.001 |
| Rottweiler | | 0.002 | 0.005 | 0.004 | 0.005 | 0.004 | 0.007 | 0.002 | 0.002 | 0.003 | 0.003 | 0.002 | 0.002 | 0.002 | 0.001 | 0.002 |
| Rottweiler | | 0.002 | 0.002 | 0.002 | 0.001 | 0.001 | 0.001 | 0.002 | 0.003 | 0.001 | 0.019 | 0.002 | 0.01 | 0.003 | 0.002 | 0.003 |
| Rottweiler | | 0.002 | 0.015 | 0.005 | 0.003 | 0.003 | 0.002 | 0.005 | 0.002 | 0.006 | 0.002 | 0.002 | 0.002 | 0.024 | 0.002 | 0.004 |
| Rottweiler | | 0.002 | 0.001 | 0.004 | 0.002 | 0.001 | 0.002 | 0.002 | 0.002 | 0.001 | 0.001 | 0.003 | 0.002 | 0.011 | 0.016 | 0.001 |
| Rottweiler | | 0.001 | 0.001 | 0.001 | 0.001 | 0.001 | 0.001 | 0.001 | 0.001 | 0.001 | 0.001 | 0.003 | 0.002 | 0.002 | 0.001 | 0.002 |
| Rottweiler | | 0.004 | 0.01 | 0.004 | 0.003 | 0.001 | 0.002 | 0.009 | 0.003 | 0.003 | 0.007 | 0.004 | 0.014 | 0.002 | 0.01 | 0.002 |
| Rottweiler | | 0.001 | 0.004 | 0.004 | 0.002 | 0.001 | 0.001 | 0.002 | 0.002 | 0.001 | 0.002 | 0.001 | 0.003 | 0.001 | 0.001 | 0.002 |
| Rottweiler | | 0.002 | 0.013 | 0.002 | 0.006 | 0.001 | 0.002 | 0.002 | 0.004 | 0.003 | 0.006 | 0.002 | 0.003 | 0.002 | 0.003 | 0.001 |
| Rottweiler | | 0.001 | 0.003 | 0.003 | 0.001 | 0.001 | 0.002 | 0.002 | 0.002 | 0.001 | 0.001 | 0.001 | 0.002 | 0.002 | 0.003 | 0.002 |
| Rottweiler | | 0.003 | 0.001 | 0.002 | 0.006 | 0.002 | 0.002 | 0.006 | 0.003 | 0.003 | 0.005 | 0.003 | 0.004 | 0.004 | 0.002 | 0.003 |
| Rottweiler | | 0.015 | 0.002 | 0.008 | 0.002 | 0.013 | 0.01 | 0.007 | 0.005 | 0.001 | 0.003 | 0.002 | 0.01 | 0.005 | 0.002 | 0.002 |
| Rottweiler | | 0.001 | 0.002 | 0.004 | 0.004 | 0.002 | 0.001 | 0.001 | 0.001 | 0.001 | 0.002 | 0.001 | 0.002 | 0.002 | 0.001 | 0.002 |
| Rottweiler | | 0.001 | 0.001 | 0.002 | 0.001 | 0.001 | 0.001 | 0.001 | 0.001 | 0.001 | 0.001 | 0.001 | 0.001 | 0.001 | 0.001 | 0.001 |
| Rottweiler | | 0.001 | 0.003 | 0.005 | 0.009 | 0.002 | 0.006 | 0.004 | 0.011 | 0.005 | 0.003 | 0.002 | 0.001 | 0.003 | 0.003 | 0.002 |
| Rottweiler | | 0.001 | 0.001 | 0.002 | 0.002 | 0.002 | 0.001 | 0.002 | 0.002 | 0.003 | 0.002 | 0.004 | 0.001 | 0.003 | 0.002 | 0.001 |
| Rottweiler | | 0.001 | 0.002 | 0.001 | 0.001 | 0.001 | 0.002 | 0.001 | 0.001 | 0.002 | 0.001 | 0.002 | 0.004 | 0.002 | 0.002 | 0.002 |
| Rottweiler | | 0.001 | 0.002 | 0.005 | 0.003 | 0.001 | 0.001 | 0.003 | 0.001 | 0.003 | 0.003 | 0.002 | 0.002 | 0.004 | 0.003 | 0.002 |
| Rottweiler | | 0.002 | 0.002 | 0.002 | 0.002 | 0.002 | 0.002 | 0.002 | 0.001 | 0.001 | 0.002 | 0.002 | 0.002 | 0.003 | 0.003 | 0.001 |
| | % | | | | | | | | | | | | | | | |
| | missing | Cluster assignment | | | | | | | | | | | | | | |
| Breed | data | 1 | 2 | 3 | 4 | 5 | 6 | 7 | 8 | 9 | 10 | 11 | 12 | 13 | 14 | 15 |
| Saluki | (2) | 0.02 | 0.003 | 0.195 | 0.504 | 0.007 | 0.004 | 0.003 | 0.009 | 0.002 | 0.002 | 0.007 | 0.002 | 0.003 | 0.022 | 0.003 |
| Saluki | 0 | 0.044 | 0.002 | 0.023 | 0.724 | 0.045 | 0.004 | 0.002 | 0.004 | 0.009 | 0.004 | 0.02 | 0.005 | 0.007 | 0.002 | 0.005 |
| Saluki | (1) | 0.002 | 0.004 | 0.002 | 0.942 | 0.001 | 0.002 | 0.001 | 0.002 | 0.001 | 0.002 | 0.002 | 0.003 | 0.004 | 0.002 | 0.002 |
| Saluki | (5) | 0.003 | 0.005 | 0.006 | 0.845 | 0.002 | 0.002 | 0.002 | 0.002 | 0.002 | 0.039 | 0.009 | 0.003 | 0.006 | 0.003 | 0.006 |
| Saluki | (4) | 0.003 | 0.004 | 0.002 | 0.93 | 0.001 | 0.002 | 0.003 | 0.003 | 0.001 | 0.005 | 0.001 | 0.002 | 0.003 | 0.001 | 0.003 |
| Saluki | 0 | 0.007 | 0.095 | 0.006 | 0.635 | 0.004 | 0.007 | 0.011 | 0.005 | 0.004 | 0.007 | 0.002 | 0.012 | 0.003 | 0.001 | 0.007 |
| Saluki | 0 | 0.002 | 0.003 | 0.003 | 0.932 | 0.003 | 0.002 | 0.002 | 0.003 | 0.001 | 0.002 | 0.001 | 0.001 | 0.002 | 0.004 | 0.002 |
| Saluki | (2) | 0.003 | 0.003 | 0.003 | 0.876 | 0.002 | 0.002 | 0.004 | 0.003 | 0.002 | 0.004 | 0.002 | 0.002 | 0.016 | 0.001 | 0.006 |
| Saluki | 0 | 0.002 | 0.004 | 0.002 | 0.759 | 0.003 | 0.006 | 0.02 | 0.008 | 0.001 | 0.01 | 0.002 | 0.019 | 0.003 | 0.005 | 0.002 |
| Saluki | (3) | 0.002 | 0.004 | 0.002 | 0.94 | 0.002 | 0.002 | 0.002 | 0.002 | 0.001 | 0.003 | 0.002 | 0.001 | 0.003 | 0.001 | 0.002 |
| Saluki | 0 | 0.001 | 0.003 | 0.002 | 0.872 | 0.009 | 0.004 | 0.002 | 0.004 | 0.002 | 0.001 | 0.001 | 0.003 | 0.001 | 0.002 | 0.002 |
| Saluki | (1) | 0.003 | 0.004 | 0.411 | 0.412 | 0.005 | 0.006 | 0.002 | 0.003 | 0.001 | 0.003 | 0.004 | 0.024 | 0.003 | 0.003 | 0.008 |
| Saluki | (4) | 0.009 | 0.006 | 0.322 | 0.483 | 0.004 | 0.003 | 0.004 | 0.002 | 0.003 | 0.002 | 0.003 | 0.002 | 0.006 | 0.004 | 0.021 |
| Saluki | (1) | 0.01 | 0.004 | 0.463 | 0.411 | 0.002 | 0.008 | 0.003 | 0.003 | 0.002 | 0.002 | 0.005 | 0.003 | 0.002 | 0.002 | 0.003 |
| Saluki | 0 | 0.004 | 0.009 | 0.623 | 0.112 | 0.003 | 0.034 | 0.006 | 0.008 | 0.005 | 0.002 | 0.005 | 0.002 | 0.002 | 0.001 | 0.003 |
| Saluki | (35) | 0.004 | 0.002 | 0.302 | 0.547 | 0.003 | 0.007 | 0.002 | 0.002 | 0.002 | 0.002 | 0.004 | 0.002 | 0.001 | 0.002 | 0.002 |
| Saluki | (51) | 0.004 | 0.006 | 0.622 | 0.021 | 0.003 | 0.008 | 0.005 | 0.051 | 0.003 | 0.004 | 0.039 | 0.005 | 0.003 | 0.008 | 0.018 |
| Saluki | (43) | 0.003 | 0.006 | 0.363 | 0.151 | 0.064 | 0.006 | 0.003 | 0.027 | 0.009 | 0.004 | 0.012 | 0.018 | 0.002 | 0.005 | 0.02 |
| Saluki | (69) | 0.002 | 0.002 | 0.899 | 0.022 | 0.002 | 0.003 | 0.002 | 0.003 | 0.003 | 0.002 | 0.006 | 0.002 | 0.003 | 0.003 | 0.002 |
| Saluki | (47) | 0.006 | 0.003 | 0.644 | 0.158 | 0.008 | 0.007 | 0.004 | 0.006 | 0.003 | 0.002 | 0.004 | 0.002 | 0.002 | 0.005 | 0.006 |
| Saluki | 0 | 0.003 | 0.011 | 0.005 | 0.667 | 0.003 | 0.005 | 0.001 | 0.008 | 0.003 | 0.001 | 0.003 | 0.002 | 0.003 | 0.004 | 0.162 |
| Saluki | 0 | 0.002 | 0.006 | 0.018 | 0.586 | 0.006 | 0.011 | 0.024 | 0.017 | 0.003 | 0.002 | 0.002 | 0.012 | 0.003 | 0.006 | 0.098 |
| Saluki | (4) | 0.002 | 0.006 | 0.011 | 0.662 | 0.005 | 0.004 | 0.003 | 0.002 | 0.002 | 0.001 | 0.002 | 0.004 | 0.002 | 0.003 | 0.008 |
| Saluki | (46) | 0.009 | 0.003 | 0.498 | 0.38 | 0.006 | 0.007 | 0.002 | 0.003 | 0.006 | 0.002 | 0.011 | 0.002 | 0.004 | 0.003 | 0.006 |
| Saluki | (1) | 0.006 | 0.164 | 0.652 | 0.006 | 0.003 | 0.005 | 0.003 | 0.008 | 0.003 | 0.004 | 0.003 | 0.089 | 0.003 | 0.003 | 0.002 |
| Saluki | 0 | 0.001 | 0.001 | 0.967 | 0.002 | 0.001 | 0.001 | 0.001 | 0.001 | 0.001 | 0.001 | 0.002 | 0.001 | 0.001 | 0.001 | 0.001 |
| Saluki | 0 | 0.002 | 0.002 | 0.931 | 0.003 | 0.003 | 0.001 | 0.001 | 0.003 | 0.001 | 0.001 | 0.003 | 0.001 | 0.002 | 0.001 | 0.011 |
| Saluki | (2) | 0.003 | 0.002 | 0.007 | 0.865 | 0.002 | 0.004 | 0.004 | 0.002 | 0.001 | 0.004 | 0.005 | 0.005 | 0.002 | 0.043 | 0.002 |
| Saluki | (1) | 0.004 | 0.005 | 0.028 | 0.879 | 0.004 | 0.007 | 0.004 | 0.006 | 0.004 | 0.001 | 0.004 | 0.003 | 0.002 | 0.001 | 0.002 |
| Saluki | 0 | 0.004 | 0.095 | 0.439 | 0.317 | 0.005 | 0.005 | 0.008 | 0.005 | 0.004 | 0.003 | 0.006 | 0.002 | 0.008 | 0.002 | 0.016 |
| Saluki | 0 | 0.006 | 0.006 | 0.012 | 0.866 | 0.002 | 0.001 | 0.001 | 0.003 | 0.006 | 0.016 | 0.002 | 0.002 | 0.002 | 0.002 | 0.004 |
| Saluki | (59) | 0.002 | 0.002 | 0.87 | 0.018 | 0.013 | 0.006 | 0.004 | 0.005 | 0.005 | 0.002 | 0.005 | 0.003 | 0.001 | 0.002 | 0.004 |
| Saluki | 0 | 0.002 | 0.007 | 0.015 | 0.779 | 0.002 | 0.006 | 0.012 | 0.006 | 0.024 | 0.034 | 0.002 | 0.002 | 0.009 | 0.002 | 0.003 |
| Saluki | 0 | 0.002 | 0.002 | 0.008 | 0.906 | 0.005 | 0.003 | 0.003 | 0.003 | 0.004 | 0.001 | 0.005 | 0.006 | 0.002 | 0.003 | 0.003 |
| Saluki | (6) | 0.011 | 0.009 | 0.005 | 0.799 | 0.003 | 0.003 | 0.003 | 0.013 | 0.003 | 0.002 | 0.008 | 0.008 | 0.003 | 0.007 | 0.006 |
| Saluki | 0 | 0.008 | 0.002 | 0.004 | 0.841 | 0.006 | 0.003 | 0.001 | 0.002 | 0.002 | 0.001 | 0.036 | 0.005 | 0.002 | 0.004 | 0.007 |
| Saluki | (1) | 0.006 | 0.005 | 0.008 | 0.844 | 0.003 | 0.008 | 0.004 | 0.003 | 0.002 | 0.001 | 0.009 | 0.013 | 0.004 | 0.022 | 0.001 |
| Saluki | (49) | 0.007 | 0.002 | 0.024 | 0.887 | 0.002 | 0.004 | 0.003 | 0.003 | 0.003 | 0.001 | 0.008 | 0.002 | 0.002 | 0.005 | 0.003 |
| | | Cluster Assignment | | | | | | | | | | | | | | |
| Breed | | 16 | 17 | 18 | 19 | 20 | 21 | 22 | 23 | 24 | 25 | 26 | 27 | 28 | 29 | 30 |
| Saluki | | 0.011 | 0.002 | 0.012 | 0.03 | 0.004 | 0.003 | 0.076 | 0.002 | 0.038 | 0.003 | 0.006 | 0.002 | 0.014 | 0.004 | 0.008 |
| Saluki | | 0.004 | 0.001 | 0.004 | 0.009 | 0.015 | 0.003 | 0.009 | 0.001 | 0.024 | 0.002 | 0.005 | 0.002 | 0.011 | 0.002 | 0.006 |
| Saluki | | 0.001 | 0.002 | 0.002 | 0.002 | 0.001 | 0.001 | 0.001 | 0.002 | 0.002 | 0.003 | 0.001 | 0.004 | 0.001 | 0.002 | 0.001 |
| Saluki | | 0.001 | 0.004 | 0.003 | 0.004 | 0.005 | 0.008 | 0.005 | 0.01 | 0.002 | 0.004 | 0.004 | 0.004 | 0.006 | 0.004 | 0.003 |
| Saluki | | 0.001 | 0.002 | 0.003 | 0.002 | 0.001 | 0.006 | 0.002 | 0.003 | 0.001 | 0.005 | 0.004 | 0.002 | 0.002 | 0.001 | 0.002 |
| Saluki | | 0.002 | 0.002 | 0.009 | 0.003 | 0.009 | 0.003 | 0.019 | 0.064 | 0.005 | 0.004 | 0.025 | 0.004 | 0.003 | 0.005 | 0.036 |
| Saluki | | 0.002 | 0.002 | 0.002 | 0.002 | 0.001 | 0.003 | 0.001 | 0.003 | 0.003 | 0.003 | 0.003 | 0.004 | 0.002 | 0.004 | 0.004 |
| Saluki | | 0.002 | 0.002 | 0.002 | 0.004 | 0.002 | 0.004 | 0.001 | 0.004 | 0.002 | 0.018 | 0.003 | 0.014 | 0.003 | 0.005 | 0.008 |
| Saluki | | 0.002 | 0.003 | 0.004 | 0.059 | 0.002 | 0.015 | 0.009 | 0.026 | 0.002 | 0.005 | 0.004 | 0.001 | 0.002 | 0.001 | 0.018 |
| Saluki | | 0.001 | 0.002 | 0.002 | 0.006 | 0.002 | 0.001 | 0.002 | 0.002 | 0.001 | 0.002 | 0.002 | 0.002 | 0.001 | 0.002 | 0.001 |
| Saluki | | 0.002 | 0.002 | 0.001 | 0.067 | 0.001 | 0.003 | 0.001 | 0.002 | 0.002 | 0.002 | 0.002 | 0.001 | 0.002 | 0.001 | 0.002 |
| Saluki | | 0.002 | 0.004 | 0.005 | 0.006 | 0.006 | 0.002 | 0.002 | 0.006 | 0.02 | 0.004 | 0.006 | 0.007 | 0.02 | 0.005 | 0.015 |
| Saluki | | 0.003 | 0.003 | 0.004 | 0.003 | 0.003 | 0.004 | 0.007 | 0.004 | 0.06 | 0.003 | 0.002 | 0.003 | 0.007 | 0.003 | 0.018 |
| Saluki | | 0.002 | 0.006 | 0.007 | 0.023 | 0.002 | 0.002 | 0.002 | 0.004 | 0.003 | 0.001 | 0.005 | 0.003 | 0.003 | 0.001 | 0.013 |
| Saluki | | 0.005 | 0.004 | 0.009 | 0.128 | 0.002 | 0.002 | 0.005 | 0.01 | 0.003 | 0.001 | 0.002 | 0.001 | 0.004 | 0.002 | 0.003 |
| Saluki | | 0.002 | 0.004 | 0.002 | 0.073 | 0.001 | 0.003 | 0.007 | 0.006 | 0.003 | 0.001 | 0.001 | 0.001 | 0.002 | 0.003 | 0.004 |
| Saluki | | 0.018 | 0.037 | 0.01 | 0.005 | 0.005 | 0.009 | 0.015 | 0.005 | 0.005 | 0.015 | 0.034 | 0.011 | 0.004 | 0.023 | 0.006 |
| Saluki | | 0.018 | 0.046 | 0.086 | 0.015 | 0.008 | 0.016 | 0.01 | 0.009 | 0.014 | 0.018 | 0.009 | 0.006 | 0.009 | 0.033 | 0.007 |
| Saluki | | 0.002 | 0.002 | 0.003 | 0.007 | 0.003 | 0.003 | 0.004 | 0.002 | 0.003 | 0.002 | 0.002 | 0.002 | 0.002 | 0.003 | 0.003 |
| Saluki | | 0.007 | 0.039 | 0.007 | 0.002 | 0.004 | 0.002 | 0.005 | 0.019 | 0.021 | 0.004 | 0.003 | 0.005 | 0.004 | 0.013 | 0.004 |
| Saluki | | 0.004 | 0.002 | 0.004 | 0.018 | 0.004 | 0.002 | 0.004 | 0.003 | 0.015 | 0.006 | 0.003 | 0.017 | 0.008 | 0.019 | 0.007 |
| Saluki | | 0.034 | 0.011 | 0.007 | 0.004 | 0.024 | 0.01 | 0.002 | 0.003 | 0.03 | 0.003 | 0.002 | 0.007 | 0.023 | 0.042 | 0.002 |
| Saluki | | 0.003 | 0.002 | 0.001 | 0.005 | 0.001 | 0.002 | 0.002 | 0.004 | 0.01 | 0.002 | 0.002 | 0.003 | 0.007 | 0.237 | 0.001 |
| Saluki | | 0.003 | 0.004 | 0.003 | 0.014 | 0.004 | 0.004 | 0.003 | 0.005 | 0.002 | 0.002 | 0.002 | 0.002 | 0.002 | 0.003 | 0.004 |
| Saluki | | 0.001 | 0.002 | 0.003 | 0.013 | 0.003 | 0.004 | 0.001 | 0.004 | 0.002 | 0.005 | 0.002 | 0.002 | 0.002 | 0.001 | 0.003 |
| Saluki | | 0.001 | 0.001 | 0.001 | 0.001 | 0.001 | 0.002 | 0.001 | 0.001 | 0.001 | 0.001 | 0.001 | 0.001 | 0.002 | 0.001 | 0.001 |
| Saluki | | 0.002 | 0.003 | 0.003 | 0.004 | 0.001 | 0.003 | 0.002 | 0.001 | 0.001 | 0.001 | 0.001 | 0.001 | 0.008 | 0.003 | 0.001 |
| Saluki | | 0.001 | 0.002 | 0.002 | 0.003 | 0.003 | 0.004 | 0.002 | 0.006 | 0.003 | 0.002 | 0.007 | 0.001 | 0.011 | 0.001 | 0.002 |
| Saluki | | 0.001 | 0.001 | 0.002 | 0.013 | 0.001 | 0.002 | 0.005 | 0.002 | 0.004 | 0.003 | 0.004 | 0.002 | 0.003 | 0.001 | 0.002 |
| Saluki | | 0.003 | 0.003 | 0.006 | 0.003 | 0.016 | 0.007 | 0.004 | 0.003 | 0.003 | 0.014 | 0.003 | 0.004 | 0.002 | 0.004 | 0.005 |
| Saluki | | 0.002 | 0.004 | 0.003 | 0.007 | 0.003 | 0.002 | 0.006 | 0.027 | 0.001 | 0.003 | 0.003 | 0.002 | 0.002 | 0.003 | 0.005 |
| Saluki | | 0.002 | 0.003 | 0.002 | 0.008 | 0.005 | 0.002 | 0.006 | 0.004 | 0.004 | 0.003 | 0.003 | 0.002 | 0.005 | 0.003 | 0.005 |
| Saluki | | 0.003 | 0.002 | 0.002 | 0.005 | 0.004 | 0.007 | 0.004 | 0.005 | 0.002 | 0.007 | 0.05 | 0.001 | 0.001 | 0.001 | 0.002 |
| Saluki | | 0.001 | 0.001 | 0.002 | 0.003 | 0.002 | 0.002 | 0.004 | 0.002 | 0.004 | 0.002 | 0.005 | 0.003 | 0.011 | 0.002 | 0.003 |
| Saluki | | 0.003 | 0.005 | 0.004 | 0.016 | 0.002 | 0.009 | 0.006 | 0.004 | 0.015 | 0.003 | 0.005 | 0.025 | 0.008 | 0.007 | 0.004 |
| Saluki | | 0.007 | 0.007 | 0.004 | 0.002 | 0.002 | 0.003 | 0.005 | 0.002 | 0.008 | 0.002 | 0.003 | 0.012 | 0.014 | 0.003 | 0.003 |
| Saluki | | 0.003 | 0.004 | 0.003 | 0.004 | 0.004 | 0.007 | 0.003 | 0.003 | 0.012 | 0.002 | 0.003 | 0.002 | 0.012 | 0.002 | 0.003 |
| Saluki | | 0.001 | 0.004 | 0.005 | 0.002 | 0.001 | 0.001 | 0.003 | 0.002 | 0.006 | 0.005 | 0.001 | 0.002 | 0.005 | 0.003 | 0.002 |
| | % | | | | | | | | | | | | | | | |
| | missing | Cluster assignment | | | | | | | | | | | | | | |
| Breed | data | 1 | 2 | 3 | 4 | 5 | 6 | 7 | 8 | 9 | 10 | 11 | 12 | 13 | 14 | 15 |
| Samoyed | (1) | 0.003 | 0.031 | 0.006 | 0.006 | 0.004 | 0.002 | 0.004 | 0.004 | 0.001 | 0.006 | 0.003 | 0.002 | 0.002 | 0.8 | 0.008 |
| Samoyed | (1) | 0.004 | 0.023 | 0.003 | 0.004 | 0.035 | 0.002 | 0.002 | 0.004 | 0.004 | 0.013 | 0.003 | 0.003 | 0.001 | 0.727 | 0.024 |
| Samoyed | 0 | 0.003 | 0.055 | 0.004 | 0.003 | 0.019 | 0.006 | 0.004 | 0.006 | 0.002 | 0.014 | 0.018 | 0.002 | 0.002 | 0.734 | 0.006 |
| Samoyed | 0 | 0.003 | 0.02 | 0.004 | 0.004 | 0.004 | 0.007 | 0.01 | 0.025 | 0.001 | 0.049 | 0.004 | 0.004 | 0.011 | 0.738 | 0.003 |
| Samoyed | 0 | 0.001 | 0.006 | 0.002 | 0.004 | 0.003 | 0.011 | 0.004 | 0.006 | 0.001 | 0.004 | 0.004 | 0.006 | 0.02 | 0.792 | 0.003 |
| Samoyed | (2) | 0.01 | 0.008 | 0.003 | 0.005 | 0.003 | 0.006 | 0.007 | 0.011 | 0.002 | 0.003 | 0.012 | 0.03 | 0.01 | 0.757 | 0.004 |
| Samoyed | (3) | 0.003 | 0.006 | 0.002 | 0.002 | 0.004 | 0.002 | 0.002 | 0.002 | 0.001 | 0.002 | 0.006 | 0.001 | 0.003 | 0.916 | 0.002 |
| Samoyed | (2) | 0.003 | 0.006 | 0.003 | 0.002 | 0.001 | 0.001 | 0.002 | 0.002 | 0.001 | 0.001 | 0.003 | 0.002 | 0.001 | 0.934 | 0.002 |
| Samoyed | (2) | 0.003 | 0.002 | 0.002 | 0.001 | 0.002 | 0.001 | 0.001 | 0.002 | 0.001 | 0.001 | 0.001 | 0.002 | 0.001 | 0.955 | 0.001 |
| Samoyed | 0 | 0.001 | 0.004 | 0.002 | 0.001 | 0.001 | 0.002 | 0.001 | 0.001 | 0.001 | 0.001 | 0.003 | 0.001 | 0.002 | 0.947 | 0.002 |
| Samoyed | (1) | 0.001 | 0.002 | 0.002 | 0.001 | 0.002 | 0.001 | 0.001 | 0.002 | 0.001 | 0.001 | 0.002 | 0.001 | 0.001 | 0.955 | 0.002 |
| Samoyed | 0 | 0.007 | 0.003 | 0.002 | 0.002 | 0.003 | 0.003 | 0.003 | 0.003 | 0.001 | 0.001 | 0.005 | 0.001 | 0.002 | 0.92 | 0.002 |
| Samoyed | 0 | 0.008 | 0.001 | 0.026 | 0.017 | 0.055 | 0.001 | 0.002 | 0.002 | 0.003 | 0.004 | 0.007 | 0.006 | 0.001 | 0.77 | 0.023 |
| Samoyed | (1) | 0.004 | 0.005 | 0.001 | 0.004 | 0.003 | 0.003 | 0.005 | 0.016 | 0.004 | 0.003 | 0.004 | 0.005 | 0.015 | 0.757 | 0.002 |
| Samoyed | (8) | 0.004 | 0.002 | 0.001 | 0.004 | 0.001 | 0.006 | 0.003 | 0.006 | 0.003 | 0.01 | 0.002 | 0.004 | 0.009 | 0.781 | 0.002 |
| Samoyed | (11) | 0.006 | 0.006 | 0.002 | 0.004 | 0.002 | 0.005 | 0.004 | 0.005 | 0.006 | 0.008 | 0.003 | 0.005 | 0.01 | 0.804 | 0.004 |
| Samoyed | 0 | 0.002 | 0.002 | 0.002 | 0.005 | 0.011 | 0.005 | 0.006 | 0.003 | 0.003 | 0.008 | 0.007 | 0.002 | 0.006 | 0.825 | 0.008 |
| Samoyed | (2) | 0.004 | 0.027 | 0.001 | 0.002 | 0.005 | 0.003 | 0.012 | 0.021 | 0.004 | 0.03 | 0.004 | 0.017 | 0.021 | 0.618 | 0.004 |
| Samoyed | (1) | 0.004 | 0.005 | 0.002 | 0.006 | 0.013 | 0.003 | 0.002 | 0.003 | 0.003 | 0.003 | 0.002 | 0.001 | 0.003 | 0.759 | 0.118 |
| Samoyed | 0 | 0.001 | 0.001 | 0.002 | 0.001 | 0.001 | 0.001 | 0.001 | 0.001 | 0.001 | 0.002 | 0.002 | 0.002 | 0.001 | 0.953 | 0.001 |
| Samoyed | (1) | 0.005 | 0.002 | 0.025 | 0.002 | 0.003 | 0.004 | 0.001 | 0.003 | 0.009 | 0.003 | 0.004 | 0.001 | 0.002 | 0.875 | 0.021 |
| Samoyed | (2) | 0.002 | 0.003 | 0.002 | 0.001 | 0.001 | 0.001 | 0.002 | 0.002 | 0.001 | 0.002 | 0.002 | 0.002 | 0.001 | 0.946 | 0.001 |
| Samoyed | (2) | 0.012 | 0.003 | 0.003 | 0.007 | 0.005 | 0.003 | 0.006 | 0.008 | 0.004 | 0.003 | 0.022 | 0.003 | 0.003 | 0.835 | 0.004 |
| Samoyed | (2) | 0.003 | 0.004 | 0.001 | 0.002 | 0.002 | 0.002 | 0.002 | 0.003 | 0.005 | 0.003 | 0.006 | 0.002 | 0.002 | 0.923 | 0.004 |
| Samoyed | (8) | 0.002 | 0.001 | 0.002 | 0.001 | 0.001 | 0.001 | 0.001 | 0.002 | 0.002 | 0.001 | 0.003 | 0.001 | 0.001 | 0.956 | 0.001 |
| Samoyed | 0 | 0.001 | 0.002 | 0.004 | 0.002 | 0.001 | 0.001 | 0.002 | 0.001 | 0.001 | 0.002 | 0.003 | 0.001 | 0.002 | 0.95 | 0.002 |
| Samoyed | 0 | 0.001 | 0.002 | 0.002 | 0.001 | 0.001 | 0.001 | 0.002 | 0.002 | 0.003 | 0.003 | 0.003 | 0.002 | 0.001 | 0.934 | 0.002 |
| Samoyed | 0 | 0.001 | 0.001 | 0.001 | 0.001 | 0.001 | 0.001 | 0.001 | 0.001 | 0.002 | 0.002 | 0.001 | 0.001 | 0.001 | 0.963 | 0.001 |
| Samoyed | (1) | 0.016 | 0.002 | 0.004 | 0.003 | 0.001 | 0.002 | 0.003 | 0.006 | 0.003 | 0.002 | 0.013 | 0.002 | 0.002 | 0.875 | 0.007 |
| Samoyed | 0 | 0.001 | 0.002 | 0.002 | 0.001 | 0.001 | 0.001 | 0.001 | 0.002 | 0.001 | 0.002 | 0.002 | 0.004 | 0.001 | 0.95 | 0.001 |
| Samoyed | (1) | 0.002 | 0.001 | 0.011 | 0.002 | 0.001 | 0.001 | 0.002 | 0.004 | 0.004 | 0.002 | 0.005 | 0.002 | 0.001 | 0.933 | 0.002 |
| Samoyed | (6) | 0.002 | 0.002 | 0.018 | 0.002 | 0.001 | 0.003 | 0.001 | 0.002 | 0.006 | 0.002 | 0.002 | 0.001 | 0.001 | 0.927 | 0.003 |
| Samoyed | (10) | 0.003 | 0.003 | 0.039 | 0.002 | 0.002 | 0.002 | 0.002 | 0.003 | 0.009 | 0.003 | 0.003 | 0.003 | 0.002 | 0.874 | 0.01 |
| Samoyed | (8) | 0.002 | 0.004 | 0.001 | 0.001 | 0.001 | 0.001 | 0.003 | 0.002 | 0.002 | 0.002 | 0.003 | 0.003 | 0.001 | 0.951 | 0.001 |
| Samoyed | (4) | 0.015 | 0.006 | 0.006 | 0.019 | 0.004 | 0.006 | 0.003 | 0.021 | 0.002 | 0.012 | 0.011 | 0.002 | 0.002 | 0.622 | 0.004 |
| Samoyed | (6) | 0.002 | 0.003 | 0.003 | 0.008 | 0.001 | 0.005 | 0.004 | 0.004 | 0.001 | 0.019 | 0.002 | 0.02 | 0.009 | 0.856 | 0.002 |
| Samoyed | (9) | 0.004 | 0.003 | 0.019 | 0.02 | 0.011 | 0.002 | 0.004 | 0.003 | 0.003 | 0.004 | 0.005 | 0.002 | 0.016 | 0.73 | 0.002 |
| Samoyed | (4) | 0.002 | 0.009 | 0.007 | 0.018 | 0.002 | 0.004 | 0.008 | 0.046 | 0.004 | 0.027 | 0.002 | 0.034 | 0.005 | 0.64 | 0.001 |
| | | Cluster Assignment | | | | | | | | | | | | | | |
| Breed | | 16 | 17 | 18 | 19 | 20 | 21 | 22 | 23 | 24 | 25 | 26 | 27 | 28 | 29 | 30 |
| Samoyed | | 0.002 | 0.024 | 0.006 | 0.003 | 0.007 | 0.003 | 0.002 | 0.046 | 0.003 | 0.002 | 0.003 | 0.003 | 0.004 | 0.004 | 0.007 |
| Samoyed | | 0.003 | 0.003 | 0.004 | 0.005 | 0.026 | 0.078 | 0.007 | 0.002 | 0.004 | 0.001 | 0.002 | 0.001 | 0.003 | 0.004 | 0.005 |
| Samoyed | | 0.002 | 0.005 | 0.007 | 0.025 | 0.021 | 0.021 | 0.014 | 0.003 | 0.003 | 0.008 | 0.002 | 0.003 | 0.005 | 0.002 | 0.002 |
| Samoyed | | 0.004 | 0.002 | 0.006 | 0.009 | 0.008 | 0.02 | 0.002 | 0.007 | 0.001 | 0.029 | 0.012 | 0.004 | 0.005 | 0.001 | 0.004 |
| Samoyed | | 0.002 | 0.004 | 0.021 | 0.003 | 0.003 | 0.002 | 0.005 | 0.015 | 0.003 | 0.009 | 0.002 | 0.059 | 0.002 | 0.003 | 0.003 |
| Samoyed | | 0.004 | 0.003 | 0.007 | 0.017 | 0.002 | 0.004 | 0.005 | 0.023 | 0.006 | 0.013 | 0.009 | 0.017 | 0.003 | 0.012 | 0.003 |
| Samoyed | | 0.001 | 0.002 | 0.007 | 0.006 | 0.002 | 0.002 | 0.005 | 0.001 | 0.005 | 0.001 | 0.002 | 0.003 | 0.002 | 0.005 | 0.003 |
| Samoyed | | 0.001 | 0.004 | 0.003 | 0.002 | 0.001 | 0.002 | 0.002 | 0.002 | 0.003 | 0.002 | 0.002 | 0.003 | 0.001 | 0.006 | 0.003 |
| Samoyed | | 0.001 | 0.002 | 0.003 | 0.001 | 0.001 | 0.002 | 0.001 | 0.001 | 0.003 | 0.001 | 0.001 | 0.002 | 0.001 | 0.001 | 0.002 |
| Samoyed | | 0.001 | 0.004 | 0.003 | 0.002 | 0.002 | 0.001 | 0.002 | 0.002 | 0.003 | 0.001 | 0.001 | 0.001 | 0.001 | 0.001 | 0.002 |
| Samoyed | | 0.001 | 0.002 | 0.002 | 0.002 | 0.001 | 0.002 | 0.002 | 0.001 | 0.003 | 0.001 | 0.001 | 0.001 | 0.002 | 0.002 | 0.001 |
| Samoyed | | 0.001 | 0.002 | 0.002 | 0.002 | 0.001 | 0.005 | 0.003 | 0.002 | 0.003 | 0.002 | 0.01 | 0.002 | 0.001 | 0.004 | 0.003 |
| Samoyed | | 0.004 | 0.002 | 0.002 | 0.006 | 0.002 | 0.007 | 0.002 | 0.002 | 0.008 | 0.001 | 0.003 | 0.003 | 0.011 | 0.019 | 0.004 |
| Samoyed | | 0.002 | 0.004 | 0.005 | 0.003 | 0.063 | 0.004 | 0.008 | 0.019 | 0.003 | 0.003 | 0.004 | 0.007 | 0.041 | 0.001 | 0.001 |
| Samoyed | | 0.001 | 0.01 | 0.004 | 0.004 | 0.002 | 0.003 | 0.001 | 0.008 | 0.003 | 0.005 | 0.026 | 0.003 | 0.088 | 0.002 | 0.001 |
| Samoyed | | 0.002 | 0.018 | 0.005 | 0.003 | 0.003 | 0.002 | 0.003 | 0.003 | 0.011 | 0.004 | 0.01 | 0.026 | 0.011 | 0.026 | 0.003 |
| Samoyed | | 0.003 | 0.001 | 0.002 | 0.004 | 0.004 | 0.013 | 0.001 | 0.002 | 0.002 | 0.002 | 0.018 | 0.005 | 0.021 | 0.025 | 0.002 |
| Samoyed | | 0.002 | 0.005 | 0.005 | 0.007 | 0.006 | 0.003 | 0.005 | 0.002 | 0.006 | 0.019 | 0.01 | 0.115 | 0.005 | 0.002 | 0.033 |
| Samoyed | | 0.006 | 0.002 | 0.006 | 0.002 | 0.018 | 0.006 | 0.003 | 0.002 | 0.004 | 0.001 | 0.002 | 0.004 | 0.001 | 0.004 | 0.01 |
| Samoyed | | 0.001 | 0.003 | 0.002 | 0.001 | 0.001 | 0.001 | 0.003 | 0.001 | 0.002 | 0.002 | 0.001 | 0.002 | 0.001 | 0.001 | 0.003 |
| Samoyed | | 0.001 | 0.002 | 0.002 | 0.002 | 0.001 | 0.006 | 0.003 | 0.002 | 0.006 | 0.002 | 0.001 | 0.002 | 0.003 | 0.002 | 0.002 |
| Samoyed | | 0.001 | 0.004 | 0.002 | 0.002 | 0.001 | 0.001 | 0.002 | 0.002 | 0.002 | 0.004 | 0.001 | 0.004 | 0.001 | 0.001 | 0.002 |
| Samoyed | | 0.001 | 0.008 | 0.008 | 0.005 | 0.003 | 0.003 | 0.012 | 0.004 | 0.008 | 0.002 | 0.006 | 0.005 | 0.005 | 0.003 | 0.005 |
| Samoyed | | 0.001 | 0.004 | 0.004 | 0.002 | 0.001 | 0.002 | 0.006 | 0.002 | 0.003 | 0.002 | 0.001 | 0.002 | 0.003 | 0.002 | 0.002 |
| Samoyed | | 0.001 | 0.003 | 0.002 | 0.001 | 0.001 | 0.001 | 0.002 | 0.001 | 0.002 | 0.001 | 0.002 | 0.001 | 0.002 | 0.001 | 0.002 |
| Samoyed | | 0.001 | 0.002 | 0.003 | 0.002 | 0.001 | 0.002 | 0.002 | 0.001 | 0.001 | 0.002 | 0.001 | 0.002 | 0.002 | 0.001 | 0.001 |
| Samoyed | | 0.001 | 0.008 | 0.002 | 0.003 | 0.001 | 0.002 | 0.004 | 0.005 | 0.002 | 0.001 | 0.002 | 0.003 | 0.002 | 0.002 | 0.002 |
| Samoyed | | 0.001 | 0.001 | 0.002 | 0.001 | 0.001 | 0.002 | 0.002 | 0.001 | 0.002 | 0.001 | 0.001 | 0.001 | 0.002 | 0.001 | 0.001 |
| Samoyed | | 0.002 | 0.004 | 0.004 | 0.005 | 0.003 | 0.003 | 0.003 | 0.009 | 0.007 | 0.006 | 0.001 | 0.006 | 0.002 | 0.001 | 0.002 |
| Samoyed | | 0.001 | 0.001 | 0.003 | 0.001 | 0.001 | 0.002 | 0.001 | 0.002 | 0.004 | 0.002 | 0.002 | 0.002 | 0.001 | 0.002 | 0.002 |
| Samoyed | | 0.001 | 0.002 | 0.002 | 0.002 | 0.002 | 0.002 | 0.002 | 0.001 | 0.002 | 0.002 | 0.001 | 0.002 | 0.003 | 0.001 | 0.001 |
| Samoyed | | 0.001 | 0.002 | 0.001 | 0.001 | 0.001 | 0.003 | 0.002 | 0.002 | 0.002 | 0.001 | 0.001 | 0.001 | 0.001 | 0.005 | 0.002 |
| Samoyed | | 0.002 | 0.005 | 0.002 | 0.003 | 0.002 | 0.002 | 0.004 | 0.002 | 0.006 | 0.003 | 0.001 | 0.002 | 0.003 | 0.003 | 0.002 |
| Samoyed | | 0.002 | 0.002 | 0.002 | 0.002 | 0.001 | 0.002 | 0.001 | 0.002 | 0.002 | 0.001 | 0.002 | 0.001 | 0.002 | 0.001 | 0.002 |
| Samoyed | | 0.003 | 0.021 | 0.016 | 0.025 | 0.006 | 0.086 | 0.004 | 0.011 | 0.005 | 0.003 | 0.004 | 0.006 | 0.065 | 0.005 | 0.004 |
| Samoyed | | 0.001 | 0.003 | 0.003 | 0.004 | 0.004 | 0.008 | 0.006 | 0.002 | 0.005 | 0.006 | 0.006 | 0.003 | 0.002 | 0.004 | 0.005 |
| Samoyed | | 0.004 | 0.048 | 0.003 | 0.004 | 0.012 | 0.004 | 0.028 | 0.003 | 0.003 | 0.022 | 0.005 | 0.026 | 0.003 | 0.005 | 0.001 |
| Samoyed | | 0.003 | 0.019 | 0.009 | 0.007 | 0.004 | 0.002 | 0.007 | 0.005 | 0.003 | 0.065 | 0.04 | 0.013 | 0.007 | 0.001 | 0.006 |
| | % | | | | | | | | | | | | | | | |
| | missing | Cluster assignment | | | | | | | | | | | | | | |
| Breed | data | 1 | 2 | 3 | 4 | 5 | 6 | 7 | 8 | 9 | 10 | 11 | 12 | 13 | 14 | 15 |
| Shetland | (7) | 0.001 | 0.001 | 0.001 | 0.001 | 0.001 | 0.001 | 0.001 | 0.002 | 0.962 | 0.001 | 0.002 | 0.001 | 0.001 | 0.002 | 0.001 |
| Sheepdog | | | | | | | | | | | | | | | | |
| Shetland | (8) | 0.002 | 0.001 | 0.001 | 0.001 | 0.001 | 0.001 | 0.002 | 0.001 | 0.951 | 0.001 | 0.001 | 0.002 | 0.001 | 0.002 | 0.001 |
| Sheepdog | | | | | | | | | | | | | | | | |
| Shetland | (11) | 0.001 | 0.001 | 0.001 | 0.002 | 0.002 | 0.001 | 0.002 | 0.001 | 0.956 | 0.001 | 0.001 | 0.001 | 0.002 | 0.001 | 0.001 |
| Sheepdog | | | | | | | | | | | | | | | | |
| Shetland | 0 | 0.001 | 0.001 | 0.001 | 0.001 | 0.001 | 0.001 | 0.001 | 0.001 | 0.972 | 0.002 | 0.001 | 0.001 | 0.001 | 0.001 | 0.001 |
| Sheepdog | | | | | | | | | | | | | | | | |
| Shetland | 0 | 0.002 | 0.002 | 0.001 | 0.001 | 0.002 | 0.002 | 0.001 | 0.001 | 0.945 | 0.002 | 0.003 | 0.002 | 0.002 | 0.002 | 0.002 |
| Sheepdog | | | | | | | | | | | | | | | | |
| Shetland | (1) | 0.004 | 0.006 | 0.003 | 0.003 | 0.005 | 0.003 | 0.003 | 0.002 | 0.9 | 0.006 | 0.001 | 0.002 | 0.004 | 0.001 | 0.007 |
| Sheepdog | | | | | | | | | | | | | | | | |
| Shetland | 0 | 0.005 | 0.008 | 0.003 | 0.002 | 0.001 | 0.013 | 0.076 | 0.003 | 0.804 | 0.008 | 0.004 | 0.005 | 0.006 | 0.002 | 0.008 |
| Sheepdog | | | | | | | | | | | | | | | | |
| Shetland | 0 | 0.004 | 0.005 | 0.002 | 0.002 | 0.001 | 0.007 | 0.002 | 0.002 | 0.909 | 0.002 | 0.013 | 0.001 | 0.004 | 0.006 | 0.001 |
| Sheepdog | | | | | | | | | | | | | | | | |
| Shetland | 0 | 0.003 | 0.005 | 0.001 | 0.002 | 0.005 | 0.002 | 0.003 | 0.002 | 0.932 | 0.001 | 0.001 | 0.002 | 0.001 | 0.001 | 0.003 |
| Sheepdog | | | | | | | | | | | | | | | | |
| Shetland | 0 | 0.001 | 0.001 | 0.001 | 0.001 | 0.002 | 0.001 | 0.001 | 0.001 | 0.968 | 0.001 | 0.001 | 0.001 | 0.001 | 0.001 | 0.001 |
| Sheepdog | | | | | | | | | | | | | | | | |
| Shetland | (1) | 0.016 | 0.004 | 0.008 | 0.004 | 0.007 | 0.001 | 0.002 | 0.005 | 0.855 | 0.008 | 0.012 | 0.001 | 0.003 | 0.002 | 0.003 |
| Sheepdog | | | | | | | | | | | | | | | | |
| Shetland | 0 | 0.003 | 0.002 | 0.001 | 0.001 | 0.001 | 0.002 | 0.002 | 0.001 | 0.96 | 0.001 | 0.002 | 0.001 | 0.001 | 0.001 | 0.001 |
| Sheepdog | | | | | | | | | | | | | | | | |
| Shetland | (2) | 0.009 | 0.001 | 0.001 | 0.001 | 0.001 | 0.002 | 0.003 | 0.001 | 0.926 | 0.004 | 0.002 | 0.003 | 0.002 | 0.001 | 0.002 |
| Sheepdog | | | | | | | | | | | | | | | | |
| Shetland | (1) | 0.004 | 0.004 | 0.009 | 0.019 | 0.001 | 0.006 | 0.006 | 0.007 | 0.831 | 0.003 | 0.003 | 0.008 | 0.009 | 0.007 | 0.004 |
| Sheepdog | | | | | | | | | | | | | | | | |
| Shetland | 0 | 0.001 | 0.001 | 0.001 | 0.001 | 0.001 | 0.001 | 0.001 | 0.001 | 0.973 | 0.001 | 0.001 | 0.001 | 0.001 | 0.001 | 0.001 |
| Sheepdog | | | | | | | | | | | | | | | | |
| Shetland | 0 | 0.001 | 0.001 | 0.001 | 0.001 | 0.001 | 0.002 | 0.001 | 0.001 | 0.961 | 0.002 | 0.002 | 0.001 | 0.001 | 0.001 | 0.001 |
| Sheepdog | | | | | | | | | | | | | | | | |
| Shetland | (1) | 0.001 | 0.003 | 0.002 | 0.001 | 0.001 | 0.001 | 0.002 | 0.002 | 0.946 | 0.002 | 0.001 | 0.002 | 0.002 | 0.001 | 0.002 |
| Sheepdog | | | | | | | | | | | | | | | | |
| Shetland | 0 | 0.002 | 0.002 | 0.002 | 0.002 | 0.001 | 0.002 | 0.002 | 0.002 | 0.938 | 0.002 | 0.002 | 0.002 | 0.003 | 0.001 | 0.003 |
| Sheepdog | | | | | | | | | | | | | | | | |
| Shetland | 0 | 0.005 | 0.002 | 0.004 | 0.003 | 0.003 | 0.001 | 0.003 | 0.002 | 0.941 | 0.001 | 0.001 | 0.003 | 0.002 | 0.001 | 0.003 |
| Sheepdog | | | | | | | | | | | | | | | | |
| Shetland | 0 | 0.01 | 0.005 | 0.003 | 0.005 | 0.001 | 0.006 | 0.003 | 0.009 | 0.826 | 0.034 | 0.008 | 0.002 | 0.005 | 0.005 | 0.003 |
| Sheepdog | | | | | | | | | | | | | | | | |
| Shetland | 0 | 0.003 | 0.004 | 0.004 | 0.002 | 0.002 | 0.002 | 0.002 | 0.006 | 0.911 | 0.001 | 0.006 | 0.001 | 0.002 | 0.004 | 0.004 |
| Sheepdog | | | | | | | | | | | | | | | | |
| Shetland | 0 | 0.001 | 0.001 | 0.001 | 0.001 | 0.001 | 0.001 | 0.001 | 0.001 | 0.97 | 0.001 | 0.002 | 0.001 | 0.001 | 0.001 | 0.001 |
| Sheepdog | | | | | | | | | | | | | | | | |
| Shetland | 0 | 0.001 | 0.002 | 0.002 | 0.001 | 0.001 | 0.002 | 0.001 | 0.002 | 0.949 | 0.004 | 0.004 | 0.001 | 0.003 | 0.002 | 0.002 |
| Sheepdog | | | | | | | | | | | | | | | | |
| Shetland | 0 | 0.002 | 0.003 | 0.003 | 0.002 | 0.001 | 0.001 | 0.003 | 0.004 | 0.924 | 0.004 | 0.002 | 0.002 | 0.005 | 0.002 | 0.007 |
| Sheepdog | | | | | | | | | | | | | | | | |
| Shetland | 0 | 0.002 | 0.007 | 0.003 | 0.002 | 0.001 | 0.007 | 0.004 | 0.002 | 0.9 | 0.004 | 0.005 | 0.003 | 0.016 | 0.012 | 0.001 |
| Sheepdog | | | | | | | | | | | | | | | | |
| Shetland | 0 | 0.001 | 0.001 | 0.001 | 0.001 | 0.001 | 0.001 | 0.001 | 0.002 | 0.962 | 0.002 | 0.002 | 0.001 | 0.001 | 0.001 | 0.001 |
| Sheepdog | | | | | | | | | | | | | | | | |
| Shetland | (2) | 0.002 | 0.002 | 0.006 | 0.006 | 0.002 | 0.004 | 0.002 | 0.001 | 0.918 | 0.003 | 0.008 | 0.002 | 0.001 | 0.002 | 0.003 |
| Sheepdog | | | | | | | | | | | | | | | | |
| Shetland | 0 | 0.001 | 0.002 | 0.002 | 0.002 | 0.002 | 0.004 | 0.002 | 0.003 | 0.921 | 0.002 | 0.01 | 0.001 | 0.004 | 0.002 | 0.002 |
| Sheepdog | | | | | | | | | | | | | | | | |
| Shetland | 0 | 0.005 | 0.002 | 0.006 | 0.004 | 0.001 | 0.002 | 0.002 | 0.003 | 0.896 | 0.002 | 0.006 | 0.002 | 0.007 | 0.002 | 0.003 |
| Sheepdog | | | | | | | | | | | | | | | | |
| Shetland | 0 | 0.013 | 0.009 | 0.002 | 0.013 | 0.003 | 0.004 | 0.003 | 0.002 | 0.836 | 0.008 | 0.006 | 0.011 | 0.01 | 0.006 | 0.003 |
| Sheepdog | | | | | | | | | | | | | | | | |
| Shetland | 0 | 0.004 | 0.002 | 0.005 | 0.002 | 0.003 | 0.002 | 0.004 | 0.003 | 0.913 | 0.003 | 0.005 | 0.003 | 0.005 | 0.002 | 0.002 |
| Sheepdog | | | | | | | | | | | | | | | | |
| Shetland | 0 | 0.002 | 0.002 | 0.002 | 0.001 | 0.001 | 0.002 | 0.003 | 0.003 | 0.941 | 0.001 | 0.003 | 0.002 | 0.003 | 0.002 | 0.001 |
| Sheepdog | | | | | | | | | | | | | | | | |
| Shetland | (1) | 0.002 | 0.003 | 0.002 | 0.001 | 0.001 | 0.001 | 0.007 | 0.003 | 0.913 | 0.003 | 0.002 | 0.001 | 0.004 | 0.002 | 0.002 |
| Sheepdog | | | | | | | | | | | | | | | | |
| Shetland | 0 | 0.004 | 0.003 | 0.003 | 0.004 | 0.002 | 0.004 | 0.002 | 0.005 | 0.874 | 0.005 | 0.011 | 0.002 | 0.002 | 0.005 | 0.005 |
| Sheepdog | | | | | | | | | | | | | | | | |
| Shetland | 0 | 0.004 | 0.003 | 0.004 | 0.002 | 0.027 | 0.004 | 0.002 | 0.002 | 0.857 | 0.002 | 0.002 | 0.004 | 0.001 | 0.007 | 0.005 |
| Sheepdog | | | | | | | | | | | | | | | | |
| Shetland | 0 | 0.001 | 0.001 | 0.002 | 0.001 | 0.001 | 0.002 | 0.002 | 0.001 | 0.943 | 0.008 | 0.001 | 0.003 | 0.002 | 0.002 | 0.002 |
| Sheepdog | | | | | | | | | | | | | | | | |
| Shetland | 0 | 0.001 | 0.002 | 0.002 | 0.002 | 0.001 | 0.002 | 0.002 | 0.003 | 0.911 | 0.002 | 0.029 | 0.002 | 0.003 | 0.005 | 0.001 |
| Sheepdog | | | | | | | | | | | | | | | | |
| Shetland | 0 | 0.003 | 0.003 | 0.001 | 0.001 | 0.001 | 0.007 | 0.006 | 0.023 | 0.871 | 0.002 | 0.008 | 0.005 | 0.008 | 0.001 | 0.003 |
| Sheepdog | | | | | | | | | | | | | | | | |
| | | Cluster Assignment | | | | | | | | | | | | | | |
| Breed | | 16 | 17 | 18 | 19 | 20 | 21 | 22 | 23 | 24 | 25 | 26 | 27 | 28 | 29 | 30 |
| Shetland | | 0.001 | 0.002 | 0.002 | 0.001 | 0.001 | 0.001 | 0.001 | 0.001 | 0.001 | 0.006 | 0.001 | 0.002 | 0.001 | 0.001 | 0.001 |
| Sheepdog | | | | | | | | | | | | | | | | |
| Shetland | | 0.001 | 0.002 | 0.001 | 0.002 | 0.001 | 0.001 | 0.001 | 0.002 | 0.003 | 0.002 | 0.001 | 0.002 | 0.009 | 0.001 | 0.002 |
| Sheepdog | | | | | | | | | | | | | | | | |
| Shetland | | 0.001 | 0.002 | 0.001 | 0.001 | 0.001 | 0.001 | 0.002 | 0.001 | 0.001 | 0.002 | 0.002 | 0.002 | 0.005 | 0.001 | 0.001 |
| Sheepdog | | | | | | | | | | | | | | | | |
| Shetland | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Sheepdog | | | | | | | | | | | | | | | | |
| Shetland | | 0.001 | 0.001 | 0.003 | 0.002 | 0.002 | 0.002 | 0.003 | 0.002 | 0.003 | 0.002 | 0.001 | 0.002 | 0.003 | 0.001 | 0.003 |
| Sheepdog | | | | | | | | | | | | | | | | |
| Shetland | | 0.001 | 0.002 | 0.003 | 0.005 | 0.002 | 0.008 | 0.001 | 0.003 | 0.003 | 0.002 | 0.005 | 0.001 | 0.005 | 0.003 | 0.005 |
| Sheepdog | | | | | | | | | | | | | | | | |
| Shetland | | 0.002 | 0.001 | 0.004 | 0.001 | 0.002 | 0.002 | 0.003 | 0.004 | 0.002 | 0.005 | 0.003 | 0.003 | 0.002 | 0.017 | 0.002 |
| Sheepdog | | | | | | | | | | | | | | | | |
| Shetland | | 0.002 | 0.002 | 0.004 | 0.001 | 0.001 | 0.004 | 0.001 | 0.006 | 0.002 | 0.002 | 0.004 | 0.004 | 0.003 | 0.002 | 0.002 |
| Sheepdog | | | | | | | | | | | | | | | | |
| Shetland | | 0.001 | 0.002 | 0.002 | 0.002 | 0.006 | 0.004 | 0.003 | 0.002 | 0.003 | 0.001 | 0.001 | 0.001 | 0.003 | 0.002 | 0.004 |
| Sheepdog | | | | | | | | | | | | | | | | |
| Shetland | | 0.001 | 0.001 | 0.001 | 0.001 | 0.002 | 0.002 | 0.003 | 0.001 | 0.001 | 0.001 | 0.001 | 0.001 | 0.001 | 0.001 | 0.002 |
| Sheepdog | | | | | | | | | | | | | | | | |
| Shetland | | 0.003 | 0.003 | 0.003 | 0.005 | 0.003 | 0.003 | 0.01 | 0.005 | 0.002 | 0.007 | 0.004 | 0.004 | 0.001 | 0.002 | 0.016 |
| Shecpdog | | | | | | | | | | | | | | | | |
| Shetland | | 0.001 | 0.003 | 0.001 | 0.001 | 0.001 | 0.001 | 0.001 | 0.001 | 0.001 | 0.001 | 0.001 | 0.001 | 0.001 | 0.001 | 0.002 |
| Sheepdog | | | | | | | | | | | | | | | | |
| Shetland | | 0.019 | 0.002 | 0.002 | 0.001 | 0.001 | 0.001 | 0.001 | 0.001 | 0.002 | 0.002 | 0.002 | 0.002 | 0.001 | 0.001 | 0.001 |
| Sheepdog | | | | | | | | | | | | | | | | |
| Shetland | | 0.001 | 0.003 | 0.009 | 0.005 | 0.012 | 0.005 | 0.014 | 0.002 | 0.007 | 0.009 | 0.001 | 0.002 | 0.003 | 0.002 | 0.005 |
| Sheepdog | | | | | | | | | | | | | | | | |
| Shetland | | 0.001 | 0.001 | 0.001 | 0.001 | 0.001 | 0.001 | 0.001 | 0.001 | 0.001 | 0.001 | 0.001 | 0.001 | 0.001 | 0.001 | 0.001 |
| Sheepdog | | | | | | | | | | | | | | | | |
| Shetland | | 0.001 | 0.001 | 0.003 | 0.002 | 0.002 | 0.001 | 0.002 | 0.002 | 0.001 | 0.002 | 0.001 | 0.002 | 0.001 | 0.001 | 0.002 |
| Sheepdog | | | | | | | | | | | | | | | | |
| Shetland | | 0.001 | 0.002 | 0.002 | 0.003 | 0.003 | 0.001 | 0.001 | 0.003 | 0.001 | 0.003 | 0.001 | 0.001 | 0.002 | 0.001 | 0.004 |
| Sheepdog | | | | | | | | | | | | | | | | |
| Shetland | | 0.001 | 0.004 | 0.002 | 0.003 | 0.003 | 0.002 | 0.005 | 0.003 | 0.002 | 0.002 | 0.001 | 0.003 | 0.001 | 0.001 | 0.001 |
| Sheepdog | | | | | | | | | | | | | | | | |
| Shetland | | 0.001 | 0.001 | 0.002 | 0.002 | 0.002 | 0.001 | 0.005 | 0.002 | 0.001 | 0.002 | 0.001 | 0.002 | 0.001 | 0.001 | 0.004 |
| Sheepdog | | | | | | | | | | | | | | | | |
| Shetland | | 0.001 | 0.005 | 0.003 | 0.002 | 0.003 | 0.005 | 0.011 | 0.009 | 0.002 | 0.006 | 0.008 | 0.002 | 0.011 | 0.004 | 0.004 |
| Sheepdog | | | | | | | | | | | | | | | | |
| Shetland | | 0.002 | 0.003 | 0.006 | 0.003 | 0.001 | 0.004 | 0.002 | 0.001 | 0.003 | 0.006 | 0.001 | 0.002 | 0.003 | 0.003 | 0.004 |
| Sheepdog | | | | | | | | | | | | | | | | |
| Shetland | | 0.001 | 0.001 | 0.002 | 0.001 | 0.001 | 0.001 | 0.001 | 0.001 | 0.001 | 0.001 | 0.001 | 0.001 | 0.001 | 0.001 | 0.002 |
| Sheepdog | | | | | | | | | | | | | | | | |
| Shetland | | 0.001 | 0.001 | 0.001 | 0.001 | 0.001 | 0.001 | 0.001 | 0.003 | 0.001 | 0.002 | 0.005 | 0.002 | 0.002 | 0.001 | 0.001 |
| Sheepdog | | | | | | | | | | | | | | | | |
| Shetland | | 0.001 | 0.001 | 0.002 | 0.001 | 0.002 | 0.001 | 0.002 | 0.005 | 0.001 | 0.006 | 0.002 | 0.006 | 0.001 | 0.002 | 0.001 |
| Sheepdog | | | | | | | | | | | | | | | | |
| Shetland | | 0.002 | 0.001 | 0.003 | 0.001 | 0.002 | 0.002 | 0.003 | 0.004 | 0.002 | 0.003 | 0.004 | 0.002 | 0.001 | 0.001 | 0.001 |
| Sheepdog | | | | | | | | | | | | | | | | |
| Shetland | | 0.001 | 0.002 | 0.002 | 0.001 | 0.001 | 0.001 | 0.001 | 0.001 | 0.001 | 0.001 | 0.001 | 0.001 | 0.002 | 0.001 | 0.001 |
| Sheepdog | | | | | | | | | | | | | | | | |
| Shetland | | 0.001 | 0.002 | 0.002 | 0.002 | 0.003 | 0.003 | 0.002 | 0.003 | 0.002 | 0.001 | 0.003 | 0.002 | 0.005 | 0.006 | 0.002 |
| Sheepdog | | | | | | | | | | | | | | | | |
| Shetland | | 0.001 | 0.001 | 0.003 | 0.004 | 0.001 | 0.003 | 0.002 | 0.002 | 0.002 | 0.002 | 0.001 | 0.001 | 0.013 | 0.002 | 0.003 |
| Sheepdog | | | | | | | | | | | | | | | | |
| Shetland | | 0.001 | 0.006 | 0.002 | 0.003 | 0.009 | 0.002 | 0.012 | 0.004 | 0.003 | 0.002 | 0.001 | 0.005 | 0.003 | 0.001 | 0.002 |
| Sheepdog | | | | | | | | | | | | | | | | |
| Shetland | | 0.001 | 0.002 | 0.005 | 0.006 | 0.004 | 0.006 | 0.005 | 0.006 | 0.012 | 0.003 | 0.002 | 0.002 | 0.002 | 0.001 | 0.013 |
| Sheepdog | | | | | | | | | | | | | | | | |
| Shetland | | 0.001 | 0.003 | 0.004 | 0.001 | 0.004 | 0.002 | 0.006 | 0.003 | 0.002 | 0.007 | 0.003 | 0.001 | 0.002 | 0.001 | 0.004 |
| Sheepdog | | | | | | | | | | | | | | | | |
| Shetland | | 0.001 | 0.005 | 0.002 | 0.002 | 0.003 | 0.002 | 0.003 | 0.004 | 0.001 | 0.003 | 0.002 | 0.001 | 0.004 | 0.001 | 0.001 |
| Sheepdog | | | | | | | | | | | | | | | | |
| Shetland | | 0.001 | 0.011 | 0.004 | 0.003 | 0.005 | 0.002 | 0.008 | 0.004 | 0.002 | 0.003 | 0.002 | 0.002 | 0.003 | 0.002 | 0.003 |
| Sheepdog | | | | | | | | | | | | | | | | |
| Shetland | | 0.001 | 0.003 | 0.011 | 0.004 | 0.003 | 0.004 | 0.005 | 0.002 | 0.006 | 0.013 | 0.002 | 0.002 | 0.006 | 0.002 | 0.004 |
| Sheepdog | | | | | | | | | | | | | | | | |
| Shetland | | 0.004 | 0.005 | 0.002 | 0.005 | 0.002 | 0.006 | 0.003 | 0.006 | 0.023 | 0.004 | 0.002 | 0.001 | 0.004 | 0.001 | 0.005 |
| Sheepdog | | | | | | | | | | | | | | | | |
| Shetland | | 0.005 | 0.005 | 0.001 | 0.001 | 0.001 | 0.001 | 0.003 | 0.002 | 0.001 | 0.001 | 0.002 | 0.001 | 0.003 | 0.001 | 0.001 |
| Sheepdog | | | | | | | | | | | | | | | | |
| Shetland | | 0.001 | 0.003 | 0.004 | 0.004 | 0.001 | 0.003 | 0.002 | 0.002 | 0.001 | 0.002 | 0.001 | 0.001 | 0.006 | 0.001 | 0.001 |
| Sheepdog | | | | | | | | | | | | | | | | |
| Shetland | | 0.003 | 0.004 | 0.011 | 0.003 | 0.002 | 0.002 | 0.001 | 0.002 | 0.001 | 0.009 | 0.001 | 0.007 | 0.003 | 0.005 | 0.002 |
| Sheepdog | | | | | | | | | | | | | | | | |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Siberian Husky | (6) | 0.01 | 0.003 | 0.002 | 0.002 | 0.002 | 0.004 | 0.002 | 0.003 | 0.001 | 0.002 | 0.003 | 0.001 | 0.001 | 0.004 | 0.013 |
| Siberian Husky | (4) | 0.007 | 0.007 | 0.006 | 0.006 | 0.011 | 0.004 | 0.002 | 0.004 | 0.004 | 0.002 | 0.008 | 0.004 | 0.003 | 0.014 | 0.012 |
| Siberian Husky | 0 | 0.004 | 0.004 | 0.004 | 0.002 | 0.018 | 0.005 | 0.003 | 0.002 | 0.002 | 0.002 | 0.002 | 0.002 | 0.002 | 0.006 | 0.017 |
| Siberian Husky | (2) | 0.003 | 0.001 | 0.001 | 0.002 | 0.004 | 0.001 | 0.001 | 0.002 | 0.001 | 0.001 | 0.002 | 0.001 | 0.001 | 0.006 | 0.004 |
| Siberian Husky | (2) | 0.077 | 0.014 | 0.002 | 0.002 | 0.001 | 0.003 | 0.025 | 0.004 | 0.011 | 0.015 | 0.002 | 0.007 | 0.003 | 0.002 | 0.006 |
| Siberian Husky | (1) | 0.004 | 0.003 | 0.002 | 0.001 | 0.002 | 0.003 | 0.002 | 0.004 | 0.002 | 0.001 | 0.006 | 0.001 | 0.001 | 0.216 | 0.002 |
| Siberian Husky | (3) | 0.004 | 0.002 | 0.006 | 0.003 | 0.002 | 0.002 | 0.001 | 0.002 | 0.005 | 0.002 | 0.004 | 0.01 | 0.004 | 0.002 | 0.002 |
| Siberian Husky | (7) | 0.004 | 0.002 | 0.007 | 0.005 | 0.002 | 0.004 | 0.003 | 0.003 | 0.012 | 0.002 | 0.007 | 0.017 | 0.01 | 0.004 | 0.003 |
| Siberian Husky | (2) | 0.003 | 0.008 | 0.01 | 0.002 | 0.001 | 0.001 | 0.003 | 0.004 | 0.003 | 0.004 | 0.002 | 0.003 | 0.003 | 0.019 | 0.01 |
| Siberian Husky | (2) | 0.008 | 0.002 | 0.003 | 0.007 | 0.009 | 0.007 | 0.01 | 0.03 | 0.003 | 0.001 | 0.003 | 0.007 | 0.002 | 0.001 | 0.002 |
| Siberian Husky | 0 | 0.028 | 0.003 | 0.004 | 0.004 | 0.002 | 0.004 | 0.003 | 0.005 | 0.002 | 0.002 | 0.005 | 0.002 | 0.003 | 0.003 | 0.003 |
| Siberian Husky | (2) | 0.008 | 0.001 | 0.003 | 0.002 | 0.001 | 0.001 | 0.001 | 0.001 | 0.001 | 0.001 | 0.002 | 0.002 | 0.001 | 0.002 | 0.002 |
| Siberian Husky | (2) | 0.088 | 0.002 | 0.007 | 0.002 | 0.001 | 0.001 | 0.001 | 0.002 | 0.005 | 0.001 | 0.002 | 0.004 | 0.001 | 0.002 | 0.002 |
| Siberian Husky | (10) | 0.04 | 0.002 | 0.004 | 0.003 | 0.002 | 0.002 | 0.003 | 0.007 | 0.002 | 0.002 | 0.004 | 0.006 | 0.002 | 0.003 | 0.005 |
| Siberian Husky | (3) | 0.001 | 0.003 | 0.005 | 0.003 | 0.003 | 0.003 | 0.004 | 0.003 | 0.003 | 0.001 | 0.001 | 0.002 | 0.001 | 0.001 | 0.022 |
| Siberian Husky | (5) | 0.001 | 0.002 | 0.001 | 0.002 | 0.001 | 0.002 | 0.002 | 0.002 | 0.001 | 0.001 | 0.001 | 0.003 | 0.001 | 0.004 | 0.003 |
| Siberian Husky | (1) | 0.005 | 0.003 | 0.007 | 0.003 | 0.004 | 0.002 | 0.002 | 0.002 | 0.006 | 0.025 | 0.002 | 0.003 | 0.001 | 0.002 | 0.005 |
| Siberian Husky | (1) | 0.002 | 0.016 | 0.007 | 0.006 | 0.01 | 0.021 | 0.006 | 0.009 | 0.002 | 0.003 | 0.005 | 0.002 | 0.001 | 0.026 | 0.082 |
| Siberian Husky | (4) | 0.005 | 0.003 | 0.004 | 0.005 | 0.003 | 0.018 | 0.005 | 0.002 | 0.003 | 0.005 | 0.003 | 0.003 | 0.004 | 0.002 | 0.065 |
| Siberian Husky | (4) | 0.007 | 0.002 | 0.003 | 0.002 | 0.01 | 0.002 | 0.003 | 0.002 | 0.002 | 0.001 | 0.002 | 0.001 | 0.001 | 0.004 | 0.024 |
| Siberian Husky | 0 | 0.002 | 0.001 | 0.002 | 0.003 | 0.009 | 0.002 | 0.002 | 0.002 | 0.002 | 0.001 | 0.022 | 0.003 | 0.002 | 0.006 | 0.005 |
| Siberian Husky | (1) | 0.036 | 0.016 | 0.004 | 0.011 | 0.003 | 0.004 | 0.004 | 0.007 | 0.002 | 0.003 | 0.001 | 0.001 | 0.002 | 0.008 | 0.006 |
| Siberian Husky | (1) | 0.006 | 0.028 | 0.003 | 0.004 | 0.001 | 0.005 | 0.008 | 0.003 | 0.001 | 0.002 | 0.002 | 0.002 | 0.005 | 0.015 | 0.005 |
| Siberian Husky | 0 | 0.022 | 0.013 | 0.002 | 0.005 | 0.005 | 0.008 | 0.016 | 0.006 | 0.002 | 0.004 | 0.008 | 0.006 | 0.005 | 0.005 | 0.025 |
| Siberian Husky | 0 | 0.005 | 0.006 | 0.002 | 0.004 | 0.001 | 0.001 | 0.003 | 0.003 | 0.002 | 0.003 | 0.001 | 0.003 | 0.002 | 0.006 | 0.003 |
| Siberian Husky | (3) | 0.002 | 0.008 | 0.008 | 0.008 | 0.003 | 0.006 | 0.003 | 0.002 | 0.002 | 0.003 | 0.005 | 0.003 | 0.002 | 0.008 | 0.053 |
| Siberian Husky | (2) | 0.002 | 0.003 | 0.013 | 0.002 | 0.002 | 0.002 | 0.002 | 0.002 | 0.002 | 0.001 | 0.002 | 0.002 | 0.001 | 0.034 | 0.002 |
| Siberian Husky | (1) | 0.003 | 0.002 | 0.002 | 0.002 | 0.016 | 0.003 | 0.002 | 0.007 | 0.002 | 0.003 | 0.005 | 0.003 | 0.001 | 0.009 | 0.005 |
| Siberian Husky | (1) | 0.002 | 0.001 | 0.004 | 0.004 | 0.009 | 0.003 | 0.001 | 0.002 | 0.002 | 0.003 | 0.028 | 0.013 | 0.002 | 0.081 | 0.015 |
| Siberian Husky | (1) | 0.002 | 0.002 | 0.002 | 0.001 | 0.008 | 0.002 | 0.001 | 0.001 | 0.001 | 0.001 | 0.004 | 0.004 | 0.002 | 0.006 | 0.023 |
| Siberian Husky | (2) | 0.003 | 0.002 | 0.002 | 0.002 | 0.008 | 0.001 | 0.006 | 0.025 | 0.001 | 0.001 | 0.005 | 0.003 | 0.001 | 0.006 | 0.003 |
| Siberian Husky | (2) | 0.009 | 0.003 | 0.003 | 0.002 | 0.003 | 0.004 | 0.018 | 0.018 | 0.003 | 0.003 | 0.016 | 0.003 | 0.002 | 0.005 | 0.01 |
| Siberian Husky | (6) | 0.002 | 0.004 | 0.002 | 0.003 | 0.041 | 0.003 | 0.003 | 0.007 | 0.001 | 0.001 | 0.004 | 0.004 | 0.002 | 0.018 | 0.003 |
| Siberian Husky | (19) | 0.051 | 0.004 | 0.004 | 0.006 | 0.001 | 0.002 | 0.003 | 0.004 | 0.002 | 0.003 | 0.002 | 0.003 | 0.002 | 0.005 | 0.004 |
| Siberian Husky | (2) | 0.002 | 0.002 | 0.001 | 0.002 | 0.004 | 0.002 | 0.001 | 0.001 | 0.001 | 0.001 | 0.001 | 0.001 | 0.001 | 0.001 | 0.009 |
| Siberian Husky | (3) | 0.001 | 0.002 | 0.002 | 0.002 | 0.014 | 0.002 | 0.002 | 0.003 | 0.002 | 0.001 | 0.003 | 0.002 | 0.002 | 0.006 | 0.003 |
| Siberian Husky | (1) | 0.006 | 0.004 | 0.002 | 0.006 | 0.001 | 0.002 | 0.002 | 0.002 | 0.002 | 0.002 | 0.002 | 0.001 | 0.001 | 0.002 | 0.004 |
| Siberian Husky | (3) | 0.003 | 0.001 | 0.002 | 0.002 | 0.002 | 0.001 | 0.001 | 0.001 | 0.002 | 0.001 | 0.001 | 0.001 | 0.001 | 0.002 | 0.003 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Siberian Husky | | 0.001 | 0.001 | 0.003 | 0.004 | 0.002 | 0.003 | 0.002 | 0.003 | 0.913 | 0.002 | 0.002 | 0.002 | 0.004 | 0.002 | 0.004 |
| Siberian Husky | | 0.001 | 0.005 | 0.003 | 0.011 | 0.001 | 0.008 | 0.003 | 0.006 | 0.844 | 0.004 | 0.003 | 0.003 | 0.006 | 0.003 | 0.004 |
| Siberian Husky | | 0.001 | 0.001 | 0.001 | 0.005 | 0.002 | 0.005 | 0.002 | 0.002 | 0.894 | 0.002 | 0.002 | 0.002 | 0.002 | 0.001 | 0.003 |
| Siberian Husky | | 0.001 | 0.002 | 0.002 | 0.001 | 0.001 | 0.002 | 0.002 | 0.002 | 0.942 | 0.002 | 0.001 | 0.003 | 0.002 | 0.002 | 0.002 |
| Siberian Husky | | 0.002 | 0.002 | 0.002 | 0.002 | 0.002 | 0.001 | 0.003 | 0.006 | 0.773 | 0.005 | 0.002 | 0.011 | 0.002 | 0.009 | 0.002 |
| Siberian Husky | | 0.001 | 0.002 | 0.003 | 0.003 | 0.002 | 0.002 | 0.003 | 0.002 | 0.714 | 0.01 | 0.002 | 0.001 | 0.001 | 0.001 | 0.002 |
| Siberian Husky | | 0.001 | 0.001 | 0.001 | 0.002 | 0.003 | 0.001 | 0.002 | 0.001 | 0.923 | 0.004 | 0.001 | 0.004 | 0.003 | 0.001 | 0.003 |
| Siberian Husky | | 0.003 | 0.002 | 0.002 | 0.003 | 0.004 | 0.001 | 0.003 | 0.002 | 0.868 | 0.004 | 0.002 | 0.012 | 0.005 | 0.002 | 0.001 |
| Siberian Husky | | 0.001 | 0.003 | 0.002 | 0.003 | 0.003 | 0.002 | 0.002 | 0.006 | 0.883 | 0.007 | 0.002 | 0.005 | 0.002 | 0.002 | 0.002 |
| Siberian Husky | | 0.007 | 0.002 | 0.001 | 0.008 | 0.006 | 0.004 | 0.002 | 0.009 | 0.841 | 0.005 | 0.006 | 0.004 | 0.006 | 0.004 | 0.001 |
| Siberian Husky | | 0.003 | 0.003 | 0.01 | 0.004 | 0.002 | 0.004 | 0.013 | 0.005 | 0.86 | 0.003 | 0.003 | 0.002 | 0.006 | 0.003 | 0.009 |
| Siberian Husky | | 0.001 | 0.001 | 0.002 | 0.001 | 0.002 | 0.001 | 0.001 | 0.001 | 0.951 | 0.002 | 0.001 | 0.001 | 0.001 | 0.001 | 0.001 |
| Siberian Husky | | 0.001 | 0.004 | 0.002 | 0.002 | 0.001 | 0.001 | 0.002 | 0.004 | 0.85 | 0.004 | 0.001 | 0.002 | 0.002 | 0.001 | 0.002 |
| Siberian Husky | | 0.002 | 0.001 | 0.005 | 0.004 | 0.004 | 0.002 | 0.001 | 0.003 | 0.859 | 0.021 | 0.001 | 0.003 | 0.003 | 0.002 | 0.002 |
| Siberian Husky | | 0.002 | 0.002 | 0.002 | 0.005 | 0.004 | 0.001 | 0.003 | 0.005 | 0.909 | 0.001 | 0.001 | 0.001 | 0.004 | 0.002 | 0.001 |
| Siberian Husky | | 0.001 | 0.003 | 0.002 | 0.002 | 0.001 | 0.002 | 0.002 | 0.004 | 0.949 | 0.001 | 0.002 | 0.001 | 0.001 | 0.001 | 0.002 |
| Siberian Husky | | 0.002 | 0.002 | 0.003 | 0.003 | 0.006 | 0.002 | 0.003 | 0.088 | 0.8 | 0.001 | 0.009 | 0.002 | 0.002 | 0.002 | 0.003 |
| Siberian Husky | | 0.001 | 0.004 | 0.004 | 0.033 | 0.003 | 0.006 | 0.003 | 0.007 | 0.726 | 0.001 | 0.001 | 0.002 | 0.004 | 0.004 | 0.004 |
| Siberian Husky | | 0.001 | 0.001 | 0.002 | 0.006 | 0.006 | 0.002 | 0.001 | 0.004 | 0.825 | 0.001 | 0.001 | 0.002 | 0.003 | 0.009 | 0.005 |
| Siberian Husky | | 0.002 | 0.001 | 0.002 | 0.007 | 0.003 | 0.002 | 0.003 | 0.002 | 0.898 | 0.003 | 0.001 | 0.003 | 0.002 | 0.001 | 0.002 |
| Siberian Husky | | 0.001 | 0.002 | 0.002 | 0.005 | 0.003 | 0.004 | 0.001 | 0.002 | 0.904 | 0.003 | 0.001 | 0.002 | 0.005 | 0.001 | 0.002 |
| Siberian Husky | | 0.003 | 0.01 | 0.003 | 0.01 | 0.006 | 0.012 | 0.003 | 0.153 | 0.641 | 0.005 | 0.003 | 0.004 | 0.002 | 0.008 | 0.028 |
| Siberian Husky | | 0.003 | 0.006 | 0.002 | 0.006 | 0.003 | 0.006 | 0.003 | 0.017 | 0.823 | 0.003 | 0.003 | 0.006 | 0.003 | 0.018 | 0.007 |
| Siberian Husky | | 0.002 | 0.002 | 0.003 | 0.009 | 0.002 | 0.003 | 0.009 | 0.003 | 0.775 | 0.004 | 0.003 | 0.003 | 0.003 | 0.044 | 0.005 |
| Siberian Husky | | 0.001 | 0.003 | 0.002 | 0.004 | 0.002 | 0.006 | 0.003 | 0.015 | 0.895 | 0.002 | 0.002 | 0.003 | 0.003 | 0.006 | 0.006 |
| Siberian Husky | | 0.001 | 0.002 | 0.004 | 0.005 | 0.004 | 0.011 | 0.002 | 0.026 | 0.807 | 0.001 | 0.002 | 0.001 | 0.005 | 0.005 | 0.01 |
| Siberian Husky | | 0.001 | 0.001 | 0.002 | 0.001 | 0.001 | 0.001 | 0.002 | 0.001 | 0.902 | 0.004 | 0.002 | 0.002 | 0.002 | 0.002 | 0.002 |
| Siberian Husky | | 0.001 | 0.004 | 0.001 | 0.025 | 0.011 | 0.001 | 0.004 | 0.003 | 0.866 | 0.001 | 0.001 | 0.003 | 0.006 | 0.003 | 0.002 |
| Siberian Husky | | 0.001 | 0.001 | 0.001 | 0.006 | 0.003 | 0.003 | 0.003 | 0.004 | 0.794 | 0.005 | 0.002 | 0.001 | 0.003 | 0.003 | 0.001 |
| Siberian Husky | | 0.001 | 0.001 | 0.002 | 0.002 | 0.001 | 0.001 | 0.002 | 0.002 | 0.916 | 0.001 | 0.001 | 0.001 | 0.002 | 0.002 | 0.006 |
| Siberian Husky | | 0.001 | 0.002 | 0.002 | 0.002 | 0.002 | 0.003 | 0.002 | 0.003 | 0.9 | 0.004 | 0.001 | 0.001 | 0.003 | 0.002 | 0.001 |
| Siberian Husky | | 0.003 | 0.006 | 0.006 | 0.006 | 0.016 | 0.004 | 0.006 | 0.007 | 0.811 | 0.014 | 0.002 | 0.006 | 0.003 | 0.002 | 0.006 |
| Siberian Husky | | 0.004 | 0.002 | 0.002 | 0.005 | 0.001 | 0.002 | 0.001 | 0.005 | 0.856 | 0.006 | 0.006 | 0.004 | 0.001 | 0.003 | 0.001 |
| Siberian Husky | | 0.001 | 0.002 | 0.002 | 0.002 | 0.001 | 0.002 | 0.002 | 0.004 | 0.872 | 0.005 | 0.002 | 0.004 | 0.002 | 0.003 | 0.002 |
| Siberian Husky | | 0.001 | 0.001 | 0.002 | 0.001 | 0.003 | 0.002 | 0.002 | 0.001 | 0.948 | 0.001 | 0.001 | 0.002 | 0.001 | 0.001 | 0.002 |
| Siberian Husky | | 0.002 | 0.006 | 0.002 | 0.004 | 0.002 | 0.003 | 0.005 | 0.005 | 0.909 | 0.002 | 0.002 | 0.004 | 0.003 | 0.005 | 0.002 |
| Siberian Husky | | 0.001 | 0.002 | 0.002 | 0.002 | 0.003 | 0.004 | 0.001 | 0.003 | 0.934 | 0.001 | 0.001 | 0.001 | 0.001 | 0.005 | 0.001 |
| Siberian Husky | | 0.001 | 0.002 | 0.001 | 0.001 | 0.002 | 0.001 | 0.002 | 0.002 | 0.955 | 0.001 | 0.001 | 0.002 | 0.001 | 0.001 | 0.002 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| St Bernard | 0 | 0.002 | 0.002 | 0.001 | 0.002 | 0.001 | 0.002 | 0.006 | 0.002 | 0.047 | 0.035 | 0.003 | 0.001 | 0.004 | 0.003 | 0.001 |
| St Bernard | 0 | 0.002 | 0.001 | 0.002 | 0.002 | 0.002 | 0.002 | 0.002 | 0.002 | 0.002 | 0.002 | 0.001 | 0.001 | 0.002 | 0.002 | 0.001 |
| St Bernard | 0 | 0.002 | 0.001 | 0.004 | 0.003 | 0.001 | 0.002 | 0.001 | 0.002 | 0.001 | 0.002 | 0.003 | 0.002 | 0.002 | 0.001 | 0.002 |
| St Bernard | 0 | 0.001 | 0.002 | 0.001 | 0.001 | 0.001 | 0.001 | 0.002 | 0.002 | 0.001 | 0.001 | 0.001 | 0.001 | 0.001 | 0.002 | 0.001 |
| St Bernard | 0 | 0.004 | 0.001 | 0.003 | 0.007 | 0.002 | 0.004 | 0.002 | 0.005 | 0.002 | 0.001 | 0.004 | 0.001 | 0.003 | 0.004 | 0.001 |
| St Bernard | 0 | 0.002 | 0.003 | 0.003 | 0.005 | 0.005 | 0.002 | 0.001 | 0.001 | 0.002 | 0.002 | 0.002 | 0.002 | 0.002 | 0.001 | 0.002 |
| St Bernard | 0 | 0.001 | 0.001 | 0.003 | 0.002 | 0.002 | 0.001 | 0.001 | 0.003 | 0.001 | 0.001 | 0.002 | 0.004 | 0.001 | 0.001 | 0.002 |
| St Bernard | 0 | 0.001 | 0.004 | 0.007 | 0.003 | 0.002 | 0.004 | 0.002 | 0.002 | 0.002 | 0.005 | 0.006 | 0.01 | 0.004 | 0.007 | 0.002 |
| St Bernard | 0 | 0.003 | 0.003 | 0.002 | 0.001 | 0.002 | 0.002 | 0.002 | 0.003 | 0.002 | 0.003 | 0.003 | 0.001 | 0.002 | 0.002 | 0.004 |
| St Bernard | (9) | 0.005 | 0.001 | 0.003 | 0.002 | 0.001 | 0.001 | 0.003 | 0.003 | 0.021 | 0.017 | 0.002 | 0.001 | 0.001 | 0.001 | 0.001 |
| St Bernard | 0 | 0.002 | 0.006 | 0.002 | 0.002 | 0.003 | 0.006 | 0.004 | 0.004 | 0.003 | 0.063 | 0.003 | 0.002 | 0.004 | 0.001 | 0.004 |
| St Bernard | 0 | 0.005 | 0.009 | 0.002 | 0.001 | 0.003 | 0.004 | 0.008 | 0.008 | 0.002 | 0.006 | 0.004 | 0.002 | 0.002 | 0.001 | 0.002 |
| St Bernard | 0 | 0.002 | 0.002 | 0.002 | 0.003 | 0.003 | 0.006 | 0.001 | 0.002 | 0.004 | 0.001 | 0.002 | 0.001 | 0.002 | 0.003 | 0.004 |
| St Bernard | 0 | 0.006 | 0.004 | 0.003 | 0.002 | 0.006 | 0.002 | 0.003 | 0.003 | 0.002 | 0.001 | 0.005 | 0.003 | 0.005 | 0.002 | 0.014 |
| St Bernard | 0 | 0.005 | 0.006 | 0.002 | 0.005 | 0.001 | 0.004 | 0.005 | 0.003 | 0.003 | 0.002 | 0.002 | 0.002 | 0.006 | 0.044 | 0.002 |
| St Bernard | 0 | 0.001 | 0.002 | 0.001 | 0.001 | 0.001 | 0.001 | 0.001 | 0.001 | 0.001 | 0.001 | 0.002 | 0.001 | 0.001 | 0.001 | 0.001 |
| St Bernard | 0 | 0.003 | 0.006 | 0.002 | 0.003 | 0.002 | 0.003 | 0.003 | 0.002 | 0.001 | 0.002 | 0.001 | 0.002 | 0.004 | 0.003 | 0.002 |
| St Bernard | (7) | 0.002 | 0.002 | 0.001 | 0.002 | 0.001 | 0.002 | 0.003 | 0.002 | 0.001 | 0.001 | 0.001 | 0.001 | 0.002 | 0.001 | 0.001 |
| St Bernard | (1) | 0.001 | 0.002 | 0.002 | 0.001 | 0.001 | 0.002 | 0.002 | 0.002 | 0.002 | 0.001 | 0.001 | 0.001 | 0.002 | 0.002 | 0.002 |
| St Bernard | 0 | 0.006 | 0.003 | 0.002 | 0.002 | 0.001 | 0.002 | 0.001 | 0.002 | 0.002 | 0.001 | 0.008 | 0.001 | 0.001 | 0.003 | 0.003 |
| St Bernard | 0 | 0.001 | 0.005 | 0.002 | 0.002 | 0.001 | 0.001 | 0.001 | 0.001 | 0.004 | 0.005 | 0.002 | 0.003 | 0.002 | 0.003 | 0.003 |
| St Bernard | (2) | 0.002 | 0.001 | 0.004 | 0.003 | 0.001 | 0.001 | 0.001 | 0.002 | 0.002 | 0.001 | 0.002 | 0.002 | 0.002 | 0.001 | 0.001 |
| St Bernard | 0 | 0.002 | 0.011 | 0.002 | 0.002 | 0.001 | 0.003 | 0.007 | 0.002 | 0.001 | 0.003 | 0.003 | 0.001 | 0.002 | 0.003 | 0.004 |
| St Bernard | 0 | 0.007 | 0.003 | 0.028 | 0.005 | 0.001 | 0.002 | 0.004 | 0.007 | 0.004 | 0.001 | 0.004 | 0.003 | 0.002 | 0.002 | 0.001 |
| St Bernard | 0 | 0.028 | 0.009 | 0.047 | 0.013 | 0.003 | 0.004 | 0.001 | 0.013 | 0.001 | 0.003 | 0.021 | 0.002 | 0.004 | 0.004 | 0.003 |
| St Bernard | 0 | 0.022 | 0.005 | 0.003 | 0.011 | 0.011 | 0.006 | 0.003 | 0.002 | 0.003 | 0.002 | 0.012 | 0.015 | 0.004 | 0.001 | 0.002 |
| St Bernard | 0 | 0.003 | 0.002 | 0.004 | 0.001 | 0.001 | 0.002 | 0.002 | 0.002 | 0.001 | 0.002 | 0.004 | 0.002 | 0.002 | 0.004 | 0.001 |
| St Bernard | 0 | 0.001 | 0.002 | 0.001 | 0.001 | 0.001 | 0.001 | 0.004 | 0.005 | 0.002 | 0.002 | 0.002 | 0.002 | 0.001 | 0.002 | 0.001 |
| St Bernard | 0 | 0.003 | 0.005 | 0.008 | 0.002 | 0.002 | 0.003 | 0.002 | 0.003 | 0.004 | 0.007 | 0.021 | 0.001 | 0.007 | 0.017 | 0.003 |
| St Bernard | (2) | 0.001 | 0.001 | 0.001 | 0.001 | 0.001 | 0.002 | 0.001 | 0.001 | 0.001 | 0.001 | 0.001 | 0.001 | 0.001 | 0.001 | 0.001 |
| St Bernard | 0 | 0.002 | 0.002 | 0.002 | 0.003 | 0.001 | 0.005 | 0.002 | 0.003 | 0.003 | 0.003 | 0.004 | 0.001 | 0.002 | 0.001 | 0.001 |
| St Bernard | 0 | 0.001 | 0.002 | 0.001 | 0.001 | 0.001 | 0.001 | 0.001 | 0.002 | 0.001 | 0.002 | 0.001 | 0.001 | 0.001 | 0.001 | 0.001 |
| St Bernard | 0 | 0.004 | 0.007 | 0.003 | 0.007 | 0.001 | 0.007 | 0.003 | 0.003 | 0.021 | 0.004 | 0.018 | 0.007 | 0.003 | 0.003 | 0.002 |
| St Bernard | 0 | 0.003 | 0.002 | 0.003 | 0.002 | 0.002 | 0.004 | 0.004 | 0.002 | 0.012 | 0.003 | 0.002 | 0.003 | 0.002 | 0.001 | 0.001 |
| St Bernard | 0 | 0.002 | 0.002 | 0.001 | 0.001 | 0.001 | 0.003 | 0.002 | 0.003 | 0.001 | 0.003 | 0.004 | 0.001 | 0.001 | 0.003 | 0.001 |
| St Bernard | (1) | 0.007 | 0.004 | 0.027 | 0.013 | 0.002 | 0.002 | 0.001 | 0.005 | 0.002 | 0.003 | 0.01 | 0.013 | 0.102 | 0.001 | 0.002 |
| St Bernard | 0 | 0.003 | 0.003 | 0.003 | 0.003 | 0.002 | 0.002 | 0.002 | 0.001 | 0.001 | 0.004 | 0.003 | 0.003 | 0.001 | 0.002 | 0.003 |
| St Bernard | 0 | 0.003 | 0.002 | 0.002 | 0.003 | 0.001 | 0.002 | 0.001 | 0.002 | 0.002 | 0.001 | 0.003 | 0.001 | 0.001 | 0.002 | 0.002 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| St Bernard | | 0.001 | 0.84 | 0.004 | 0.003 | 0.002 | 0.001 | 0.003 | 0.004 | 0.002 | 0.009 | 0.001 | 0.012 | 0.002 | 0.001 | 0.002 |
| St Bernard | | 0.001 | 0.951 | 0.002 | 0.001 | 0.002 | 0.002 | 0.001 | 0.001 | 0.002 | 0.002 | 0.002 | 0.002 | 0.002 | 0.002 | 0.002 |
| St Bernard | | 0.001 | 0.942 | 0.004 | 0.002 | 0.001 | 0.004 | 0.002 | 0.002 | 0.002 | 0.002 | 0.001 | 0.001 | 0.003 | 0.002 | 0.002 |
| St Bernard | | 0.001 | 0.963 | 0.002 | 0.001 | 0.001 | 0.001 | 0.001 | 0.002 | 0.001 | 0.003 | 0.001 | 0.001 | 0.001 | 0.001 | 0.001 |
| St Bernard | | 0.013 | 0.885 | 0.004 | 0.002 | 0.001 | 0.002 | 0.001 | 0.002 | 0.002 | 0.008 | 0.001 | 0.003 | 0.025 | 0.003 | 0.004 |
| St Bernard | | 0.002 | 0.932 | 0.001 | 0.008 | 0.002 | 0.003 | 0.001 | 0.001 | 0.001 | 0.001 | 0.001 | 0.003 | 0.002 | 0.004 | 0.001 |
| St Bernard | | 0.001 | 0.953 | 0.002 | 0.001 | 0.001 | 0.001 | 0.006 | 0.001 | 0.001 | 0.001 | 0.001 | 0.001 | 0.001 | 0.001 | 0.001 |
| St Bernard | | 0.001 | 0.887 | 0.004 | 0.004 | 0.002 | 0.002 | 0.009 | 0.01 | 0.002 | 0.004 | 0.002 | 0.002 | 0.002 | 0.003 | 0.003 |
| St Bernard | | 0.002 | 0.919 | 0.003 | 0.002 | 0.007 | 0.001 | 0.004 | 0.006 | 0.005 | 0.002 | 0.002 | 0.004 | 0.002 | 0.005 | 0.002 |
| St Bernard | | 0.016 | 0.874 | 0.008 | 0.002 | 0.001 | 0.007 | 0.001 | 0.002 | 0.001 | 0.004 | 0.002 | 0.009 | 0.003 | 0.003 | 0.002 |
| St Bernard | | 0.003 | 0.887 | 0.003 | 0.01 | 0.003 | 0.005 | 0.003 | 0.01 | 0.002 | 0.001 | 0.002 | 0.001 | 0.014 | 0.004 | 0.001 |
| St Bernard | | 0.003 | 0.886 | 0.009 | 0.003 | 0.005 | 0.003 | 0.002 | 0.002 | 0.002 | 0.003 | 0.002 | 0.003 | 0.006 | 0.006 | 0.002 |
| St Bernard | | 0.002 | 0.915 | 0.005 | 0.002 | 0.001 | 0.005 | 0.005 | 0.003 | 0.009 | 0.002 | 0.002 | 0.001 | 0.003 | 0.001 | 0.005 |
| St Bernard | | 0.002 | 0.852 | 0.008 | 0.008 | 0.002 | 0.005 | 0.002 | 0.003 | 0.025 | 0.002 | 0.003 | 0.002 | 0.016 | 0.003 | 0.005 |
| St Bernard | | 0.004 | 0.834 | 0.002 | 0.001 | 0.002 | 0.01 | 0.006 | 0.01 | 0.011 | 0.003 | 0.009 | 0.004 | 0.001 | 0.007 | 0.004 |
| St Bernard | | 0.001 | 0.969 | 0.002 | 0.001 | 0.001 | 0.001 | 0.001 | 0.001 | 0.001 | 0.001 | 0.001 | 0.001 | 0.001 | 0.001 | 0.001 |
| St Bernard | | 0.002 | 0.921 | 0.002 | 0.002 | 0.002 | 0.003 | 0.006 | 0.003 | 0.005 | 0.001 | 0.002 | 0.003 | 0.003 | 0.002 | 0.004 |
| St Bernard | | 0.002 | 0.951 | 0.002 | 0.002 | 0.003 | 0.002 | 0.001 | 0.002 | 0.002 | 0.001 | 0.001 | 0.002 | 0.001 | 0.001 | 0.002 |
| St Bernard | | 0.001 | 0.951 | 0.002 | 0.002 | 0.002 | 0.001 | 0.002 | 0.003 | 0.002 | 0.002 | 0.001 | 0.005 | 0.002 | 0.001 | 0.002 |
| St Bernard | | 0.002 | 0.902 | 0.002 | 0.002 | 0.007 | 0.003 | 0.002 | 0.004 | 0.023 | 0.003 | 0.002 | 0.004 | 0.001 | 0.002 | 0.002 |
| St Bernard | | 0.002 | 0.932 | 0.002 | 0.001 | 0.002 | 0.001 | 0.001 | 0.004 | 0.003 | 0.001 | 0.006 | 0.003 | 0.001 | 0.003 | 0.001 |
| St Bernard | | 0.001 | 0.949 | 0.003 | 0.002 | 0.001 | 0.002 | 0.001 | 0.002 | 0.001 | 0.002 | 0.001 | 0.004 | 0.003 | 0.001 | 0.001 |
| St Bernard | | 0.002 | 0.9 | 0.003 | 0.001 | 0.002 | 0.004 | 0.005 | 0.003 | 0.004 | 0.002 | 0.001 | 0.006 | 0.008 | 0.008 | 0.003 |
| St Bernard | | 0.002 | 0.851 | 0.007 | 0.012 | 0.002 | 0.001 | 0.024 | 0.004 | 0.002 | 0.002 | 0.004 | 0.002 | 0.005 | 0.001 | 0.005 |
| St Bernard | | 0.006 | 0.728 | 0.038 | 0.009 | 0.004 | 0.004 | 0.002 | 0.007 | 0.002 | 0.008 | 0.008 | 0.012 | 0.008 | 0.006 | 0.002 |
| St Bernard | | 0.002 | 0.764 | 0.004 | 0.061 | 0.022 | 0.002 | 0.006 | 0.003 | 0.005 | 0.003 | 0.001 | 0.003 | 0.015 | 0.002 | 0.006 |
| St Bernard | | 0.001 | 0.936 | 0.006 | 0.002 | 0.002 | 0.002 | 0.002 | 0.001 | 0.004 | 0.002 | 0.002 | 0.002 | 0.003 | 0.001 | 0.002 |
| St Bernard | | 0.001 | 0.94 | 0.002 | 0.001 | 0.002 | 0.002 | 0.002 | 0.004 | 0.002 | 0.002 | 0.002 | 0.002 | 0.004 | 0.002 | 0.003 |
| St Bernard | | 0.001 | 0.871 | 0.005 | 0.001 | 0.004 | 0.002 | 0.002 | 0.002 | 0.002 | 0.005 | 0.003 | 0.009 | 0.004 | 0.001 | 0.002 |
| St Bernard | | 0.001 | 0.967 | 0.002 | 0.001 | 0.002 | 0.001 | 0.002 | 0.001 | 0.002 | 0.001 | 0.001 | 0.001 | 0.001 | 0.002 | 0.001 |
| St Bernard | | 0.001 | 0.93 | 0.004 | 0.003 | 0.002 | 0.003 | 0.002 | 0.002 | 0.002 | 0.002 | 0.002 | 0.002 | 0.004 | 0.003 | 0.002 |
| St Bernard | | 0.001 | 0.966 | 0.002 | 0.001 | 0.001 | 0.001 | 0.002 | 0.002 | 0.001 | 0.002 | 0.001 | 0.001 | 0.001 | 0.001 | 0.001 |
| St Bernard | | 0.001 | 0.781 | 0.037 | 0.026 | 0.002 | 0.001 | 0.003 | 0.012 | 0.012 | 0.001 | 0.018 | 0.002 | 0.004 | 0.003 | 0.003 |
| St Bernard | | 0.002 | 0.816 | 0.013 | 0.013 | 0.049 | 0.001 | 0.003 | 0.03 | 0.003 | 0.003 | 0.01 | 0.002 | 0.006 | 0.001 | 0.002 |
| St Bernard | | 0.001 | 0.934 | 0.005 | 0.002 | 0.001 | 0.002 | 0.003 | 0.003 | 0.001 | 0.006 | 0.002 | 0.002 | 0.002 | 0.004 | 0.002 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Whippet | 0 | 0.002 | 0.002 | 0.001 | 0.001 | 0.001 | 0.001 | 0.003 | 0.001 | 0.005 | 0.003 | 0.001 | 0.003 | 0.941 | 0.001 | 0.001 |
| Whippet | 0 | 0.003 | 0.003 | 0.005 | 0.004 | 0.003 | 0.005 | 0.001 | 0.002 | 0.001 | 0.006 | 0.007 | 0.001 | 0.887 | 0.005 | 0.018 |
| Whippet | 0 | 0.003 | 0.001 | 0.002 | 0.002 | 0.001 | 0.005 | 0.006 | 0.005 | 0.006 | 0.002 | 0.007 | 0.002 | 0.849 | 0.006 | 0.002 |
| Whippet | 0 | 0.003 | 0.012 | 0.003 | 0.009 | 0.004 | 0.002 | 0.004 | 0.005 | 0.001 | 0.008 | 0.002 | 0.003 | 0.846 | 0.003 | 0.012 |
| Whippet | (1) | 0.003 | 0.005 | 0.003 | 0.004 | 0.009 | 0.003 | 0.014 | 0.008 | 0.002 | 0.004 | 0.002 | 0.008 | 0.815 | 0.001 | 0.008 |
| Whippet | (3) | 0.005 | 0.012 | 0.004 | 0.007 | 0.003 | 0.005 | 0.009 | 0.003 | 0.002 | 0.004 | 0.003 | 0.003 | 0.828 | 0.004 | 0.008 |
| Whippet | 0 | 0.001 | 0.002 | 0.001 | 0.002 | 0.001 | 0.004 | 0.001 | 0.003 | 0.002 | 0.001 | 0.002 | 0.006 | 0.928 | 0.001 | 0.001 |
| Whippet | 0 | 0.002 | 0.002 | 0.001 | 0.005 | 0.002 | 0.004 | 0.004 | 0.003 | 0.004 | 0.006 | 0.003 | 0.003 | 0.918 | 0.002 | 0.002 |
| Whippet | 0 | 0.002 | 0.003 | 0.003 | 0.001 | 0.001 | 0.002 | 0.001 | 0.002 | 0.001 | 0.002 | 0.001 | 0.002 | 0.948 | 0.002 | 0.001 |
| Whippet | 0 | 0.003 | 0.003 | 0.015 | 0.002 | 0.002 | 0.024 | 0.002 | 0.002 | 0.002 | 0.004 | 0.003 | 0.002 | 0.884 | 0.002 | 0.003 |
| Whippet | 0 | 0.002 | 0.007 | 0.006 | 0.001 | 0.001 | 0.002 | 0.003 | 0.001 | 0.001 | 0.018 | 0.003 | 0.002 | 0.905 | 0.004 | 0.003 |
| Whippet | 0 | 0.007 | 0.005 | 0.006 | 0.004 | 0.001 | 0.015 | 0.002 | 0.003 | 0.004 | 0.003 | 0.03 | 0.008 | 0.784 | 0.012 | 0.002 |
| Whippet | 0 | 0.013 | 0.004 | 0.002 | 0.002 | 0.001 | 0.002 | 0.003 | 0.006 | 0.007 | 0.002 | 0.004 | 0.004 | 0.873 | 0.001 | 0.002 |
| Whippet | 0 | 0.002 | 0.001 | 0.002 | 0.002 | 0.001 | 0.001 | 0.001 | 0.003 | 0.001 | 0.001 | 0.002 | 0.001 | 0.949 | 0.001 | 0.001 |
| Whippet | 0 | 0.008 | 0.003 | 0.007 | 0.003 | 0.008 | 0.027 | 0.007 | 0.007 | 0.004 | 0.005 | 0.046 | 0.002 | 0.711 | 0.002 | 0.009 |
| Whippet | 0 | 0.002 | 0.002 | 0.014 | 0.002 | 0.002 | 0.006 | 0.002 | 0.003 | 0.001 | 0.002 | 0.003 | 0.001 | 0.904 | 0.004 | 0.002 |
| Whippet | 0 | 0.006 | 0.014 | 0.002 | 0.009 | 0.001 | 0.003 | 0.004 | 0.002 | 0.002 | 0.002 | 0.001 | 0.002 | 0.906 | 0.001 | 0.001 |
| Whippet | 0 | 0.014 | 0.002 | 0.001 | 0.002 | 0.001 | 0.003 | 0.007 | 0.004 | 0.002 | 0.004 | 0.001 | 0.003 | 0.913 | 0.001 | 0.001 |
| Whippet | 0 | 0.003 | 0.004 | 0.001 | 0.007 | 0.001 | 0.004 | 0.002 | 0.005 | 0.002 | 0.005 | 0.003 | 0.002 | 0.894 | 0.002 | 0.001 |
| Whippet | 0 | 0.003 | 0.003 | 0.002 | 0.002 | 0.001 | 0.002 | 0.001 | 0.002 | 0.001 | 0.002 | 0.002 | 0.001 | 0.942 | 0.002 | 0.002 |
| Whippet | 0 | 0.011 | 0.008 | 0.01 | 0.005 | 0.003 | 0.01 | 0.003 | 0.003 | 0.003 | 0.002 | 0.011 | 0.001 | 0.832 | 0.007 | 0.018 |
| Whippet | (1) | 0.004 | 0.017 | 0.002 | 0.009 | 0.001 | 0.025 | 0.005 | 0.013 | 0.004 | 0.005 | 0.002 | 0.001 | 0.707 | 0.003 | 0.002 |
| Whippet | 0 | 0.012 | 0.009 | 0.003 | 0.005 | 0.002 | 0.007 | 0.011 | 0.005 | 0.007 | 0.006 | 0.009 | 0.006 | 0.863 | 0.002 | 0.005 |
| Whippet | 0 | 0.001 | 0.003 | 0.003 | 0.007 | 0.001 | 0.001 | 0.001 | 0.002 | 0.002 | 0.005 | 0.002 | 0.003 | 0.932 | 0.002 | 0.001 |
| Whippet | 0 | 0.004 | 0.016 | 0.005 | 0.012 | 0.001 | 0.006 | 0.003 | 0.005 | 0.002 | 0.006 | 0.011 | 0.005 | 0.844 | 0.003 | 0.001 |
| Whippet | 0 | 0.012 | 0.007 | 0.004 | 0.008 | 0.002 | 0.004 | 0.005 | 0.005 | 0.006 | 0.003 | 0.003 | 0.012 | 0.828 | 0.003 | 0.002 |
| Whippet | 0 | 0.004 | 0.004 | 0.004 | 0.003 | 0.003 | 0.005 | 0.004 | 0.006 | 0.002 | 0.009 | 0.007 | 0.003 | 0.85 | 0.004 | 0.006 |
| Whippet | 0 | 0.002 | 0.001 | 0.002 | 0.005 | 0.004 | 0.002 | 0.001 | 0.001 | 0.003 | 0.003 | 0.001 | 0.002 | 0.907 | 0.002 | 0.002 |
| Whippet | 0 | 0.001 | 0.003 | 0.004 | 0.002 | 0.002 | 0.007 | 0.002 | 0.001 | 0.001 | 0.001 | 0.001 | 0.001 | 0.943 | 0.001 | 0.001 |
| Whippet | (1) | 0.002 | 0.013 | 0.002 | 0.002 | 0.001 | 0.004 | 0.006 | 0.009 | 0.003 | 0.002 | 0.002 | 0.011 | 0.889 | 0.008 | 0.003 |
| Whippet | 0 | 0.005 | 0.007 | 0.019 | 0.011 | 0.003 | 0.002 | 0.004 | 0.01 | 0.007 | 0.005 | 0.004 | 0.011 | 0.804 | 0.006 | 0.002 |
| Whippet | 0 | 0.005 | 0.015 | 0.002 | 0.003 | 0.002 | 0.007 | 0.004 | 0.007 | 0.002 | 0.002 | 0.01 | 0.001 | 0.729 | 0.003 | 0.002 |
| Whippet | 0 | 0.005 | 0.008 | 0.004 | 0.001 | 0.002 | 0.006 | 0.001 | 0.002 | 0.001 | 0.003 | 0.005 | 0.009 | 0.803 | 0.011 | 0.003 |
| Whippet | 0 | 0.007 | 0.004 | 0.004 | 0.016 | 0.004 | 0.028 | 0.005 | 0.005 | 0.004 | 0.002 | 0.006 | 0.003 | 0.561 | 0.003 | 0.004 |
| Whippet | 0 | 0.006 | 0.001 | 0.003 | 0.003 | 0.002 | 0.003 | 0.004 | 0.003 | 0.011 | 0.002 | 0.01 | 0.001 | 0.899 | 0.002 | 0.002 |
| Whippet | 0 | 0.002 | 0.002 | 0.003 | 0.001 | 0.001 | 0.002 | 0.002 | 0.002 | 0.002 | 0.01 | 0.003 | 0.001 | 0.934 | 0.003 | 0.001 |
| Whippet | 0 | 0.002 | 0.001 | 0.002 | 0.002 | 0.002 | 0.001 | 0.001 | 0.001 | 0.001 | 0.001 | 0.001 | 0.001 | 0.964 | 0.001 | 0.001 |
| Whippet | 0 | 0.002 | 0.002 | 0.003 | 0.004 | 0.003 | 0.006 | 0.002 | 0.001 | 0.004 | 0.004 | 0.007 | 0.002 | 0.89 | 0.004 | 0.003 |
| | | Cluster | | | | | | | | | | | | | | |
| | | Assignment | | | | | | | | | | | | | | |
| Breed | | 16 | 17 | 18 | 19 | 20 | 21 | 22 | 23 | 24 | 25 | 26 | 27 | 28 | 29 | 30 |
| Whippet | | 0.002 | 0.002 | 0.002 | 0.001 | 0.004 | 0.001 | 0.001 | 0.002 | 0.002 | 0.001 | 0.001 | 0.008 | 0.001 | 0.001 | 0.003 |
| Whippet | | 0.002 | 0.003 | 0.002 | 0.007 | 0.002 | 0.007 | 0.005 | 0.003 | 0.004 | 0.002 | 0.001 | 0.003 | 0.002 | 0.003 | 0.001 |
| Whippet | | 0.002 | 0.024 | 0.028 | 0.002 | 0.001 | 0.002 | 0.003 | 0.003 | 0.004 | 0.005 | 0.003 | 0.013 | 0.005 | 0.002 | 0.002 |
| Whippet | | 0.001 | 0.003 | 0.003 | 0.003 | 0.03 | 0.008 | 0.015 | 0.002 | 0.003 | 0.003 | 0.001 | 0.004 | 0.002 | 0.004 | 0.003 |
| Whippet | | 0.002 | 0.005 | 0.005 | 0.003 | 0.04 | 0.004 | 0.015 | 0.001 | 0.002 | 0.012 | 0.002 | 0.011 | 0.001 | 0.004 | 0.002 |
| Whippet | | 0.002 | 0.01 | 0.004 | 0.002 | 0.007 | 0.007 | 0.008 | 0.002 | 0.012 | 0.008 | 0.002 | 0.022 | 0.003 | 0.006 | 0.003 |
| Whippet | | 0.006 | 0.001 | 0.006 | 0.001 | 0.006 | 0.001 | 0.002 | 0.001 | 0.001 | 0.004 | 0.002 | 0.001 | 0.008 | 0.001 | 0.002 |
| Whippet | | 0.001 | 0.001 | 0.002 | 0.002 | 0.002 | 0.003 | 0.006 | 0.002 | 0.001 | 0.006 | 0.003 | 0.004 | 0.002 | 0.002 | 0.004 |
| Whippet | | 0.001 | 0.002 | 0.002 | 0.001 | 0.003 | 0.001 | 0.003 | 0.001 | 0.002 | 0.002 | 0.001 | 0.003 | 0.001 | 0.002 | 0.001 |
| Whippet | | 0.003 | 0.007 | 0.003 | 0.002 | 0.006 | 0.002 | 0.004 | 0.001 | 0.004 | 0.001 | 0.002 | 0.001 | 0.008 | 0.002 | 0.002 |
| Whippet | | 0.001 | 0.003 | 0.003 | 0.002 | 0.004 | 0.002 | 0.005 | 0.003 | 0.004 | 0.004 | 0.001 | 0.004 | 0.003 | 0.002 | 0.001 |
| Whippet | | 0.002 | 0.033 | 0.014 | 0.005 | 0.01 | 0.003 | 0.004 | 0.005 | 0.006 | 0.006 | 0.003 | 0.006 | 0.002 | 0.007 | 0.007 |
| Whippet | | 0.005 | 0.018 | 0.011 | 0.002 | 0.007 | 0.002 | 0.005 | 0.003 | 0.002 | 0.003 | 0.002 | 0.005 | 0.002 | 0.004 | 0.002 |
| Whippet | | 0.001 | 0.001 | 0.001 | 0.001 | 0.001 | 0.002 | 0.002 | 0.001 | 0.001 | 0.002 | 0.002 | 0.009 | 0.002 | 0.001 | 0.002 |
| Whippet | | 0.002 | 0.014 | 0.007 | 0.032 | 0.006 | 0.004 | 0.054 | 0.005 | 0.004 | 0.003 | 0.002 | 0.004 | 0.01 | 0.002 | 0.003 |
| Whippet | | 0.001 | 0.003 | 0.005 | 0.005 | 0.002 | 0.006 | 0.004 | 0.002 | 0.002 | 0.007 | 0.002 | 0.002 | 0.003 | 0.002 | 0.002 |
| Whippet | | 0.007 | 0.005 | 0.009 | 0.001 | 0.001 | 0.003 | 0.001 | 0.006 | 0.001 | 0.002 | 0.002 | 0.002 | 0.001 | 0.001 | 0.001 |
| Whippet | | 0.002 | 0.001 | 0.003 | 0.001 | 0.002 | 0.003 | 0.002 | 0.005 | 0.001 | 0.003 | 0.008 | 0.004 | 0.002 | 0.001 | 0.004 |
| Whippet | | 0.001 | 0.008 | 0.008 | 0.005 | 0.001 | 0.003 | 0.002 | 0.003 | 0.003 | 0.004 | 0.002 | 0.005 | 0.008 | 0.008 | 0.002 |
| Whippet | | 0.001 | 0.002 | 0.002 | 0.001 | 0.002 | 0.002 | 0.003 | 0.004 | 0.002 | 0.002 | 0.003 | 0.006 | 0.001 | 0.001 | 0.002 |
| Whippet | | 0.003 | 0.004 | 0.007 | 0.002 | 0.002 | 0.004 | 0.005 | 0.005 | 0.007 | 0.006 | 0.003 | 0.009 | 0.002 | 0.004 | 0.012 |
| Whippet | | 0.003 | 0.017 | 0.01 | 0.005 | 0.004 | 0.01 | 0.007 | 0.006 | 0.004 | 0.094 | 0.006 | 0.002 | 0.024 | 0.004 | 0.007 |
| Whippet | | 0.002 | 0.002 | 0.005 | 0.003 | 0.003 | 0.002 | 0.002 | 0.012 | 0.002 | 0.003 | 0.002 | 0.004 | 0.003 | 0.004 | 0.002 |
| Whippet | | 0.002 | 0.002 | 0.001 | 0.002 | 0.001 | 0.001 | 0.004 | 0.002 | 0.001 | 0.005 | 0.003 | 0.003 | 0.001 | 0.003 | 0.002 |
| Whippet | | 0.001 | 0.003 | 0.005 | 0.005 | 0.003 | 0.002 | 0.005 | 0.004 | 0.003 | 0.002 | 0.025 | 0.002 | 0.006 | 0.006 | 0.003 |
| Whippet | | 0.01 | 0.006 | 0.01 | 0.002 | 0.023 | 0.002 | 0.002 | 0.005 | 0.003 | 0.002 | 0.011 | 0.006 | 0.003 | 0.005 | 0.004 |
| Whippet | | 0.003 | 0.003 | 0.003 | 0.007 | 0.002 | 0.019 | 0.003 | 0.004 | 0.001 | 0.009 | 0.003 | 0.002 | 0.001 | 0.012 | 0.012 |
| Whippet | | 0.003 | 0.003 | 0.002 | 0.012 | 0.004 | 0.002 | 0.004 | 0.008 | 0.007 | 0.005 | 0.003 | 0.003 | 0.002 | 0.002 | 0.002 |
| Whippet | | 0.002 | 0.002 | 0.003 | 0.002 | 0.002 | 0.002 | 0.001 | 0.002 | 0.001 | 0.001 | 0.001 | 0.001 | 0.002 | 0.001 | 0.003 |
| Whippet | | 0.001 | 0.005 | 0.005 | 0.003 | 0.001 | 0.004 | 0.002 | 0.003 | 0.003 | 0.007 | 0.001 | 0.002 | 0.001 | 0.003 | 0.002 |
| Whippet | | 0.002 | 0.002 | 0.003 | 0.014 | 0.001 | 0.009 | 0.006 | 0.029 | 0.003 | 0.003 | 0.001 | 0.008 | 0.002 | 0.007 | 0.011 |
| Whippet | | 0.002 | 0.068 | 0.033 | 0.036 | 0.001 | 0.01 | 0.006 | 0.002 | 0.004 | 0.018 | 0.002 | 0.004 | 0.002 | 0.004 | 0.011 |
| Whippet | | 0.001 | 0.018 | 0.025 | 0.038 | 0.003 | 0.002 | 0.005 | 0.002 | 0.031 | 0.002 | 0.002 | 0.001 | 0.004 | 0.001 | 0.002 |
| Whippet | | 0.005 | 0.009 | 0.093 | 0.003 | 0.005 | 0.002 | 0.002 | 0.003 | 0.004 | 0.009 | 0.002 | 0.005 | 0.199 | 0.002 | 0.003 |
| Whippet | | 0.002 | 0.003 | 0.002 | 0.002 | 0.004 | 0.003 | 0.002 | 0.002 | 0.001 | 0.003 | 0.003 | 0.013 | 0.005 | 0.001 | 0.003 |
| Whippet | | 0.001 | 0.001 | 0.002 | 0.002 | 0.003 | 0.001 | 0.001 | 0.002 | 0.001 | 0.003 | 0.005 | 0.004 | 0.003 | 0.001 | 0.001 |
| Whippet | | 0.001 | 0.001 | 0.001 | 0.002 | 0.001 | 0.001 | 0.001 | 0.001 | 0.001 | 0.002 | 0.001 | 0.001 | 0.001 | 0.001 | 0.001 |
| Whippet | | 0.001 | 0.002 | 0.005 | 0.002 | 0.002 | 0.003 | 0.008 | 0.003 | 0.004 | 0.01 | 0.011 | 0.005 | 0.002 | 0.003 | 0.003 |
| | % | Cluster | | | | | | | | | | | | | | |
| | missing | assignment | | | | | | | | | | | | | | |
| Breed | data | 1 | 2 | 3 | 4 | 5 | 6 | 7 | 8 | 9 | 10 | 11 | 12 | 13 | 14 | 15 |
| Yorkshire | 0 | 0.006 | 0.86 | 0.004 | 0.005 | 0.003 | 0.005 | 0.007 | 0.007 | 0.01 | 0.001 | 0.002 | 0.001 | 0.002 | 0.002 | 0.003 |
| Terrier | | | | | | | | | | | | | | | | |
| Yorkshire | (2) | 0.006 | 0.898 | 0.007 | 0.009 | 0.007 | 0.003 | 0.002 | 0.002 | 0.001 | 0.004 | 0.002 | 0.003 | 0.004 | 0.002 | 0.002 |
| Terrier | | | | | | | | | | | | | | | | |
| Yorkshire | (1) | 0.001 | 0.821 | 0.002 | 0.007 | 0.002 | 0.007 | 0.007 | 0.003 | 0.047 | 0.003 | 0.004 | 0.003 | 0.005 | 0.002 | 0.002 |
| Terrier | | | | | | | | | | | | | | | | |
| Yorkshire | 0 | 0.003 | 0.701 | 0.003 | 0.002 | 0.002 | 0.002 | 0.01 | 0.007 | 0.003 | 0.004 | 0.003 | 0.016 | 0.043 | 0.043 | 0.008 |
| Terrier | | | | | | | | | | | | | | | | |
| Yorkshire | 0 | 0.005 | 0.768 | 0.003 | 0.002 | 0.001 | 0.016 | 0.003 | 0.007 | 0.004 | 0.007 | 0.005 | 0.006 | 0.003 | 0.002 | 0.005 |
| Terrier | | | | | | | | | | | | | | | | |
| Yorkshire | 0 | 0.005 | 0.73 | 0.006 | 0.003 | 0.004 | 0.004 | 0.021 | 0.01 | 0.007 | 0.006 | 0.019 | 0.001 | 0.006 | 0.008 | 0.008 |
| Terrier | | | | | | | | | | | | | | | | |
| Yorkshire | 0 | 0.001 | 0.954 | 0.001 | 0.001 | 0.001 | 0.001 | 0.001 | 0.002 | 0.001 | 0.002 | 0.001 | 0.002 | 0.001 | 0.002 | 0.002 |
| Terrier | | | | | | | | | | | | | | | | |
| Yorkshire | 0 | 0.002 | 0.963 | 0.002 | 0.002 | 0.001 | 0.001 | 0.001 | 0.001 | 0.001 | 0.001 | 0.001 | 0.001 | 0.001 | 0.001 | 0.002 |
| Terrier | | | | | | | | | | | | | | | | |
| Yorkshire | 0 | 0.005 | 0.673 | 0.006 | 0.02 | 0.004 | 0.044 | 0.025 | 0.051 | 0.007 | 0.005 | 0.015 | 0.006 | 0.011 | 0.007 | 0.004 |
| Terrier | | | | | | | | | | | | | | | | |
| Yorkshire | 0 | 0.004 | 0.578 | 0.019 | 0.006 | 0.007 | 0.023 | 0.002 | 0.003 | 0.002 | 0.001 | 0.002 | 0.002 | 0.004 | 0.002 | 0.014 |
| Terrier | | | | | | | | | | | | | | | | |
| Yorkshire | 0 | 0.005 | 0.728 | 0.002 | 0.002 | 0.007 | 0.012 | 0.002 | 0.004 | 0.004 | 0.001 | 0.002 | 0.002 | 0.003 | 0.003 | 0.004 |
| Terrier | | | | | | | | | | | | | | | | |
| Yorkshire | 0 | 0.007 | 0.815 | 0.002 | 0.009 | 0.007 | 0.007 | 0.004 | 0.002 | 0.013 | 0.014 | 0.002 | 0.004 | 0.018 | 0.004 | 0.002 |
| Terrier | | | | | | | | | | | | | | | | |
| Yorkshire | 0 | 0.006 | 0.775 | 0.022 | 0.004 | 0.002 | 0.006 | 0.007 | 0.007 | 0.004 | 0.007 | 0.016 | 0.003 | 0.027 | 0.006 | 0.007 |
| Terrier | | | | | | | | | | | | | | | | |
| Yorkshire | 0 | 0.003 | 0.877 | 0.002 | 0.002 | 0.001 | 0.007 | 0.004 | 0.005 | 0.015 | 0.006 | 0.001 | 0.001 | 0.004 | 0.006 | 0.005 |
| Terrier | | | | | | | | | | | | | | | | |
| Yorkshire | 0 | 0.011 | 0.594 | 0.01 | 0.002 | 0.004 | 0.11 | 0.005 | 0.008 | 0.009 | 0.004 | 0.062 | 0.003 | 0.004 | 0.004 | 0.005 |
| Terrier | | | | | | | | | | | | | | | | |
| Yorkshire | 0 | 0.002 | 0.473 | 0.002 | 0.003 | 0.007 | 0.013 | 0.012 | 0.041 | 0.006 | 0.008 | 0.046 | 0.006 | 0.004 | 0.164 | 0.003 |
| Terrier | | | | | | | | | | | | | | | | |
| Yorkshire | 0 | 0.007 | 0.814 | 0.007 | 0.004 | 0.003 | 0.008 | 0.005 | 0.009 | 0.003 | 0.003 | 0.005 | 0.004 | 0.003 | 0.002 | 0.011 |
| Terrier | | | | | | | | | | | | | | | | |
| Yorkshire | (1) | 0.015 | 0.781 | 0.012 | 0.027 | 0.003 | 0.005 | 0.009 | 0.007 | 0.003 | 0.002 | 0.003 | 0.006 | 0.005 | 0.002 | 0.004 |
| Terrier | | | | | | | | | | | | | | | | |
| Yorkshire | 0 | 0.003 | 0.931 | 0.004 | 0.002 | 0.002 | 0.001 | 0.001 | 0.002 | 0.003 | 0.002 | 0.002 | 0.004 | 0.001 | 0.005 | 0.003 |
| Terrier | | | | | | | | | | | | | | | | |
| Yorkshire | 0 | 0.005 | 0.816 | 0.011 | 0.007 | 0.003 | 0.006 | 0.006 | 0.005 | 0.002 | 0.009 | 0.005 | 0.004 | 0.003 | 0.005 | 0.001 |
| Terrier | | | | | | | | | | | | | | | | |
| Yorkshire | 0 | 0.005 | 0.895 | 0.005 | 0.002 | 0.001 | 0.002 | 0.002 | 0.005 | 0.001 | 0.002 | 0.004 | 0.004 | 0.004 | 0.008 | 0.007 |
| Terrier | | | | | | | | | | | | | | | | |
| Yorkshire | 0 | 0.015 | 0.742 | 0.002 | 0.003 | 0.002 | 0.065 | 0.031 | 0.014 | 0.003 | 0.002 | 0.027 | 0.001 | 0.007 | 0.006 | 0.008 |
| Terrier | | | | | | | | | | | | | | | | |
| Yorkshire | 0 | 0.097 | 0.707 | 0.006 | 0.005 | 0.009 | 0.012 | 0.007 | 0.008 | 0.004 | 0.007 | 0.009 | 0.008 | 0.004 | 0.011 | 0.003 |
| Terrier | | | | | | | | | | | | | | | | |
| Yorkshire | 0 | 0.002 | 0.778 | 0.002 | 0.001 | 0.002 | 0.003 | 0.074 | 0.012 | 0.008 | 0.012 | 0.002 | 0.002 | 0.008 | 0.002 | 0.006 |
| Terrier | | | | | | | | | | | | | | | | |
| Yorkshire | 0 | 0.002 | 0.92 | 0.001 | 0.002 | 0.004 | 0.004 | 0.002 | 0.003 | 0.003 | 0.002 | 0.002 | 0.004 | 0.002 | 0.001 | 0.002 |
| Terrier | | | | | | | | | | | | | | | | |
| Yorkshire | (1) | 0.003 | 0.751 | 0.003 | 0.003 | 0.006 | 0.008 | 0.016 | 0.004 | 0.007 | 0.003 | 0.007 | 0.003 | 0.003 | 0.004 | 0.002 |
| Terrier | | | | | | | | | | | | | | | | |
| Yorkshire | 0 | 0.002 | 0.814 | 0.076 | 0.004 | 0.003 | 0.002 | 0.002 | 0.006 | 0.002 | 0.002 | 0.004 | 0.004 | 0.003 | 0.003 | 0.009 |
| Terrier | | | | | | | | | | | | | | | | |
| Yorkshire | 0 | 0.002 | 0.928 | 0.002 | 0.002 | 0.002 | 0.002 | 0.002 | 0.001 | 0.001 | 0.005 | 0.002 | 0.003 | 0.003 | 0.002 | 0.004 |
| Terrier | | | | | | | | | | | | | | | | |
| Yorkshire | 0 | 0.002 | 0.647 | 0.004 | 0.003 | 0.003 | 0.269 | 0.004 | 0.004 | 0.004 | 0.002 | 0.005 | 0.002 | 0.002 | 0.001 | 0.003 |
| Terrier | | | | | | | | | | | | | | | | |
| Yorkshire | 0 | 0.006 | 0.668 | 0.003 | 0.002 | 0.002 | 0.21 | 0.016 | 0.003 | 0.004 | 0.002 | 0.014 | 0.002 | 0.007 | 0.001 | 0.002 |
| Terrier | | | | | | | | | | | | | | | | |
| Yorkshire | 0 | 0.003 | 0.841 | 0.023 | 0.008 | 0.008 | 0.004 | 0.004 | 0.004 | 0.002 | 0.006 | 0.017 | 0.003 | 0.007 | 0.006 | 0.006 |
| Terrier | | | | | | | | | | | | | | | | |
| Yorkshire | 0 | 0.004 | 0.755 | 0.008 | 0.002 | 0.002 | 0.002 | 0.008 | 0.075 | 0.001 | 0.006 | 0.002 | 0.018 | 0.015 | 0.002 | 0.004 |
| Terrier | | | | | | | | | | | | | | | | |
| Yorkshire | 0 | 0.006 | 0.73 | 0.005 | 0.003 | 0.009 | 0.007 | 0.03 | 0.004 | 0.003 | 0.031 | 0.056 | 0.048 | 0.005 | 0.003 | 0.002 |
| Terrier | | | | | | | | | | | | | | | | |
| Yorkshire | 0 | 0.003 | 0.742 | 0.006 | 0.009 | 0.005 | 0.003 | 0.006 | 0.008 | 0.005 | 0.006 | 0.012 | 0.016 | 0.009 | 0.017 | 0.002 |
| Terrier | | | | | | | | | | | | | | | | |
| Yorkshire | 0 | 0.003 | 0.847 | 0.003 | 0.004 | 0.003 | 0.007 | 0.006 | 0.005 | 0.002 | 0.003 | 0.005 | 0.002 | 0.02 | 0.004 | 0.006 |
| Terrier | | | | | | | | | | | | | | | | |
| Yorkshire | 0 | 0.004 | 0.73 | 0.004 | 0.003 | 0.002 | 0.002 | 0.007 | 0.018 | 0.002 | 0.003 | 0.098 | 0.001 | 0.005 | 0.024 | 0.006 |
| Terrier | | | | | | | | | | | | | | | | |
| Yorkshire | 0 | 0.019 | 0.575 | 0.014 | 0.007 | 0.007 | 0.004 | 0.015 | 0.082 | 0.002 | 0.002 | 0.104 | 0.004 | 0.005 | 0.005 | 0.005 |
| Terrier | | | | | | | | | | | | | | | | |
| Yorkshire | 0 | 0.003 | 0.864 | 0.002 | 0.002 | 0.002 | 0.004 | 0.011 | 0.005 | 0.004 | 0.003 | 0.001 | 0.002 | 0.021 | 0.003 | 0.002 |
| Terrier | | | | | | | | | | | | | | | | |

| | Cluster | | | | | | | | | | | | | | |
|---|---|---|---|---|---|---|---|---|---|---|---|---|---|---|---|
| | Assignment | | | | | | | | | | | | | | |
| Breed | 16 | 17 | 18 | 19 | 20 | 21 | 22 | 23 | 24 | 25 | 26 | 27 | 28 | 29 | 30 |
| Yorkshire Terrier | 0.007 | 0.002 | 0.004 | 0.002 | 0.007 | 0.002 | 0.008 | 0.003 | 0.006 | 0.006 | 0.015 | 0.002 | 0.002 | 0.004 | 0.014 |
| Yorkshire Terrier | 0.001 | 0.002 | 0.003 | 0.006 | 0.002 | 0.002 | 0.004 | 0.002 | 0.007 | 0.001 | 0.003 | 0.003 | 0.003 | 0.007 | 0.003 |
| Yorkshire Terrier | 0.002 | 0.004 | 0.029 | 0.002 | 0.003 | 0.002 | 0.004 | 0.002 | 0.002 | 0.003 | 0.016 | 0.004 | 0.004 | 0.002 | 0.005 |
| Yorkshire Terrier | 0.002 | 0.005 | 0.009 | 0.002 | 0.003 | 0.02 | 0.006 | 0.014 | 0.007 | 0.004 | 0.007 | 0.049 | 0.001 | 0.003 | 0.018 |
| Yorkshire Terrier | 0.004 | 0.005 | 0.003 | 0.034 | 0.002 | 0.003 | 0.004 | 0.013 | 0.009 | 0.019 | 0.011 | 0.021 | 0.002 | 0.022 | 0.009 |
| Yorkshire Terrier | 0.003 | 0.012 | 0.058 | 0.01 | 0.002 | 0.015 | 0.011 | 0.009 | 0.002 | 0.006 | 0.004 | 0.003 | 0.004 | 0.011 | 0.012 |
| Yorkshire Terrier | 0.001 | 0.001 | 0.001 | 0.001 | 0.001 | 0.003 | 0.004 | 0.001 | 0.001 | 0.001 | 0.003 | 0.001 | 0.001 | 0.001 | 0.004 |
| Yorkshire Terrier | 0.001 | 0.001 | 0.001 | 0.001 | 0.001 | 0.001 | 0.003 | 0.001 | 0.002 | 0.001 | 0.001 | 0.001 | 0.001 | 0.002 | 0.002 |
| Yorkshire Terrier | 0.002 | 0.011 | 0.005 | 0.026 | 0.002 | 0.008 | 0.013 | 0.005 | 0.003 | 0.011 | 0.009 | 0.004 | 0.009 | 0.004 | 0.005 |
| Yorkshire Terrier | 0.002 | 0.005 | 0.003 | 0.249 | 0.009 | 0.003 | 0.028 | 0.005 | 0.007 | 0.002 | 0.002 | 0.002 | 0.005 | 0.001 | 0.007 |
| Yorkshire Terrier | 0.002 | 0.003 | 0.003 | 0.168 | 0.003 | 0.01 | 0.004 | 0.003 | 0.006 | 0.003 | 0.001 | 0.002 | 0.008 | 0.001 | 0.003 |
| Yorkshire Terrier | 0.005 | 0.01 | 0.005 | 0.002 | 0.006 | 0.004 | 0.004 | 0.017 | 0.004 | 0.002 | 0.009 | 0.009 | 0.002 | 0.008 | 0.003 |
| Yorkshire Terrier | 0.003 | 0.032 | 0.009 | 0.003 | 0.003 | 0.002 | 0.015 | 0.004 | 0.002 | 0.004 | 0.002 | 0.004 | 0.009 | 0.002 | 0.006 |
| Yorkshire Terrier | 0.001 | 0.002 | 0.003 | 0.002 | 0.001 | 0.007 | 0.006 | 0.011 | 0.009 | 0.004 | 0.005 | 0.002 | 0.003 | 0.002 | 0.003 |
| Yorkshire Terrier | 0.009 | 0.025 | 0.008 | 0.012 | 0.018 | 0.011 | 0.001 | 0.003 | 0.015 | 0.003 | 0.007 | 0.009 | 0.002 | 0.008 | 0.034 |
| Yorkshire Terrier | 0.004 | 0.009 | 0.056 | 0.007 | 0.006 | 0.003 | 0.009 | 0.003 | 0.007 | 0.007 | 0.03 | 0.003 | 0.054 | 0.004 | 0.01 |
| Yorkshire Terrier | 0.002 | 0.025 | 0.003 | 0.006 | 0.008 | 0.012 | 0.004 | 0.004 | 0.006 | 0.003 | 0.003 | 0.003 | 0.015 | 0.015 | 0.004 |
| Yorkshire Terrier | 0.001 | 0.008 | 0.003 | 0.027 | 0.003 | 0.003 | 0.004 | 0.041 | 0.001 | 0.002 | 0.003 | 0.003 | 0.009 | 0.004 | 0.003 |
| Yorkshire Terrier | 0.001 | 0.002 | 0.002 | 0.001 | 0.001 | 0.002 | 0.003 | 0.003 | 0.006 | 0.002 | 0.002 | 0.001 | 0.001 | 0.004 | 0.002 |
| Yorkshire Terrier | 0.007 | 0.011 | 0.004 | 0.002 | 0.003 | 0.002 | 0.003 | 0.05 | 0.004 | 0.006 | 0.003 | 0.003 | 0.002 | 0.001 | 0.011 |
| Yorkshire Terrier | 0.001 | 0.004 | 0.012 | 0.002 | 0.002 | 0.003 | 0.009 | 0.002 | 0.005 | 0.002 | 0.002 | 0.003 | 0.002 | 0.003 | 0.002 |
| Yorkshire Terrier | 0.003 | 0.002 | 0.004 | 0.002 | 0.003 | 0.004 | 0.002 | 0.003 | 0.014 | 0.007 | 0.002 | 0.017 | 0.003 | 0.001 | 0.004 |
| Yorkshire Terrier | 0.003 | 0.007 | 0.021 | 0.003 | 0.007 | 0.012 | 0.01 | 0.003 | 0.01 | 0.001 | 0.003 | 0.002 | 0.009 | 0.001 | 0.009 |
| Yorkshire Terrier | 0.002 | 0.006 | 0.009 | 0.003 | 0.007 | 0.01 | 0.007 | 0.013 | 0.005 | 0.002 | 0.006 | 0.001 | 0.006 | 0.006 | 0.003 |
| Yorkshire Terrier | 0.001 | 0.002 | 0.002 | 0.003 | 0.009 | 0.001 | 0.002 | 0.006 | 0.002 | 0.001 | 0.002 | 0.002 | 0.002 | 0.003 | 0.007 |
| Yorkshire Terrier | 0.003 | 0.012 | 0.003 | 0.005 | 0.006 | 0.003 | 0.003 | 0.018 | 0.003 | 0.003 | 0.004 | 0.031 | 0.002 | 0.006 | 0.074 |
| Yorkshire Terrier | 0.001 | 0.005 | 0.004 | 0.004 | 0.005 | 0.005 | 0.014 | 0.002 | 0.002 | 0.001 | 0.003 | 0.001 | 0.008 | 0.003 | 0.003 |
| Yorkshire Terrier | 0.001 | 0.004 | 0.003 | 0.002 | 0.003 | 0.001 | 0.006 | 0.001 | 0.002 | 0.004 | 0.003 | 0.002 | 0.002 | 0.002 | 0.002 |
| Yorkshire Terrier | 0.003 | 0.001 | 0.005 | 0.006 | 0.008 | 0.002 | 0.001 | 0.004 | 0.002 | 0.002 | 0.002 | 0.001 | 0.003 | 0.002 | 0.003 |
| Yorkshire Terrier | 0.002 | 0.002 | 0.01 | 0.004 | 0.004 | 0.002 | 0.001 | 0.007 | 0.002 | 0.003 | 0.002 | 0.003 | 0.009 | 0.004 | 0.002 |
| Yorkshire Terrier | 0.001 | 0.002 | 0.006 | 0.007 | 0.004 | 0.005 | 0.01 | 0.002 | 0.002 | 0.004 | 0.002 | 0.003 | 0.005 | 0.003 | 0.001 |
| Yorkshire Terrier | 0.021 | 0.02 | 0.003 | 0.005 | 0.001 | 0.004 | 0.005 | 0.004 | 0.002 | 0.002 | 0.011 | 0.003 | 0.002 | 0.005 | 0.007 |
| Yorkshire Terrier | 0.004 | 0.006 | 0.003 | 0.005 | 0.002 | 0.002 | 0.002 | 0.004 | 0.004 | 0.002 | 0.002 | 0.004 | 0.006 | 0.001 | |
| Yorkshire Terrier | 0.005 | 0.002 | 0.005 | 0.003 | 0.003 | 0.007 | 0.003 | 0.003 | 0.012 | 0.003 | 0.003 | 0.003 | 0.009 | 0.089 | 0.003 |
| Yorkshire Terrier | 0.006 | 0.005 | 0.007 | 0.003 | 0.002 | 0.008 | 0.003 | 0.001 | 0.002 | 0.01 | 0.006 | 0.009 | 0.004 | 0.007 | 0.005 |
| Yorkshire Terrier | 0.002 | 0.004 | 0.007 | 0.003 | 0.002 | 0.034 | 0.002 | 0.002 | 0.002 | 0.003 | 0.002 | 0.003 | 0.004 | 0.02 | 0.002 |
| Yorkshire Terrier | 0.004 | 0.002 | 0.007 | 0.008 | 0.003 | 0.051 | 0.002 | 0.002 | 0.006 | 0.006 | 0.002 | 0.006 | 0.034 | 0.016 | 0.003 |
| Yorkshire Terrier | 0.004 | 0.004 | 0.011 | 0.001 | 0.002 | 0.004 | 0.001 | 0.015 | 0.002 | 0.002 | 0.007 | 0.008 | 0.002 | 0.004 | 0.001 |

TABLE 6
Percentage of animals correctly assigned to breed based on
probability of assignment.

| | | Probability | | |
|---|---|---|---|---|
| | | of assignment | | |
| Breed | N | >.9 | >.8 | >.7 |
| Afghan Hound | 38 | 0.68 | 0.97 | 1.00 |
| Basenji | 38 | 0.84 | 0.97 | 1.00 |
| Basset Hound | 38 | 0.45 | 0.84 | 0.97 |
| Beagle | 38 | 0.34 | 0.79 | 1.00 |
| Belgian Tervuren | 38 | 0.37 | 0.92 | 0.97 |
| Bernese Mountain Dog | 38 | 0.71 | 0.95 | 1.00 |
| Borzoi | 38 | 0.37 | 0.66 | 0.84 |
| Chihuahua | 38 | 0.13 | 0.24 | 0.53 |
| Chinese Shar-Pei | 38 | 0.13 | 0.50 | 0.84 |
| Cocker Spaniel | 38 | 0.50 | 0.89 | 0.97 |
| Dachshund - Cluster 1 | 38 | 0.03 | 0.16 | 0.26 |
| Dachshund - Cluster 2 | 38 | 0.13 | 0.13 | 0.18 |
| Doberman Pinscher | 38 | 0.74 | 0.97 | 1.00 |
| German Shepherd Dog | 38 | 0.66 | 0.92 | 1.00 |
| German Shorthaired Pointer | 38 | 0.08 | 0.55 | 0.63 |
| Golden Retriever | 38 | 0.42 | 0.79 | 0.87 |
| Labrador Retriever | 38 | 0.26 | 0.63 | 0.84 |
| Mastiff | 38 | 0.45 | 0.89 | 1.00 |
| Miniature Schnauzer | 38 | 0.66 | 0.95 | 1.00 |
| Poodle | 38 | 0.24 | 0.68 | 0.76 |
| Pug | 38 | 0.76 | 1.00 | 1.00 |
| Rottweiler | 38 | 0.58 | 0.92 | 0.92 |
| Saluki | 38 | 0.13 | 0.37 | 0.47 |
| Samoyed | 38 | 0.45 | 0.63 | 0.92 |
| Shetland Sheepdog | 38 | 0.71 | 1.00 | 1.00 |
| Siberian Husky | 38 | 0.34 | 0.82 | 0.97 |
| St Bernard | 38 | 0.58 | 0.89 | 1.00 |
| Whippet | 38 | 0.39 | 0.87 | 0.97 |
| Yorkshire Terrier | 38 | 0.13 | 0.45 | 0.82 |

TABLE 7

Oligonucleotide marker sets for canine SNPs.
PCR primers and extension primers are provided below.

| | | | |
|---|---|---|---|
| Accession | Forward Primer (SEQ ID NOS:102-203) | Reverse Primer (SEQ ID NOS:204-305) | Extension Primer (SEQ ID NOS:306-407) |

| | | | |
|---|---|---|---|
| ss9048431 | TATTGACTCTATACCTCTAAAGAATCGC | AGAGTTTCATACTGGGGTAACTTTG | AGACTTTTAAAGTTTAAATGAATTA |
| ss9053109 | TCAGTGAGAAGAGAATCATCCG | TGGCACCAGAAGTAGGTGG | TCTTTACAGACCAGCTTTTGTCTCC |
| ss9067589 | ATTATGATCTGCATCTTCAGAGAC | ATATGGTCTGGGCATGGTTA | AGATAGAGCCAGTCTGTTGGCAAGC |
| ss9069201 | ATTTTGTGGAGAAGTGTGCAG | TTATGCGGGTACTGCTTTATACA | TCTCAGAAAAATGGAAAACAAGAGC |
| ss9084075 | CAGGACGTCTGGAGGTGT | TACAGCTCACTGGCACCTC | CCTGGGCTAGAGTTACAAACATCAG |
| ss9090942 | AAAGCACTCCATACTTAAAGTCA | TTTGTTATCCTAATTCTCTTGGTTTATT | ATTTGCCTTCTTTCACATCCACATG |
| ss9101730 | ATTATATGGTCTCACTTATATGGGGA | ATTTCCCAGAGTTAGGAGTCTCTC | AAAAATAGTGGAAGGGGTTATAGGG |
| ss9108332 | TTAATGGGGATGGTTTGAGA | CAACTGTATCTCTATACATCTCAATCTATCT | GTTAATAAGGGGAAGTAGAGTTTCA |
| ss9132982 | TCTTCCTAAAGGGAAGTTGGG | AATGTAGGAAGTGCCTACGTGA | AGGTGACCTGTGGCACTGGGGTCTT |
| ss9139126 | ACTCAAAGCAGCGCCATG | TGGTTGTGTGGTTGGGGC | TAGTGGATTTCACAGATGAGGGAAC |
| ss9142796 | TGTCCAGTCCAATCCCTACC | AGGAGACAGGGATCATCACC | ATCACCTCATGTTATAGACTGGGAA |
| ss9152677 | ATACCCAGAATAGCCGATTTC | ATGATCTTAGAATAGATCTATGCCTTTT | TAATTTCCTGACATGCTTGCTCTGC |
| ss9156891 | CTGCCCRAACCTTCAGAA | AGCCAGGATGGGCCTATT | GTCCAGAGGTACTGCCCAGCAGACA |
| ss9171081 | TATAACCTACCTCCTGCTGC | TTCACATTTAAAACTCCATTTTTG | ACACAGGCTCTCATTCTTACCAATC |
| ss9177956 | TTCAAAAGTAACTGTAGCTTACCAAA | TTCTTTAGATGGAAATTATTTTTAGGC | CCTTAGAAAAGAGGTTAAAAGAAAA |
| ss9186525 | TTCAGTCTTACRACACTTAGCCC | TRGGTAGGTAATGGGAAGGG | TCTGCCTCATOCATTCAGCTGTACT |
| ss9191087 | AATCCACCTGCCCAGCCC | AATATTAGAAAACACAGCAAGCTTCT | CCGGATTTAATTAAGGTAGTCTTAC |
| ss9200241 | CCTATCTTTGCACTCACTGTTA | ATTCACTCATTGAAATATATATCCCC | TTCATTCGAGATTTAGTTTATGACA |
| ss9230071 | TTTCTGGTAATCATACAGATAAAGG | ATCCCTGAAGACACATTTCATG | TAGCTGGAGCATTCATTAGGATCRC |
| ss9233837 | TGAACATTTTCTTCAAGTATCPAATC | CTTTGTATATTTACAAGGACAAGGC | CAAGTAGGCATGTTAATATCATAAA |
| ss9235114 | ATGCTTCATCCTCTGGTGTG | TATATGGGAAATTAGGAAGGTATTGT | TGTTCATAGGCCAATACTCGTACCA |
| ss9244345 | TTAATACCCATTACCTAGTTCCCC | TCTAAAGGCAGGATCAGAGG | TAAAAAGAAATGTCTACCTCACTGA |
| ss9245977 | AGAGGTTGCCCTTTGCCA | AACCCACATGTAAAAATAGTAGTGC | CTTAATTAGTTACATGCTGCCTTGG |
| ss9251154 | TGGACCTTAGAGTCTTCGAGC | AACAGGCTGCCCTAGAAGAT | ACAGAAGGTCTCCCATTGGCTGCTC |
| ss9259716 | ATATCCCATAACCAAGACTGCA | TTGACAGCTCTTGGCAACA | GGTCCTTGTTTATCTTTTCCAAAAA |
| ss9270557 | CTGAAGGCCCACAACTTG | ATTAGTCTTGAATATCTCAATCAATATGG | ATTATTAAACAATCATATGTAGC |
| ss9278814 | ATTGGAAAGGAAAAGAATTAGTAT | ATGTGGGAAAAGAGACTTAAATGT | TTTAGTACATTAACPACATCCTCAA |
| ss9281595 | GCTAAGAGGAGGAAGAAAATG | AATTGTGGATTATGGGTGA | CATGGGAATAATAGAGGAAATTGTA |
| ss9282411 | TTCTGGTCTAGAGAGAGGTTTTTT | ATGAAATATCTCCAGACCTGGG | CACCATGTGGCCCAACATCAGTGCT |
| ss9285114 | TCCCCAGGAGCACACTGT | ACCTGGGAGGTGGGGAAC | ATAGAAGGTACACGTGAAAACACCA |
| ss9290112 | TTCTAGTTGATTAGGATATAGCAATGC | TACTACTGCATATTCTCTTTATCCTGTG | TTCATTTTTGTCAGGAGGTAAAGAT |
| ss9290361 | ATCTCCCAAGCAGCAACC | TTGGAAAGAAGACTGGGC | TTCTTTCATTTTGCTGTGGGCGGCT |
| ss9292376 | AAAAGTGGAAAATTGATTCATATG | ATTGGACTTGCTACAGCTCTG | CTATCCGGTCAACAAATGTGCCCAT |
| ss9294456 | ATATTTCTGTTTTCTGCATGTTGC | ATTCTTTCTAACTTCCTACTGGGG | ATGTCACTTTGCAAGTATAAACTGA |
| ss9296487 | AAGAGGGTGATGAATAGTATCCT | AGGATATAGTTTCTAGCTTGCCAG | CATGAGGCCATTGTTTGCAGAAGCA |
| ss9300915 | CAGTCTCCCTGAGTGAAGTAAATATT | AGAAATCACACACAAAAAGCATATC | GTAGTGGCATTTCCATCTAGGAGAG |
| ss9301348 | TATGTACTTCCCAGATCTCCACTC | AGCATATTTTTCCTCAGTCCAA | CATTTATCTCTCTTAGAGTTTTAGG |
| ss9307596 | AAAAGGTGGCCAGGGACT | TGGGGCTGAGGACTAATGT | CTTTACACAGGACCTGAAGGCTCAC |
| ss9308314 | AAGAAAGAGATAGAAGCAGGGATC | ATAGAGTTATTGGGATTATTGCAAGA | CTAGGGAGGCTCAAGACCTTTAGGT |
| ss9313462 | GGAAGACTTGGTGCTGAATTC | ATCAGAGTGAAAACCTCTGACC | GYTACCTGGGATGTTAGGTCATTAA |
| ss9313564 | TGCTCTGTGTCTTGCCTATTT | ATGTTAGAGCAGATCAAGGACCT | TATTGATTTGCCGTGGGTTCTGTCC |
| ss9313781 | ACTAGATTGAACTGACAATGTGCC | AAGAAAAGGACGTCAACTCACTT | AGAGAGTGCTTCGACTCAACGCCCA |
| ss9328275 | AGGGCAGTGTGTGCGCTACTA | GCTACTAGAACTCTCAGCTGGA | CCTCAGACCAAAATGGGACCAGAGC |
| ss9335917 | AGCAATGGATATCATAGGACAATA | TTTTCTCTCCCACTGACAGAAC | GTGAAGGAGATTCAGAGTAGCATAG |
| ss9339680 | AACTGTATITATCTGCCTGATTTTAATT | AAAGATAAGGGAAAGGACCTGT | GATAAGAGTTCYCCTAAGOTGGATT |
| ss9362797 | AAAACAGCCTTCCTTAAGACAG | TGCTTGGCTCTTGAGGTATAG | GCATTGTACTGAGCACATCCTCGSG |
| ss9366135 | TCTTTATCGGCTACTAGAAAATT | AATATTCTATGTATATATACCAGCCCTG | TAAGCTACAGATGTGAATTGCATTT |
| ss9366251 | AGTTCACTGGTCTCCTAGGATTT | AAGAAAAGCCCCAAAGCTT | ATACTGTTGTAGAAGCTTTTGTTTT |
| ss9378306 | TCAACCAATATACATATTTTGAAGATG | ACTGATTTAAAAAAGAACAAAACCAC | GGTGAACCCTTCTGTTTCTGTGAAA |
| ss9380511 | CGTGCAGGTCGTATGCAT | AGATTCTGAGACTCTATATAGTAACACGC | ACGCATATATATGTATGACGAGTGC |
| ss9382377 | TGTATTGCTTCTACATGGCTTTT | GATTATAACTTGGTATTTTAAGGGACTTT | GTGGGTATCTTTCCTCCAGTGYCTC |
| ss9389583 | TGATTTCTTGTATTATCTGTTGCAG | TTTTATAAGCCACCCCTTTATTC | AGCTCTGGACTAGACCCTTCTCTTG |
| ss9398291 | ATGGAACCAACTGAAAAATAACA | TAAATGTCTGTTTTGGAATGGAA | ATTTGCAAACAGGATAGGTTGCAGT |
| ss9403022 | TCTTTTAAAATTTGCCACTGATTT | AGATGTGAAGCCAGTTTAAATTATTAA | ATTTCCTCCCATTAGAAGTCCTCTG |
| ss9406226 | TGAGAGGAGAGAGGAGCCT | ACCCTTGCTTGCAATGTG | GGCTATTGTGTGGAGGGGAGAGTGG |
| ss9409752 | ATAGACAAGGGCTTTCCAGG | AAATTCCATCATATGGGAGTGA | TTCTAGCTTCTTCATTAACCTAAAT |
| ss9419451 | TCTACATGCAAAAAGAGAAATGT | TATTTCAGGGATAGTGACTAATTGAC | AGATCCAGATGGTTGATATTTGAGA |
| ss9419768 | AAATGCTAACTGCTAAAACAGATG | ATTGTCTTCAAATTAGGCAGTATITAT | AGGTAATTTTAACAGCATACCAGTG |
| ss9423342 | AACATTAGTTACAGAGTAAACAGTACTTTCC | TTTCAACTGACTTGGAAGCAG | AAGACTGACAGGGTCAGCTGCTCTG |
| ss9427809 | TAAGAAAGCCAGCCTCACTG | AAATGGGTAAACTICTGTAATCTGC | GTAGGTCAGCCACGAGAAACAA |
| ss9432314 | GGTGACTAAATAAGTCTGAAGTTCAC | ACAACATGCTTAGGTCCTTGTT | AGCCAGTAATCAAGAAGAAAGGATC |
| ss9438029 | AAGAAGACTTCTCAGTTAACCCAA | AAATGTAGGAGGAAAGGATGCT | ACTTGTTATGCAGCAGAGGCTAACC |
| ss9441594 | TTAGAGTGTTCCCTGGAGGC | TGTGTGTAATGTGTCTCTTTCCTC | CCAAATCCAAAAAACAAAGAAAACC |
| ss9442450 | TGATGTAACAAGCCCTGAGAG | AATTCTACTTGACTTCCCTCCTC | CAGCTAGTGCTGGGGGAAGGAGGGG |
| ss9451328 | TGACGTCAGAAGACATCCC | TGGATCCCAGAAGTAAATTTCTAAATATT | AATTTAATTAGAAATAGATGCTACC |
| ss9454084 | ATTATAAGCTAAGAAAATCAACTAACCTAATG | TGTTCTTTATAGTTTCTACATTGTTTGC | AACTGCAATATGCCTTTCTAAGTGA |
| ss9475014 | AAAAGTTAGTCATGTGGTTTTCAGA | TTAACTGAGCTATGCACTTCTCTTT | GTTGGAGGTGGGACTGGTATTATAT |
| ss9480981 | ATGTGGTTGCTGTAAAGATGG | ATATGCTAATAACTACCACTTACGGAA | TGGTAATGCGCATCAAGTGCTTCAA |
| ss9490183 | TTTGTAGCTTCATTTAATAGCATTTATC | AAACAATTCTAGCCAGTACAATTAAAA | ATATTTGTCTCCTACCACCGAAAAA |
| ss9496479 | TTTCTATTGCTCCCTGACCA | GGGCGGTGGGGAAAAGGT | TTTCCCATGGATACCACTGAACTGA |
| ss9502221 | TACTGGGTAAGAACTGGTAGATGG | TAACTCCTAGGTCTTTTCAGTGGA | GGAGAGAAGACAAAAGAAGAAACAG |
| ss9519462 | TGGATACTGATGTAGAGTTATAAGATAATG | GAACTGGAGGGGPAAGGG | CTGAAGAGGTCTGGGCACACCTGAG |
| ss9527721 | AATTCTGTTTTGTTCTAGGCAGAT | TTTTGAGTAACGGTCAGAGGC | AAAAAAGTCCTCTGGAAAAGTCAGC |
| ss9550651 | AGAACATAGTTAGGGCATCC | ATGTGATTGTCTCAGGCTCAA | TTTTTATTACTGGTCTCCATCCTGA |
| ss9565630 | CCACAAATCCAATTTATTCAATCT | CTTGAATTTTCCCCCTTATCTG | GACTTAAGCATTCCACATAGCCCTC |
| ss9574955 | AATAAARGCAAATCCACAGCC | ATGAAAGAGAKAAGAAAAAGAGTGAGA | CACAATTTCCACAAATTGATTTCCC |
| ss9586065 | TTACCCTCTATGGTCCTCC | CTTTCACATTTCTAATAGAGCAGGA | CCATCTTTATCACAAGCCCTGCTGA |
| ss9595292 | ATATGATTAACCTCATTTTACTGACAAG | ATAATGAAGCTGGACAAGAATGA | AGTAGAGAGTGCAAGTGTTTGCCTC |
| ss9602306 | TTGTACAAGACCTTGTAAATTTGG | ACCACCTCATCTGGAAAGC | CTGAGGGCTGAGTAAAACCGCGGGA |
| ss9609977 | TGAGTGGTAAATAGCCAGCTG | TAAGCTGACGATGTGAGTGG | CATTCACTTTAAAAGCATCCACTGC |
| ss9627150 | TTTTCATAAAACTTGAGCACTCTG | TTAAAATGTCTAAATAACTACACGTGATG | TCCTTGGGTCAAAATCAATGCTGTT |
| ss9628837 | TAAACATCAAGGTAACACTGTAGAACTC | AAAAGTCTTTCATTTTTATGATACCC | AAAGAGGTAAATCTTTGTAACCTTG |
| ss9641213 | CTAACTCATTGCTGAGATGGTCT | ACTTCCTGGACTGGCCTC | CAGTGTGGATTTTGAGAAGACAAGT |
| ss9645529 | TGGGTATTTTGATCCTCTTACCT | AGTGAGGAGAAGGAAGGAAGAA | ATGCACTAGCTTTTCTTYCCACCGC |
| ss9646032 | AAGGCAGCACTGAGCAGC | GAAATCAAGAGTTGGACGTTTAAC | AAATATGTATCAAAAGTGACACATG |
| ss9652166 | TTCATCTGTAGGAGGAGCTT | GACGTGTCCAACCTAGGC | TCACGTGGGCCTTGGGAGGCATGAG |
| ss9671733 | ATTATATACAGCCTTGTGGACA | TAAGAACCATTCCCCACG | CATTCATGCCTCTCRGTTTCACAGA |
| ss9672435 | TAGAACCCCCAGCTCATG | GGTCAGAGCATGTCACTCCTA | CTATGCTGACTCCTTCATGTCTTCT |
| ss9678528 | AATCAAAATGTTGGTTCTGCC | TTGATAGTAGTAGTAATAGCCACTCATATG | AATTGAGCTATGGTCTCAAAGGATG |
| ss9684533 | ATAATAGTGCTAATGTTTTATGTAGCCC | AAACCCTGAAATCTCATTGC | AAGCTGATGAGGAATGCCAGTGTCC |
| ss9695373 | TTTCCTTFFTCAGACCCTAGTC | AACTAGGCTTAGGAAACATCTTTAATTA | AAATAATCGGATCTCCAGAGATTTC |
| ss9705100 | ATCTGTCGGAAGGAAGCAA | TCAAAATTTTCCCAGGACCT | AGCATGTGAAGATAGCAAGAGCACT |
| ss9714487 | ACCGTAAATAATAGAGTGCTGAATTT | AAAGCAGAGGAAACCTCACA | AAAGGCAGTGTTACTGTGATATACC |
| ss9719095 | TCTCCTCATGCACTTGTCG | AGGAAGAGGCTGCCATCC | TGTAAACTTGTCTTTCACTGACTAC |
| ss9733605 | AGCTAACTAACTAAATTGTTGTTGCC | TTTAGAGCCATTGCAAAGATTC | TGCTCTGCAGCCTCTACTTCTGGAA |
| ss9734846 | ATTATCTTTCTAAAGCTAGGTGTTGTTG | AGGAAACTTCCCCTAAAAGAGG | CTATCACTGTTGAGATCCTGGC |
| ss9735989 | TTCAATGAATGCAGTGTTTTTTAT | AAAATTTTAAGCTAACCAACCCA | TTGATTCACTTACTGTAACAACTAA |
| ss9759816 | ATATTCATCGCTACTCGGTTTG | TGGAAGAAAGGAAATACAGATGTAA | CTAAGCTTTTACTGTAGACAGACAG |
| ss9780984 | TACATAGAGGTGTGCTGACTTGC | ACAAAATGCAGGTGATAATACCG | ACAGAGCTAACCAGAGCAGAGCCTG |
| ss9788546 | AAAAACTGGGAGAGCTGGAG | TTAGAGAATTlAAGAGAATTTCAACAGG | CAGTTGATGGGTTTCTGAGTTTGCT |
| ss9800286 | AATTTTCATTGAGAATCCAGGG | AGATGTACACCAGATTCCCGT | GTGATAAGTTACTACATAAATAGAA |
| ss9048431 | TATTGACTCTATACCTCTAAAGAATCGC | AGAGTTTCATACTGGGGTAACTTTG | AGACTTTTAAAGTTTAAATGAATTA |
| ss9053109 | TCAGTGAGAAGAGAATCATCCG | TGGCACCAGAAGTAGGTGG | TCTTTACAGACCAGCTTTTGTCTCC |
| ss9067589 | ATTATGATCTGCATCTTCAGAGAC | ATATGGTCTGGGCATGGTTA | AGATAGAGCCAGTCTGTTGGCAAGC |
| ss9069201 | ATTTTGTGGAGAAGTGTGCAG | TTATGCGGGTACTGCTTTATACA | TCTCAGAAAAATGGAAAACAAGAGC |
| ss9084075 | CAGGACGTCTGGAGGTGT | TACAGCTCACTGGCACCTC | CCTGGGCTAGAGTTACAAACATCAG |
| ss9090942 | AAAGCACTCCATACTTAAAGTCA | TTTGTTATCCTAATTCTCTTGGTTTATT | ATTTGCCTTCTTTCACATCCACATG |
| ss9101730 | ATTATATGGTCTCACTTATATGGGGA | ATTTCCCAGAGTTAGGAGTCTCTC | AAAAATAGTGGAAGGGGTTATAGGG |
| ss9108332 | TTAATGGGGATGGTTTGAGA | CAACTGTATCTCTATACATCTCAATCTATCT | GTTAATAAGGGGAAGTAGAGTTTCA |
| ss9132982 | TCTTCCTAAAGGGAAGTTGGG | AATGTAGGAACTGCCTACGTGA | AGGTGACCTGTGGCACTGGGGTCTT |
| ss9139126 | ACTCAAAGCAGCGCCATG | TGGTTGTGTGGTTGGGGC | TAGTGGATTTCACAGATGAGGGAAC |
| ss9142796 | TGTCCAGTCCAATCCCTACC | AGGAGACAGGGATCATCACC | ATCACCTCATGTTATAGACTGGGAA |
| ss9152677 | ATACCCAGAATAGCCGATTTC | ATGATCTTAGAATAGATCTATGCCTTTT | TAATTTCCTGACATGCTTGCTCTGC |
| ss9156891 | CTGCCCRAACCTTCAGAA | AGCCAGGATGGGCCTATT | GTCCAGAGGTACTGCCCAGCAGACA |
| ss9171081 | TATAACCTACCTCTTTCTGCTGC | TTCACATTTAAAACTCCATTTTTG | ACACAGGCTCTCATTCTTACCAATC |
| ss9177956 | TTCAAAAGTAACTGTAGCTTACCAAA | TTCTTTAGATGGAAATTATTTTTAGGC | CCTTAGAAAAGAGGTTAAAAGAAAA |
| ss9186525 | TTCAGTCTTACRACACTTAGCCC | TRGGTAGGTAATGGGAAGGG | TCTGCCTCATCCATTCAGCTGTACT |
| ss9191087 | AATCCACCTGCCCAGCCC | AATATTAGAAAACACAGCAAGCTTCT | CCGGATTTAATTAAGGTAGTCTTAC |
| ss9200241 | CCTATCTTTGCACTCACTGTTA | ATTCACTCATTGAAATATATATCCCC | TTCATTCGAGATTTAGTTTATGACA |
| ss9230071 | TTTCTGGTAATCATACAGATAAAGG | ATCCCTGAAGACACATTTCATG | TAGCTGGAGCATTCATTAGGATCRC |
| ss9233837 | TGAACATTTTCTTCAAGTATCAAATC | CTTTGTATATTTACAAGGACAAGGC | CAAGTAGGCATGTTAATATCATAAA |
| ss9235114 | ATGCTTCATCCTCTGGTGTG | TATATGGGAAATTAGGAAGGTATTGT | TGTTCATAGGCCAATACTCGTACCA |
| ss9244345 | TTAATACCCATTACCTAGTTCCCC | TCTAAAGGCAGGATCAGAGG | TAAAAAGAAATGTCTACCTCACTGA |
| ss9245977 | AGAGGTTGCCCTTTGCCA | AACCCACATGTAAAAATACTAGTGC | CTTAATTAGTTACATGCTGCCTTGG |
| ss9251154 | TGGACCTTAGAGTCTTCGAGC | AACAGGCTGCCCTAGAAGAT | ACAGAAGGTCTCCCATTGGCTGCTC |
| ss9259716 | ATATCCCATAACCAAGACTGCA | TTGACAGCTCTTGGCAACA | GGTCCTTGTTTATCTTTTCCAAAAA |
| ss9270557 | CTGAAGGCCCACAACTTG | ATTAGTCTTGAATATCTCAATCAATATGG | ATTATTAAACAATCATATTTGTAGC |
| ss9278814 | TTTGGAAAGGAAAAGAATTAGTAT | ATGTGGGAAAAGAGACTTAAATGT | TTTAGTACATTAACAACATCCTCAA |
| ss9281595 | GCTAAGAGGAGGAAGAAAATG | AATTGTGGATTATGGGTGA | CATGGGAATAATAGAGGAAATTGTA |
| ss9282411 | TTCTGGTCTAGAGAGAGGTTTTTT | ATGAAATATCTCCAGACCTGGG | CACCATGTGGCCCAACATCAGTGCT |

TABLE 8
5′ Nucleic acid sequence containing SNP.

| | |
|---|---|
| Sequence | Breed Identity Markers |

| | | |
|---|---|---|
| 5′TCTATACCTCTAAAGAATCGCTGCTACTTTGTGCAAGACTTTTAAAG | (SEQ ID NO:1) | ss9048431 |
| TTTAAATGAATTA A/G 3′ | | |
| 5′GTTTATGGTTTTATGTGTCTGATAACAGGAAGAACTTTACAGACCAGCT | (SEQ ID NO:2) | ss9053109 |
| TTTGTCTCC A/G 3′ | | |
| 5′TGATCTGCATCTTCAGAGACCATGATCCAGAAAGAAGATAGAGCCAGTCTG | (SEQ ID NO:3) | ss9067589 |
| TTGGCAAGC A/G 3′ | | |
| 5′AGAAGGAGGAGGAATCATAAAGGAAGCAAAATAATTCTCAGAAAAATGGAA | (SEQ ID NO:4) | ss9069201 |
| AACAAGAGC A/G 3′ | | |
| 5′TACTGGCCTCCACACCAGGACGTCTGGAGGTGTCTCCTGGGCTAGAGTTAC | (SEQ ID NO:5) | ss9084075 |
| AAACATCAG A/G 3′ | | |
| 5′GGAAAGTGTTCAAAGCACTCCATACTTAAAGTCACATTGCCTTCTTTCAC | (SEQ ID NO:6) | ss9090942 |
| ATCCACATG A/G 3′ | | |
| 5′CAAACATTATATGGTCTCACTTATATGGGGAATATAAAAATAGTGGAAGGG | (SEQ ID NO:7) | ss9101730 |
| GTTATAGGG A/G 3′ | | |
| 5′GAGAGAGATGGCTTTTAATGGGGATGGTTTGAGAGGTTAATAAGGGGAAGT | (SEQ ID NO:8) | ss9108332 |
| AGAGTTTCA A/G 3′ | | |
| 5′GCACCTGAAGCTCTTCCTAAAGGGAAGTTGGGCAGAGGTGACCTGTGGCAC | (SEQ ID NO:9) | ss9132982 |
| TGGGGTCTT A/G 3′ | | |
| 5′TTTACCTCTTACAACACTCAAAGCAGCGCCATGACATAGTGGATTTCACAGA | (SEQ ID NO:10) | ss9139126 |
| TGAGGGAAC A/G 3′ | | |
| 5′GAAGGGACTCTTCCTGTCCAGTCCAATCCCTACCCATCACCTCATGTTATA | (SEQ ID NO:11) | ss9142796 |
| GACTGGGAA A/G 3′ | | |
| 5′CCATAAAAGTTTTAGAACAGATCATCTTAAAAAGGTTAATTTCCTGACATGC | (SEQ ID NO:12) | ss9152677 |
| TTGCTCTGC A/G 3′ | | |
| 5′CCTTCCTTCTCAGGCCTGCCCAAACCTTCAGAATGGTCCAGAGGTACTGCC | (SEQ ID NO:13) | ss9156891 |
| CAGCAGACA A/G 3′ | | |
| 5′GGTCTCCTATAACCTACCTCTTTCTGCTGCCTGCCACACAGGCTCTCATTC | (SEQ ID NO:14) | ss9171081 |
| TTACCAATC A/G 3′ | | |
| 5′CAAAATGCTTACTCATAGGCCTTAATAAATAAGCACCTTAGAAAAGAGGTT | (SEQ ID NO:15) | ss9177956 |
| AAAAGAAAA A/G 3′ | | |
| 5′AGGATAGACAGGGATTTGGTCCCC3TTATCCTCATTCTGCCTCATCCATTC | (SEQ ID NO:16) | ss9186525 |
| AGCTGTACT A/G 3′ | | |
| 5′TCACAGGGCCCAATCCACCTGCCCAGCCCTCACTCCCGGATTTAATTAAGG | (SEQ ID NO:17) | ss9191087 |
| TAGTCTTAC A/G 3′ | | |
| 5′GATTTCTTTCTTTCTTCCTTTCTTTCATTCATTCATTCATTCGAGATTTAG | (SEQ ID NO:18) | ss9200241 |
| TTTATGACA A/G 3′ | | |
| 5′GGGTCAGGGTTTCTGGTAATCATACAGATAAAGGGTAGCTGGAGCATTCAT | (SEQ ID NO:19) | ss9230071 |
| TAGGATCAC A/G 3′ | | |
| 5′TTGAACATTTTCTTCAAGTATCAAATCTTAATCCTCAAGTAGGCATGTTAA | (SEQ ID NO:20) | ss9233837 |
| TATCATAAA A/G 3′ | | |
| 5′CTTAAAAGATGCTTCATCCTCTGGTGTGCATTCCATGTTCATAGGCCAATA | (SEQ ID NO:21) | ss9235114 |
| CTCGTACCA A/G 3′ | | |
| 5′CTAGTTCCCCATCTCCCCACTCACCTCCTCTGTTTTAAAAAGAAATGTCTA | (SEQ ID NO:22) | ss9244345 |
| CCTCACTGA A/G 3′ | | |
| 5′TACCCACTGTGACACAGAGGTTGCCCTTTGCCAAACTTAATTAGTTACATG | (SEQ ID NO:23) | ss9245977 |
| CTGCCTTGG A/G 3′ | | |
| 5′GACATTGGAGCCGTGGACCTTAGAGTCTTCGAGCCACAGAAGGTCTCCCAT | (SEQ ID NO:24) | ss9251154 |
| TGGCTGCTC A/G 3′ | | |
| 5′GAGTCTTCTCATATCCCATAACCAAGACTGCAAATGGTCCTTGTTTATCTT | (SEQ ID NO:25) | ss9259716 |
| TTCCAAAAA A/G 3′ | | |
| 5′TCACCTTTTCTAGCCTGAAGGCCCACAACTTGTGAATTATTAAACAATCAT | (SEQ ID NO:26) | ss9270557 |
| ATTTGTAGC A/G 3′ | | |
| 5′GACTCAATTCTTTGGAAAGGAAAAGAATTAGTATATTTAGTACATTAACAA | (SEQ ID NO:27) | ss9278814 |
| CATCCTCAA A/G 3′ | | |
| 5′TGAATCTTCTTTGGCTAAGAGGAGGAAGAAAATGCCATGGGAATAATAGAG | (SEQ ID NO:28) | ss9281595 |
| GAAATTGTA A/G 3′ | | |
| 5′TCTAGAGAGAGGTTTTTTGTTTCTTCTCAGGGATCCACCATGTGGCCCAAC | (SEQ ID NO:29) | ss9282411 |
| ATCAGTGCT A/G 3′ | | |
| 5′CTTTTACAGATCCTTCCCCAGGAGCACACTGTGTCGTCACATTTTGAAACA | (SEQ ID NO:30) | ss9285114 |
| ACCTTAGCT A/G 3′ | | |
| 5′AATGCACAGAACAAAGTCCCTGCTTTCATGTAGATTTCATTTTTGTCAGGA | (SEQ ID NO:31) | ss9290112 |
| GGTAAAGAT A/G 3′ | | |
| 5′CAAGCAGCAACCCTGCACCCTCCCTGTCAACTTGGTTCTTTCATTTTGCTG | (SEQ ID NO:32) | ss9290361 |
| TGGGCGGCT A/G 3′ | | |
| 5′GAACTGCAAAAAGTGGAAAATTGATTCATATGCTACTATCCGGTCAACAAA | (SEQ ID NO:33) | ss9292376 |
| TGTGCCCAT A/G 3′ | | |
| 5′AGATAATTTCATATTTCTGTTTTCTGCATGTTGCTATGTCACTTTGCAAGT | (SEQ ID NO:34) | ss9294456 |
| ATAAACTGA A/G 3′ | | |
| 5′ACTAGTGAGGCAAGAGGGTGATGAATAGTATCCTACATGAGGCCATTGTTT | (SEQ ID NO:35) | ss9296487 |
| GCAGAAGCA A/G 3′ | | |
| 5′TCCCTGAGTGAAGTAAATATTGAATAAGTTTATCTGTAGTGGCATTTCCAT | (SEQ ID NO:36) | ss9300915 |
| CTAGGAGAG A/G 3′ | | |
| 5′GCAGATCTCCACTCCCTTTTCCACTCTACAATTTGCATTTATCTCTCTTAG | (SEQ ID NO:37) | ss9301348 |
| AGTTTTAGG A/G 3′ | | |
| 5′CTAGCATATTCCACATTTGGAAGTTAATCTGATTTCTTTACACAGGACCTG | (SEQ ID NO:38) | ss9307596 |
| AAGGCTCAC A/G 3′ | | |
| 5′GCAGGGATCTTGGAAGTGGACCTGCAGGCATGAACTAGGGAGGCTCAAGA | (SEQ ID NO:39) | ss9308314 |
| CCTTTAGGT A/G 3′ | | |
| 5′CTGAATTCATGAATGTTAGTTTATTCTTATTGTTTGCTACCTGGGATGTTA | (SEQ ID NO:40) | ss9313462 |
| GGTCATTAA A/G 3′ | | |
| 5′CCTTTTCTCCACATAGCTGTTAAGTGTCCATGTCTTATTGATTTGCCGTGG | (SEQ ID NO:41) | ss9313564 |
| GTTCTGTCC A/G 3′ | | |
| 5′ATTGACTCTCTCAAATAATCATCTGGATTGACCTCAGAGAGTGCTTCGACT | (SEQ ID NO:42) | ss9313781 |
| CAACGCCCA A/G 3′ | | |
| 5′TCTCTGGGTGCTCCCAGGGCAGTGTGTGCGCTATTCCTCAGACCAAAATGG | (SEQ ID NO:43) | ss9328275 |
| GACCAGACC A/G 3′ | | |
| 5′CATAGGACAATAGTTTTTTTTTTTTTTAATCTTTAGTGAAGGAGATTCAGA | (SEQ ID NO:44) | ss9335917 |
| GTAGCATAG A/G 3′ | | |
| 5′GCAACTGTATTTATCTGCCTGATTTTAATTATGATGATAAGAGTTCCCCTA | (SEQ ID NO:45) | ss9339680 |
| AGCTGGATT A/G 3′ | | |
| 5′AGCCTTCCTTAAGACAGGTGAGAATCCTGACAGCGGCATTGTACTGAGCAC | (SEQ ID NO:46) | ss9362797 |
| ATCCTCGCG A/G 3′ | | |
| 5′TCACTTGATTCTCTTTATCGGCTACTAGAAAATTTTAAGCTACAGATGTGA | (SEQ ID NO:47) | ss9366135 |
| ATTGCATTT A/G 3′ | | |
| 5′AGGAGTTCACTGGTCTCCTAGGATTTGCTCATTTAATACTGTTGTAGAAGC | (SEQ ID NO:48) | ss9366251 |
| TTTTGTTTT A/G 3′ | | |
| 5′TAAAATTCAACCAATATACATATTTTGAAGATGTGGGTGAACCCTTCTGTT | (SEQ ID NO:49) | ss9378306 |
| TCTGTGAAA A/G 3′ | | |
| 5′TTGTATTTCCCGTGCAGGTCGTATGCATATCATCCACGTACCACACACGTT | (SEQ ID NO:50) | ss9380511 |
| ATACACGTA A/G 3′ | | |
| 5′CATGGCTTTTAGATCTTTAATTTCCTGATATTTCAGTGGGTATCTTTCCTC | (SEQ ID NO:51) | ss9382377 |
| CAGTGTCTC A/G 3′ | | |
| 5′TCATGGTCCAGTAAGAAACAGACGACTTGCATGAAAGCTCTGGACTAGACC | (SEQ ID NO:52) | ss9389583 |
| CTTCTCTTC A/G 3′ | | |
| 5′TGGAACCAACTGAAAAATAACAATGTTTTTTCTACAThTGCAAACAGGATA | (SEQ ID NO:53) | ss9398291 |
| GGTTGCAGT A/G 3′ | | |
| 5′AGTCTTTTAAAATTTGCCACTGATTTTACAGGATCATTTCCTCCCATTAGA | (SEQ ID NQ:54) | ss9403022 |
| AGTCCTCTG A/G 3′ | | |
| 5′CCACAAGGTCAGGACTGAGAGGAGAGAGGAGCCTGGGCTATTGTGTGGAGG | (SEQ ID NO:55) | ss9406226 |
| GGAGAGTGG A/G 3′ | | |
| 5′CATACCCCATCATAGACAAGGGCTTTCCAGGAATGTTCTACCTTCTTCATT | (SEQ ID NO:56) | ss9409752 |
| AACCTAAAT A/G 3′ | | |
| 5′CATGCAAAAATGAGAAATGTGGGAGCATAGGGCAGAGATCCAGATGGTTGA | (SEQ ID NO:57) | ss9419451 |
| TATTTGAGA A/G 3′ | | |
| 5′AAATGGATGGAAATGCTAACTGCTAAAACAGATGAAGGTAATTTTAACAGC | (SEQ ID NO:58) | ss9419768 |
| ATACCAGTG A/G 3′ | | |
| 5′TCCAACATTAGTTACAGAGTAAACAGTACTTTCCAAAGACTGACAGGGTCA | (SEQ ID NO:59) | ss9423342 |
| GCTGCTCTG A/G 3′ | | |
| 5′ATCTTTCCAACTAAGAAAGCCAGCCTCACTGGGTATTTGTAGGTCAGCCAC | (SEQ ID NO:60) | ss9427809 |
| GAGAAACAA A/G 3′ | | |
| 5′CGTGATGGGTGACTAAATAAGTCTGAAGTTCACAGAGCCAGTAATCAAGAA | (SEQ ID NO:61) | ss9432314 |
| GAAAGGATC A/G 3′ | | |
| 5′CACGTTTGCTGTTTCACACCACTAAGTTTGGCATCACTTGTTATGCAGCAG | (SEQ ID NO:62) | ss9438029 |
| AGGCTAACC A/G 3′ | | |
| 5′GTGTTCCCTGGAGGCTCCAAGAGGCTCAGTGATCGCCAAATCCAAAAAACA | (SEQ ID NO:63) | ss9441594 |
| AAGAAAACC A/G 3′ | | |
| 5′AGAGAGCTAGAGCTGATGTAACAAGCCCTGAGAGCCAGCTAGTGCTGGGGG | (SEQ ID NO:64) | ss9442450 |
| AAGGAGGGG A/G 3′ | | |
| 5′ACATCCCCAAGCTTCCTGAAACTGATCTGCGGCAGAATTTAATTAGAAATA | (SEQ ID NO:65) | ss9451328 |
| GATGCTACC A/G 3′ | | |
| 5′TGAAATGGGCCAGACTAGCATCTGCTCTAGTTCCTAACTGCAATATGCCTT | (SEQ ID NO:66) | ss9454084 |
| TCTAAGTGA A/G 3′ | | |
| 5′GAAGATGAAAGCTTAAGATAAAGCCTGAGAGTGATGTTGGAGGTGGGACTG | (SEQ ID NO:67) | ss9475014 |
| GTATTATAT A/G 3′ | | |
| 5′CCTGCATCATGTGGTTGCTGTAAAGATGGTAACGTTGGTAATGCGCATCAA | (SEQ ID NO:68) | ss9480981 |
| GTGCTTCAA A/G 3′ | | |
| 5′TTTAATAGCATTTATCACCATCTAATATCCAATGCATATTTGTCTCCTACC | (SEQ ID NO:69) | ss9490183 |
| ACCGAAAAA A/G 3′ | | |
| 5′ACCTTTTCAAACACCCTTTACCATATGTATTGGTGTTTCCCATGGATACCA | (SEQ ID NO:70) | ss9496479 |
| CTGAACTGA A/G 3′ | | |
| 5′CTGGTAGATGGATGCAAGGAAAGAAAAAAGAAAAAGGAGAGAAGACAAAA | (SEQ ID NO:71) | ss9502221 |
| GAAGAAACAG A/G 3′ | | |
| 5′TCGTTGGATACTGATGTAGAGTTATAAGATAATGTCTGAAGAGGTCTGGGC | (SEQ ID NO:72) | ss9519462 |
| ACACCTGAG A/G 3′ | | |
| 5′TTGTTCTAGGCAGATTTAAAAACGAAACTCTGACCAAAAAAGTCCTCTGGA | (SEQ ID NO:73) | ss9527721 |
| AAAGTCAGC A/G 3′ | | |
| 5′TCTCTTTGGCATGCAGAACATAGTTAGGGCATCCCTTTTTATTACTGGTCT | (SEQ ID NO:74) | ss9550651 |
| CCATCCTGA A/G 3′ | | |
| 5′CTCCCCCACAAATCCAATTTATTCAATCTATTTTAGACTTAAGCATTCCAC | (SEQ ID NO:75) | ss9565630 |
| ATAGCCCTC A/G 3′ | | |
| 5′AAAGCAAATCCACAGCCTGTCGCCCCGACAAACATCACAATTTCCACAAAT | (SEQ ID NO:76) | ss9574955 |
| TGATTTCCC A/G 3′ | | |
| 5′GTGTCCCATTCGTCCTTACCCTCTATGGTCCTCCTCCATCTTTATCACAAG | (SEQ ID NO:77) | ss9586065 |
| CCCTGCTGA A/G 3′ | | |
| 5′TGATTAACCTCATTTTACTGACAAGAAAGCTGTGTAGTAGAGAGTGCAAGT | (SEQ ID NO:78) | ss9595292 |
| GTTTGCCTC A/G 3′ | | |
| 5′CCTTGTAAATTTGGGCAAGGAATTTGATTTTTATTCTGAGGGCTGAGTAAA | (SEQ ID NO:79) | ss9602306 |
| ACCGCGGGA A/G 3′ | | |
| 5′ATCAGTAACTGAGTGGTAAATAGCCAGCTGGTCAACATTCACTTTAAAAGC | (SEQ ID NO:80) | ss9609977 |
| ATCCACTGC A/G 3′ | | |
| 5′TTTTTAGCTTTTCATAAAACTTGAGCACTCTGAGGTCCTTGGGTCAAAATC | (SEQ ID NO:81) | ss9627150 |
| AATGCTGTT A/G 3′ | | |
| 5′ATCAAGGTAACACTGTAGAACTCTTAGAATAAAACAAAGAGGTAAATCTTT | (SEQ ID NO:82) | ss9628837 |
| GTAACCTTG A/G 3′ | | |
| 5′ATTGCTGAGATGGTCTCAAGATGGAGAGTTTGTGGCAGTGTGGATTTTGAG | (SEQ ID NO:83) | ss9641213 |
| AAGACAAGT A/G 3′ | | |
| 5′TATGATGGCTCTGGTGCATAACATTCTTCAATTTGATGCACTAGCTTTTCT | (SEQ ID NO:84) | ss9645529 |
| TTCCACCGC A/G 3′ | | |
| 5′AGAATATCTGATCAGAAGGCAGCACTGAGCAGCAGAAATATGTATCAAAAG | (SEQ ID NO:85) | ss9646032 |
| TGACACATG A/G 3′ | | |
| 5′GGCACCTCTGGTTCTTCATCTGTAGGAGGAGCTTGTCACGTGGGCCTTGGG | (SEQ ID NO:86) | ss9652166 |
| AGGCATGAG A/G 3′ | | |
| 5′CCCAGACAGCTCATTATATACAGCCTTGTGGACAACATTCATGCCTCTCGG | (SEQ ID NO:87) | ss9671733 |
| TTTCACAGA A/G 3′ | | |
| 5′TGAGCCAGATGAGGTAGAACCCCCAGCTCATGGTGAGAGTTTGTGAGTTTA | (SEQ ID NO:88) | ss9672435 |
| TTCTGAGCT A/G 3′ | | |
| 5′TGGACTAGAACAGGCAGAGTAAACGTGCTGGCCCTAATTGAGCTATGGTCT | (SEQ ID NO:89) | ss9678528 |
| CAAAGGATG A/G 3′ | | |
| 5′TACACCTGTGAGGCCTGCCCCGGCTCTTCATCCCCAAGCTGATGAGGAATG | (SEQ ID NO:90) | ss9684533 |
| CCAGTGTCC A/G 3′ | | |
| 5′TCAGCTTCCTAATTTCCTTTTTCAGACCCTAGTCTAAATAATCGGATCTCC | (SEQ ID NO:91) | ss9695373 |
| AGAGATTTC A/G 3′ | | |

Although the invention has been described with reference to the above example, it will be understood that modifications and variations are encompassed within the spirit and scope of the invention. Accordingly, the invention is limited only by the following claims.

1-19. (canceled)

20. A method for inferring a phenotype or genetic trait of a canine subject from a target nucleic acid sample of the subject, the method comprising identifying, in the nucleic acid sample, at least one nucleotide occurrence of a single nucleotide polymorphism (SNP) corresponding to the first nucleotide, or complement thereof, in the most 3′ position of any one of SEQ ID NOs:1-101, wherein the nucleotide occurrence is associated with the phenotype or genetic trait.

21. The method of claim 20, wherein the nucleotide occurrence of at least 2 SNPs is determined.

22. The method of claim 21, wherein the at least 2 SNPs comprise a haplotype, wherein the method identifies a haplotype allele that is associated with the trait.

23. The method of claim 22, comprising identifying a diploid pair of haplotype alleles.

24. The method of claim 20, wherein the trait is disease resistance or disease susceptibility.

25. The method of claim 20, wherein the sample is isolated from a tissue or a bodily fluid.

26. The method of claim 20, wherein the target nucleic acid molecule is a DNA molecule.

27. The method of claim 26, wherein the DNA molecule is genomic DNA.

28. The method of claim 26, wherein the DNA molecule is double-stranded DNA.

29. The method of claim 26, wherein the DNA molecule is single-stranded DNA.

30. The method of claim 26, wherein the nucleic acid molecule is an RNA molecule.

31. A method for identifying a phenotype or genetic trait of a canine test subject, the method comprising:
a) obtaining a target nucleic acid sample from the test subject by a method comprising identifying in the nucleic acid sample at least one single nucleotide polymorphism (SNP) corresponding to the first nucleotide, or complement thereof, in the most 3′ position of any one of SEQ ID NOs:1-101;
b) repeating a) for additional subjects;
c) determining the allele frequency corresponding to each SNP identified; and
d) comparing the allele frequency of the test subject with each additional subject.

32-37. (canceled)

38. A database comprising each single nucleotide polymorphism (SNP) corresponding to the first nucleotide, or complement thereof, in the most 3′ position of any one of SEQ ID NOs:1-101.

39. A database comprising allele frequencies generated by analyzing the database of claim 38.

40. A method for inferring a phenotype or genetic trait of a canine subject from a target nucleic acid sample of the subject, the method comprising identifying, in the nucleic acid sample, at least one nucleotide occurrence of a single nucleotide polymorphism (SNP) corresponding to the first nucleotide, or complement thereof, in the most 3′ position of any one of the sequences set forth in the GenBank Accession numbers of Table 8, wherein the nucleotide occurrence is associated with the phenotype or genetic trait.

41. A computer-based method for identifying or inferring a trait of a canine test subject, the method comprising:
a) obtaining a nucleic acid sample from the subject;
b) identifying in the nucleic acid sample at least one nucleotide occurrence of at least one single nucleotide polymorphism (SNP) corresponding to the first nucleotide, or complement thereof, in the most 3′ position of any one of SEQ ID NOs:1-101;
c) searching a database comprising allele frequencies of claim 39;
d) retrieving the information from database;
e) optionally storing the information in a memory location associated with a user such that the information may be subsequently accessed and viewed by the user; and
f) identifying the trait of a canine subject.

42. A method for identifying or inferring a trait of a canine test subject from a nucleic acid sample obtained from the subject, the method comprising:
a) contacting the nucleic acid sample with a pair of oligonucleotides that comprise a primer pair, wherein amplified target nucleic acid molecules are produced;
b) hybridizing at least one oligonucleotide primer selected from the group consisting of SEQ ID NOS:304-404 to one or more amplified target nucleic acid molecules, wherein each oligonucleotide primer is complementary to a specific and unique region of each target nucleic acid molecule such that the 3′ end of each primer is proximal to a specific and unique target nucleotide of interest;
c) extending each oligonucleotide with a template-dependent polymerase; and
d) determining the identity of each nucleotide of interest by determining, for each extension primer employed, the identity of the nucleotide proximal to the 3′ end of each primer.

43. The method of claim 41, wherein the primer pair is any of the forward and reverse primer pairs listed in Table 7.

44. The method of claim 41, wherein a first primer of the primer pair is selected from SEQ ID NOS:102-202 and the second primer of the primer pair is selected from SEQ ID NOS:203-303.

45. (canceled)

46. (canceled)

47. An isolated single nucleotide polymorphism (SNP) corresponding to the first nucleotide, or complement thereof, in the most 3′ position of any one of SEQ ID NOs:1-101.

48-51. (canceled)

52. A panel comprising at least one single nucleotide polymorphism (SNP) corresponding to the first nucleotide, or complement thereof, in the most 3′ position of any one of SEQ ID NOs:1-101.

53. (canceled)

54. (canceled)

55. A method to infer breed or line of a canine test subject from a nucleic acid sample obtained from the subject, comprising identifying in the nucleic acid sample, at least one nucleotide occurrence of at least one single nucleotide polymorphism (SNP) corresponding to the first nucleotide, or complement thereof, in the most 3′ position of any one of SEQ ID NOs:1-101, wherein the SNP is associated with a breed, thereby inferring the breed of the canine subject.

56. A method of generating a genome discovery map comprising:
a) selecting a plurality of single nucleotide polymorphism (SNP) markers selected from at least two of the SNP markers corresponding to the first nucleotide, or complement thereof, in the most 3′ position of any one of SEQ ID NOs:1-101, wherein each marker in the series will be separated by approximately 150,000 bp; and
b) generating the genome discovery map based upon the selected markers.

57. The method of claim 56, wherein the genome discovery map is a whole genome discovery map.

58. The method of claim 56, wherein the plurality of single nucleotide polymorphism (SNP) markers includes about 10 markers.

59. The method of claim 56, wherein the plurality of single nucleotide polymorphism (SNP) markers includes about 100 markers.

60. The method of claim 56, wherein the plurality of single nucleotide polymorphism (SNP) markers includes about 1000 markers.

61. The method of claim 56, wherein the plurality of single nucleotide polymorphism (SNP) markers includes about 5000 markers.

62. The method of claim 56, wherein the plurality of single nucleotide polymorphism (SNP) markers includes about 10000 markers.

63. The method of claim 56, wherein the discovery map is a canine discovery map.

64. The method of claim 56, wherein the plurality of single nucleotide polymorphism (SNP) markers, or the number of markers indicated by the amount of linkage disequilibrium in a canine species, are further selected based upon dispersion across the entire genome.

65. (canceled)

66. A method to infer parentage or breed of a canine test subject from a nucleic acid sample obtained from the subject, comprising identifying in the nucleic acid sample, at least one nucleotide occurrence of at least one single nucleotide polymorphism (SNP) corresponding to the first nucleotide, or complement thereof, in the most 3′ position of any one of SEQ ID NOs:1-101, wherein the SNP is associated with a breed, thereby inferring the breed of the canine subject.

67. (canceled)

68. A database for determining breed or parentage of a canine subject, the database comprising each single nucleotide polymorphism (SNP) corresponding to the first nucleotide, or complement thereof, in the most 3′ position of any one of SEQ ID NOs:1-101.
